(19) United States
(12) Patent Application Publication
(10) Pub. No.: US 2023/0133504 A1
(43) Pub. Date: May 4, 2023

(54) METHOD OF TREATING GRAFT-VERSUS-HOST DISEASE

(71) Applicant: REGENTS OF THE UNIVERSITY OF MICHIGAN, Ann Arbor, MI (US)

(72) Inventors: Pavan Reddy, Bloomfield Hills, MI (US); Haibin Zhou, Ann Arbor, MI (US); Dongchang Zhao, Ann Arbor, MI (US); Shaomeng Wang, Superior Township, MI (US); Longchuan Bai, Ann Arbor, MI (US)

(21) Appl. No.: 17/886,776

(22) Filed: Aug. 12, 2022

Related U.S. Application Data
(60) Provisional application No. 63/233,012, filed on Aug. 13, 2021.

(51) Int. Cl. A61K 31/661; A61P 37/06

(52) U.S. Cl. CPC A61K 31/661; A61P 37/06

(57) ABSTRACT

The present disclosure relates to methods of treating or preventing graft-versus-host disease (GVHD) comprising administering to the subject a pharmaceutically effective amount of a compound disclosed herein. The present disclosure also relates to pharmaceutical compositions and pharmaceutical kits suitable for the treatment or prevention.

### RELATED APPLICATION

This application claims priority to, and the benefit of, U.S. Provisional Application No. 63/233,012, filed Aug. 13, 2021, the entire contents of which are incorporated herein by reference.

### BACKGROUND

Graft-versus-host disease (GVHD) is a condition that occurs following a transplant in a subject when immune competent T cells in the graft recognize the host as foreign. GVHD is most commonly observed in the context of an allogeneic transplants, including hematopoietic cell transplantation (HCT) for treatment for many hematological malignancies. A standardized therapeutic is still lacking, and many patients fail to respond to first-line therapy.

Acute graft versus host disease (GVHD) remains a big barrier to cure malignant or genetic diseases treated with allogeneic hematopoietic stem cell transplantation (HSCT). Generally, GVHD pathophysiology is a reaction of donor T cells against host tissues, which including at least three major of target organs: skin, liver, and gut. Although this pathogenesis is complicated, STAT3 signaling in donor T cells is considered to play a critical role during GVHD development (Blood, 2008; 112: 5254-5258; The Journal of Immunology, 2010, 184: 764-774; Immunity, 2012; 37: 209-222; J. Leukoc. Biol., 2015, 97: 807-819). A standardized therapeutic is still lacking, and many patients fail to respond to first-line therapy. Thus, there is a need for compositions and methods to treat and/or prevent GVHD. The present disclosure provides compositions and methods for treating and preventing GVHD by use of compounds presented herein.

### SUMMARY

In some aspects, the present disclosure provides a method of treating or preventing graft-versus-host disease (GVHD) in a subject, comprising administering to the subject a pharmaceutically effective amount of a compound disclosed herein.

In some aspects, the present disclosure provides a method of treating graft-versus-host disease (GVHD) in a subject, comprising administering to the subject a pharmaceutically effective amount of a compound disclosed herein.

In some aspects, the present disclosure provides a compound disclosed herein for treating or preventing GVHD in a subject.

In some aspects, the present disclosure provides a compound disclosed herein for treating GVHD in a subject.

In some aspects, the present disclosure provides use of a compound disclosed herein in the manufacture of a medicament for treating or preventing GVHD in a subject.

In some aspects, the present disclosure provides use of a compound disclosed herein in the manufacture of a medicament for treating GVHD in a subject.

In some aspects, the present disclosure provides a pharmaceutical composition for treating or preventing GVHD in a subject, comprising a compound disclosed herein.

In some aspects, the present disclosure provides a pharmaceutical composition for treating GVHD in a subject, comprising a compound disclosed herein.

In some aspects, the present disclosure provides a pharmaceutical kit for treating or preventing GVHD in a subject, comprising a compound disclosed herein.

In some aspects, the present disclosure provides a pharmaceutical kit for treating GVHD in a subject, comprising a compound disclosed herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTIONS OF FIGURES

FIGS. 1A-1C. Compound No. 36 treatment of acute GVHD model. BALB/c recipient mice on day 0 received 5×10⁶ of bone marrow cells and 1×10⁶ of purified splenic T cells from allogeneic C57BL/6 donor or syngeneic BALB/c donor following lethal irradiation with 800 rads on day −1. Recipients received intraperitoneal injections of 25 mg/kg of Compound No. 36 or vector starting on day −1, then three times weekly, from day −1 to day 21. Mice were monitored weekly for survival and GVHD score. FIG. 1A shows body weight changes of animals measured weekly for each group as indicated. FIG. 1B shows GVHD scoring of animals followed weekly for progression of disease. FIG. 1C shows the survival curve based on daily observations of the animals. Data in *, P<0.05; **, P<0.01. Syn—syngenic; Allo—allogeneic.

FIGS. 2A-2C. Compound No. 36 treatment of acute GVHD model challenged with A20 tumor cells. Using the same method as described in FIGS. 1A-1C, BALB/c recipient mice underwent major histocompatibility complex (MHC) major mismatch allogeneic bone marrow transplantation (BMT) with C57BL/6 donor or with BALB/c donor as syngeneic control. BALB/c recipient mice on day 0 received 5×10⁶ of bone marrow cells and 1×10⁶ of purified splenic T cells with or without 0.5×10⁶ A20 cells inoculum. As indicated here, recipients received intraperitoneal injections of 25 mg/kg of Compound No. 36 or vector starting on day −1, then three time weekly, from day −1 to day 21. Mice were monitored weekly for survival and GVHD score. FIG. 2A indicates body weight changes of animals measured weekly. FIG. 2B shows tumor-related death curve based on daily observations of the animals.

FIG. 2C shows the survival curve based on daily observations of the animals. Data in *, P<0.05; **, P<0.01. Syn—syngenic; Allo—allogeneic.

FIGS. 3A-3D display the effect of Compound No. 36 on intestinal organoids culture in vitro. Intestinal organoids were established from culture of small intestinal crypts in GFP-Lgr5 transgene B6 mice. 3-5 passages of organoids were used to test the effect of Compound No. 36 in vitro. After dissociation of organoids, 100 crypts per well mixed with Matrigel-culture medium were plated in 24-well plate. After the Matrigel polymerized, 500 μL of culture medium with different concentrations of Compound No. 36 or vector were added to each well. The growth of the organoids was checked daily. Three days after coculture, organoid numbers and budding numbers were counted under the microscope and were photographed (FIG. 3A). Then, organoids were mechanically disrupted and dissociated into single cells with TrypLE Express. Cells were stained with anti-CD326, annexin V, and 7-AAD to analyze GFP-Lgr5^{hi} cells apoptosis by flow cytometry (FIGS. 3B-3D).

### DETAILED DESCRIPTION

### Methods and Uses of the Present Disclosure

In some aspects, the present disclosure provides a method of treating or preventing GVHD in a subject, comprising administering to the subject a pharmaceutically effective amount of a compound disclosed herein.

In some aspects, the present disclosure provides a method of treating GVHD in a subject, comprising administering to the subject a pharmaceutically effective amount of a compound disclosed herein.

In some aspects, the present disclosure provides a compound disclosed herein for treating or preventing GVHD in a subject.

In some aspects, the present disclosure provides a compound disclosed herein for treating GVHD in a subject.

In some aspects, the present disclosure provides use of a compound disclosed herein in the manufacture of a medicament for treating or preventing GVHD in a subject.

In some aspects, the present disclosure provides use of a compound disclosed herein in the manufacture of a medicament for treating GVHD in a subject.

In some aspects, the present disclosure provides a pharmaceutical composition for treating or preventing GVHD in a subject, comprising a compound disclosed herein.

In some aspects, the present disclosure provides a pharmaceutical composition for treating GVHD in a subject, comprising a compound disclosed herein.

In some aspects, the present disclosure provides a pharmaceutical kit for treating or preventing GVHD in a subject, comprising a compound disclosed herein.

In some aspects, the present disclosure provides a pharmaceutical kit for treating GVHD in a subject, comprising a compound disclosed herein.

### Suitable Subjects and Diseases

In some embodiments, the subject is a mammal.

In some embodiments, the subject is a human.

In some embodiments, the GVHD is acute GVHD.

In some embodiments, the GVHD is late acute GVHD.

In some embodiments, the GVHD is chronic GVHD.

In some embodiments, the GVHD is late chronic GVHD.

In some embodiments, the administration results in reduced STAT3 activation in alloreactive T Cells.

In some embodiments, the administration results in preserved graft-versus-leukemia (GVL) effect.

In some embodiments, the graft-versus-leukemia (GVL) effect is preserved.

In some embodiments, the graft-versus tumor (GVT) effect is preserved.

### Administrations

In some embodiments, a pharmaceutical composition comprising the compound is administered.

In some embodiments, a pharmaceutical kit comprising the compound is administered.

In some embodiments, the compound is administered by enternal administration.

In some embodiments, the compound is administered by oral administration.

In some embodiments, the compound is administered by parenteral administration.

In some embodiments, the compound is administered by intravenous administration.

In some embodiments, the compound is administered once daily.

In some embodiments, the compound is administered twice daily.

In some embodiments, the compound is administered three or more times daily.

In some embodiments, the compound is administered with one or more drug holidays.

In some embodiments, the compound is administered without any drug holiday.

In some embodiments, a second therapeutic agent is administered to the subject.

### Compounds of the Present Disclosure

Without wishing to be bound by theory, the compound disclosed herein may be STAT3 protein inhibitors and/or STAT3 protein degraders. In some embodiments, the compound degrades one or more additional STAT proteins (e.g., STAT1). In some embodiments, the compound is a dual STAT3/STAT1 degrader.

In some embodiments, the compound is of Formula (I):

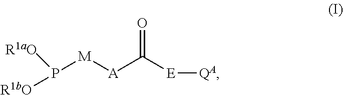

or a pharmaceutically acceptable salt or solvate thereof, wherein:

R^{1a} and R^{1b} are independently selected from the group consisting of hydrogen, C₁-C₄ alkyl, aralkyl, and —CH₂OC(═O)R^{e};

R^{1e} is selected from the group consisting of C₁-C₆ alkyl, C₃-C₆ cycloalkyl, and C₁-C₆ alkoxy;

M is selected from the group consisting of —O— and —C(R^{2a})(R^{2b})—;

each R^{2a} and R^{2b} are independently selected from the group consisting of hydrogen and fluoro; or

R^{2a} and R^{2b} taken together with the carbon atom to which they are attached form a —C(═O)— group;

A is selected from the group consisting of:

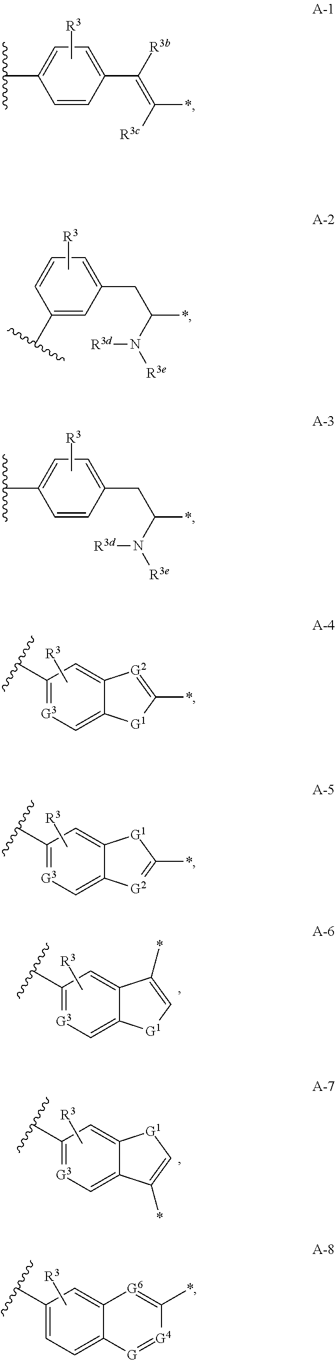

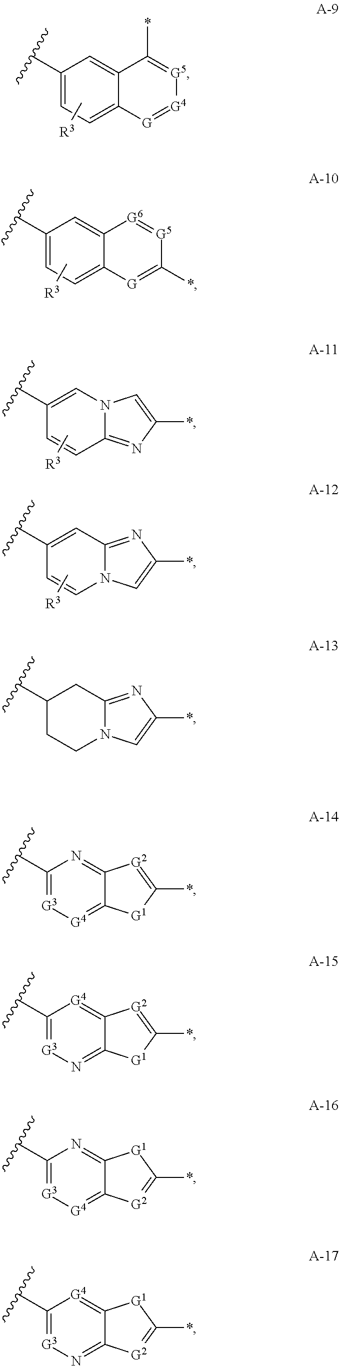

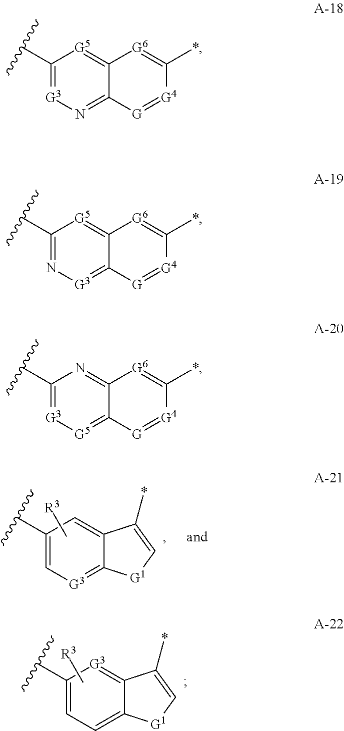

wherein the bond designated with an “*” is attached to —C(═O)-E-Q^{A};

G¹ is selected from the group consisting of —O—, —S—, and —NR¹⁷—;

G² is selected from the group consisting of —N═ and —CR^{18a}═;

G³ is selected from the group consisting of —N═ and —CR^{18b}═;

G⁴ is selected from the group consisting of —N═ and —CR^{18c}═;

G⁵ is selected from the group consisting of —N═ and —CR^{18d}═;

G⁶ is selected from the group consisting of —N═ and —CR^{18e}═;

G is selected from the group consisting of —N═ and —CR^{18f}═;

R³ is selected from the group consisting of hydrogen, halo, C₁-C₄ alkyl, C₃-C₆ cycloalkyl, —C(═O)R^{3a}, and aralkyl;

R^{3a} is C₁-C₄ alkyl;

R^{3b} and R^{3c} are independently selected from the group consisting of hydrogen and C₁-C₄ alkyl;

R^{3d} is selected from the group consisting of hydrogen, C₁-C₆ alkyl, and —C(═O)R^{3f};

R^{3e} is selected from the group consisting of hydrogen and C₁-C₄ alkyl;

R^{3f} is selected from the group consisting of C₁-C₁₂ alkyl, C₁-C₆ alkoxy, and aralkyloxy;

R¹⁷ is selected from the group consisting of hydrogen, C₁-C₄ alkyl, C₃-C₆ cycloalkyl, aralkyl, and —C(═O)R^{17a};

R^{17a} is C₁-C₄ alkyl;

R^{18a}, R^{18b}, R^{18c}, R^{18d}, R^{18e}, and R^{18f} are each independently selected from the group consisting of hydrogen, halo, and C₁-C₄ alkyl,

E is:

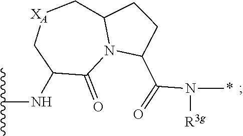

wherein the bond designated with an “*” is attached to Q^{A};

R^{3g} is selected from the group consisting of hydrogen and C₁-C₄ alkyl;

X_{A} is selected from the group consisting of —N(R⁸)CH₂—, —CH₂N(R⁸)—, and —CH₂CH₂—;

R⁸ is selected from the group consisting of hydrogen, C₁-C₆ alkyl, C₁-C₆ haloalkyl, (heterocyclo)alkyl, —C(═O)R⁹, alkylsulfonyl, and -L-B;

R⁹ is selected from the group consisting of C₁-C₆ alkyl, amino, C₁-C₆ alkoxy, aralkyloxy, optionally substituted C₃-C₁₀ cycloalkyl, optionally substituted 4- to 8-membered heterocyclo, optionally substituted aryl, optionally substituted 5- to 10-membered heteroaryl, aralkyl, and (heteroaryl)alkyl;

Q^{A} is selected from the group consisting of:

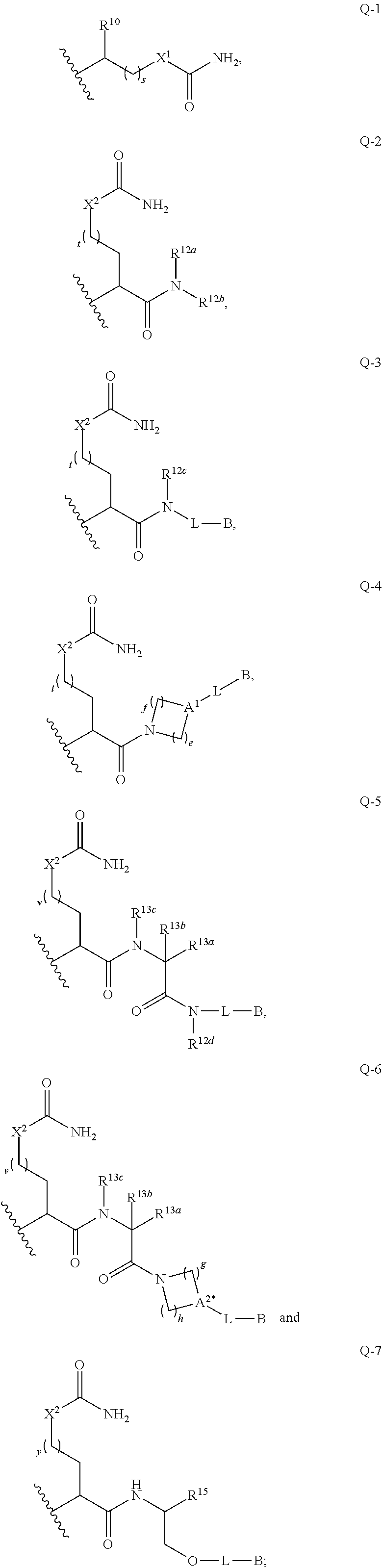

X¹ is selected from the group consisting of —CH₂—, —O—, and —N(R^{11a})—; or

X¹ is absent;

R¹⁰ is selected from the group consisting of hydrogen, C₁-C₆ alkyl, optionally substituted aralkyl, optionally substituted C₃-C₆ cycloalkyl, optionally substituted 4- to 8-membered heterocyclo, and optionally substituted aryl;

R^{11a} is selected from the group consisting of hydrogen and C₁-C₃ alkyl;

s is 1, 2, 3, or 4;

X² is selected from the group consisting of —CH₂—, —O—, and —N(R^{11b})—; or

X² is absent;

t is 0, 1, 2, 3, or 4;

R^{11b} is selected from the group consisting of hydrogen and C₁-C₃ alkyl;

R^{12a} is selected from the group consisting of hydrogen, C₁-C₆ alkyl, optionally substituted C₂-C₆ alkynyl, aralkyl, (heteroaryl)alkyl, optionally substituted C₃-C₆ cycloalkyl, optionally substituted 4- to 8-membered heterocyclo, optionally substituted aryl, (amido)(aryl)alkyl, (amino)(aryl)alkyl, (amino)(heteroaryl)alkyl, and (cycloalkyl)alkyl;

R^{12b} is selected from the group consisting of hydrogen, C₁-C₄ alkyl, optionally substituted aryl, and aralkyl; or

R^{12a} and R^{12b} taken together with the nitrogen atom to which they are attached form a 4- to 8-membered optionally substituted heterocyclo;

R^{12c} is selected from the group consisting of hydrogen, C₁-C₄ alkyl, and aralkyl;

A¹ is selected from the group consisting of —C(R^{14a})— and —N—;

R^{14a} is selected from the group consisting of hydrogen and C₁-C₃ alkyl;

e is 1, 2, or 3;

f is 1, 2, or 3;

X⁴ is selected from the group consisting of —CH₂—, —O—, and —N(R^{11d})—; or

X⁴ is absent;

v is 0, 1, 2, 3, or 4;

R^{11d} is selected from the group consisting of hydrogen and C₁-C₃ alkyl;

R^{12d} is selected from the group consisting of hydrogen, C₁-C₄ alkyl, and aralkyl;

R^{13a} is selected from the group consisting of hydrogen, C₁-C₆ alkyl, optionally substituted C₃-C₁₂ cycloalkyl, optionally substituted aryl, aralkyl, (heteroaryl)alkyl, (cycloalkyl)alkyl, and optionally substituted 5- to 9-membered heteroaryl;

R^{13b} is selected from the group consisting of hydrogen and C₁-C₄ alkyl;

R^{13c} is selected from the group consisting of hydrogen and C₁-C₄ alkyl; or

R^{13a} and R^{13b} taken together form a C₃-C₈ optionally substituted cycloalkyl or C₄-C₉ optionally substituted heterocyclo; or

R^{13b} and R^{13c} taken together form a 4- to 9-membered optionally substituted heterocyclo;

A^{2*} is selected from the group consisting of —C(R^{14b})— and —N—;

R^{14b} is selected from the group consisting of hydrogen and C₁-C₃ alkyl;

g is 1, 2, or 3;

h is 1, 2, or 3;

X⁵ is selected from the group consisting of —CH₂—, —O—, and —N(R^{11e})—; or

X⁵ is absent;

y is 0, 1, 2, 3, or 4;

R^{11e} is selected from the group consisting of hydrogen and C₁-C₃ alkyl;

R¹⁵ is selected from the group consisting of hydrogen, C₁-C₆ alkyl, C₃-C₈ cycloalkyl, optionally substituted aryl, and optionally substituted 5- to 9-membered heteroaryl;

L is -J¹-Y¹-J²-Y²-J³-Z—;

J¹ is selected from the group consisting of alkylenyl, heteroalkylenyl, cycloalkylenyl, heterocyclenyl, phenylenyl, and heteroarylenyl; or

J¹ is absent;

Y¹ is selected from the group consisting of —(CH₂)ₘ—, —C≡C—, —CH═CH—, —N(R^{16a})—, —C(═O)—, —S(═O)₂—, —C(═O)O—, —OC(═O)—, —C(═O)N(R^{16b})—, and —N(R^{16b})C(═O)—;

m is 0, 1, 2, or 3;

R^{16a} is selected from the group consisting of hydrogen, C₁-C₄ alkyl, and aralkyl;

R^{16b} is selected from the group consisting of hydrogen and C₁-C₄ alkyl;

J² is selected from the group consisting of alkylenyl, heteroalkylenyl, cycloalkylenyl, heterocyclenyl, phenylenyl, and heteroarylenyl; or

J² is absent;

Y² is selected from the group consisting of —(CH₂)ₙ—, —C≡C—, —CH═CH—, —N(R^{16a})—, —C(═O)—, —S(═O)₂—, —C(═O)O—, —OC(═O)—, —C(═O)N(R^{16b}), and —(R^{16b})C(═O)N—;

n is 0, 1, 2, 3, 4, 5, or 6;

R^{16a} is selected from the group consisting of hydrogen, C₁-C₄ alkyl, and aralkyl;

R^{16b} is selected from the group consisting of hydrogen and C₁-C₄ alkyl;

J³ is selected from the group consisting of alkylenyl, heteroalkylenyl, cycloalkylenyl, heterocyclenyl, phenylenyl, and heteroarylenyl; or

J³ is absent;

Z is selected from the group consisting of —(CH₂)_{d}—, —C≡C—, —CH═CH—, —C(═O)—, —O—, —S—, —N(R^{16c})—, —C(═O)N(R^{16d})—, —N(R^{16d})C(═O)—, —N(R^{16e})C(═O)CH₂O—, and —N(R^{16f})C(═O)CH₂N(R^{16g})—;

d is 0, 1, 2, or 3;

R^{16c}, R^{16d}, R^{16e}, R^{16f}, and R^{16g} are each independently selected from the group consisting of hydrogen, C₁-C₄ alkyl, and aralkyl;

wherein Z is attached to B;

B is selected from the group consisting of:

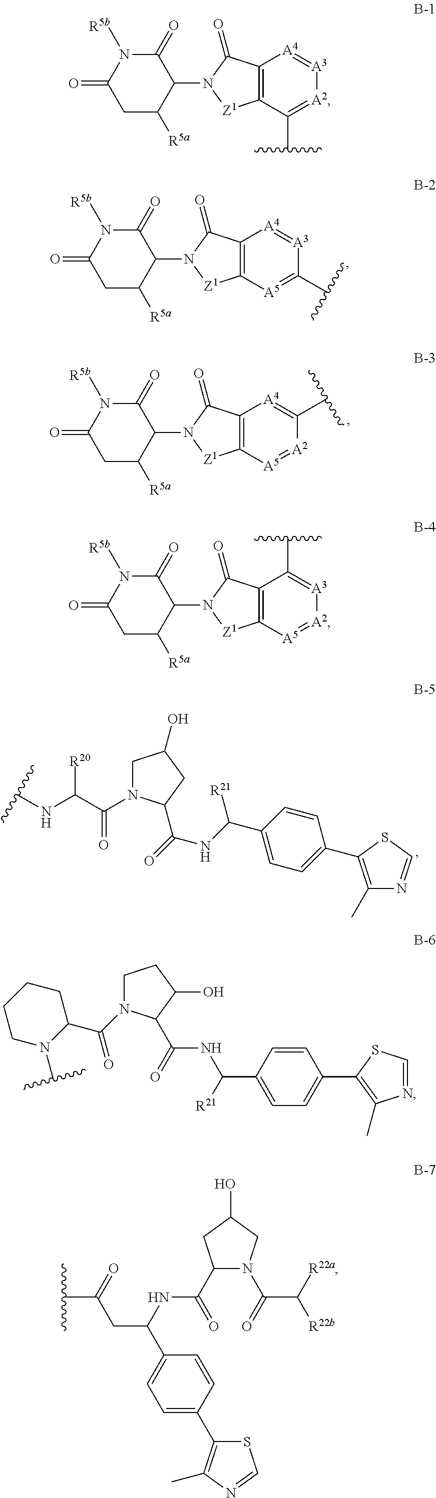

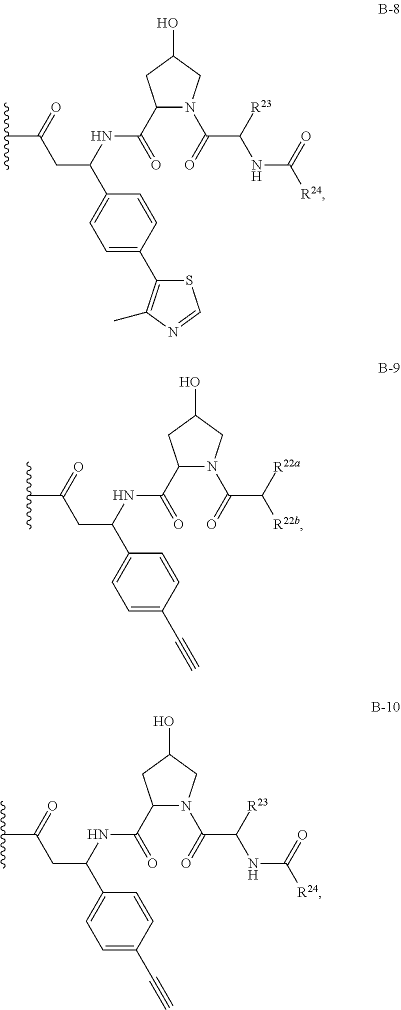

A₅ is selected from the group consisting of —C(R^{19a})═ and —N═;

A² is selected from the group consisting of —C(R^{19b})═ and —N═;

A³ is selected from the group consisting of —C(R^{19c})═ and —N═;

A⁴ is selected from the group consisting of —C(R^{19d})═ and —N═;

Z¹ is selected from the group consisting of —CH₂ and —C(═O)—;

R^{5a} is selected from the group consisting of hydrogen, methyl, and fluoro;

R^{5b} is selected from the group consisting of hydrogen and methyl;

R^{19a}, R^{19b}, R^{19c}, and R^{19d} are each independently selected from the group consisting of hydrogen, halo, and C₁₋₄ alkyl;

R²⁰ is C₁-C₆ alkyl;

R²¹ is selected from the group consisting of hydrogen and C₁-C₄ alkyl;

R^{22a} is selected from the group consisting of C₁-C₆ alkyl and optionally substituted C₃-C₆ cycloalkyl;

R^{22b} is selected from the group consisting of C₁-C₆ alkyl, optionally substituted C₃-C₆ cycloalkyl, optionally substituted aryl, and optionally substituted heteroaryl; and

R²³ is selected from the group consisting of C₁-C₆ alkyl and optionally substituted C₃-C₆ cycloalkyl; and

R²⁴ is selected from the group consisting of C₁-C₆ alkyl, optionally substituted C₃-C₆ cycloalkyl, optionally substituted aryl, and optionally substituted heteroaryl.

In some embodiments, the compound is of Formula (I) a pharmaceutically acceptable salt or solvate thereof, wherein:

(1) when X_{A} is —CH₂CH₂—, then Q^{A} is selected from the group consisting of Q-3, Q-4, Q-5, Q-6, and Q-7;

(2) when X_{A} is —N(R⁸)CH₂— or —CH₂N(R⁸)—, and R⁸ is selected from the group consisting of hydrogen, C₁-C₆ alkyl, C₁-C₆ haloalkyl, (heterocyclo)alkyl, alkylsulfonyl, and —C(═O)R⁹, then Q^{A} is selected from the group consisting of Q-3, Q-4, Q-5, Q-6, and Q-7; and/or

(3) when X_{A} is —N(R⁸)CH₂— or —CH₂N(R⁸)—, and R⁸ is -L-B, then Q^{A} is selected from the group consisting of Q-1 and Q-2.

In some embodiments, R^{1e} is C₁-C₆ alkyl.

In some embodiments, R⁸ is selected from the group consisting of hydrogen, C₁-C₆ alkyl, —C(═O)R⁹, and -L-B.

In some embodiments, R⁸ is selected from the group consisting of hydrogen, C₁-C₆ alkyl, —C(═O)R⁹, alkylsulfonyl, and -L-B.

In some embodiments, R^{13a} is selected from the group consisting of hydrogen, C₁-C₆ alkyl, optionally substituted C₃-C₁₂ cycloalkyl, optionally substituted aryl, aralkyl, and optionally substituted 5- to 9-membered heteroaryl.

In some embodiments, R^{13a} is aralkyl.

In some embodiments, M is —O—.

In some embodiments, M is —C(R^{2a})(R^{2b})—.

In some embodiments, the compound is of Formula (II):

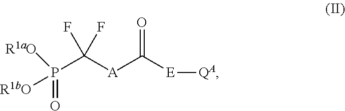

or a pharmaceutically acceptable salt or solvate thereof.

In some embodiments, the compound is of Formula (III):

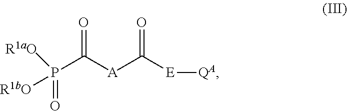

or a pharmaceutically acceptable salt or solvate thereof.

In some embodiments, A is A-1.

In some embodiments, A is A-2.

In some embodiments, A is A-3.

In some embodiments, A is A-4.

In some embodiments, A is A-5.

In some embodiments, A is A-6.

In some embodiments, A is A-7.

In some embodiments, A is A-8.

In some embodiments, A is A-9.

In some embodiments, A is A-10.

In some embodiments, A is A-11.

In some embodiments, A is A-12.

In some embodiments, A is A-13.

In some embodiments, A is A-14.

In some embodiments, A is A-15.

In some embodiments, A is A-16.

In some embodiments, A is A-17.

In some embodiments, A is A-18.

In some embodiments, A is A-19.

In some embodiments, A is A-20.

In some embodiments, A is A-21.

In some embodiments, A is A-22.

In some embodiments, A is selected from the group consisting of:

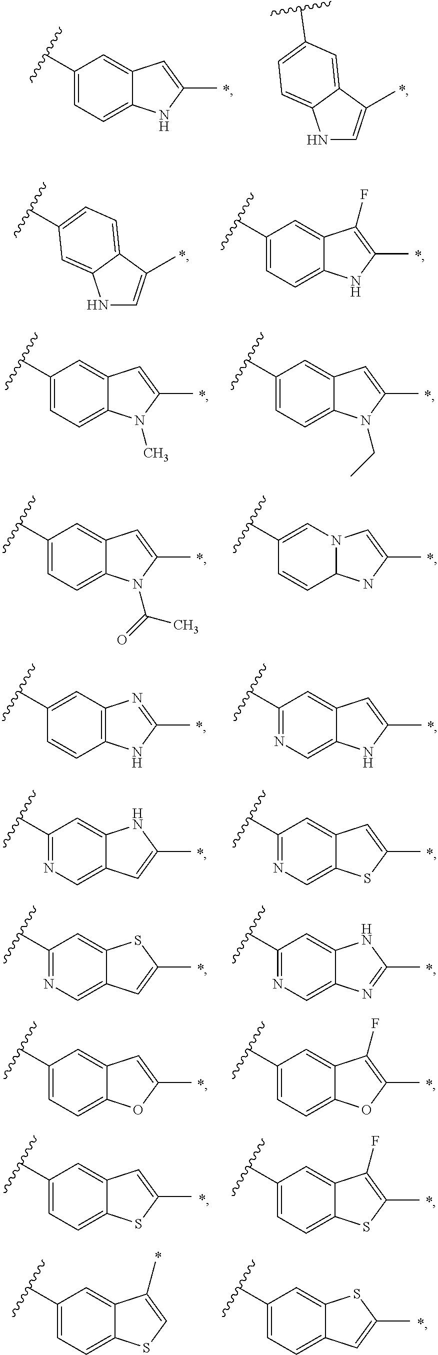

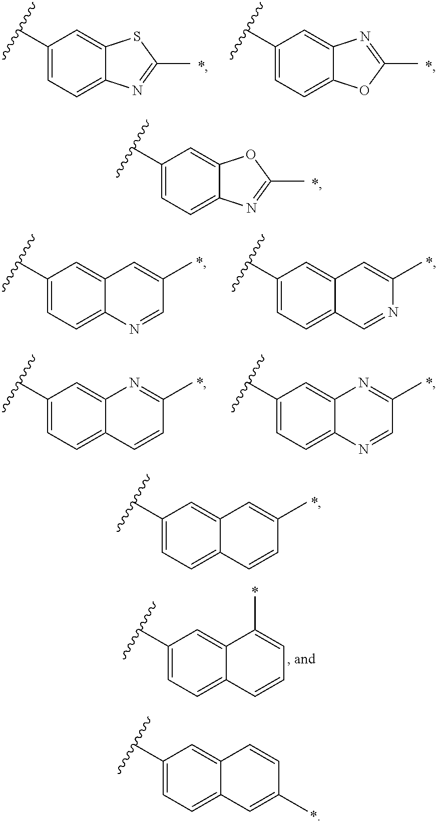

In some embodiments, A is selected from the group consisting of:

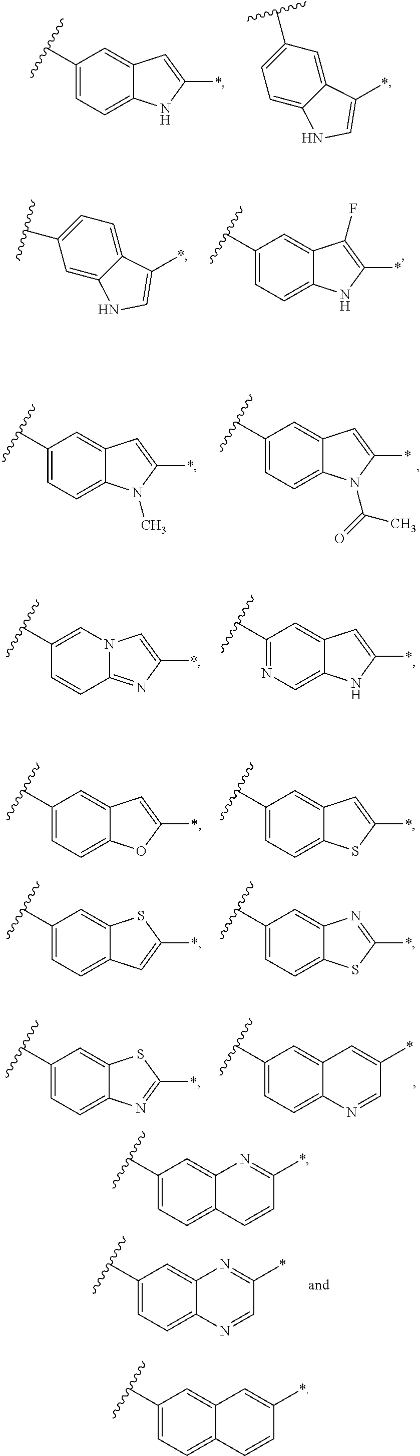

In some embodiments, A is selected from the group consisting of:

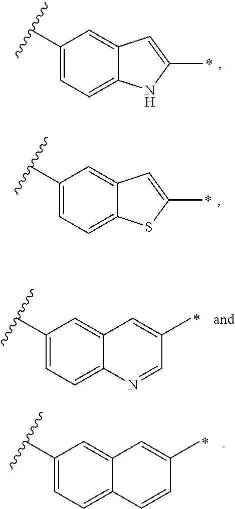 [SMILES: *c1ccc2[nH]c(C)cc2c1 ; *c1ccc2sc(C)cc2c1 ; *c1ccc2ncc(C)cc2c1 ; *c1ccc2ccc(C)cc2c1]

In some embodiments, A is

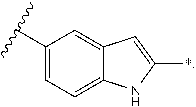

In some embodiments, A is

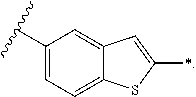

In some embodiments, A is

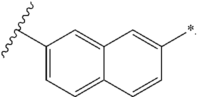

In some embodiments, R^{3g} is C₁-C₄ alkyl.

In some embodiments, R^{3g} is hydrogen.

In some embodiments, E is:

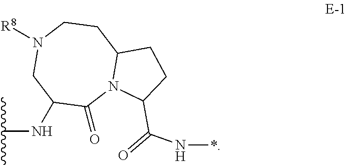

In some embodiments, E-1 is selected from the group consisting of:

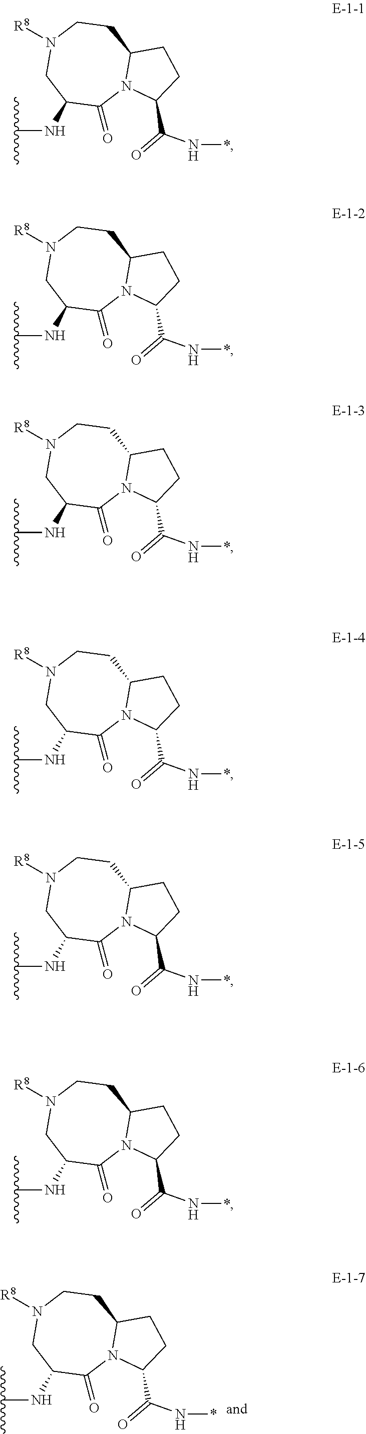

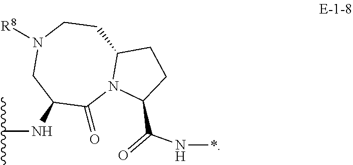

In some embodiments, E-1 is E-1-1.

In some embodiments, E-1 is E-1-2.

In some embodiments, E-1 is E-1-3.

In some embodiments, E-1 is E-1-4.

In some embodiments, E-1 is E-1-5.

In some embodiments, E-1 is E-1-6.

In some embodiments, E-1 is E-1-7.

In some embodiments, E-1 is E-1-8.

In some embodiments, E is:

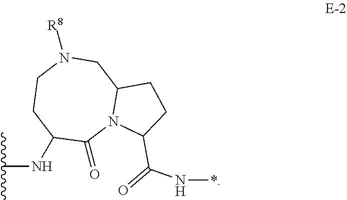

In some embodiments, E-2 is selected from the group consisting of:

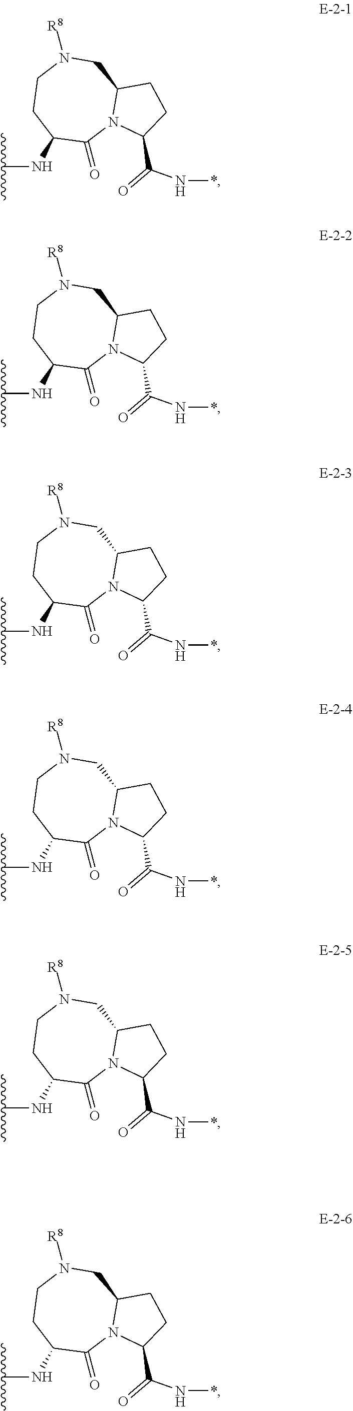

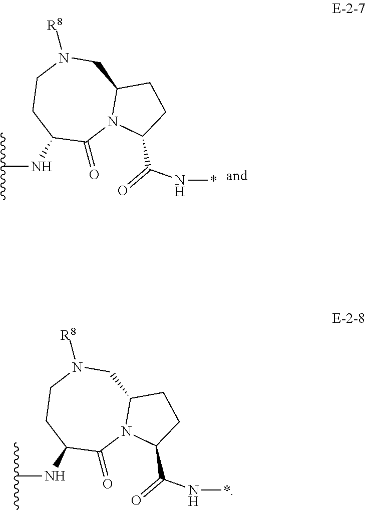

In some embodiments, E-2 is E-2-1.

In some embodiments, E-2 is E-2-2.

In some embodiments, E-2 is E-2-3.

In some embodiments, E-2 is E-2-4.

In some embodiments, E-2 is E-2-5.

In some embodiments, E-2 is E-2-6.

In some embodiments, E-2 is E-2-7.

In some embodiments, E-2 is E-2-8.

In some embodiments, E is:

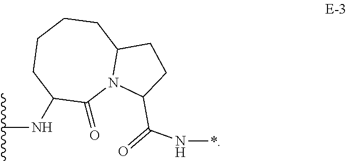

In some embodiments, E-3 is selected from the group consisting of:

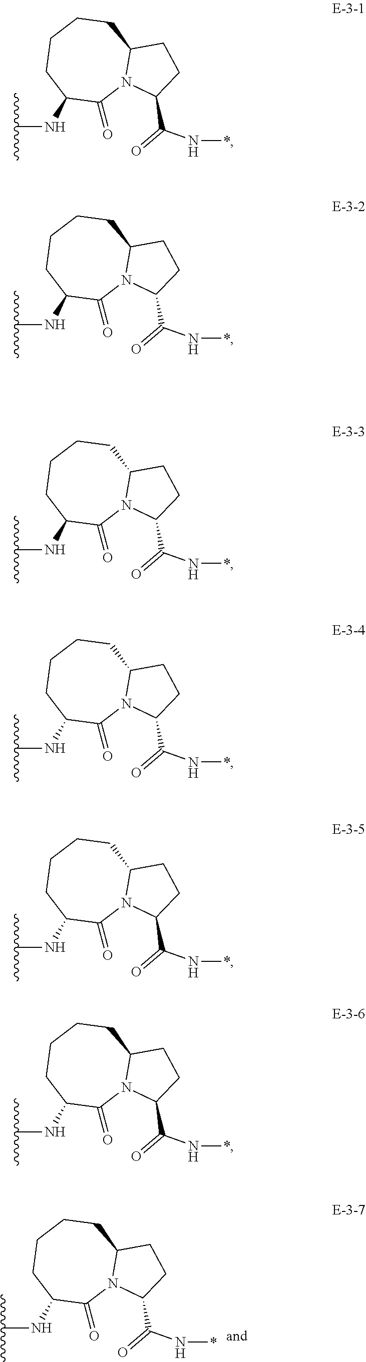

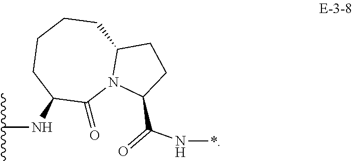

In some embodiments, E-3 is E-3-1.

In some embodiments, E-3 is E-3-2.

In some embodiments, E-3 is E-3-3.

In some embodiments, E-3 is E-3-4.

In some embodiments, E-3 is E-3-5.

In some embodiments, E-3 is E-3-6.

In some embodiments, E-3 is E-3-7.

In some embodiments, E-3 is E-3-8.

In some embodiments, E is E-1, E-1-1, E-1-2, E-1-3, E-1-4, E-1-5, E-1-6, E-1-7, E-1-8, E-2, E-2-1, E-2-2, E-2-3, E-2-4, E-2-5, E-2-6, E-2-7, or E-2-8, R⁸ is -L-B, Q^{A} is Q-1, and R¹⁰ is hydrogen. In some embodiments, E is E-1-1. In some embodiments, E is E-2-1. In some embodiments, X¹ is selected from the group consisting of —CH₂— and —N(H)—. In some embodiments, X¹ is —CH₂—. In some embodiments, s is 0 or 1. In some embodiments, s is 0.

In some embodiments, E is E-1, E-1-1, E-1-2, E-1-3, E-1-4, E-1-5, E-1-6, E-1-7, E-1-8, E-2, E-2-1, E-2-2, E-2-3, E-2-4, E-2-5, E-2-6, E-2-7, or E-2-8, R⁸ is -L-B, Q^{A} is Q-2. In some embodiments, E is E-1-1. In some embodiments, E is E-2-1.

In some embodiments, Q-2 is:

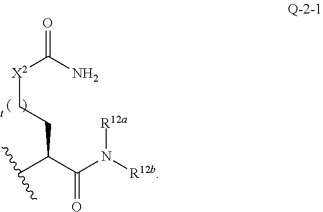

In some embodiments, X² is selected from the group consisting of —CH₂—, —O—, and —N(H)—. In some embodiments, X² is —CH₂—. In some embodiments, t is 0 or 1. In some embodiments, t is 0. In some embodiments, R^{12b} is hydrogen. In some embodiments, R^{12a} is aralkyl.

In some embodiments, E is E-1, E-1-1, E-1-2, E-1-3, E-1-4, E-1-5, E-1-6, E-1-7, E-1-8, E-2, E-2-1, E-2-2, E-2-3, E-2-4, E-2-5, E-2-6, E-2-7, or E-2-8, R⁸ is selected from the group consisting of hydrogen, C₁-C₆ alkyl, C₁-C₆ haloalkyl, (heterocyclo)alkyl, alkylsulfonyl, and —C(═O)R⁹, and Q^{A} is Q-3. In some embodiments, E is E-1-1. In some embodiments, E is E-2-1. In some embodiments, R⁸ is C₁-C₄ alkyl.

In some embodiments, E is E-3, E-3-1, E-3-2, E-3-3, E-3-4, E-3-5, E-3-6, E-3-7, or E-3-8; and Q^{A} is Q-3. In some embodiments, E is E-3-1.

In some embodiments, Q-3 is Q-3-1:

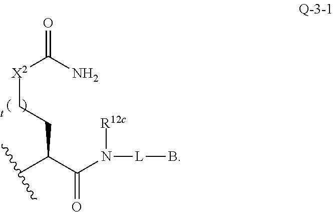

In some embodiments, X² is —CH₂—. In some embodiments, t is 0 or 1. In some embodiments, t is 0. In some embodiments, R^{12c} is hydrogen.

In some embodiments, E is E-1, E-1-1, E-1-2, E-1-3, E-1-4, E-1-5, E-1-6, E-1-7, E-1-8, E-2, E-2-1, E-2-2, E-2-3, E-2-4, E-2-5, E-2-6, E-2-7, or E-2-8, R⁸ is selected from the group consisting of hydrogen, C₁-C₆ alkyl, C₁-C₆ haloalkyl, (heterocyclo)alkyl, alkylsulfonyl, and —C(═O)R⁹, and Q^{A} is Q-4. In some embodiments, E is E-1-1. In some embodiments, E is E-2-1. In some embodiments, R⁸ is C₁-C₄ alkyl.

In some embodiments, E is E-3, E-3-1, E-3-2, E-3-3, E-3-4, E-3-5, E-3-6, E-3-7, or E-3-8; and Q^{A} is Q-4. In some embodiments, E is E-3-1.

In some embodiments, Q-4 is Q-4-1:

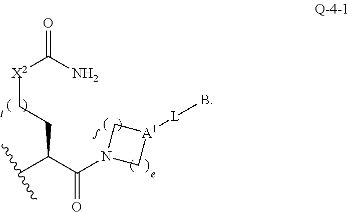

In some embodiments, X² is —CH₂—. In some embodiments, t is 0 or 1. In some embodiments, t is 0. In some embodiments, f and e are each 1 or 2. In some embodiments, f and e are each 2. In some embodiments, A¹ is —C(H)—.

In some embodiments, E is E-1, E-1-1, E-1-2, E-1-3, E-1-4, E-1-5, E-1-6, E-1-7, E-1-8, E-2, E-2-1, E-2-2, E-2-3, E-2-4, E-2-5, E-2-6, E-2-7, or E-2-8, R⁸ is selected from the group consisting of hydrogen, C₁-C₆ alkyl, C₁-C₆ haloalkyl, (heterocyclo)alkyl, alkylsulfonyl, and —C(═O)R⁹, and Q^{A} is Q-5. In some embodiments, E is E-1-1. In some embodiments, E is E-2-1. In some embodiments, R⁸ is C₁-C₄ alkyl.

In some embodiments, E is E-3, E-3-1, E-3-2, E-3-3, E-3-4, E-3-5, E-3-6, E-3-7, or E-3-8; and Q^{A} is Q-5. In some embodiments, E is E-3-1.

In some embodiments, Q-5 is Q-5-1:

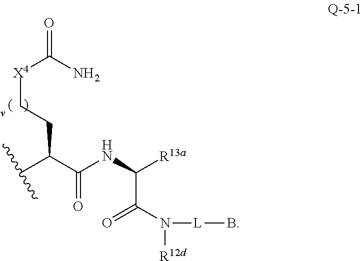

In some embodiments, X⁴ is —CH₂—. In some embodiments, v is 0 or 1. In some embodiments, v is 0. In some embodiments, R^{12d} is hydrogen. In some embodiments, R^{13a} is selected from the group consisting of optionally substituted C₃-C₁₂ cycloalkyl, aralkyl, and optionally substituted phenyl. In some embodiments, R^{13a} is optionally substituted C₃-C₆ cycloalkyl. In some embodiments, R^{13a} is optionally substituted phenyl. In some embodiments, R^{13a} is aralkyl.

In some embodiments, Q-5 is Q-5-2:

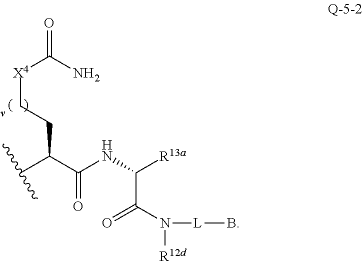

In some embodiments, X⁴ is —CH₂—. In some embodiments, v is 0 or 1. In some embodiments, v is 0. In some embodiments, R^{12d} is hydrogen. In some embodiments, R^{13a} is selected from the group consisting of optionally substituted C₃-C₁₂ cycloalkyl, aralkyl, and optionally substituted phenyl. In some embodiments, R^{13a} is optionally substituted C₃-C₆ cycloalkyl. In some embodiments, R^{13a} is optionally substituted phenyl. In some embodiments, R^{13a} is aralkyl.

In some embodiments, E is E-1, E-1-1, E-1-2, E-1-3, E-1-4, E-1-5, E-1-6, E-1-7, E-1-8, E-2, E-2-1, E-2-2, E-2-3, E-2-4, E-2-5, E-2-6, E-2-7, or E-2-8, R⁸ is selected from the group consisting of hydrogen, C₁-C₆ alkyl, C₁-C₆ haloalkyl, (heterocyclo)alkyl, alkylsulfonyl, and —C(═O)R⁹, and Q^{A} is Q-6. In some embodiments, E is E-1-1. In some embodiments, E is E-2-1. In some embodiments, R⁸ is C₁-C₄ alkyl.

In some embodiments, E is E-3, E-3-1, E-3-2, E-3-3, E-3-4, E-3-5, E-3-6, E-3-7, or E-3-8; and Q^{A} is Q-6. In some embodiments, E is E-3-1.

In some embodiments, Q-6 is Q-6-1:

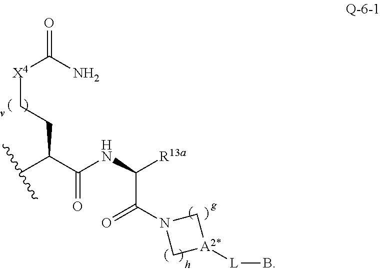

In some embodiments, X⁴ is —CH₂—. In some embodiments, v is 0 or 1. In some embodiments, v is 0. In some embodiments, R^{13a} is selected from the group consisting of optionally substituted C₃-C₁₂ cycloalkyl, aralkyl, and optionally substituted phenyl. In some embodiments, R^{13a} is optionally substituted C₃-C₆ cycloalkyl. In some embodiments, R^{13a} is optionally substituted phenyl. In some embodiments, R^{13a} is aralkyl. In some embodiments, g and h are each 1 or 2. In some embodiments, g and h are 2. In some embodiments, A^{2*} is —C(H)—. In some embodiments, A^{2*} is —N—.

In some embodiments, Q-6-1 is Q-6-1-A or Q-6-1-B:

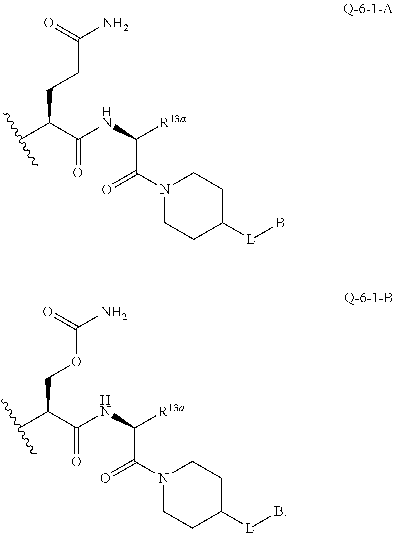

In some embodiments, Q-6 is Q-6-2:

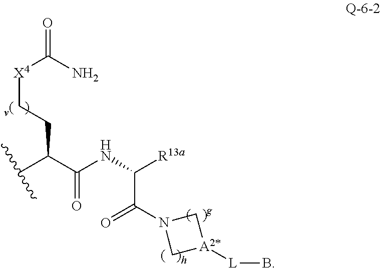

In some embodiments, X⁴ is —CH₂—. In some embodiments, v is 0 or 1. In some embodiments, v is 0. In some embodiments, R^{13a} is selected from the group consisting of optionally substituted C₃-C₁₂ cycloalkyl, aralkyl, and optionally substituted phenyl. In some embodiments, R^{13a} is optionally substituted C₃-C₆ cycloalkyl. In some embodiments, R^{13a} is optionally substituted phenyl. In some embodiments, R^{13a} is aralkyl. In some embodiments, g and h are each 1 or 2. In some embodiments, g and h are 2. In some embodiments, A^{2*} is —C(H)—.

In some embodiments, E is E-1, E-1-1, E-1-2, E-1-3, E-1-4, E-1-5, E-1-6, E-1-7, E-1-8, E-2, E-2-1, E-2-2, E-2-3, E-2-4, E-2-5, E-2-6, E-2-7, or E-2-8, R⁸ is selected from the group consisting of hydrogen, C₁-C₆ alkyl, C₁-C₆ haloalkyl, (heterocyclo)alkyl, alkylsulfonyl, and —C(═O)R⁹, and Q^{A} is Q-7. In some embodiments, E is E-1-1. In some embodiments, E is E-2-1. In some embodiments, R⁸ is C₁-C₄ alkyl.

In some embodiments, E is E-3, E-3-1, E-3-2, E-3-3, E-3-4, E-3-5, E-3-6, E-3-7, or E-3-8; and Q^{A} is Q-7. In some embodiments, E is E-3-1.

In some embodiments, Q-7 is Q-7-1;

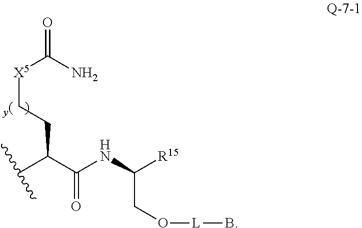

In some embodiments, X⁵ is —CH₂—. In some embodiments, y is 0 or 1. In some embodiments, y is 0. In some embodiments, R¹⁵ is optionally substituted phenyl.

In some embodiments, Q-7 is Q-7-2;

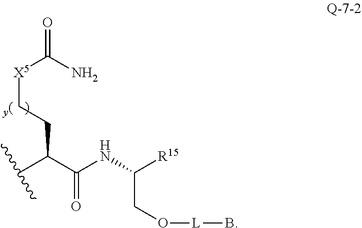

In some embodiments, X⁵ is —CH₂—. In some embodiments, y is 0 or 1. In some embodiments, y is 0. In some embodiments, R¹⁵ is optionally substituted phenyl.

In some embodiments, L is —Y¹-J²-Y²-J³-Z—. In some embodiments, L is —Y¹—Y²-J³-Z—. In some embodiments, L is —Y¹-J²-Y²—Z—. In some embodiments, L is —Y¹—Y²—Z—. In some embodiments, Y¹ is selected from the group consisting of —(CH₂)ₘ— and —C(═O)—; m is 1, 2, or 3; Y² is —(CH₂)ₙ—; n is 1, 2, 3, 4, 5, or 6; and Z is selected from the group consisting of —(CH₂)—, —C≡C—, and —N(H)—.

In some embodiments, wherein:

R⁸ is -L-B;

L is selected from the group consisting of:

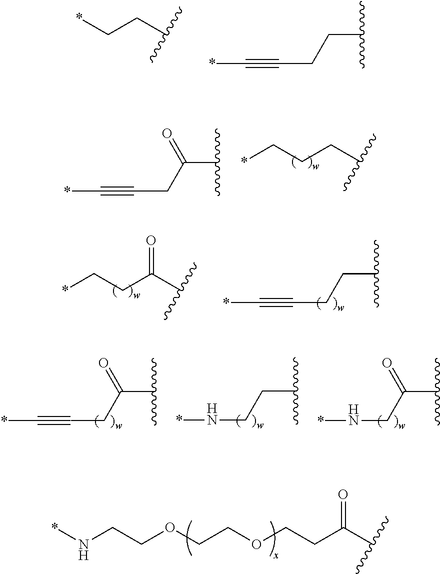

wherein the bond designated with an “*” is attached to B;

w is 1, 2, 3, 4, 5, 6, 7, 8, 9, 10, 11, or 12; and

x is 1, 2, 3, 4, 5, or 6.

In some embodiments, wherein:

R⁸ is selected from the group consisting of hydrogen, C₁-C₆ alkyl, C₁-C₆ haloalkyl, (heterocyclo)alkyl, alkylsulfonyl, and —C(═O)R⁹;

L is selected from the group consisting of:

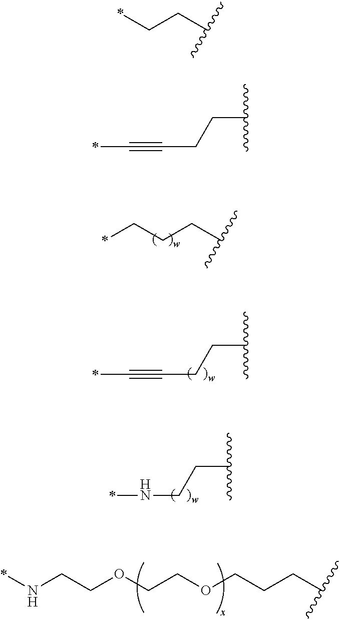

wherein the bond designated with an “*” is attached to B;

w is 1, 2, 3, 4, 5, 6, 7, 8, 9, 10, 11, or 12; and

x is 1, 2, 3, 4, 5, or 6.

In some embodiments, B is B-1, or a pharmaceutically acceptable salt or solvate thereof. In some embodiments, R^{5b} is hydrogen. In some embodiments, A², A³, and A⁴ are —CH═. In some embodiments, Z¹ is —C(═O)—. In some embodiments, Z¹ is —CH₂—.

In some embodiments, B-1 is:

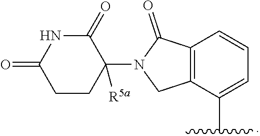

In some embodiments, B-1 is:

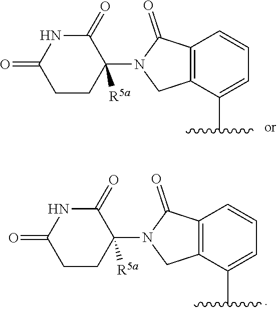

In some embodiments, R^{5a} is fluoro. In some embodiments, R^{5a} is deuterium. In some embodiments, R^{5a} is hydrogen.

In some embodiments, B is B-2.

In some embodiments, B is B-3.

In some embodiments, B is B-4.

In some embodiments, B is B-5.

In some embodiments, B is B-6.

In some embodiments, B is B-7.

In some embodiments, B is B-8.

In some embodiments, B is B-9.

In some embodiments, B is B-10.

In some embodiments, the compound is of Formula (IV):

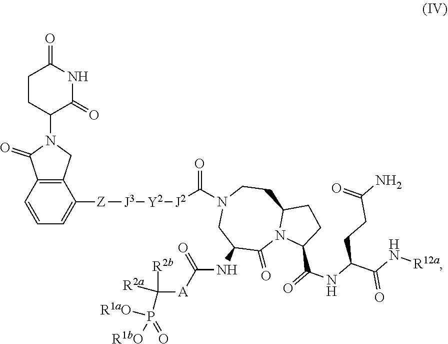

or a pharmaceutically acceptable salt or solvate thereof.

In some embodiments, the compound is of Formula (V):

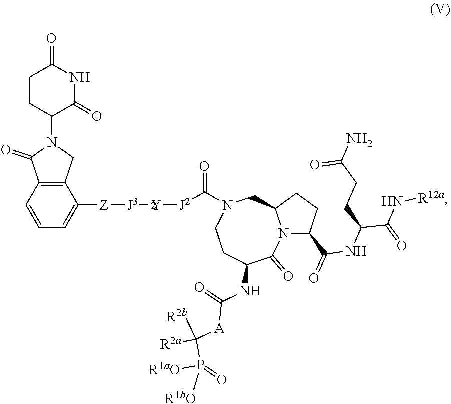

or a pharmaceutically acceptable salt or solvate thereof.

In some embodiments, the compound is of Formula (IV-A):

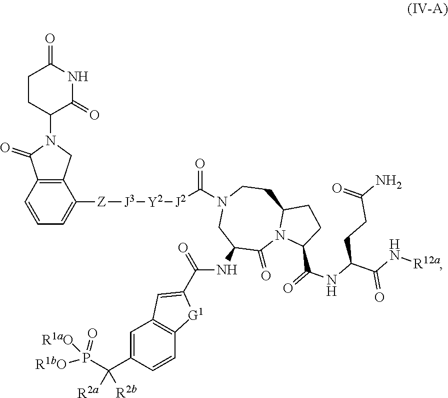

or a pharmaceutically acceptable salt or solvate thereof.

In some embodiments, the compound is of Formula (V-A):

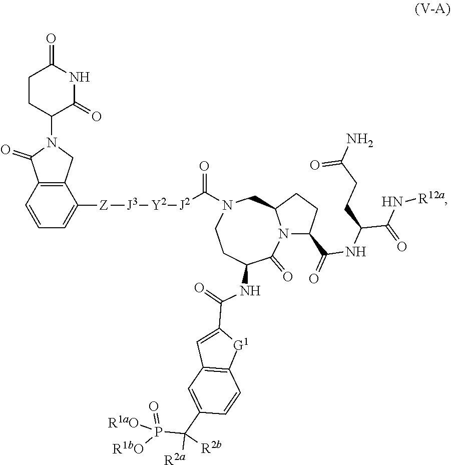

or a pharmaceutically acceptable salt or solvate thereof.

In some embodiments, R^{12a} is aralkyl.

In some embodiments, R^{12a} is:

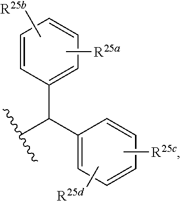

wherein R^{25a}, R^{25b}, R^{25c}, and R^{25d} are each independently selected from the group consisting of hydrogen, halo, C₁-C₄ alkyl, and C₁-C₄ alkoxy.

In some embodiments, G¹ is —S—.

In some embodiments, G¹ is —NH—.

In some embodiments, R^{2a} and R^{2b} are fluoro.

In some embodiments, R^{2a} and R^{2b} taken together with the carbon to which they are attached form a —C(═O)— group.

In some embodiments, J² is absent, Y² is —(CH₂)ₙ—, n is 3, 4, or 5, and J³ is absent.

In some embodiments, Z is —C≡C—.

In some embodiments, the compound is of Formula (VI):

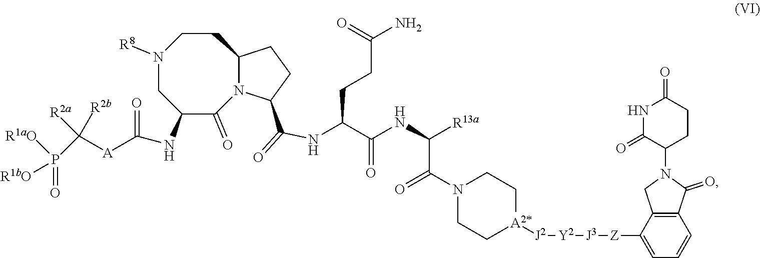

or a pharmaceutically acceptable salt or solvate thereof.

In some embodiments, the compound is of Formula (VII):

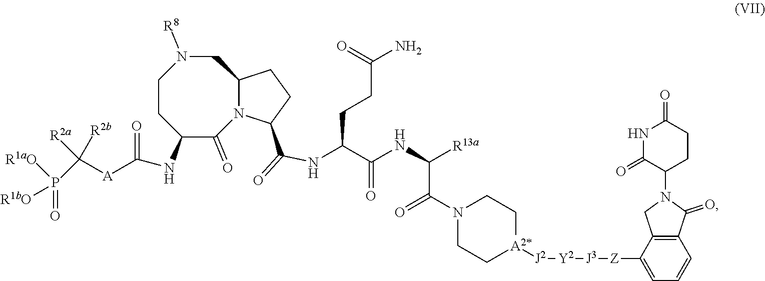

or a pharmaceutically acceptable salt or solvate thereof.

In some embodiments, the compound is of Formula (VI-A):

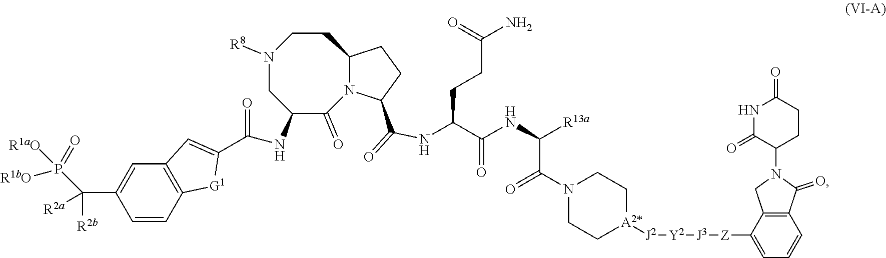

or a pharmaceutically acceptable salt or solvate thereof.

In some embodiments, the compound is of Formula (VII-A):

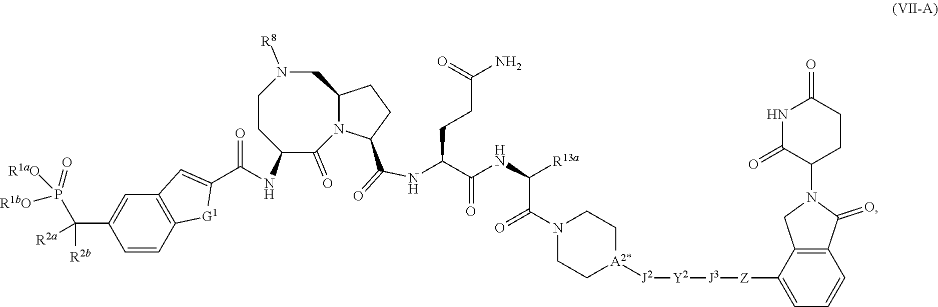

or a pharmaceutically acceptable salt or solvate thereof.

In some embodiments, the compound is of Formula (VI-B):

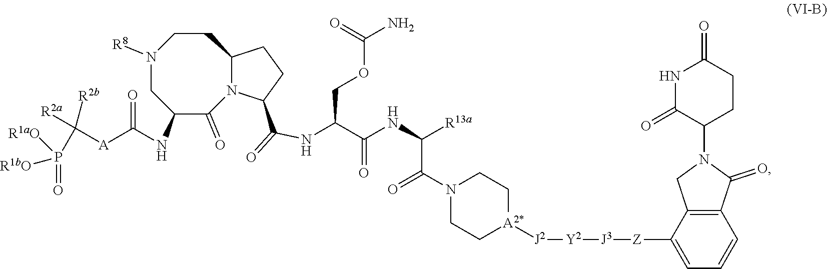

or a pharmaceutically acceptable salt or solvate thereof.

In some embodiments, the compound is of Formula (VII-B):

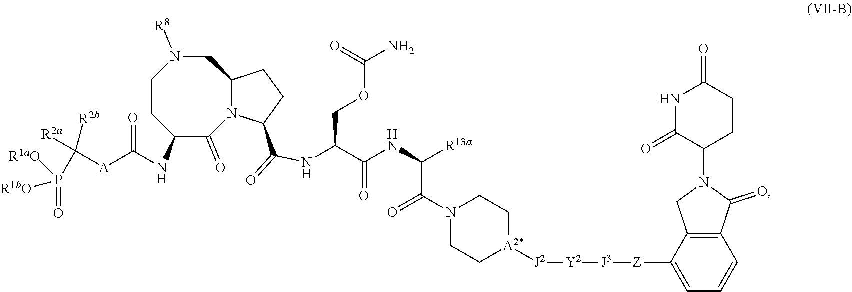

or a pharmaceutically acceptable salt or solvate thereof.

In some embodiments, the compound is of Formula (VI-C):

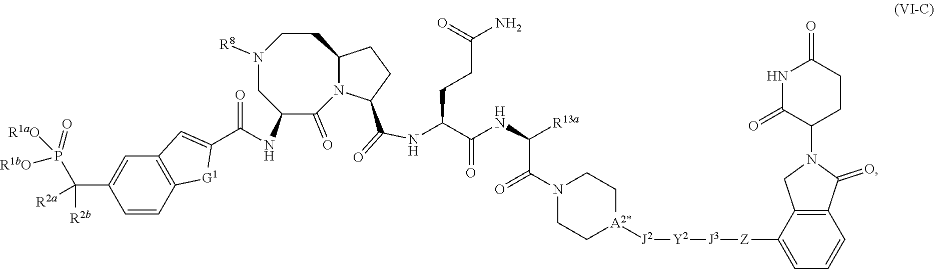

or a pharmaceutically acceptable salt or solvate thereof.

In some embodiments, the compound is of Formula (VII-C):

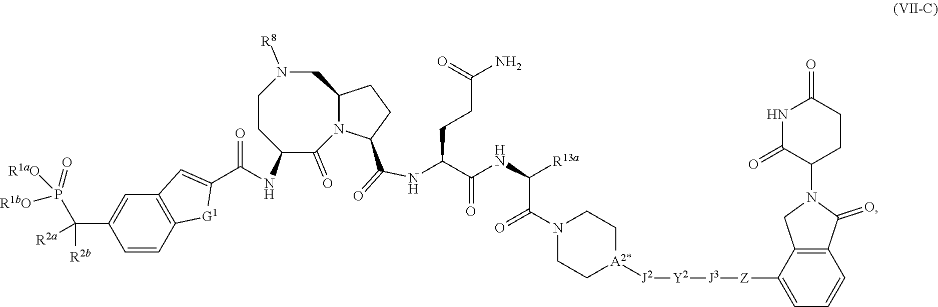

or a pharmaceutically acceptable salt or solvate thereof.

In some embodiments, R^{13a} is selected from the group consisting of optionally substituted cyclohexyl, aralkyl, (heteroaryl)alkyl, and optionally substituted phenyl. In some embodiments, R^{13a} is selected from the group consisting of optionally substituted cyclohexyl, aralkyl, and optionally substituted phenyl. In some embodiments, R^{13a} is optionally substituted phenyl. In some embodiments, R^{13a} is aralkyl. In some embodiments, R^{13a} is (heteroaryl)alkyl.

In some embodiments, R⁸ is selected from the group consisting of C₁-C₄ alkyl, C₁-C₄ haloalkyl, (heterocyclo)alkyl, alkylsulfonyl, and —C(═O)R⁹; and R⁹ is selected from the group consisting of C₁-C₄ alkyl, alkylamino, dialkylamino, and C₁-C₄ alkoxy. In some embodiments, R⁸ is selected from the group consisting of C₁-C₄ alkyl, C₁-C₄ haloalkyl, alkylsulfonyl, and —C(═O)R⁹; and R⁹ is selected from the group consisting of C₁-C₄ alkyl, alkylamino, dialkylamino, and C₁-C₄ alkoxy. In some embodiments, R⁸ is C₁-C₄ alkyl. In some embodiments, R⁸ is methyl. In some embodiments, R⁸ is methyl, ethyl, isopropyl, —CH₂CHF₂, —CH₂CF₃, —C(═O)OCH₃, —C(═O)CH₃, —C(═O)NHCH₃, —C(═O)N(CH₃)₂, —S(═O)₂Me, —S(═O)₂Et, or —SO₂iPr. In some embodiments, R⁸ is methyl, ethyl, —CH₂CHF₂, or —C(═O)OCH₃.

In some embodiments, A^{2*} is —CH—.

In some embodiments, A^{2*} is —N—.

In some embodiments, G¹ is —S—.

In some embodiments, G¹ is —NH—.

In some embodiments, R^{2a} and R^{2b} are fluoro.

In some embodiments, R^{2a} and R^{2b} taken together with the carbon to which they are attached form a —C(═O)— group.

In some embodiments, wherein J² is absent, Y² is —(CH₂)ₙ—, n is 2, 3, or 4, and J³ is absent.

In some embodiments, Z is —C≡C—.

In some embodiments, the compound is of Formula (VII-D):

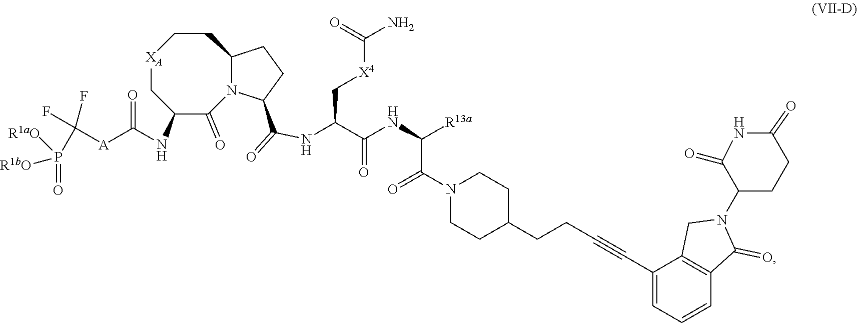

or a pharmaceutically acceptable salt or solvate thereof.

In some embodiments, X_{A} is —N(R⁸)CH₂—; and R⁸ is selected from the group consisting of hydrogen, C₁-C₆ alkyl, C₁-C₆ haloalkyl, (heterocyclo)alkyl, alkylsulfonyl, and —C(═O)R⁹. In some embodiments, R⁸ is selected from the group consisting of C₁-C₄ alkyl, C₁-C₄ haloalkyl, and —C(═O)R⁹; and R⁹ is selected from the group consisting of C₁-C₄ alkyl, alkylamino, dialkylamino, and C₁-C₄ alkoxy. In some embodiments, R⁹ is selected from the group consisting of C₁-C₃ alkyl, alkylamino, dialkylamino, and C₁-C₃ alkoxy. In some embodiments, R⁸ is methyl, ethyl, isopropyl, —CH₂CHF₂, —CH₂CF₃, —C(═O)OCH₃, —C(═O)CH₃, —C(═O)NHCH₃, —C(═O)N(CH₃)₂, —S(═O)₂Me, —S(═O)₂Et, or -SO₂iPr. In some embodiments, R⁸ is methyl, ethyl, —CH₂CHF₂, or —C(═O)OCH₃.

In some embodiments, X_{A} is —CH₂N(R⁸)—; and R⁸ is selected from the group consisting of hydrogen, C₁-C₆ alkyl, C₁-C₆ haloalkyl, (heterocyclo)alkyl, alkylsulfonyl, and —C(═O)R⁹. In some embodiments, R⁸ is selected from the group consisting of C₁-C₄ alkyl, C₁-C₄ haloalkyl, and —C(═O)R⁹; and R⁹ is selected from the group consisting of C₁-C₄ alkyl, alkylamino, dialkylamino, and C₁-C₄ alkoxy. In some embodiments, R⁹ is selected from the group consisting of C₁-C₃ alkyl, alkylamino, dialkylamino, and C₁-C₃ alkoxy. In some embodiments, R⁸ is methyl, ethyl, isopropyl, —CH₂CHF₂, —CH₂CF₃, —C(═O)OCH₃, —C(═O)CH₃, —C(═O)NHCH₃, —C(═O)N(CH₃)₂, —S(═O)₂Me, —S(═O)₂Et, or -SO₂iPr. In some embodiments, R⁸ is methyl, ethyl, —CH₂CHF₂, or —C(═O)OCH₃.

In some embodiments, X_{A} is —CH₂CH₂—.

In some embodiments, X⁴ is —CH₂—.

In some embodiments, X⁴ is —O—.

In some embodiments, the compound is of Formula (VII-E):

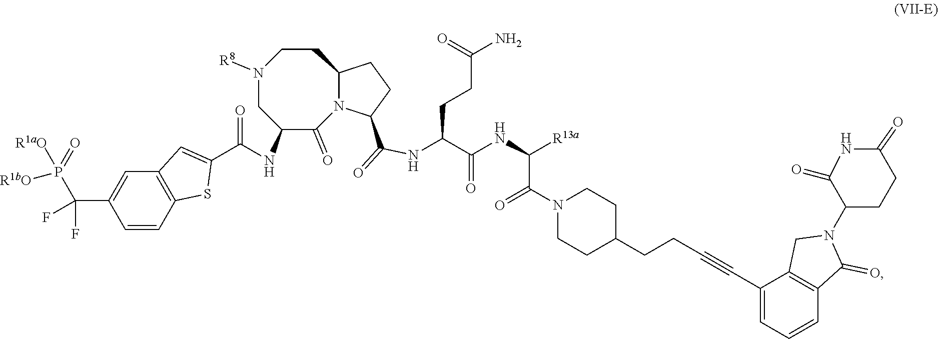

or a pharmaceutically acceptable salt or solvate thereof.

In some embodiments, R⁸ is selected from the group consisting of hydrogen, C₁-C₆ alkyl, C₁-C₆ haloalkyl, (heterocyclo)alkyl, alkylsulfonyl, and —C(═O)R⁹. In some embodiments, R⁸ is selected from the group consisting of C₁-C₄ alkyl, C₁-C₄ haloalkyl, alkylsulfonyl, and —C(═O)R⁹; and R⁹ is selected from the group consisting of C₁-C₄ alkyl, alkylamino, dialkylamino, and C₁-C₄ alkoxy. In some embodiments, R⁹ is selected from the group consisting of C₁-C₃ alkyl, alkylamino, dialkylamino, and C₁-C₃ alkoxy. In some embodiments, R⁸ is methyl, ethyl, isopropyl, —CH₂CHF₂, —CH₂CF₃, —C(═O)OCH₃, —C(═O)CH₃, —C(═O)NHCH₃, —C(═O)N(CH₃)₂, —S(═O)₂Me, —S(═O)₂Et, or -SO₂iPr. In some embodiments, R⁸ is methyl, ethyl, —CH₂CHF₂, or —C(═O)OCH₃.

In some embodiments, R^{13a} is selected from the group consisting optionally substituted C₃-C₁₂ cycloalkyl, optionally substituted aryl, aralkyl, (heteroaryl)alkyl, (cycloalkyl)alkyl, and optionally substituted 5- to 9-membered heteroaryl.

In some embodiments, R^{13a} is selected from the group consisting of:

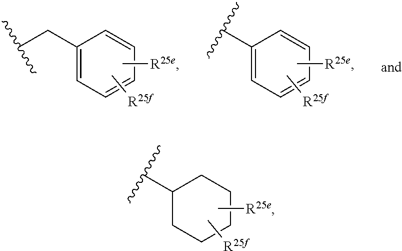

wherein R^{25e} and R^{25f} are independently selected from the group consisting of hydrogen, halo, C₁-C₄ alkyl, C₁-C₄ haloalkyl, and C₁-C₄ alkoxy.

In some embodiments, R^{1a} and R^{1b} are C₁-C₄ alkyl.

In some embodiments, R^{1a} and R^{1b} are hydrogen.

In some embodiments, R^{1a} and R^{1b} are —CH₂OC(═O)R^{1e}; and each R^{1e} is independently selected from the group consisting of C₁-C₆ alkyl and C₁-C₆ alkoxy. In some embodiments, each R^{1e} is C₁-C₆ alkyl. In some embodiments, each R^{1e} is C₁-C₆ alkoxy.

In some embodiments, R^{1a} and R^{1b} are selected from the group consisting of:

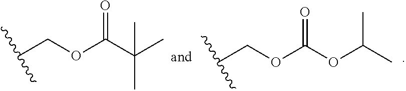

In some embodiments, wherein:

R^{13a} is selected from the group consisting of:

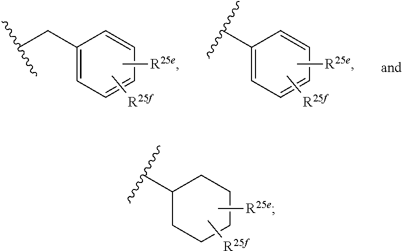

and

R^{25e} and R^{25f} are independently selected from the group consisting of hydrogen, halo, C₁-C₆ alkyl, optionally substituted C₃-C₆ cycloalkyl, optionally substituted aryl, C₁-C₄ haloalkyl, C₁-C₄ alkoxy, carboxamido, sulfonamido, alkylsulfonyl, arylsulfonyl, —C(═O)R⁵⁷, —S(═O)₂R⁵⁸, and —N(R^{56c})S(═O)₂R^{56d}.

In some embodiments, wherein:

R^{13a} is:

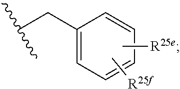

R^{25e} is selected from the group consisting of hydrogen, halo, C₁-C₆ alkyl, —C(═O)NR^{50c}R^{50d}, C₁-C₆ alkylsulfonyl, arylsulfonyl, —N(R^{56c})S(═O)₂R^{56d}, and —S(═O)₂R⁵⁸,

R^{25f} is selected from the group consisting of hydrogen and halo;

R^{50c} is selected from the group consisting of C₁-C₆ alkyl, optionally substituted C₃-C₆ cycloalkyl, optionally substituted 5- or 6-membered heterocyclo, optionally substituted phenyl, optionally substituted 5- to 9-membered heteroaryl, aralkyl, (heteroaryl)C₁-C₄ alkyl, and (heterocyclo)C₁-C₄ alkyl;

R^{50d} is selected from the group consisting of hydrogen and C₁-C₃ alkyl; or

R^{50c} and R^{50d} taken together with the nitrogen to which they are attached form a 3- to 8-membered optionally substituted heterocyclo group;

R⁵⁸ is optionally substituted C₃-C₆ cycloalkyl;

R^{56c} is selected from the group consisting of hydrogen and C₁-C₃ alkyl; and

R^{56d} is selected from the group consisting of optionally substituted C₃-C₆ cycloalkyl, optionally substituted phenyl, and optionally substituted 5- to 9-membered heteroaryl.

In some embodiments, the compound is of Formula (XXII):

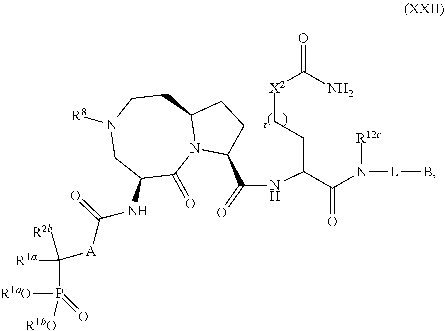

or a pharmaceutically acceptable salt or solvate thereof, wherein:

R^{1a} and R^{1b} are independently selected from the group consisting of hydrogen, C₁-C₆ alkyl, and aralkyl; and

R⁸ is selected from the group consisting of hydrogen, C₁-C₆ alkyl, C₁-C₆ haloalkyl, (heterocyclo)alkyl, alkylsulfonyl, and —C(═O)R⁹.

In some embodiments, the compound is of Formula (XXIII):

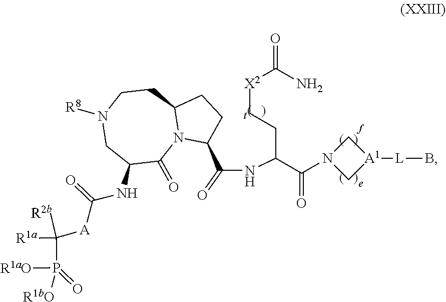

or a pharmaceutically acceptable salt or solvate thereof, wherein:

R^{1a} and R^{1b} are independently selected from the group consisting of hydrogen, C₁-C₆ alkyl, and aralkyl; and

R⁸ is selected from the group consisting of hydrogen, C₁-C₆ alkyl, C₁-C₆ haloalkyl, (heterocyclo)alkyl, alkylsulfonyl, and —C(═O)R⁹.

In some embodiments, the compound is of Formula (XXIV):

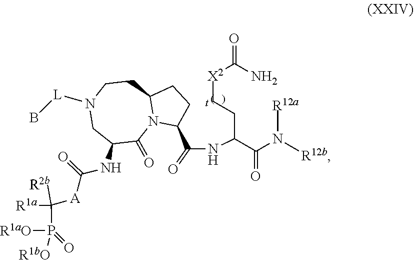

or a pharmaceutically acceptable salt or solvate thereof, wherein R^{1a} and R^{1b} are independently selected from the group consisting of hydrogen, C₁-C₆ alkyl, and aralkyl.

In some embodiments, the compound is of Formula XXVI:

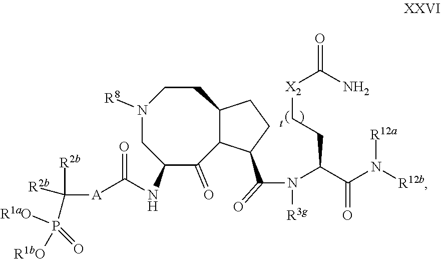

or a pharmaceutically acceptable salt or solvate thereof, wherein R^{1a} and R^{1b} are independently selected from the group consisting of hydrogen, C₁-C₆ alkyl, and aralkyl.

In some embodiments, the compound is selected from the compounds described in Tables 1, 1A and 1B, pharmaceutically acceptable salts thereof, and solvates thereof.

In some embodiments, the compound is selected from the compounds described in Tables 1 and 1A, pharmaceutically acceptable salts thereof, and solvates thereof.

In some embodiments, the compound is selected from the compounds described in Table 1, pharmaceutically acceptable salts thereof, and solvates thereof.

In some embodiments, the compound is selected from the compounds described in Table 1A, pharmaceutically acceptable salts thereof, and solvates thereof.

In some embodiments, the compound is selected from the compounds described in Table 1B, pharmaceutically acceptable salts thereof, and solvates thereof.

The chemical names in Tables 1, 1A, and 1B were generated by Chemdraw® Professional version 17.0.0.206 (121). In the event of any ambiguity between their chemical structure and chemical name, Compounds of the Disclosure are defined by their chemical structure.

TABLE 1

| Cpd. | | |
|---|---|---|
| No. | Structure | Name |
| 1 | 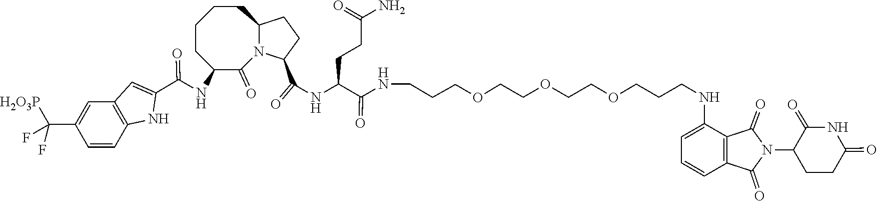 | ((2-(((3S,6S,10aS)-3-(((16S)-19-amino-1-((2-(2,6- dioxopiperidin-3-yl)-1,3-dioxoisoindolin-4-yl)amino)- 15,19-dioxo-4,7,10-trioxa-14-azanonadecan-16- yl)carbamoyl)-5-oxodecahydropyrrolo[1,2-a]azocin-6- yl)carbamoyl)-1H-indol-5-yl)difluoromethyl)phosphonic acid |
| 2 | 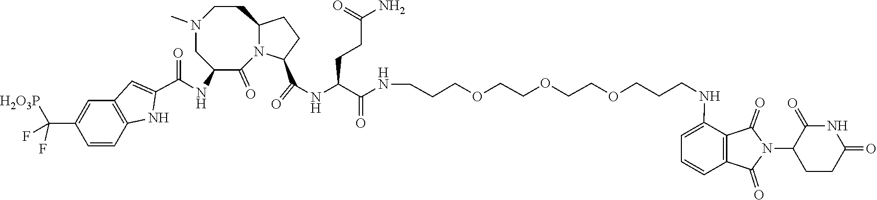 | ((2-(((5S,8S,10aR)-8-(((16S)-19-amino-1-((2-(2,6- dioxopiperidin-3-yl)-1,3-dioxoisoindolin-4-yl)amino)- 15,19-dioxo-4,7,10-trioxa-14-azanonadecan-16- yl)carbamoyl)-3-methyl-6-oxodecahydropyrrolo[1,2- a][1,5]diazocin-5-yl)carbamoyl)-1H-indol-5- yl)difluoromethyl)phosphonic acid |
| 3 | 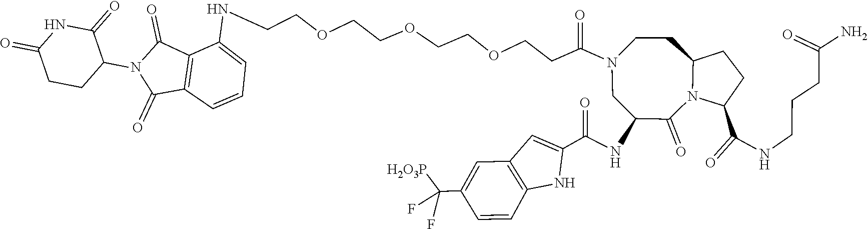 | ((2-(((5S,8S,10aR)-8-((4-amino-4-oxobutyl)carbamoyl)- 3-(3-(2-(2-(2-((2-(2,6-dioxopiperidin-3-yl)-1,3- dioxoisoindolin-4- yl)amino)ethoxy)ethoxy)ethoxy)propanoyl)-6- oxodecahydropyrrolo[1,2-a][1,5]diazocin-5- yl)carbamoyl)-1H-indol-5-yl)difluoromethyl)phosphonic acid |
| 4 | 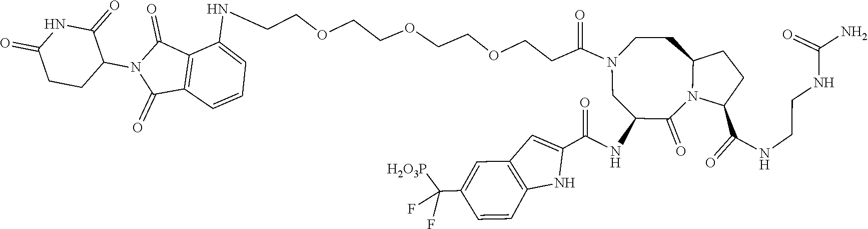 | ((2-(((5S,8S,10aR)-3-(3-(2-(2-(2-((2-(2,6-dioxopiperidin- 3-yl)-1,3-dioxoisoindolin-4- yl)amino)ethoxy)ethoxy)ethoxy)propanoyl)-6-oxo-8-((2- ureidoethyl)carbamoyl)decahydropyrrolo[1,2- a][1,5]diazocin-5-yl)carbamoyl)-1H-indol-5- yl)difluoromethyl)phosphonic acid |
| 5 | 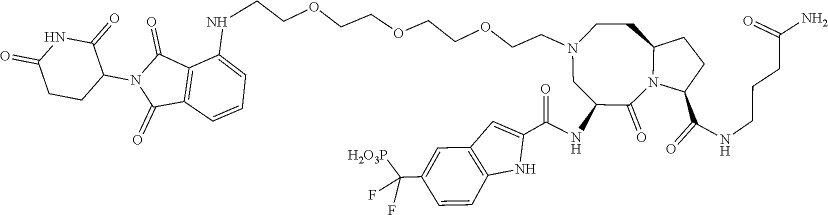 | ((2-(((5S,8S,10aR)-8-((4-amino-4-oxobutyl)carbamoyl)- 3-(2-(2-(2-(2-((2-(2,6-dioxopiperidin-3-yl)-1,3- dioxoisoindolin-4- yl)amino)ethoxy)ethoxy)ethoxy)ethyl)-6- oxodecahydropyrrolo[1,2-a][1,5]diazocin-5- yl)carbamoyl)-1H-indol-5-yl)difluoromethyl)phosphonic acid |
| 6 | 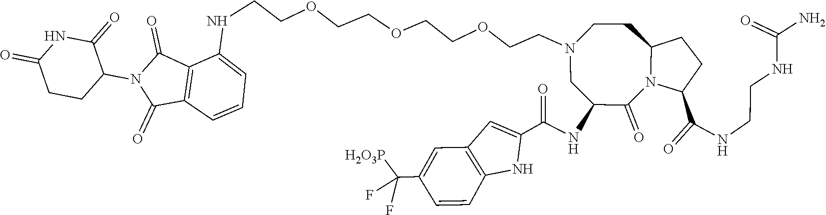 | ((2-(((5S,8S,10aR)-3-(2-(2-(2-(2-((2-(2,6-dioxopiperidin- 3-yl)-1,3-dioxoisoindolin-4- yl)amino)ethoxy)ethoxy)ethoxy)ethyl)-6-oxo-8-((2- ureidoethyl)carbamoyl)decahydropyrrolo[1,2- a][1,5]diazocin-5-yl)carbamoyl)-1H-indol-5- yl)difluoromethyl)phosphonic acid |
| 7 | 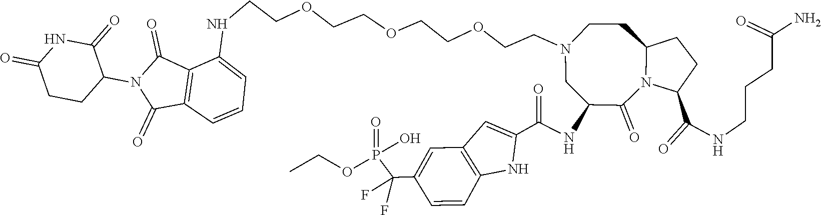 [SMILES: CCOP(=O)(O)C(F)(F)c1ccc2[nH]c(C(=O)N[C@H]3CN(CCOCCOCCOCCNc4cccc5c4C(=O)N(C4CCC(=O)NC4=O)C5=O)CC[C@H]4CC[C@@H](C(=O)NCCCC(N)=O)N4C3=O)cc2c1] | ethyl hydrogen ((2-(((5S,8S,10aR)-8-((4-amino-4- oxobutyl)carbamoyl)-3-(2-(2-(2-(2-((2-(2,6- dioxopiperidin-3-yl)-1,3-dioxoisoindolin-4- yl)amino)ethoxy)ethoxy)ethoxy)ethyl)-6- oxodecahydropyrrolo[1,2-a][1,5]diazocin-5- yl)carbamoyl)-1H-indol-5- yl)difluoromethyl)phosphonate |
| 8 | 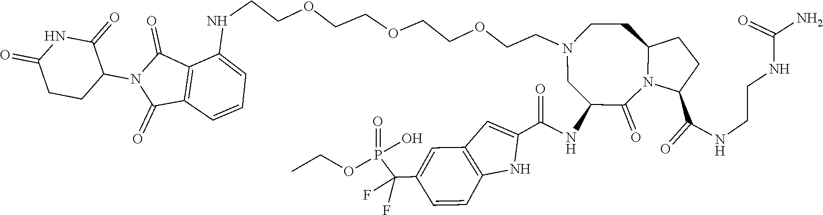 [SMILES: CCOP(=O)(O)C(F)(F)c1ccc2[nH]c(C(=O)N[C@H]3CN(CCOCCOCCOCCNc4cccc5c4C(=O)N(C4CCC(=O)NC4=O)C5=O)CC[C@H]4CC[C@@H](C(=O)NCCNC(N)=O)N4C3=O)cc2c1] | ethyl hydrogen ((2-(((5S,8S,10aR)-3-(2-(2-(2-(2-((2-(2,6- dioxopiperidin-3-yl)-1,3-dioxoisoindolin-4- yl)amino)ethoxy)ethoxy)ethoxy)ethyl)-6-oxo-8-((2- ureidoethyl)carbamoyl)decahydropyrrolo[1,2- a][1,5]diazocin-5-yl)carbamoyl)-1H-indol-5- yl)difluoromethyl)phosphonate |
| 9 | 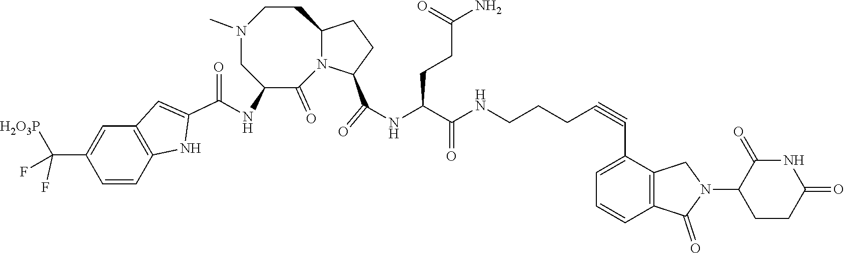 | ((2-(((5S,8S,10aR)-8-(((2S)-5-amino-1-((5-(2-(2,6- dioxopiperidin-3-yl)-1-oxoisoindolin-4-yl)pent-4-yn-1- yl)amino)-1,5-dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 10 | 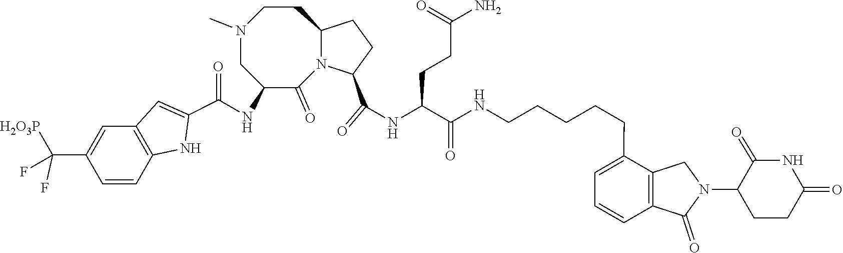 | ((2-(((5S,8S,10aR)-8-(((2S)-5-amino-1-((5-(2-(2,6- dioxopiperidin-3-yl)-1-oxoisoindolin-4-yl)pentyl)amino)- 1,5-dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 11 | 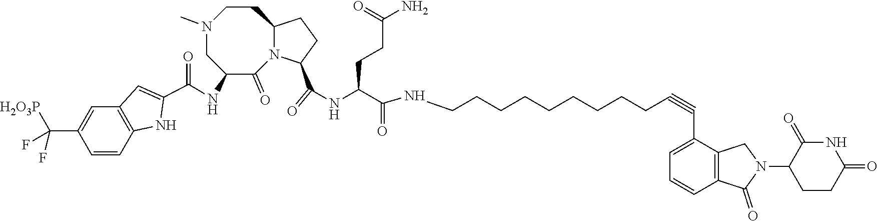 | ((2-(((5S,8S,10aR)-8-(((2S)-5-amino-1-((11-(2-(2,6- dioxopiperidin-3-yl)-1-oxoisoindolin-4-yl)undec-10-yn- 1-yl)amino)-1,5-dioxopentan-2-yl)carbamoyl)-3-methyl- 6-oxodecahydropyrrolo[1,2-a][1,5]diazocin-5- yl)carbamoyl)-1H-indol-5-yl)difluoromethyl)phosphonic acid |
| 12 | 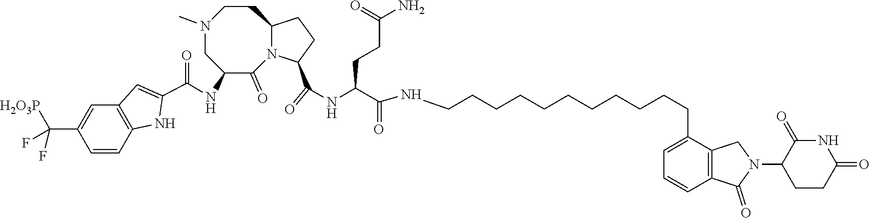 | ((2-(((5S,8S,10aR)-8-(((2S)-5-amino-1-((11-(2-(2,6- dioxopiperidin-3-yl)-1-oxoisoindolin-4- yl)undecyl)amino)-1,5-dioxopentan-2-yl)carbamoyl)-3- methyl-6-oxodecahydropyrrolo[1,2-a][1,5]diazocin-5- yl)carbamoyl)-1H-indol-5-yl)difluoromethyl)phosphonic acid |
| 13 | 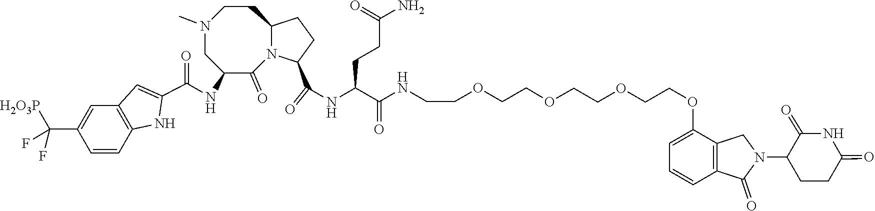 | ((2-(((5S,8S,10aR)-8-(((14S)-17-amino-1-((2-(2,6- dioxopiperidin-3-yl)-1-oxoisoindolin-4-yl)oxy)-13,17- dioxo-3,6,9-trioxa-12-azaheptadecan-14-yl)carbamoyl)- 3-methyl-6-oxodecahydropyrrolo[1,2-a][1,5]diazocin-5- yl)carbamoyl)-1H-indol-5-yl)difluoromethyl)phosphonic acid |
| 14 | 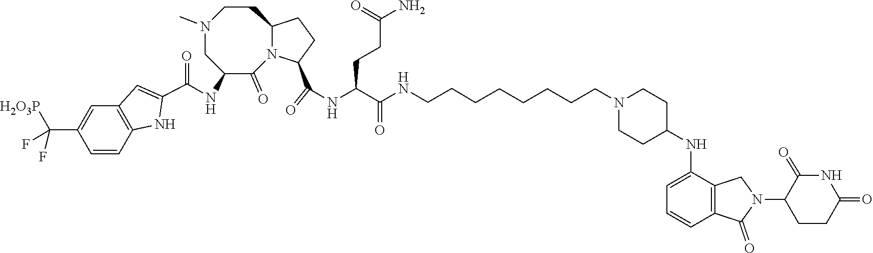 | ((2-(((5S,8S,10aR)-8-(((2S)-5-amino-1-((8-(4-((2-(2,6- dioxopiperidin-3-yl)-1-oxoisoindolin-4- yl)amino)piperidin-1-yl)octyl)amino)-1,5-dioxopentan-2- yl)carbamoyl)-3-methyl-6-oxodecahydropyrrolo[1,2- a][1,5]diazocin-5-yl)carbamoyl)-1H-indol-5- yl)difluoromethyl)phosphonic acid |
| 15 | 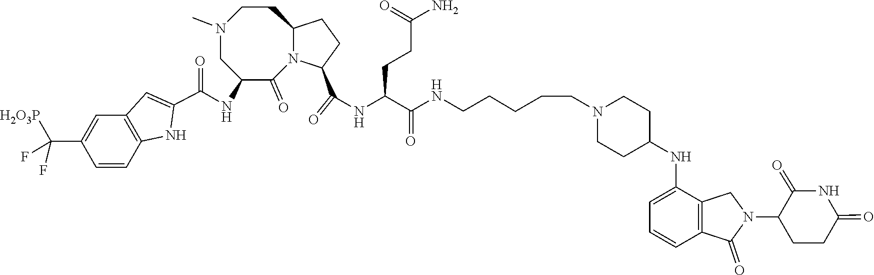 | ((2-(((5S,8S,10aR)-8-(((2S)-5-amino-1-((5-(4-((2-(2,6- dioxopiperidin-3-yl)-1-oxoisoindolin-4- yl)amino)piperidin-1-yl)pentyl)amino)-1,5-dioxopentan- 2-yl)carbamoyl)-3-methyl-6-oxodecahydropyrrolo[1,2- a][1,5]diazocin-5-yl)carbamoyl)-1H-indol-5- yl)difluoromethyl)phosphonic acid |
| 16 | 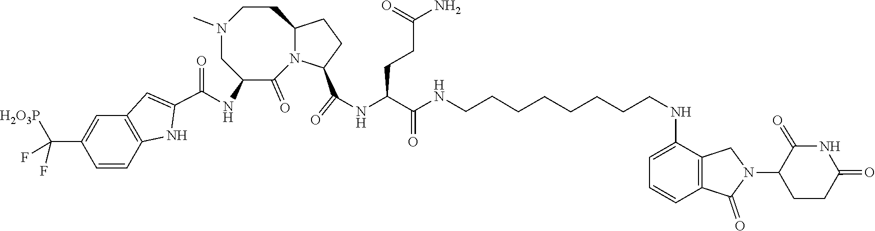 | ((2-(((5S,8S,10aR)-8-(((2S)-5-amino-1-((8-((2-(2,6- dioxopiperidin-3-yl)-1-oxoisoindolin-4- yl)amino)octyl)amino)-1,5-dioxopentan-2-yl)carbamoyl)- 3-methyl-6-oxodecahydropyrrolo[1,2-a][1,5]diazocin-5- yl)carbamoyl)-1H-indol-5-yl)difluoromethyl)phosphonic acid |
| 17 | 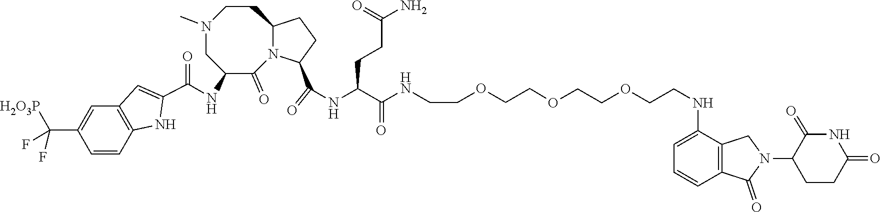 | ((2-(((5S,8S,10aR)-8-(((14S)-17-amino-1-((2-(2,6- dioxopiperidin-3-yl)-1-oxoisoindolin-4-yl)amino)-13,17- dioxo-3,6,9-trioxa-12-azaheptadecan-14-yl)carbamoyl)- 3-methyl-6-oxodecahydropyrrolo[1,2-a][1,5]diazocin-5- yl)carbamoyl)-1H-indol-5-yl)difluoromethyl)phosphonic acid |
| 18 | 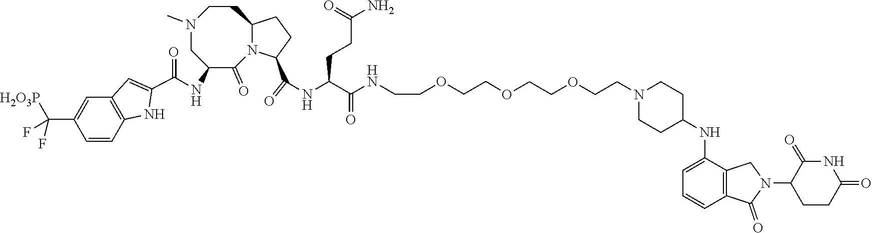 | ((2-(((5S,8S,10aR)-8-(((14S)-17-amino-1-(4-((2-(2,6- dioxopiperidin-3-yl)-1-oxoisoindolin-4- yl)amino)piperidin-1-yl)-13,17-dioxo-3,6,9-trioxa-12- azaheptadecan-14-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 19 | 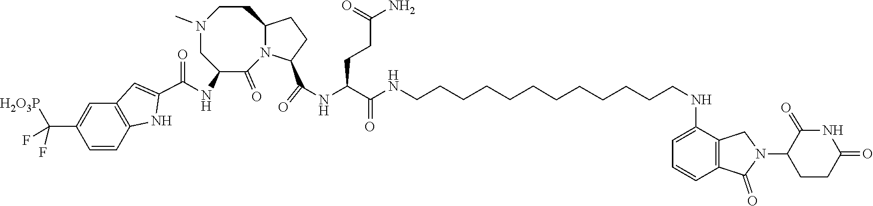 | ((2-(((5S,8S,10aR)-8-(((2S)-5-amino-1-((12-((2-(2,6- dioxopiperidin-3-yl)-1-oxoisoindolin-4- yl)amino)dodecyl)amino)-1,5-dioxopentan-2- yl)carbamoyl)-3-methyl-6-oxodecahydropyrrolo[1,2- a][1,5]diazocin-5-yl)carbamoyl)-1H-indol-5- yl)difluoromethyl)phosphonic acid |
| 20 | 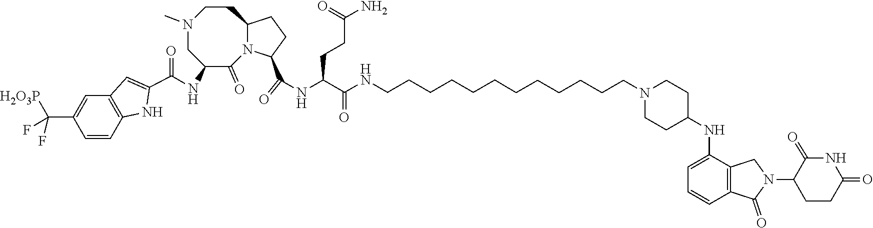 | ((2-(((5S,8S,10aR)-8-(((2S)-5-amino-1-((12-(4-((2-(2,6- dioxopiperidin-3-yl)-1-oxoisoindolin-4- yl)amino)piperidin-1-yl)dodecyl)amino)-1,5- dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 21 | 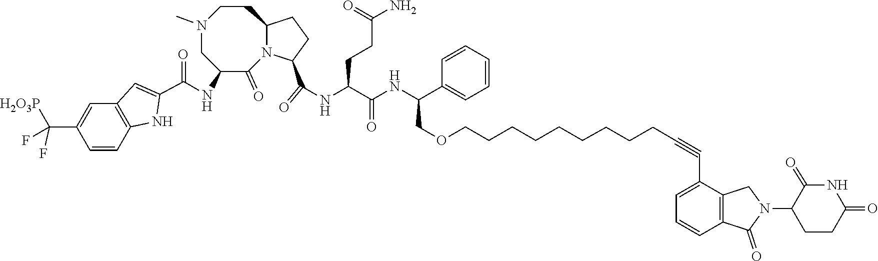 | ((2-(((5S,8S,10aR)-8-(((2S)-5-amino-1-(((1S)-2-((11-(2- (2,6-dioxopiperidin-3-yl)-1-oxoisoindolin-4-yl)undec-10- yn-1-yl)oxy)-1-phenylethyl)amino)-1,5-dioxopentan-2- yl)carbamoyl)-3-methyl-6-oxodecahydropyrrolo[1,2- a][1,5]diazocin-5-yl)carbamoyl)-1H-indol-5- yl)difluoromethyl)phosphonic acid |
| 22 | 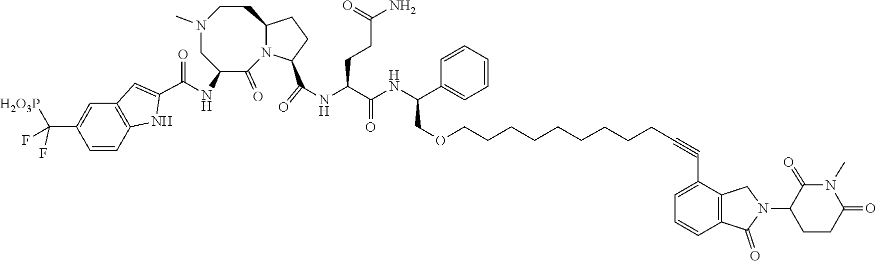 | ((2-(((5S,8S,10aR)-8-(((2S)-5-amino-1-(((1S)-2-((11-(2- (1-methyl-2,6-dioxopiperidin-3-yl)-1-oxoisoindolin-4- yl)undec-10-yn-1-yl)oxy)-1-phenylethyl)amino)-1,5- dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 23 | 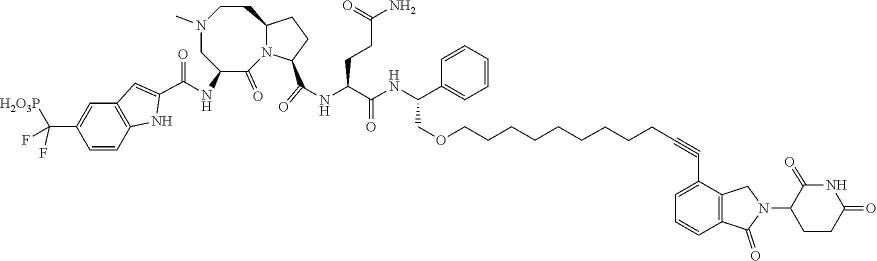 | ((2-(((5S,8S,10aR)-8-(((2S)-5-amino-1-(((1R)-2-((11-(2- (2,6-dioxopiperidin-3-yl)-1-oxoisoindolin-4-yl)undec-10- yn-1-yl)oxy)-1-phenylethyl)amino)-1,5-dioxopentan-2- yl)carbamoyl)-3-methyl-6-oxodecahydropyrrolo[1,2- a][1,5]diazocin-5-yl)carbamoyl)-1H-indol-5- yl)difluoromethyl)phosphonic acid |
| 24 | 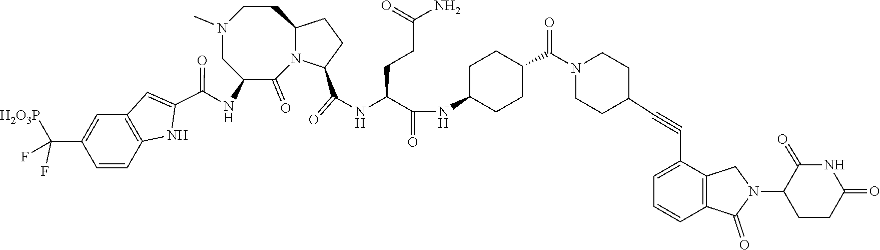 | ((2-(((5S,8S,10aR)-8-(((2S)-5-amino-1-(((1r,4S)-4-(4- ((2-(2,6-dioxopiperidin-3-yl)-1-oxoisoindolin-4- yl)ethynyl)piperidine-1-carbonyl)cyclohexyl)amino)-1,5- dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 25 | 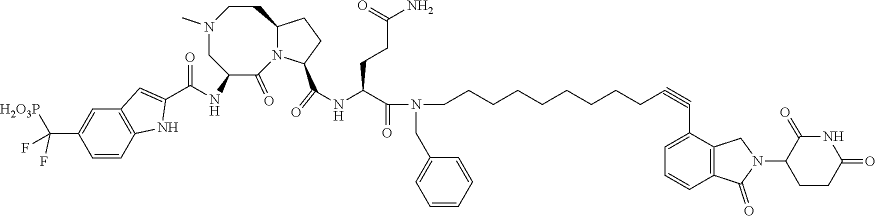 | ((2-(((5S,8S,10aR)-8-(((2S)-5-amino-1-(benzyl(11-(2- (2,6-dioxopiperidin-3-yl)-1-oxoisoindolin-4-yl)undec-10- yn-1-yl)amino)-1,5-dioxopentan-2-yl)carbamoyl)-3- methyl-6-oxodecahydropyrrolo[1,2-a][1,5]diazocin-5- yl)carbamoyl)-1H-indol-5-yl)difluoromethyl)phosphonic acid |
| 26 | 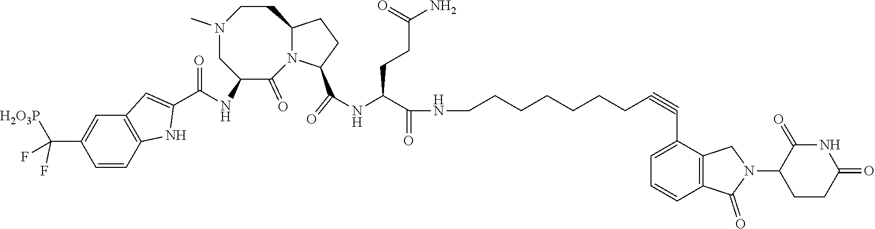 | ((2-(((5S,8S,10aR)-8-(((2S)-5-amino-1-((9-(2-(2,6- dioxopiperidin-3-yl)-1-oxoisoindolin-4-yl)non-8-yn-1- yl)amino)-1,5-dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 27 | 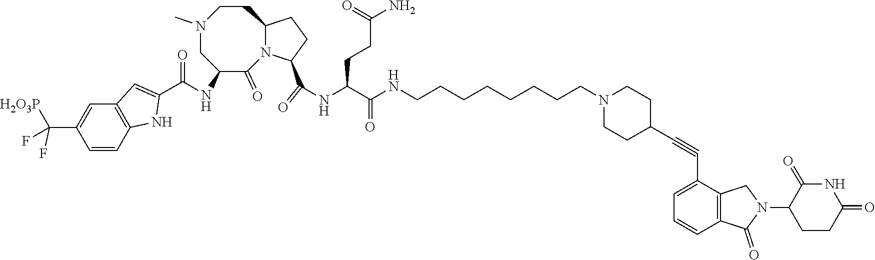 | ((2-(((5S,8S,10aR)-8-(((2S)-5-amino-1-((8-(4-((2-(2,6- dioxopiperidin-3-yl)-1-oxoisoindolin-4- yl)ethynyl)piperidin-1-yl)octyl)amino)-1,5-dioxopentan- 2-yl)carbamoyl)-3-methyl-6-oxodecahydropyrrolo[1,2- a][1,5]diazocin-5-yl)carbamoyl)-1H-indol-5- yl)difluoromethyl)phosphonic acid |
| 28 | 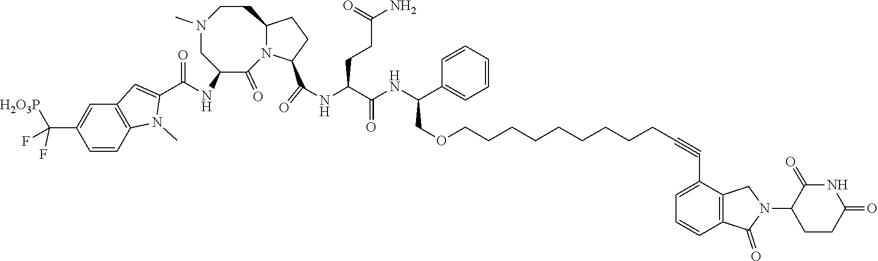 | ((2-(((5S,8S,10aR)-8-(((2S)-5-amino-1-(((1S)-2-((11-(2- (2,6-dioxopiperidin-3-yl)-1-oxoisoindolin-4-yl)undec-10- yn-1-yl)oxy)-1-phenylethyl)amino)-1,5-dioxopentan-2- yl)carbamoyl)-3-methyl-6-oxodecahydropyrrolo[1,2- a][1,5]diazocin-5-yl)carbamoyl)-1-methyl-1H-indol-5- yl)difluoromethyl)phosphonic acid |
| 29 | 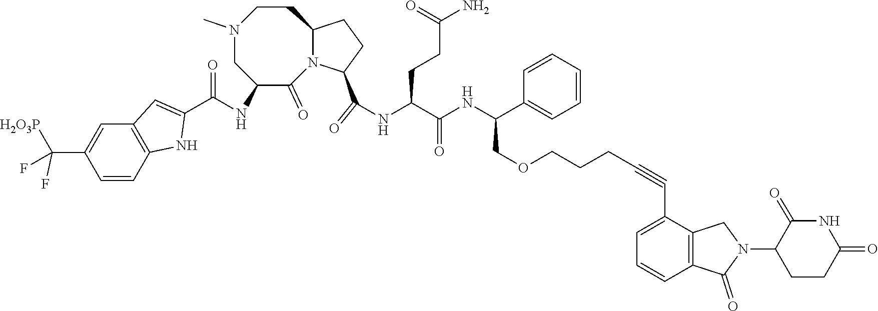 | ((2-(((5S,8S,10aR)-8-(((2S)-5-amino-1-(((1S)-2-((5-(2- (2,6-dioxopiperidin-3-yl)-1-oxoisoindolin-4-yl)pent-4- yn-1-yl)oxy)-1-phenylethyl)amino)-1,5-dioxopentan-2- yl)carbamoyl)-3-methyl-6-oxodecahydropyrrolo[1,2- a][1,5]diazocin-5-yl)carbamoyl)-1H-indol-5- yl)difluoromethyl)phosphonic acid |
| 30 | 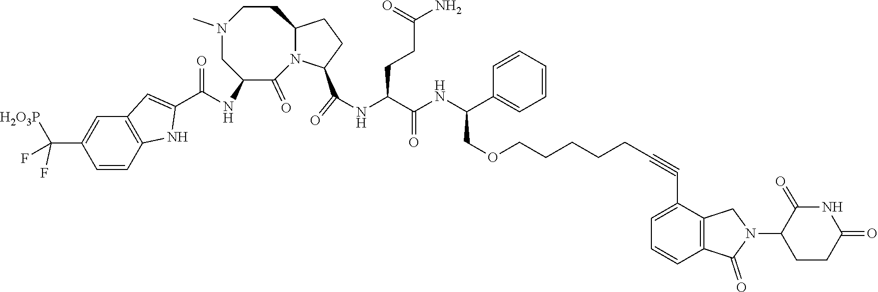 | ((2-(((5S,8S,10aR)-8-(((2S)-5-amino-1-(((1S)-2-((7-(2- (2,6-dioxopiperidin-3-yl)-1-oxoisoindolin-4-yl)hept-6- yn-1-yl)oxy)-1-phenylethyl)amino)-1,5-dioxopentan-2- yl)carbamoyl)-3-methyl-6-oxodecahydropyrrolo[1,2- a][1,5]diazocin-5-yl)carbamoyl)-1H-indol-5- yl)difluoromethyl)phosphonic acid |
| 31 | 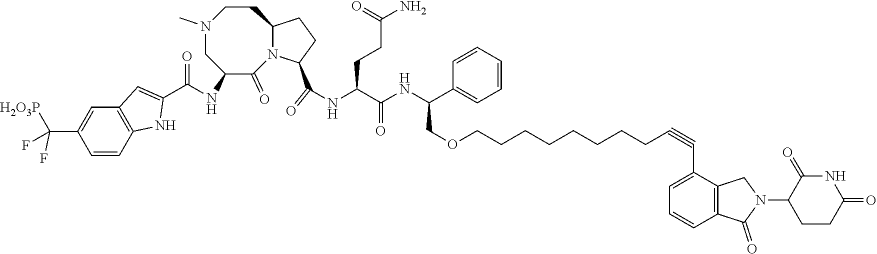 | ((2-(((5S,8S,10aR)-8-(((2S)-5-amino-1-(((1S)-2-((9-(2- (2,6-dioxopiperidin-3-yl)-1-oxoisoindolin-4-yl)non-8-yn- 1-yl)oxy)-1-phenylethyl)amino)-1,5-dioxopentan-2- yl)carbamoyl)-3-methyl-6-oxodecahydropyrrolo[1,2- a][1,5]diazocin-5-yl)carbamoyl)-1H-indol-5- yl)difluoromethyl)phosphonic acid |
| 32 | 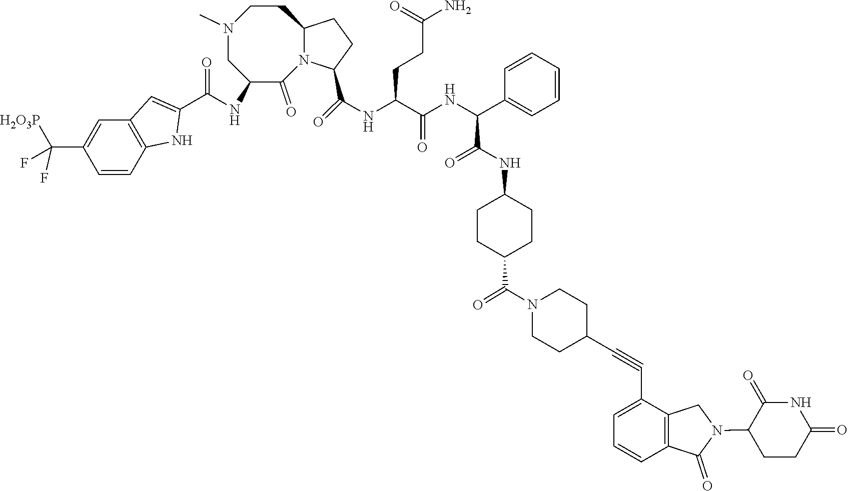 | ((2-(((5S,8S,10aR)-8-(((2S)-5-amino-1-(((1S)-2- (((1r,4S)-4-(4-((2-(2,6-dioxopiperidin-3-yl)-1- oxoisoindolin-4-yl)ethynyl)piperidine-1- carbonyl)cyclohexyl)amino)-2-oxo-1- phenylethyl)amino)-1,5-dioxopentan-2-yl)carbamoyl)-3- methyl-6-oxodecahydropyrrolo[1,2-a][1,5]diazocin-5- yl)carbamoyl)-1H-indol-5-yl)difluoromethyl)phosphonic acid |
| 33 | 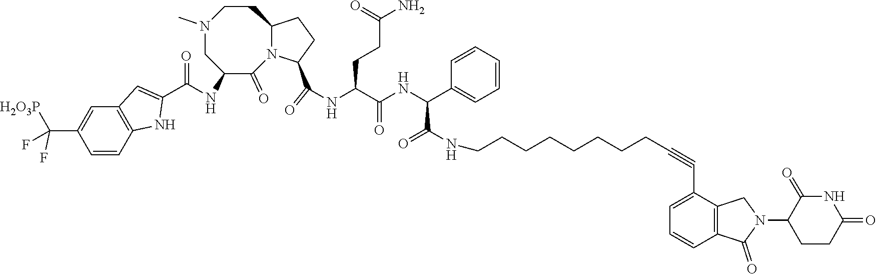 | ((2-(((5S,8S,10aR)-8-(((2S)-5-amino-1-(((1S)-2-((10-(2- (2,6-dioxopiperidin-3-yl)-1-oxoisoindolin-4-yl)dec-9-yn- 1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 34 | 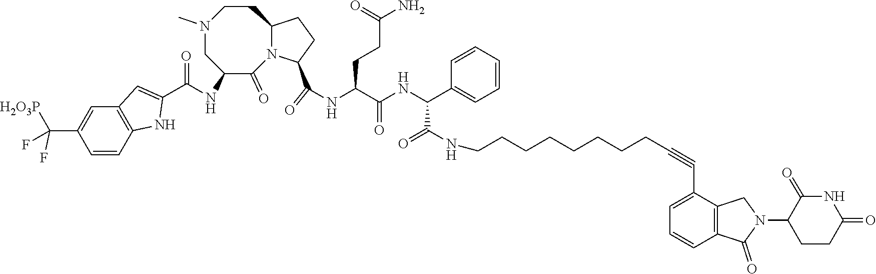 | ((2-(((5S,8S,10aR)-8-(((2S)-5-amino-1-(((1R)-2-((10-(2- (2,6-dioxopiperidin-3-yl)-1-oxoisoindolin-4-yl)dec-9-yn- 1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 35 | 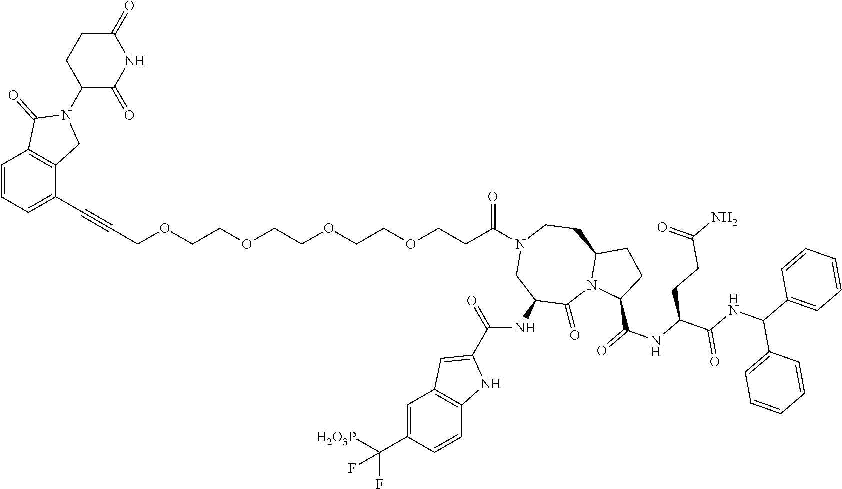 | ((2-(((5S,8S,10aR)-8-(((S)-5-amino-1- (benzhydrylamino)-1,5-dioxopentan-2-yl)carbamoyl)-3- (16-(2-(2,6-dioxopiperidin-3-yl)-1-oxoisoindolin-4-yl)- 4,7,10,13-tetraoxahexadec-15-ynoyl)-6- oxodecahydropyrrolo[1,2-a][1,5]diazocin-5- yl)carbamoyl)-1H-indol-5-yl)difluoromethyl)phosphonic acid |
| 36 | 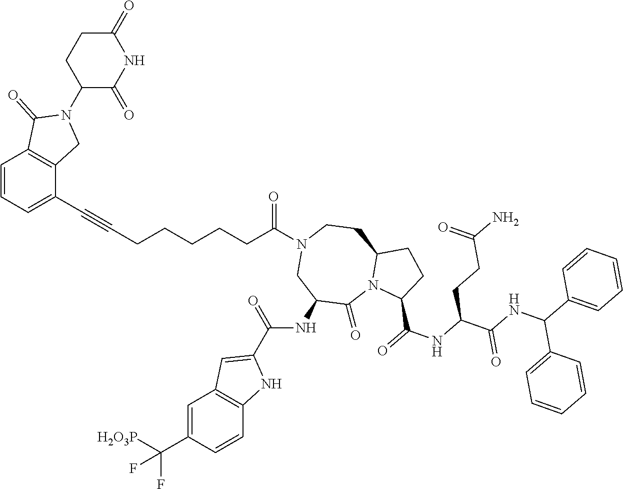 | ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)-1H-indol-5-yl)difluoromethyl)phosphonic acid |
| 37 | 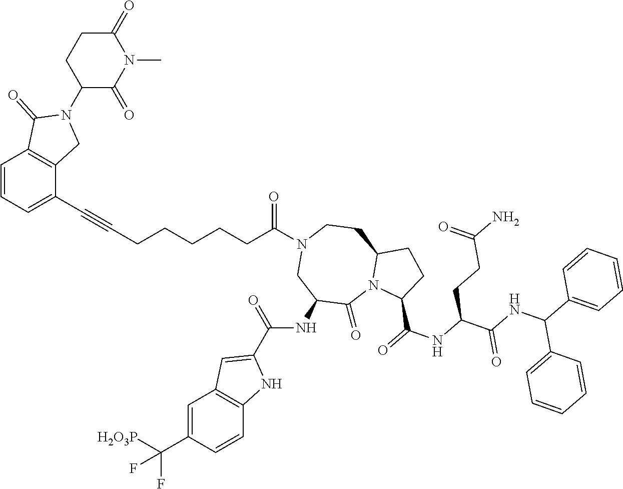 | ((2-(((5S,8S,10aR)-8-(((S)-5-amino-1- (benzhydrylamino)-1,5-dioxopentan-2-yl)carbamoyl)-3- (8-(2-(1-methyl-2,6-dioxopiperidin-3-yl)-1- oxoisoindolin-4-yl)oct-7-ynoyl)-6- oxodecahydropyrrolo[1,2-a][1,5]diazocin-5- yl)carbamoyl)-1H-indol-5-yl)difluoromethyl)phosphonic acid |
| 37E | 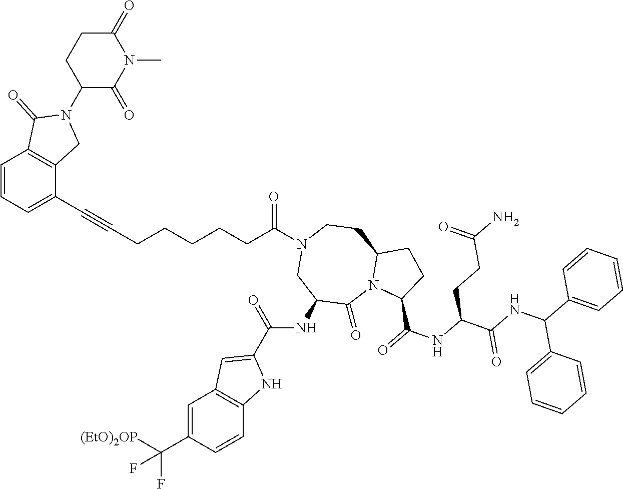 | diethyl ((2-(((5S,8S,10aR)-8-(((S)-5-amino-1- (benzhydrylamino)-1,5-dioxopentan-2-yl)carbamoyl)-3- (8-(2-(1-methyl-2,6-dioxopiperidin-3-yl)-1- oxoisoindolin-4-yl)oct-7-ynoyl)-6- oxodecahydropyrrolo[1,2-a][1,5]diazocin-5- yl)carbamoyl)-1H-indol-5- yl)difluoromethyl)phosphonate |
| 38 | 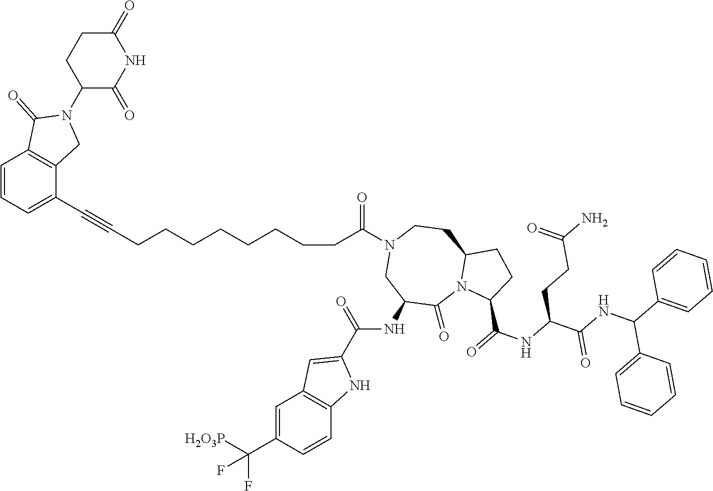 | ((2-(((5S,8S,10aR)-8-(((S)-5-amino-1- (benzhydrylamino)-1,5-dioxopentan-2-yl)carbamoyl)-3- (11-(2-(2,6-dioxopiperidin-3-yl)-1-oxoisoindolin-4- yl)undec-10-ynoyl)-6-oxodecahydropyrrolo[1,2- a][1,5]diazocin-5-yl)carbamoyl)-1H-indol-5- yl)difluoromethyl)phosphonic acid |
| 39 | 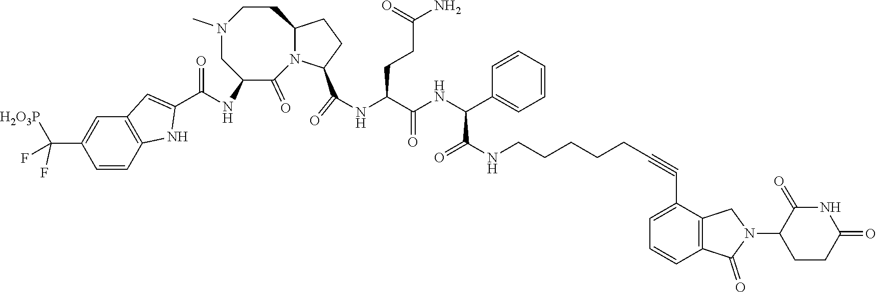 | ((2-(((5S,8S,10aR)-8-(((2S)-5-amino-1-(((1S)-2-((7-(2- (2,6-dioxopiperidin-3-yl)-1-oxoisoindolin-4-yl)hept-6- yn-1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 40 | 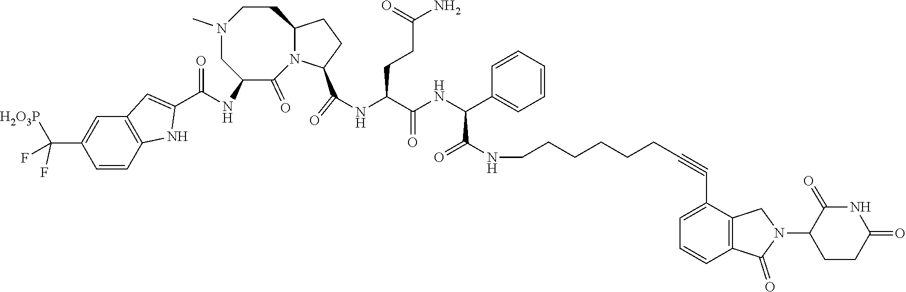 | ((2-(((5S,8S,10aR)-8-(((2S)-5-amino-1-(((1S)-2-((8-(2- (2,6-dioxopiperidin-3-yl)-1-oxoisoindolin-4-yl)oct-7-yn- 1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 41 | 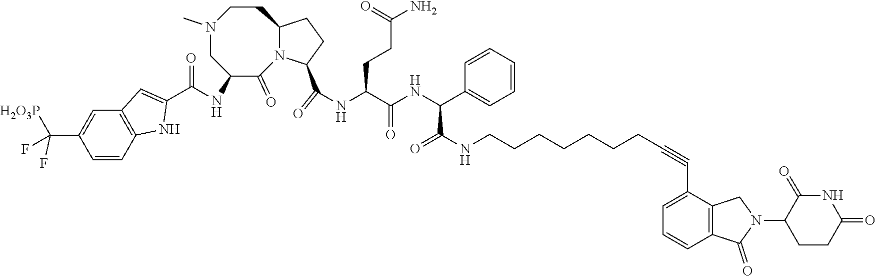 | ((2-(((5S,8S,10aR)-8-(((2S)-5-amino-1-(((1S)-2-((9-(2- (2,6-dioxopiperidin-3-yl)-1-oxoisoindolin-4-yl)non-8-yn- 1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 42 | 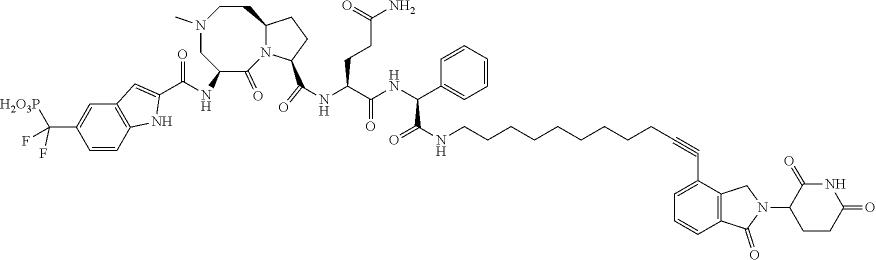 | ((2-(((5S,8S,10aR)-8-(((2S)-5-amino-1-(((1S)-2-((11-(2- (2,6-dioxopiperidin-3-yl)-1-oxoisoindolin-4-yl)undec-10- yn-1-yl)amino)-2-oxo-1-phenylethyl)amino)-1,5- dioxopentan-2-yl)carbamoyl)-3-methyl-6- oxodecahydropyrrolo[1,2-a][1,5] diazocin-5- yl)carbamoyl)-1H-indol-5-yl)difluoromethyl)phosphonic acid |
| 43 | 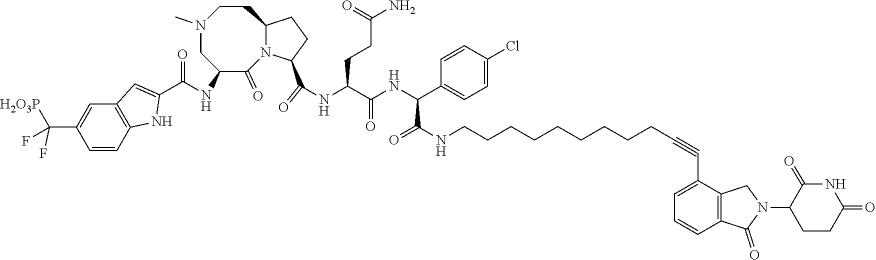 | ((2-(((5S,8S,10aR)-8-(((2S)-5-amino-1-(((1S)-1-(4- chlorophenyl)-2-((11-(2-(2,6-dioxopiperidin-3-yl)-1- oxoisoindolin-4-yl)undec-10-yn-1-yl)amino)-2- oxoethyl)amino)-1,5-dioxopentan-2-yl)carbamoyl)-3- methyl-6-oxodecahydropyrrolo[1,2-a][1,5]diazocin-5- yl)carbamoyl)-1H-indol-5-yl)difluoromethyl)phosphonic acid |
| 44 | 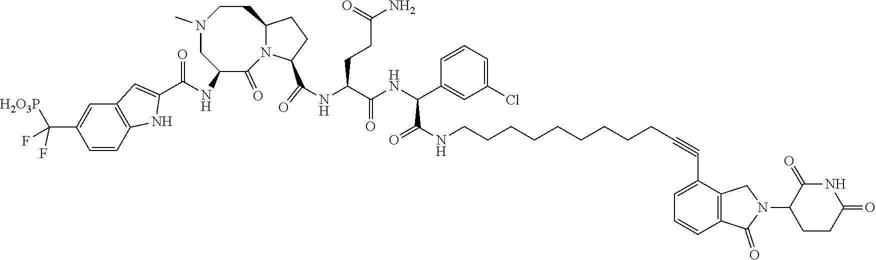 | ((2-(((5S,8S,10aR)-8-(((2S)-5-amino-1-(((1S)-1-(3- chlorophenyl)-2-((11-(2-(2,6-dioxopiperidin-3-yl)-1- oxoisoindolin-4-yl)undec-10-yn-1-yl)amino)-2- oxoethyl)amino)-1,5-dioxopentan-2-yl)carbamoyl)-3- methyl-6-oxodecahydropyrrolo[1,2-a][1,5]diazocin-5- yl)carbamoyl)-1H-indol-5-yl)difluoromethyl)phosphonic acid |
| 47 | 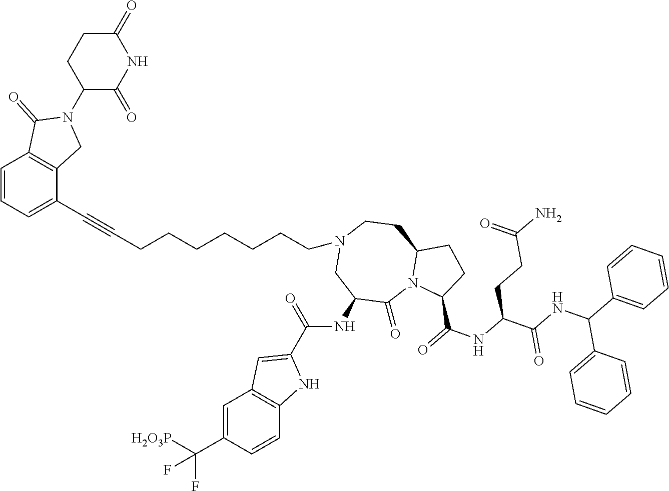 | ((2-(((5S,8S,10aR)-8-(((S)-5-amino-1- (benzhydrylamino)-1,5-dioxopentan-2-yl)carbamoyl)-3- (9-(2-(2,6-dioxopiperidin-3-yl)-1-oxoisoindolin-4- yl)non-8-yn-1-yl)-6-oxodecahydropyrrolo[1,2- a][1,5]diazocin-5-yl)carbamoyl)-1H-indol-5- yl)difluoromethyl)phosphonic acid |
| 48 | 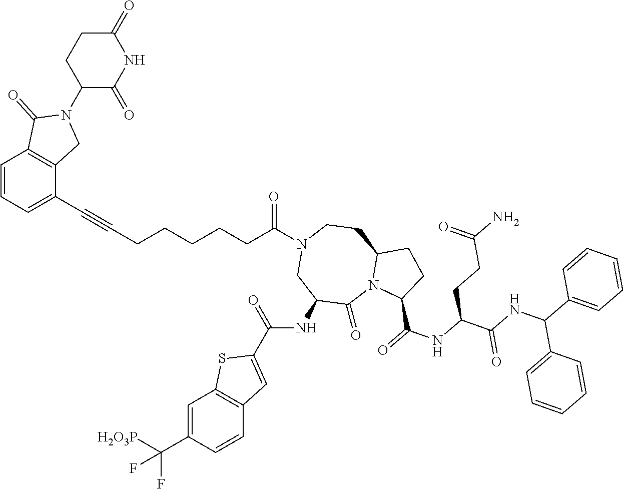 | ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)benzo[b]thiophen-6- yl)difluoromethyl)phosphonic acid |
| 49 | 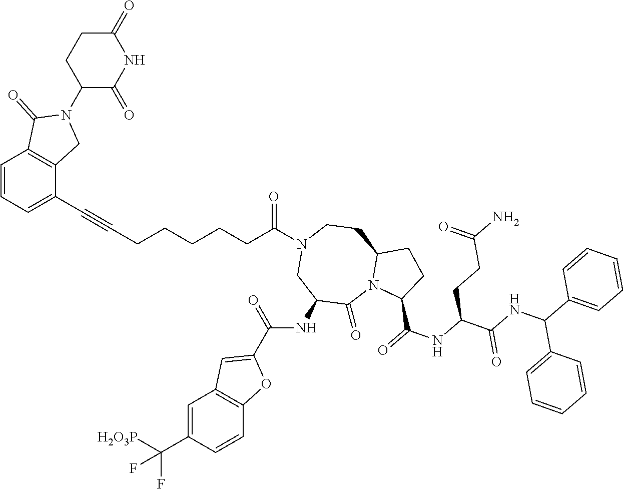 | ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)benzofuran-5- yl)difluoromethyl)phosphonic acid |
| 50 | 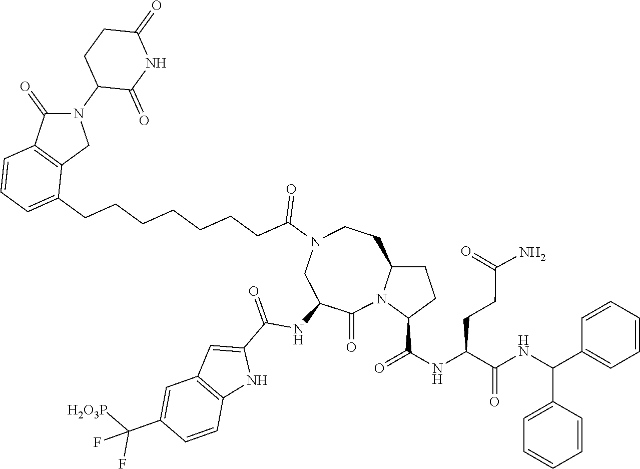 | ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4- yl)octanoyl)-6-oxodecahydropyrrolo[1,2-a][1,5]diazocin- 5-yl)carbamoyl)-1H-indol-5- yl)difluoromethyl)phosphonic acid |
| 51 | 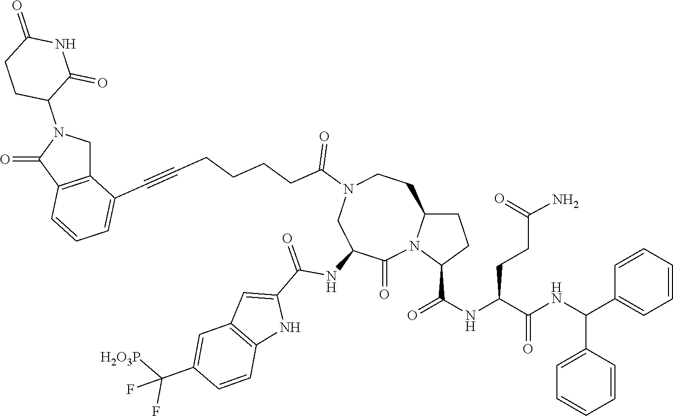 | ((2-(((5S,8S,10aR)-8-(((S)-5-amino-1- (benzhydrylamino)-1,5-dioxopentan-2-yl)carbamoyl)-3- (7-(2-(2,6-dioxopiperidin-3-yl)-1-oxoisoindolin-4- yl)hept-6-ynoyl)-6-oxodecahydropyrrolo[1,2- a][1,5]diazocin-5-yl)carbamoyl)-1H-indol-5- yl)difluoromethyl)phosphonic acid |
| 52 | 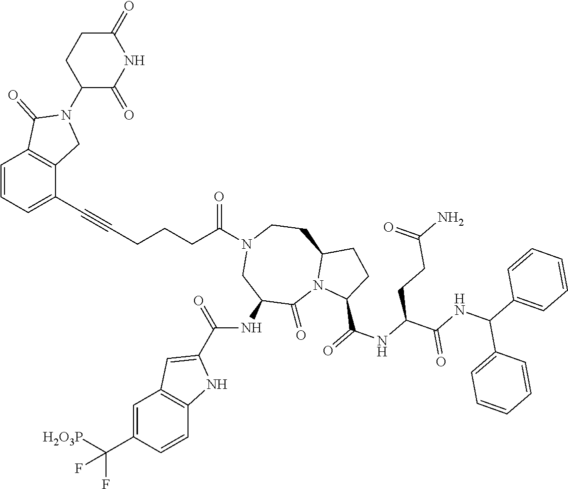 | ((2-(((5S,8S,10aR)-8-(((S)-5-amino-1- (benzhydrylamino)-1,5-dioxopentan-2-yl)carbamoyl)-3- (6-(2-(2,6-dioxopiperidin-3-yl)-1-oxoisoindolin-4-yl)hex- 5-ynoyl)-6-oxodecahydropyrrolo[1,2-a][1,5]diazocin-5- yl)carbamoyl)-1H-indol-5-yl)difluoromethyl)phosphonic acid |
| 53 | 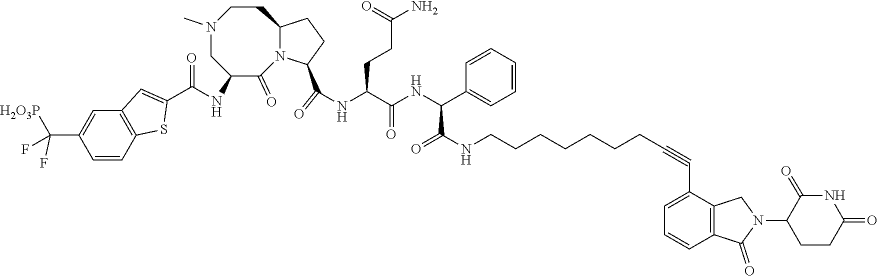 | ((2-(((5S,8S,10aR)-8-(((2S)-5-amino-1-(((1S)-2-((9-(2- (2,6-dioxopiperidin-3-yl)-1-oxoisoindolin-4-yl)non-8-yn- 1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 54 | 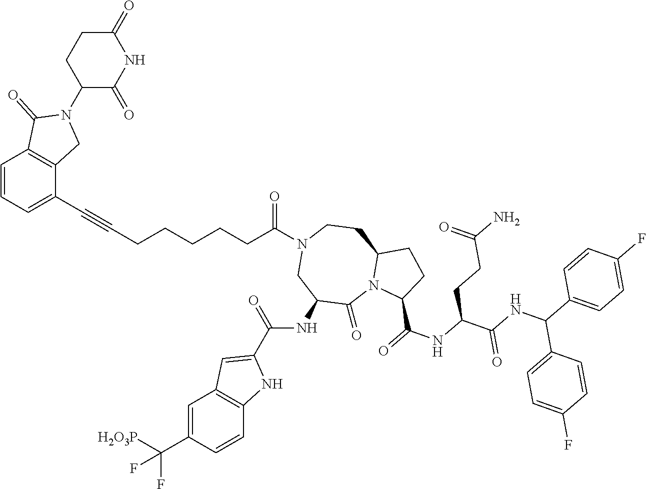 | ((2-(((5S,8S,10aR)-8-(((S)-5-amino-1-((bis(4- fluorophenyl)methyl)amino)-1,5-dioxopentan-2- yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)-1H-indol-5-yl)difluoromethyl)phosphonic acid |
| 55 | 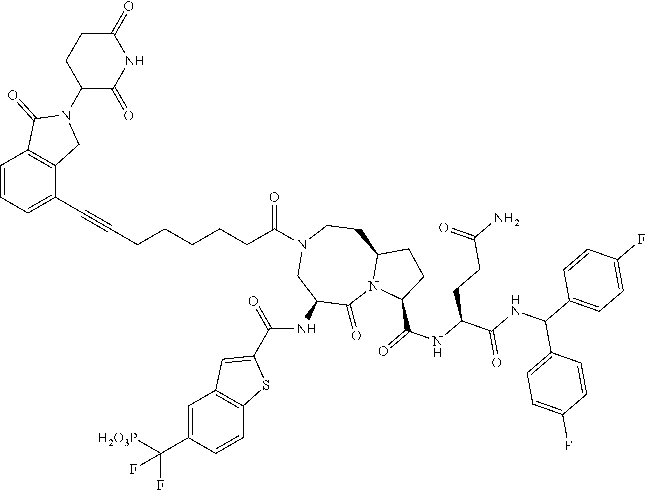 | ((2-(((5S,8S,10aR)-8-(((S)-5-amino-1-((bis(4- fluorophenyl)methyl)amino)-1,5-dioxopentan-2- yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)benzo[b]thiophen-5- yl)difluoromethyl)phosphonic acid |
| 56 | 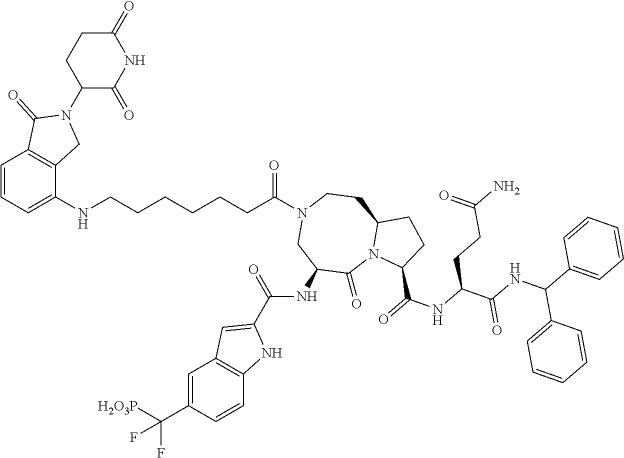 | ((2-(((5S,8S,10aR)-8-(((S)-5-amino-1- (benzhydrylamino)-1,5-dioxopentan-2-yl)carbamoyl)-3- (7-((2-(2,6-dioxopiperidin-3-yl)-1-oxoisoindolin-4- yl)amino)heptanoyl)-6-oxodecahydropyrrolo[1,2- a][1,5]diazocin-5-yl)carbamoyl)-1H-indol-5- yl)difluoromethyl)phosphonic acid |
| 57 | 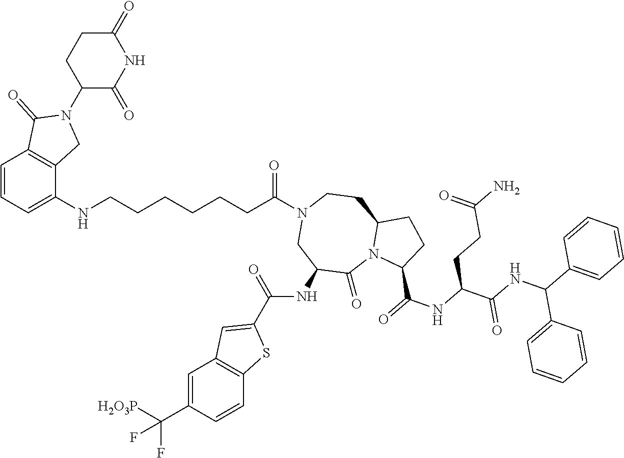 | ((2-(((5S,8S,10aR)-8-(((S)-5-amino-1- (benzhydrylamino)-1,5-dioxopentan-2-yl)carbamoyl)-3- (7-((2-(2,6-dioxopiperidin-3-yl)-1-oxoisoindolin-4- yl)amino)heptanoyl)-6-oxodecahydropyrrolo[1,2- a][1,5]diazocin-5-yl)carbamoyl)benzo[b]thiophen-5- yl)difluoromethyl)phosphonic acid |
| 58 | 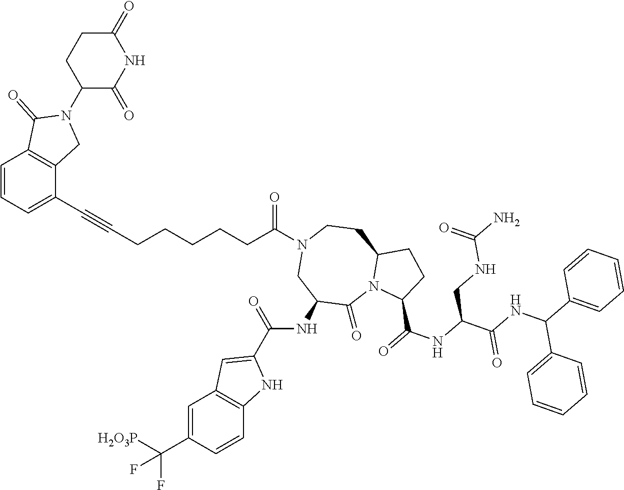 | ((2-(((5S,8S,10aR)-8-(((S)-1-(benzhydrylamino)-1-oxo- 3-ureidopropan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)-1H-indol-5-yl)difluoromethyl)phosphonic acid |
| 59 | 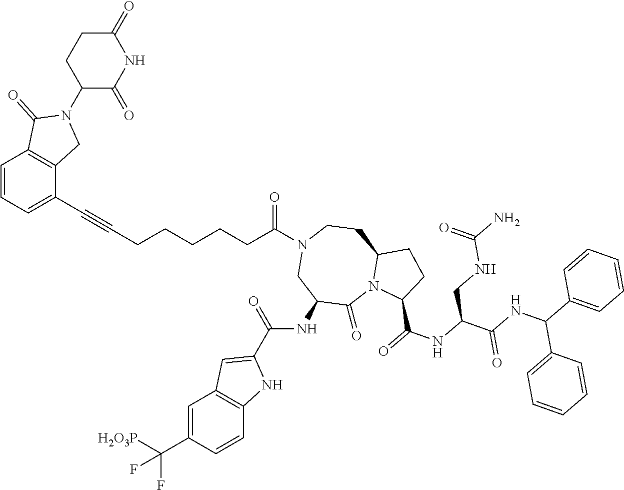 | ((2-(((5S,8S,10aR)-8-(((S)-1-(benzhydrylamino)-1-oxo- 3-ureidopropan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)-1H-indol-5-yl)difluoromethyl)phosphonic acid |
| 60 | 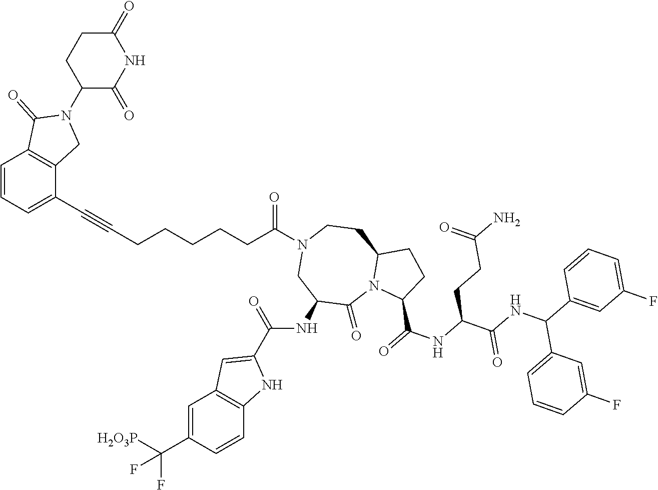 | ((2-(((5S,8S,10aR)-8-(((S)-5-amino-1-((bis(3- fluorophenyl)methyl)amino)-1,5-dioxopentan-2- yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)-1H-indol-5-yl)difluoromethyl)phosphonic acid |
| 61 | 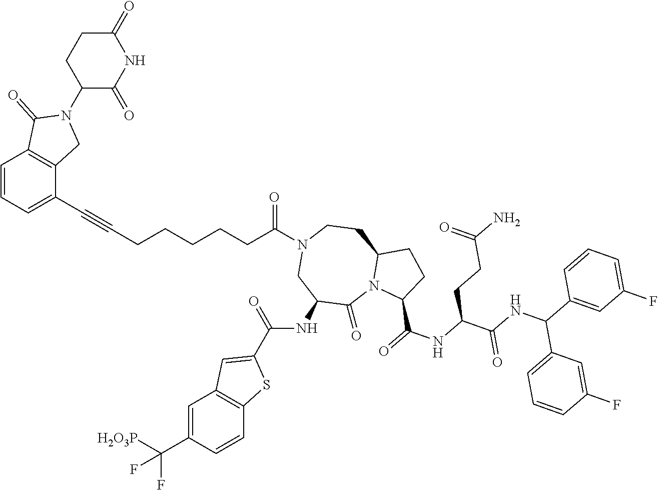 | ((2-(((5S,8S,10aR)-8-(((S)-5-amino-1-((bis(3- fluorophenyl)methyl)amino)-1,5-dioxopentan-2- yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)benzo[b]thiophen-5- yl)difluoromethyl)phosphonic acid |
| 62 | 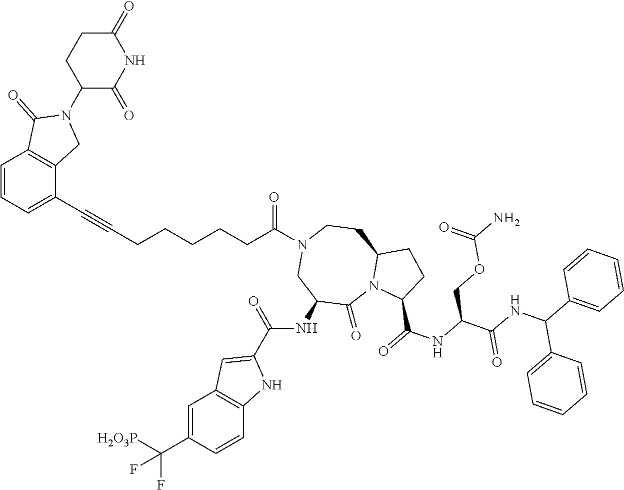 | ((2-(((5S,8S,10aR)-8-(((S)-1-(benzhydrylamino)-3- (carbamoyloxy)-1-oxopropan-2-yl)carbamoyl)-3-(8-(2- (2,6-dioxopiperidin-3-yl)-1-oxoisoindolin-4-yl)oct-7- ynoyl)-6-oxodecahydropyrrolo[1,2-a][1,5]diazocin-5- yl)carbamoyl)-1H-indol-5-yl)difluoromethyl)phosphonic acid |
| 62E | 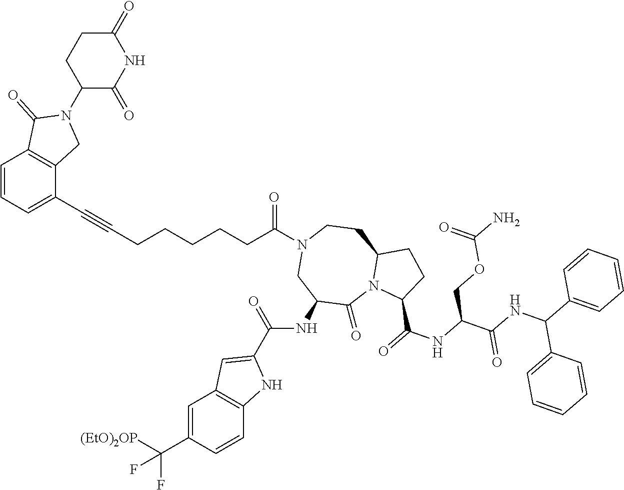 | (2S)-3-(benzhydrylamino)-2-((5S,8S,10aR)-5-(5- ((diethoxyphosphoryl)difluoromethyl)-1H-indole-2- carboxamido)-3-(8-(2-(2,6-dioxopiperidin-3-yl)-1- oxoisoindolin-4-yl)oct-7-ynoyl)-6- oxodecahydropyrrolo[1,2-a][1,5]diazocine-8- carboxamido)-3-oxopropyl carbamate |
| 63 | 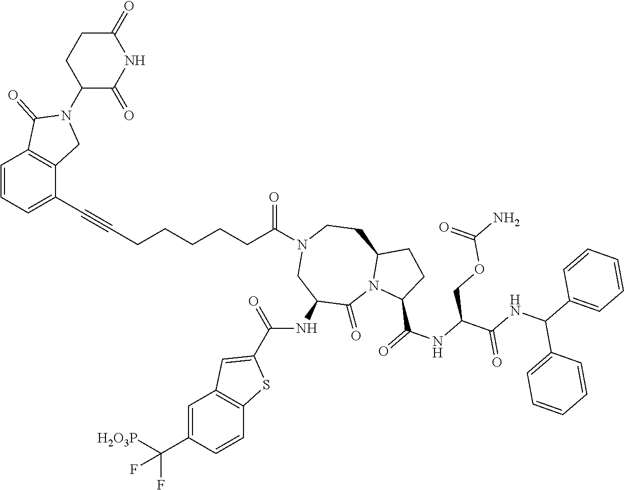 | ((2-(((5S,8S,10aR)-8-(((S)-1-(benzhydrylamino)-3- (carbamoyloxy)-1-oxopropan-2-yl)carbamoyl)-3-(8-(2- (2,6-dioxopiperidin-3-yl)-1-oxoisoindolin-4-yl)oct-7- ynoyl)-6-oxodecahydropyrrolo[1,2-a][1,5]diazocin-5- yl)carbamoyl)benzo[b]thiophen-5- yl)difluoromethyl)phosphonic acid |
| 64 | 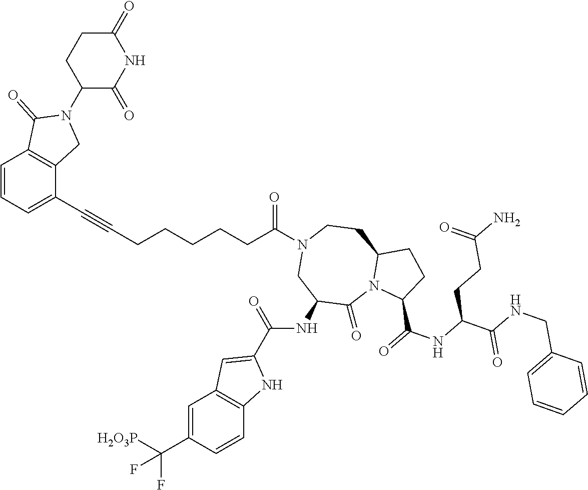 | ((2-(((5S,8S,10aR)-8-(((S)-5-amino-1-(benzylamino)- 1,5-dioxopentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)-1H-indol-5-yl)difluoromethyl)phosphonic acid |
| 65 | 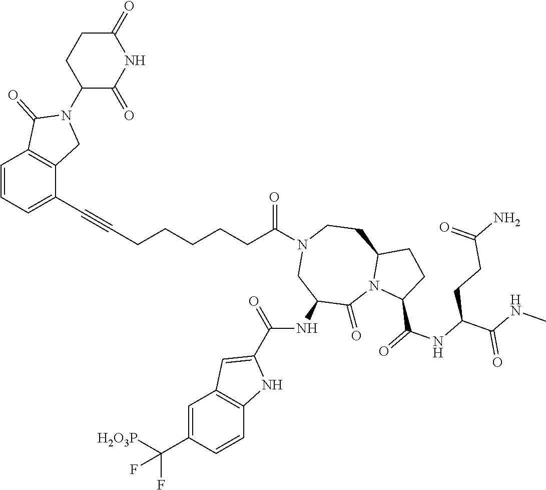 | ((2-(((5S,8S,10aR)-8-(((S)-5-amino-1-(methylamino)- 1,5-dioxopentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)-1H-indol-5-yl)difluoromethyl)phosphonic acid |
| 66 | 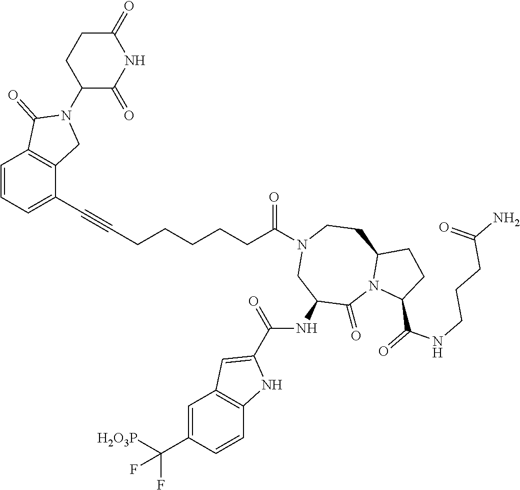 | ((2-(((5S,8S,10aR)-8-((4-amino-4-oxobutyl)carbamoyl)- 3-(8-(2-(2,6-dioxopiperidin-3-yl)-1-oxoisoindolin-4- yl)oct-7-ynoyl)-6-oxodecahydropyrrolo[1,2- a][1,5]diazocin-5-yl)carbamoyl)-1H-indol-5- yl)difluoromethyl)phosphonic acid |
| 67 | 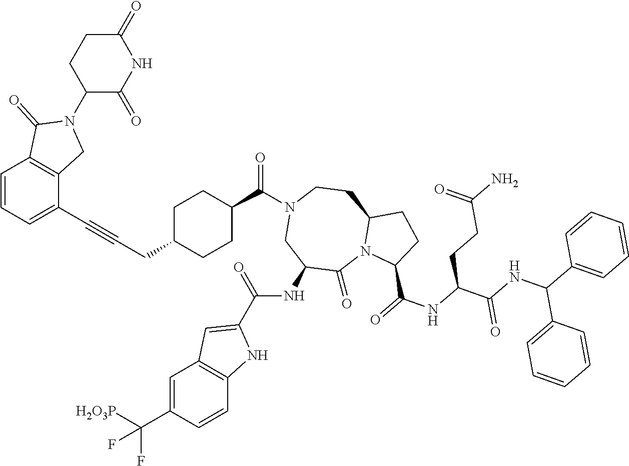 | ((2-(((5S,8S,10aR)-8-(((S)-5-amino-1- (benzhydrylamino)-1,5-dioxopentan-2-yl)carbamoyl)-3- ((1r,4S)-4-(3-(2-(2,6-dioxopiperidin-3-yl)-1- oxoisoindolin-4-yl)prop-2-yn-1-yl)cyclohexane-1- carbonyl)-6-oxodecahydropyrrolo[1,2-a][1,5]diazocin-5- yl)carbamoyl)-1H-indol-5-yl)difluoromethyl)phosphonic acid |
| 68 | 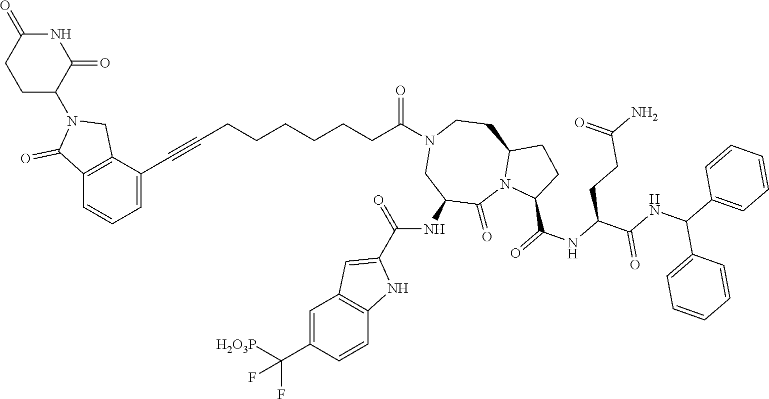 | ((2-(((5S,8S,10aR)-8-(((S)-5-amino-1- (benzhydrylamino)-1,5-dioxopentan-2-yl)carbamoyl)-3- (9-(2-(2,6-dioxopiperidin-3-yl)-1-oxoisoindolin-4- yl)non-8-ynoyl)-6-oxodecahydropyrrolo[1,2- a][1,5]diazocin-5-yl)carbamoyl)-1H-indol-5- yl)difluoromethyl)phosphonic acid |
| 69 | 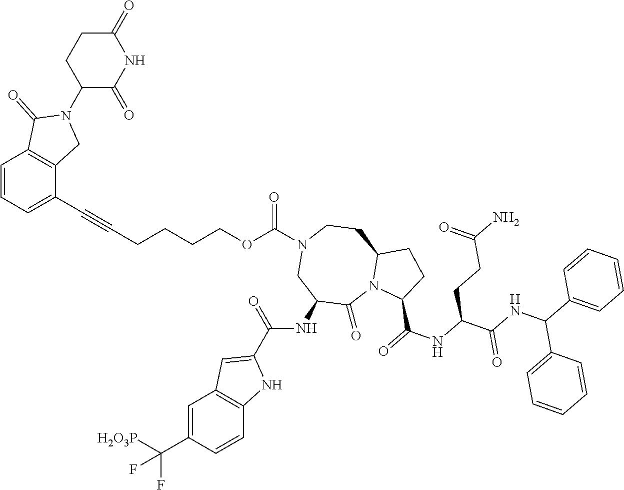 | ((2-(((5S,8S,10aR)-8-(((S)-5-amino-1- (benzhydrylamino)-1,5-dioxopentan-2-yl)carbamoyl)-3- (((6-(2-(2,6-dioxopiperidin-3-yl)-1-oxoisoindolin-4- yl)hex-5-yn-1-yl)oxy)carbonyl)-6- oxodecahydropyrrolo[1,2-a][1,5]diazocin-5- yl)carbamoyl)-1H-indol-5-yl)difluoromethyl)phosphonic acid |
| 69E | 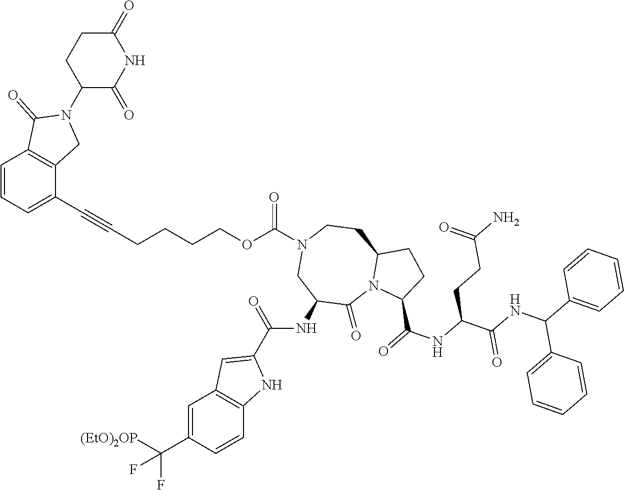 | 6-(2-(2,6-dioxopiperidin-3-yl)-1-oxoisoindolin-4-yl)hex- 5-yn-1-yl (5S,8S,10aR)-8-(((S)-5-amino-1- (benzhydrylamino)-1,5-dioxopentan-2-yl)carbamoyl)-5- (5-((diethoxyphosphoryl)difluoromethyl)-1H-indole-2- carboxamido)-6-oxooctahydropyrrolo[1,2- a][1,5]diazocine-3(4H)-carboxylate |
| 70 | 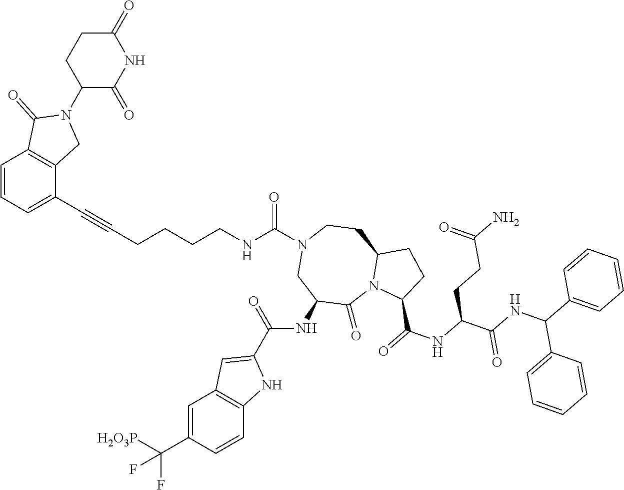 | ((2-(((5S,8S,10aR)-8-(((S)-5-amino-1- (benzhydrylamino)-1,5-dioxopentan-2-yl)carbamoyl)-3- ((6-(2-(2,6-dioxopiperidin-3-yl)-1-oxoisoindolin-4- yl)hex-5-yn-1-yl)carbamoyl)-6- oxodecahydropyrrolo[1,2-a][1,5]diazocin-5- yl)carbamoyl)-1H-indol-5-yl)difluoromethyl)phosphonic acid |
| 70E | 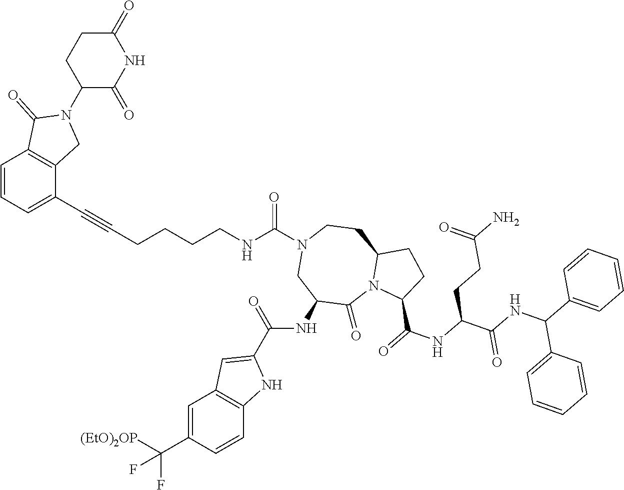 | diethyl ((2-(((5S,8S,10aR)-8-(((S)-5-amino-1- (benzhydrylamino)-1,5-dioxopentan-2-yl)carbamoyl)-3- ((6-(2-(2,6-dioxopiperidin-3-yl)-1-oxoisoindolin-4- yl)hex-5-yn-1-yl)carbamoyl)-6- oxodecahydropyrrolo[1,2-a][1,5] diazocin-5- yl)carbamoyl)-1H-indol-5- yl)difluoromethyl)phosphonate |
| 71 | 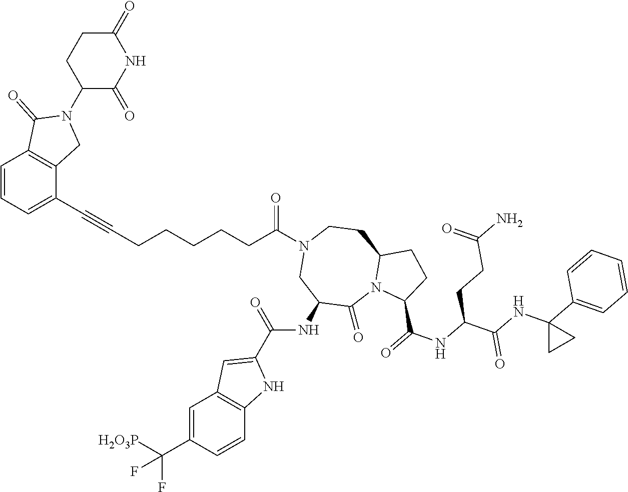 | ((2-(((5S,8S,10aR)-8-(((S)-5-amino-1,5-dioxo-1-((1- phenylcyclopropyl)amino)pentan-2-yl)carbamoyl)-3-(8- (2-(2,6-dioxopiperidin-3-yl)-1-oxoisoindolin-4-yl)oct-7- ynoyl)-6-oxodecahydropyrrolo[1,2-a][1,5]diazocin-5- yl)carbamoyl)-1H-indol-5-yl)difluoromethyl)phosphonic acid |
| 72 | 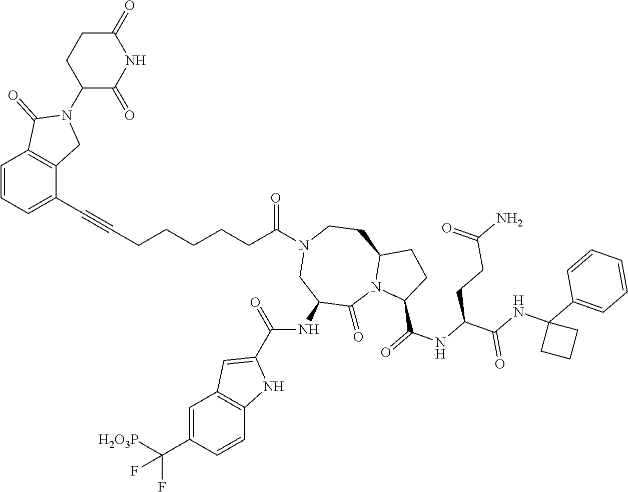 | ((2-(((5S,8S,10aR)-8-(((S)-5-amino-1,5-dioxo-1-((1- phenylcyclobutyl)amino)pentan-2-yl)carbamoyl)-3-(8-(2- (2,6-dioxopiperidin-3-yl)-1-oxoisoindolin-4-yl)oct-7- ynoyl)-6-oxodecahydropyrrolo[1,2-a][1,5]diazocin-5- yl)carbamoyl)-1H-indol-5-yl)difluoromethyl)phosphonic acid |
| 73 | 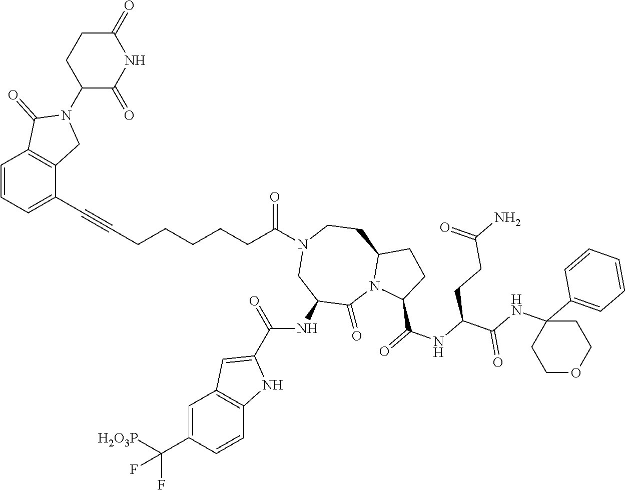 | ((2-(((5S,8S,10aR)-8-(((S)-5-amino-1,5-dioxo-1-((4- phenyltetrahydro-2H-pyran-4-yl)amino)pentan-2- yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)-1H-indol-5-yl)difluoromethyl)phosphonic acid |
| 74 | 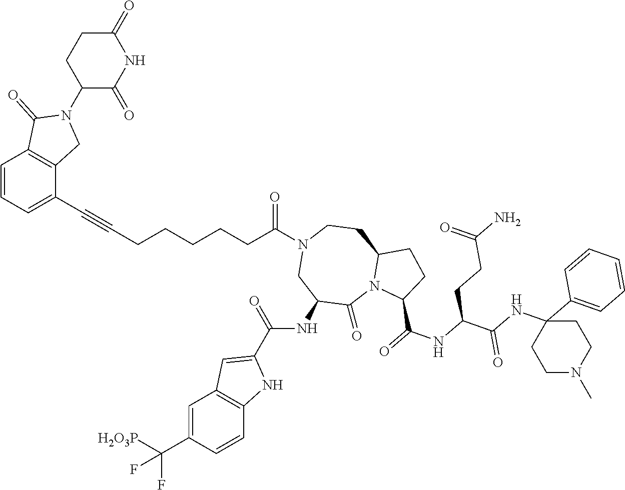 | ((2-(((5S,8S,10aR)-8-(((S)-5-amino-1-((1-methyl-4- phenylpiperidin-4-yl)amino)-1,5-dioxopentan-2- yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)-1H-indol-5-yl)difluoromethyl)phosphonic acid |
| 75 | 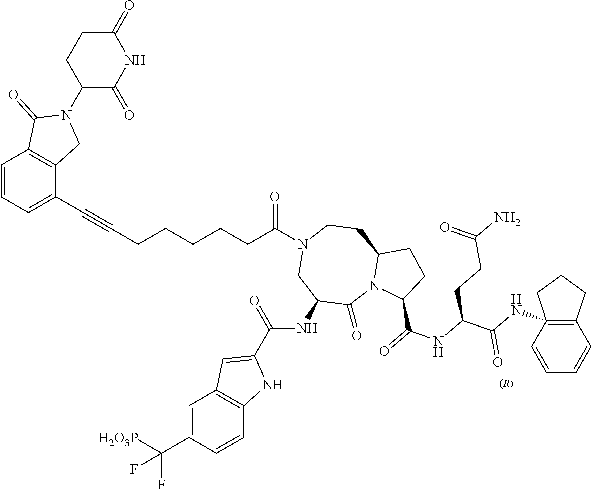 | ((2-(((5S,8S,10aR)-8-(((S)-5-amino-1-(((R)-2,3-dihydro- 1H-inden-1-yl)amino)-1,5-dioxopentan-2-yl)carbamoyl)- 3-(8-(2-(2,6-dioxopiperidin-3-yl)-1-oxoisoindolin-4- yl)oct-7-ynoyl)-6-oxodecahydropyrrolo[1,2- a][1,5]diazocin-5-yl)carbamoyl)-1H-indol-5- yl)difluoromethyl)phosphonic acid |
| 76 | 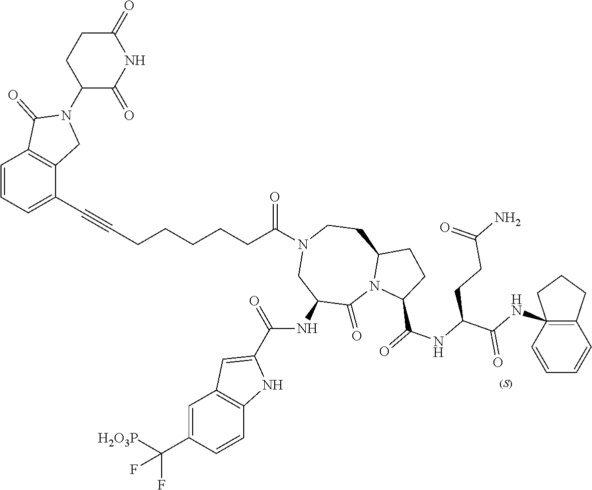 | ((2-(((5S,8S,10aR)-8-(((S)-5-amino-1-(((S)-2,3-dihydro- 1H-inden-1-yl)amino)-1,5-dioxopentan-2-yl)carbamoyl)- 3-(8-(2-(2,6-dioxopiperidin-3-yl)-1-oxoisoindolin-4- yl)oct-7-ynoyl)-6-oxodecahydropyrrolo[1,2- a][1,5]diazocin-5-yl)carbamoyl)-1H-indol-5- yl)difluoromethyl)phosphonic acid |
| 77 | 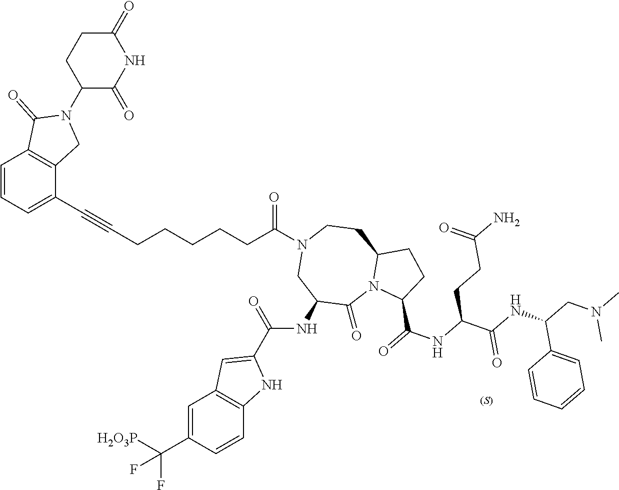 | ((2-(((5S,8S,10aR)-8-(((S)-5-amino-1-(((S)-2- (dimethylamino)-1-phenylethyl)amino)-1,5-dioxopentan- 2-yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)-1H-indol-5-yl)difluoromethyl)phosphonic acid |
| 78 | 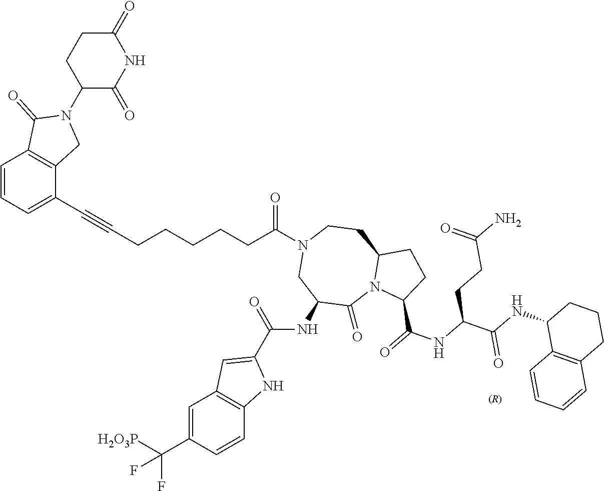 | ((2-(((5S,8S,10aR)-8-(((S)-5-amino-1,5-dioxo-1-(((R)- 1,2,3,4-tetrahydronaphthalen-1-yl)amino)pentan-2- yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)-1H-indol-5-yl)difluoromethyl)phosphonic acid |
| 79 | 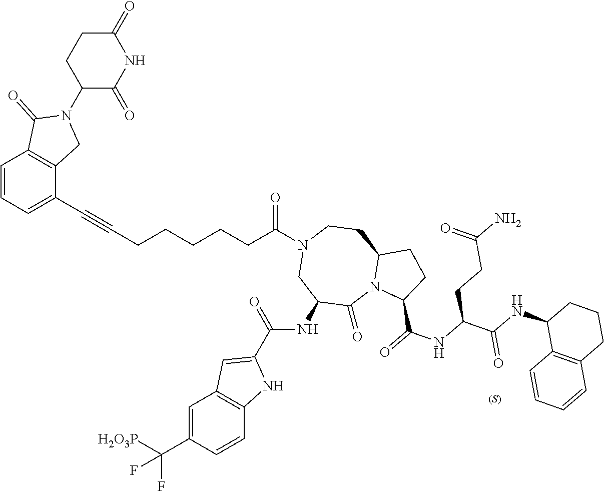 | ((2-(((5S,8S,10aR)-8-(((S)-5-amino-1,5-dioxo-1-(((S)- 1,2,3,4-tetrahydronaphthalen-1-yl)amino)pentan-2- yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)-1H-indol-5-yl)difluoromethyl)phosphonic acid |
| 80 | 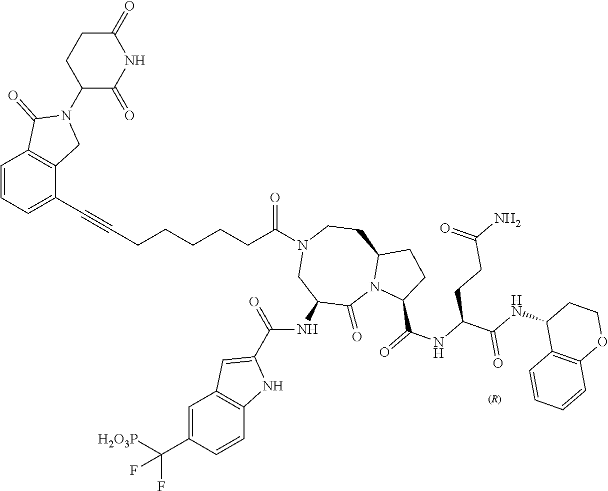 | ((2-(((5S,8S,10aR)-8-(((S)-5-amino-1-(((R)-chroman-4- yl)amino)-1,5-dioxopentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)-1H-indol-5-yl)difluoromethyl)phosphonic acid |
| 81 | 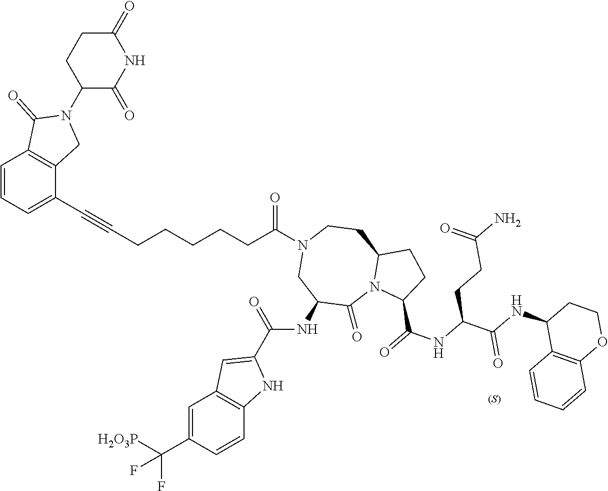 | ((2-(((5S,8S,10aR)-8-(((S)-5-amino-1-(((S)-chroman-4- yl)amino)-1,5-dioxopentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)-1H-indol-5-yl)difluoromethyl)phosphonic acid |
| 82 | 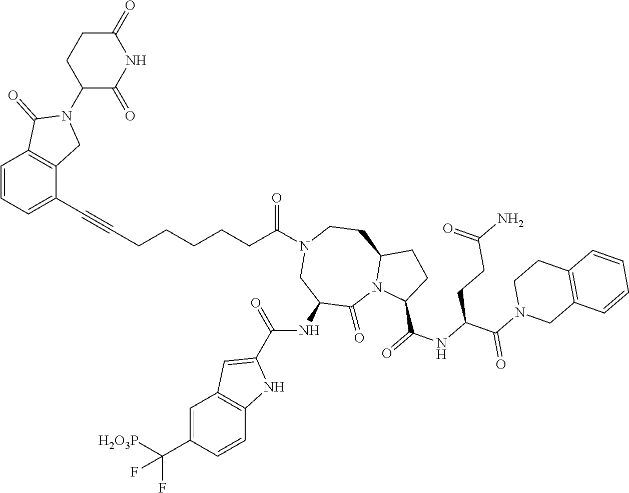 | ((2-(((5S,8S,10aR)-8-(((S)-5-amino-1-(3,4- dihydroisoquinolin-2(1H)-yl)-1,5-dioxopentan-2- yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)-1H-indol-5-yl)difluoromethyl)phosphonic acid |
| 83 | 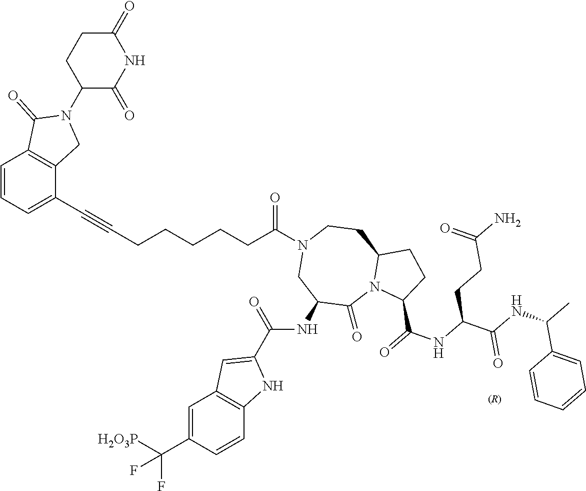 | ((2-(((5S,8S,10aR)-8-(((S)-5-amino-1,5-dioxo-1-(((R)-1- phenylethyl)amino)pentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)-1H-indol-5-yl)difluoromethyl)phosphonic acid |
| 84 | 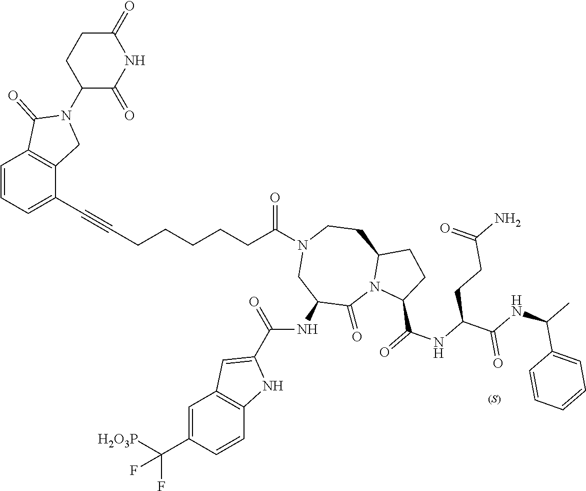 | ((2-(((5S,8S,10aR)-8-(((S)-5-amino-1,5-dioxo-1-(((S)-1- phenylethyl)amino)pentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)-1H-indol-5-yl)difluoromethyl)phosphonic acid |
| 85 | 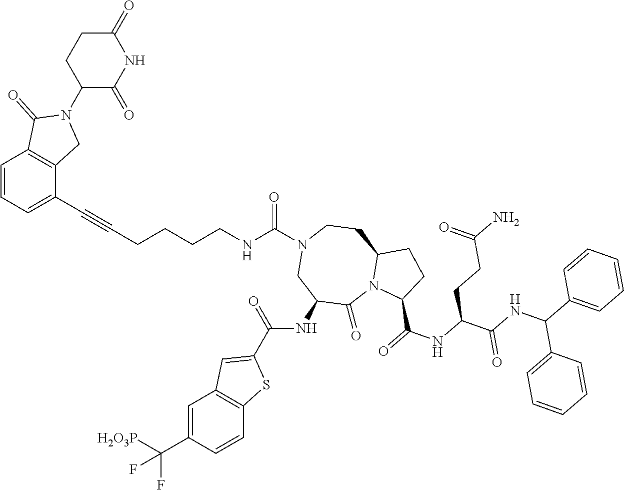 | ((2-(((5S,8S,10aR)-8-(((S)-5-amino-1- (benzhydrylamino)-1,5-dioxopentan-2-yl)carbamoyl)-3- ((6-(2-(2,6-dioxopiperidin-3-yl)-1-oxoisoindolin-4- yl)hex-5-yn-1-yl)carbamoyl)-6- oxodecahydropyrrolo[1,2-a][1,5]diazocin-5- yl)carbamoyl)benzo[b]thiophen-5- yl)difluoromethyl)phosphonic acid |
| 86 | 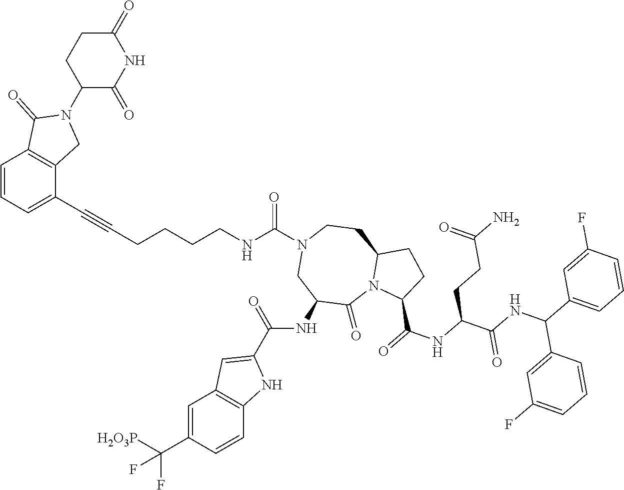 | ((2-(((5S,8S,10aR)-8-(((S)-5-amino-1-((bis(3- fluorophenyl)methyl)amino)-1,5-dioxopentan-2- yl)carbamoyl)-3-((6-(2-(2,6-dioxopiperidin-3-yl)-1- oxoisoindolin-4-yl)hex-5-yn-1-yl)carbamoyl)-6- oxodecahydropyrrolo[1,2-a][1,5]diazocin-5- yl)carbamoyl)-1H-indol-5-yl)difluoromethyl)phosphonic acid |
| 87 | 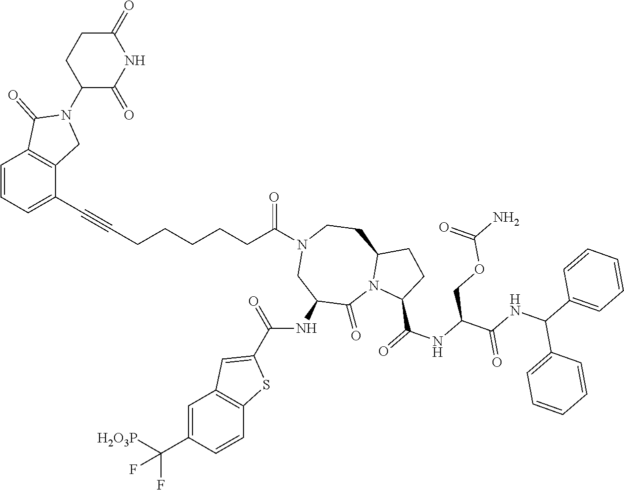 | ((2-(((5S,8S,10aR)-8-(((S)-1-(benzhydrylamino)-3- (carbamoyloxy)-1-oxopropan-2-yl)carbamoyl)-3-(8-(2- (2,6-dioxopiperidin-3-yl)-1-oxoisoindolin-4-yl)oct-7- ynoyl)-6-oxodecahydropyrrolo[1,2-a][1,5]diazocin-5- yl)carbamoyl)benzo[b]thiophen-5- yl)difluoromethyl)phosphonic acid |
| 88 | 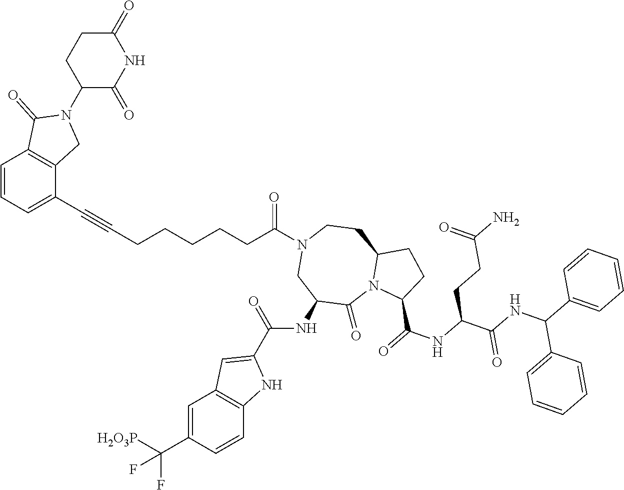 | ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1,3-dioxoisoindolin-4- yl)oct-7-ynoyl)-6-oxodecahydropyrrolo[1,2- a][1,5]diazocin-5-yl)carbamoyl)-1H-indol-5- yl)difluoromethyl)phosphonic acid |
| 88E | 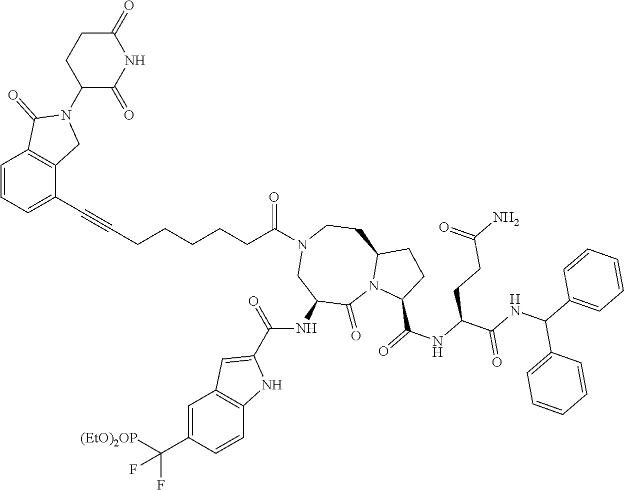 | diethyl ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1,3-dioxoisoindolin-4- yl)oct-7-ynoyl)-6-oxodecahydropyrrolo[1,2- a][1,5]diazocin-5-yl)carbamoyl)-1H-indol-5- yl)difluoromethyl)phosphonate |
| 89 | 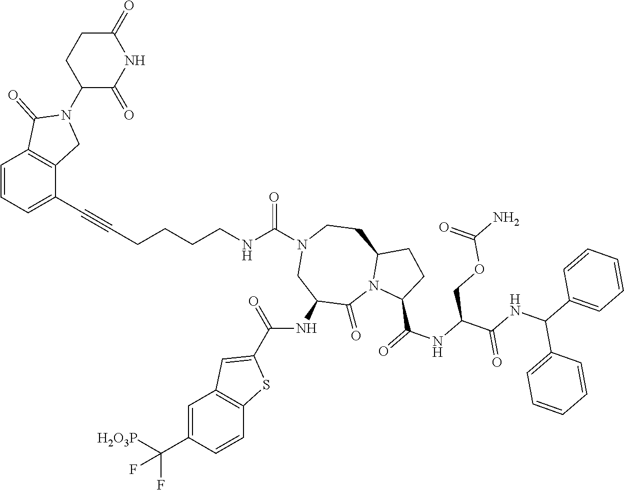 | ((2-(((5S,8S,10aR)-8-(((S)-1-(benzhydrylamino)-3- (carbamoyloxy)-1-oxopropan-2-yl)carbamoyl)-3-((6-(2- (2,6-dioxopiperidin-3-yl)-1-oxoisoindolin-4-yl)hex-5-yn- 1-yl)carbamoyl)-6-oxodecahydropyrrolo[1,2- a][1,5]diazocin-5-yl)carbamoyl)-1H-indol-5- yl)difluoromethyl)phosphonic acid |
| 91 | 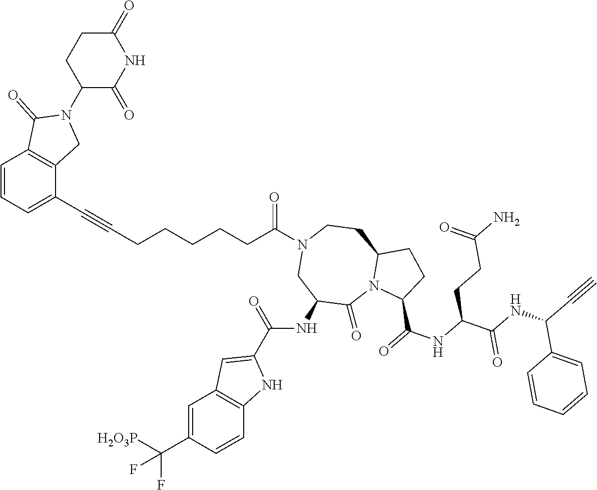 | ((2-(((5S,8S,10aR)-8-(((S)-5-amino-1,5-dioxo-1-(((R)-1- phenylprop-2-yn-1-yl)amino)pentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)-1H-indol-5-yl)difluoromethyl)phosphonic acid |
| 92 | 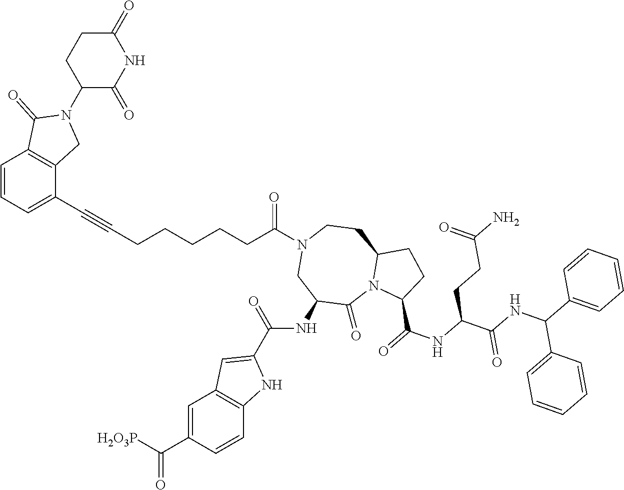 | (2-(((5S,8S,10aR)-8-(((S)-5-amino-1-(benzhydrylamino)- 1,5-dioxopentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)-1H-indole-5-carbonyl)phosphonic acid |
| 93 | 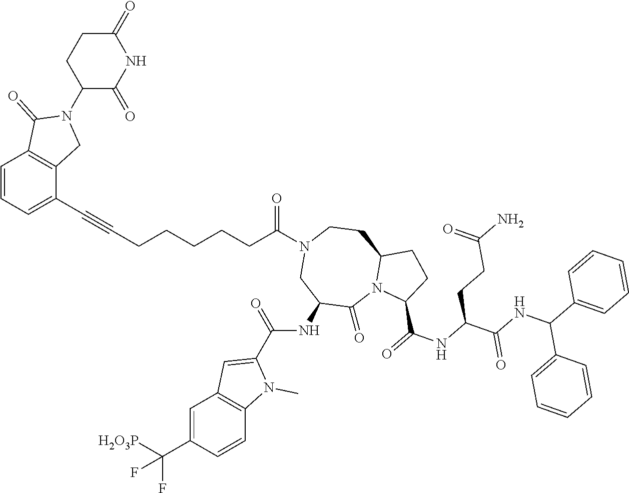 | ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)-1-methyl-1H-indol-5- yl)difluoromethyl)phosphonic acid |
| 94 | 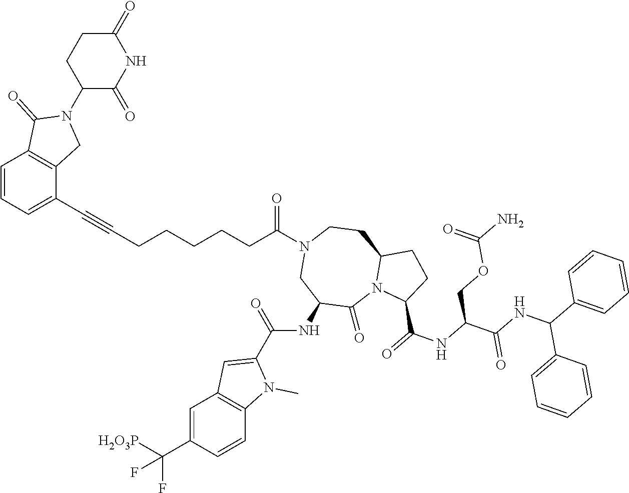 | ((2-(((5S,8S,10aR)-8-(((S)-1-(benzhydrylamino)-3- (carbamoyloxy)-1-oxopropan-2-yl)carbamoyl)-3-(8-(2- (2,6-dioxopiperidin-3-yl)-1-oxoisoindolin-4-yl)oct-7- ynoyl)-6-oxodecahydropyrrolo[1,2-a][1,5]diazocin-5- yl)carbamoyl)-1-methyl-1H-indol-5- yl)difluoromethyl)phosphonic acid |
| 95 | 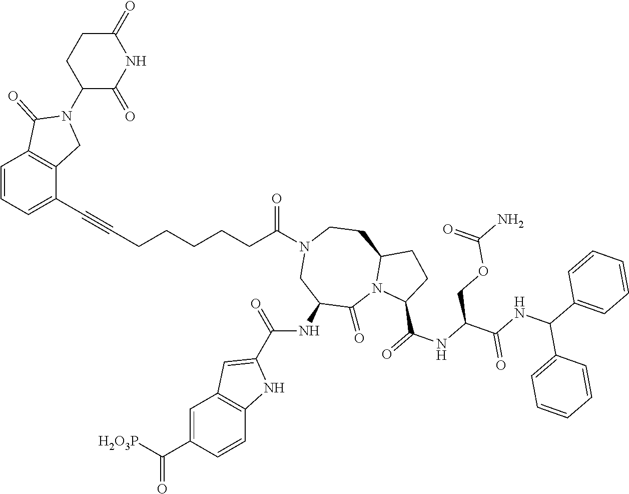 | (2-(((5S,8S,10aR)-8-(((S)-1-(benzhydrylamino)-3- (carbamoyloxy)-1-oxopropan-2-yl)carbamoyl)-3-(8-(2- (2,6-dioxopiperidin-3-yl)-1-oxoisoindolin-4-yl)oct-7- ynoyl)-6-oxodecahydropyrrolo[1,2-a][1,5]diazocin-5- yl)carbamoyl)-1H-indole-5-carbonyl)phosphonic acid |
| 96 | 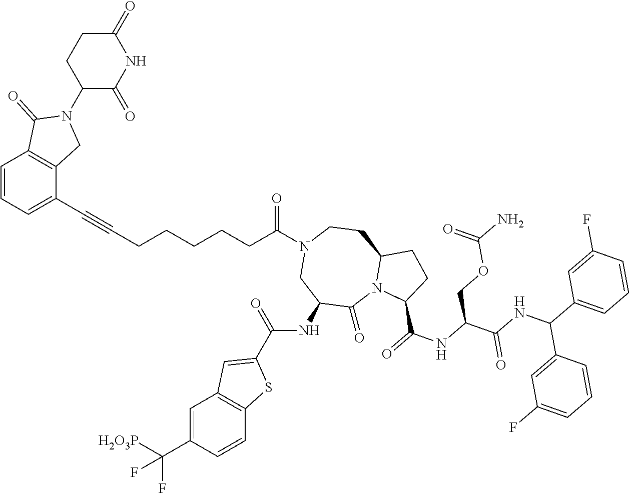 | ((2-(((5S,8S,10aR)-8-(((S)-1-((bis(3- fluorophenyl)methyl)amino)-3-(carbamoyloxy)-1- oxopropan-2-yl)carbamoyl)-3-(8-(2-(2,6-dioxopiperidin- 3-yl)-1-oxoisoindolin-4-yl)oct-7-ynoyl)-6- oxodecahydropyrrolo[1,2-a][1,5]diazocin-5- yl)carbamoyl)benzo[b]thiophen-5- yl)difluoromethyl)phosphonic acid |
| 97 | 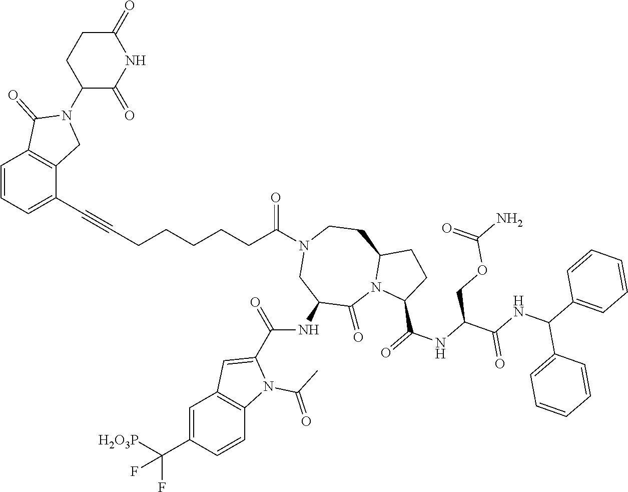 | ((1-acetyl-2-(((5S,8S,10aR)-8-(((S)-1- (benzhydrylamino)-3-(carbamoyloxy)-1-oxopropan-2- yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)-1H-indol-5-yl)difluoromethyl)phosphonic acid |
| 98 | 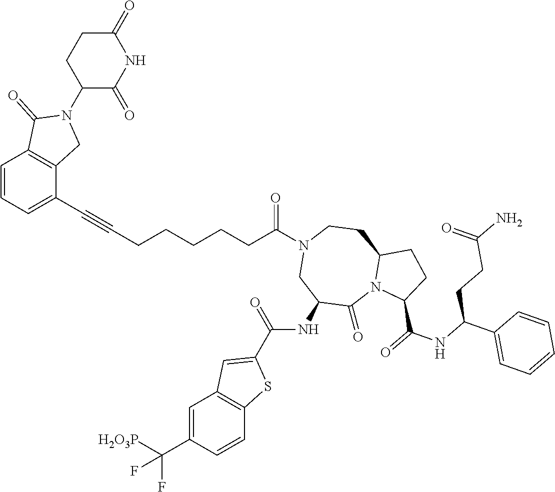 | ((2-(((5S,8S,10aR)-8-(((S)-4-amino-4-oxo-1- phenylbutyl)carbamoyl)-3-(8-(2-(2,6-dioxopiperidin-3- yl)-1-oxoisoindolin-4-yl)oct-7-ynoyl)-6- oxodecahydropyrrolo[1,2-a][1,5]diazocin-5- yl)carbamoyl)benzo[b]thiophen-5- yl)difluoromethyl)phosphonic acid |
| 99 | 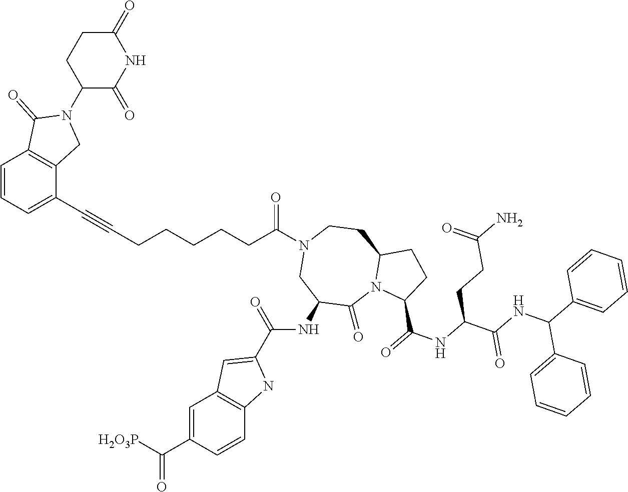 | 2-(((5S,8S,10aR)-8-(((S)-5-amino-1-(benzhydrylamino)- 1,5-dioxopentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)-1H-indole-5-carboxylic acid |
| 100 | 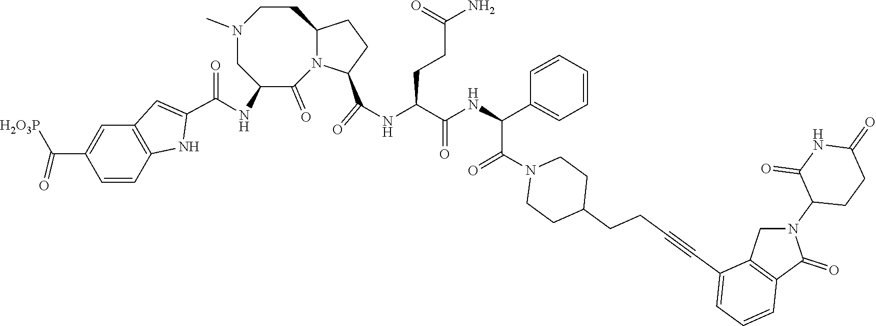 | (2-(((5S,8S,10aR)-8-(((2S)-5-amino-1-(((1S)-2-(4-(4-(2- (2,6-dioxopiperidin-3-yl)-1-oxoisoindolin-4-yl)but-3-yn- 1-yl)piperidin-1-yl)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)-1H-indole-5-carbonyl)phosphonic acid |
| 101 | 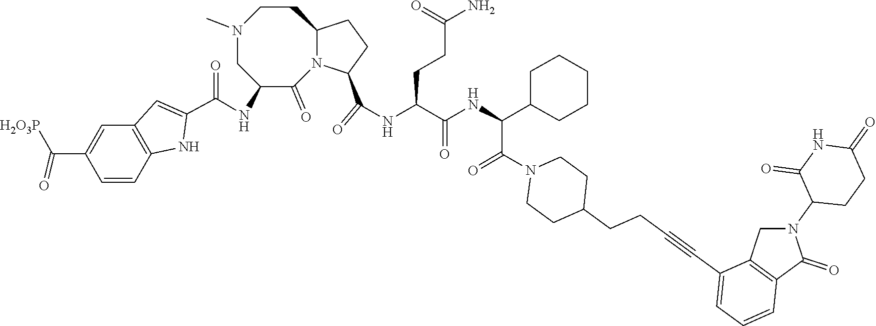 | (2-(((5S,8S,10aR)-8-(((2S)-5-amino-1-(((1S)-1- cyclohexyl-2-(4-(4-(2-(2,6-dioxopiperidin-3-yl)-1- oxoisoindolin-4-yl)but-3-yn-1-yl)piperidin-1-yl)-2- oxoethyl)amino)-1,5-dioxopentan-2-yl)carbamoyl)-3- methyl-6-oxodecahydropyrrolo[1,2-a][1,5]diazocin-5- yl)carbamoyl)-1H-indole-5-carbonyl)phosphonic acid |
| 102 | 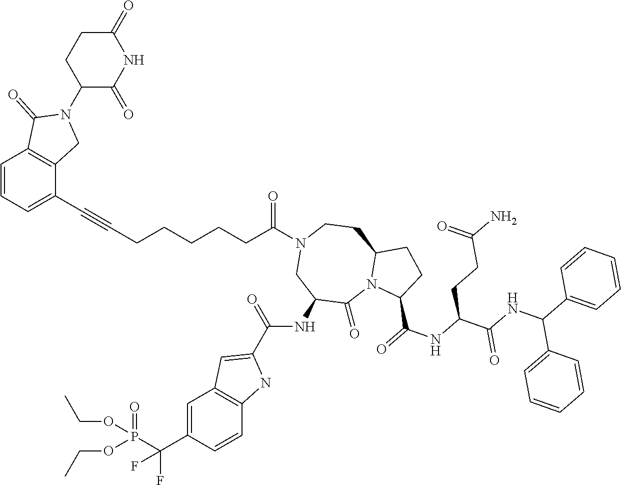 [SMILES: CCOP(=O)(OCC)C(F)(F)c1ccc2[nH]c(C(=O)N[C@H]3CN(C(=O)CCCCCC#Cc4cccc5c4CN(C4CCC(=O)NC4=O)C5=O)CC[C@H]4CC[C@@H](C(=O)N[C@@H](CCC(N)=O)C(=O)NC(c5ccccc5)c5ccccc5)N4C3=O)cc2c1] | diethyl ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)-1H-indol-5- yl)difluoromethyl)phosphonate |
| 103 | 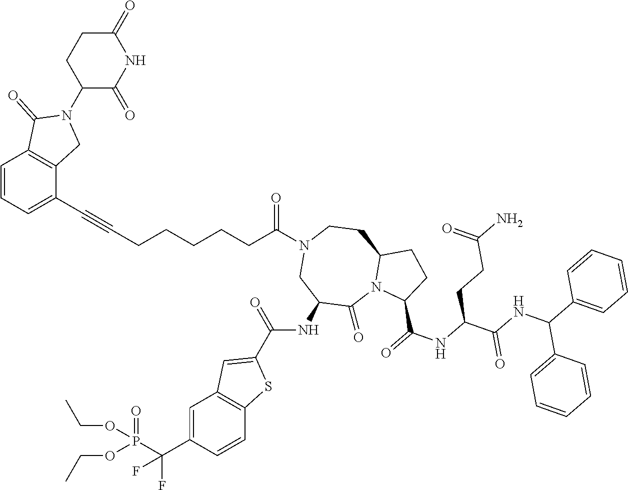 [SMILES: CCOP(=O)(OCC)C(F)(F)c1ccc2sc(C(=O)N[C@H]3CN(C(=O)CCCCCC#Cc4cccc5c4CN(C4CCC(=O)NC4=O)C5=O)CC[C@H]4CC[C@@H](C(=O)N[C@@H](CCC(N)=O)C(=O)NC(c5ccccc5)c5ccccc5)N4C3=O)cc2c1] | diethyl ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)benzo[b]thiophen-5- yl)difluoromethyl)phosphonate |
| 104 | 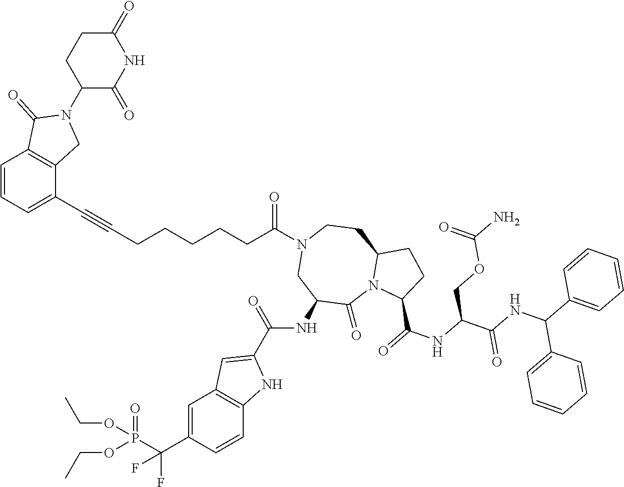 [SMILES: CCOP(=O)(OCC)C(F)(F)c1ccc2[nH]c(C(=O)N[C@H]3CN(C(=O)CCCCCC#Cc4cccc5c4CN(C4CCC(=O)NC4=O)C5=O)CC[C@H]4CC[C@@H](C(=O)N[C@@H](COC(N)=O)C(=O)NC(c5ccccc5)c5ccccc5)N4C3=O)cc2c1] | (2S)-3-(benzhydrylamino)-2-((5S,8S,10aR)-5-(5- ((diethoxyphosphoryl)difluoromethyl)-1H-indole-2- carboxamido)-3-(8-(2-(2,6-dioxopiperidin-3-yl)-1- oxoisoindolin-4-yl)oct-7-ynoyl)-6- oxodecahydropyrrolo[1,2-a][1,5]diazocine-8- carboxamido)-3-oxopropyl carbamate |
| 105 | 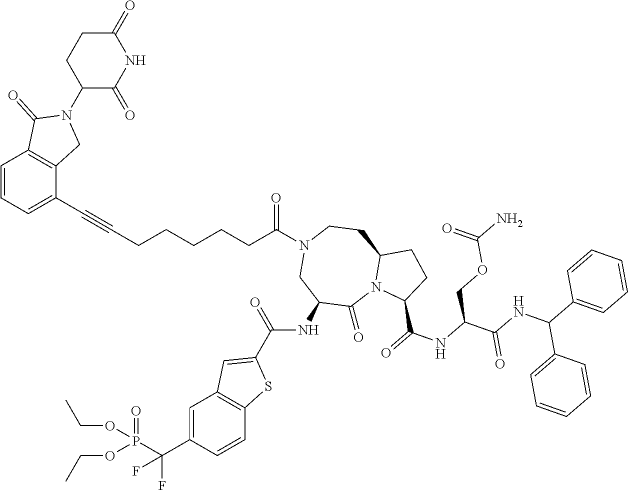 [SMILES: CCOP(=O)(OCC)C(F)(F)c1ccc2sc(C(=O)N[C@H]3CN(C(=O)CCCCCC#Cc4cccc5c4CN(C4CCC(=O)NC4=O)C5=O)CC[C@H]4CC[C@@H](C(=O)N[C@@H](COC(N)=O)C(=O)NC(c5ccccc5)c5ccccc5)N4C3=O)cc2c1] | (2S)-3-(benzhydrylamino)-2-((5S,8S,10aR)-5-(5- ((diethoxyphosphoryl)difluoromethyl)benzo[b]thiophene- 2-carboxamido)-3-(8-(2-(2,6-dioxopiperidin-3-yl)-1- oxoisoindolin-4-yl)oct-7-ynoyl)-6- oxodecahydropyrrolo[1,2-a][1,5]diazocine-8- carboxamido)-3-oxopropyl carbamate |
| 106 | 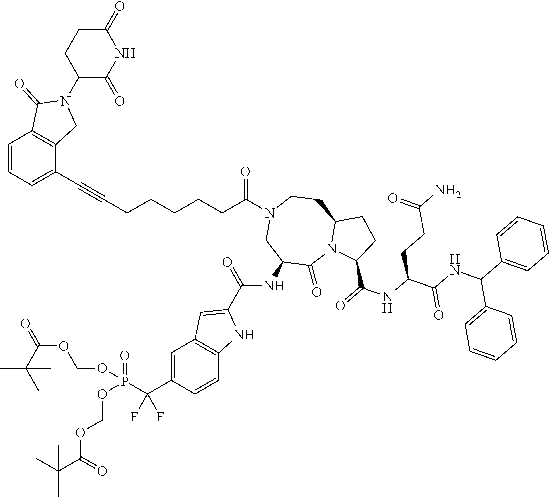 [SMILES: CC(C)(C)C(=O)OCOP(=O)(OCOC(=O)C(C)(C)C)C(F)(F)c1ccc2[nH]c(C(=O)N[C@H]3CN(C(=O)CCCCCC#Cc4cccc5c4CN(C4CCC(=O)NC4=O)C5=O)CC[C@H]4CC[C@@H](C(=O)N[C@@H](CCC(N)=O)C(=O)NC(c5ccccc5)c5ccccc5)N4C3=O)cc2c1] | ((((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)-1H-indol-5- yl)difluoromethyl)phosphoryl)bis(oxy))bis(methylene) bis(2,2-dimethylpropanoate) |
| 107 | 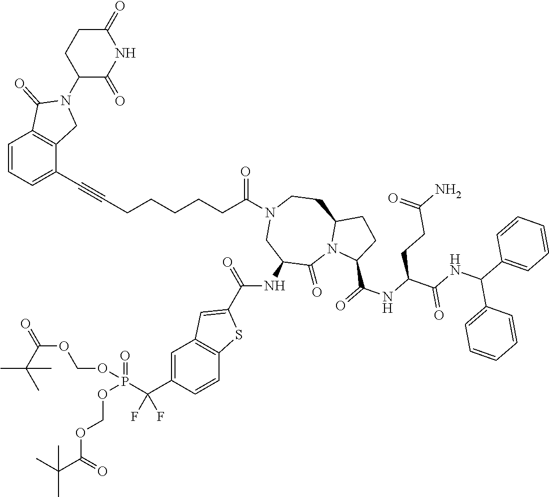 [SMILES: CC(C)(C)C(=O)OCOP(=O)(OCOC(=O)C(C)(C)C)C(F)(F)c1ccc2sc(C(=O)N[C@H]3CN(C(=O)CCCCCC#Cc4cccc5c4CN(C4CCC(=O)NC4=O)C5=O)CC[C@H]4CC[C@@H](C(=O)N[C@@H](CCC(N)=O)C(=O)NC(c5ccccc5)c5ccccc5)N4C3=O)cc2c1] | ((((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)benzo[b]thiophen-5- yl)difluoromethyl)phosphoryl)bis(oxy))bis(methylene) bis(2,2-dimethylpropanoate) |
| 108 | 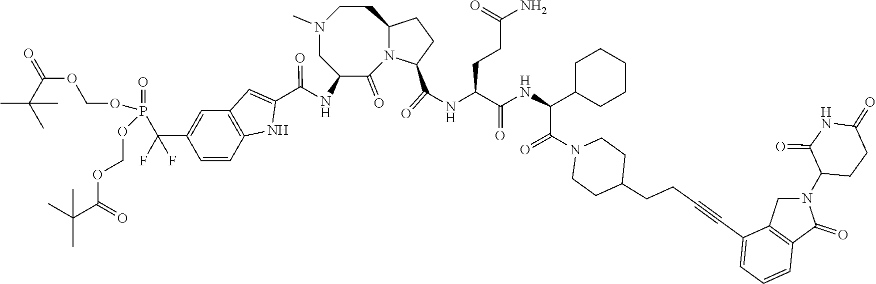 [SMILES: CN1CC[C@H]2CC[C@@H](C(=O)N[C@@H](CCC(N)=O)C(=O)N[C@H](C(=O)N3CCC(CCC#Cc4cccc5c4CN(C4CCC(=O)NC4=O)C5=O)CC3)C3CCCCC3)N2C(=O)[C@@H](NC(=O)c2cc3cc(C(F)(F)P(=O)(OCOC(=O)C(C)(C)C)OCOC(=O)C(C)(C)C)ccc3[nH]2)C1] | ((((2-(((5S,8S,10aR)-8-(((2S)-5-amino-1-(((1S)-1- cyclohexyl-2-(4-(4-(2-(2,6-dioxopiperidin-3-yl)-1- oxoisoindolin-4-yl)but-3-yn-1-yl)piperidin-1-yl)-2- oxoethyl)amino)-1,5-dioxopentan-2-yl)carbamoyl)-3- methyl-6-oxodecahydropyrrolo[1,2-a][1,5]diazocin-5- yl)carbamoyl)-1H-indol-5- yl)difluoromethyl)phosphoryl)bis(oxy))bis(methylene) bis(2,2-dimethylpropanoate) |
| 109 | 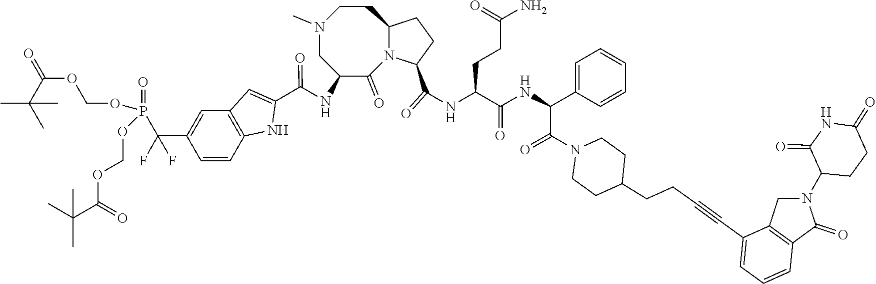 [SMILES: CN1CC[C@H]2CC[C@@H](C(=O)N[C@@H](CCC(N)=O)C(=O)N[C@H](C(=O)N3CCC(CCC#Cc4cccc5c4CN(C4CCC(=O)NC4=O)C5=O)CC3)c3ccccc3)N2C(=O)[C@@H](NC(=O)c2cc3cc(C(F)(F)P(=O)(OCOC(=O)C(C)(C)C)OCOC(=O)C(C)(C)C)ccc3[nH]2)C1] | ((((2-(((5S,8S,10aR)-8-(((2S)-5-amino-1-(((1S)-2-(4-(4- (2-(2,6-dioxopiperidin-3-yl)-1-oxoisoindolin-4-yl)but-3- yn-1-yl)piperidin-1-yl)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)-1H-indol-5- yl)difluoromethyl)phosphoryl)bis(oxy))bis(methylene) bis(2,2-dimethylpropanoate) |
| 110 | 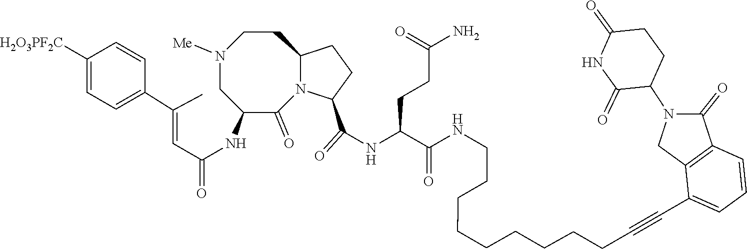 | ((4-((E)-4-(((5S,8S,10aR)-8-(((2S)-5-amino-1-((11-(2- (2,6-dioxopiperidin-3-yl)-1-oxoisoindolin-4-yl)undec-10- yn-1-yl)amino)-1,5-dioxopentan-2-yl)carbamoyl)-3- methyl-6-oxodecahydropyrrolo[1,2-a][1,5]diazocin-5- yl)amino)-4-oxobut-2-en-2- yl)phenyl)difluoromethyl)phosphonic acid |
| 111 | 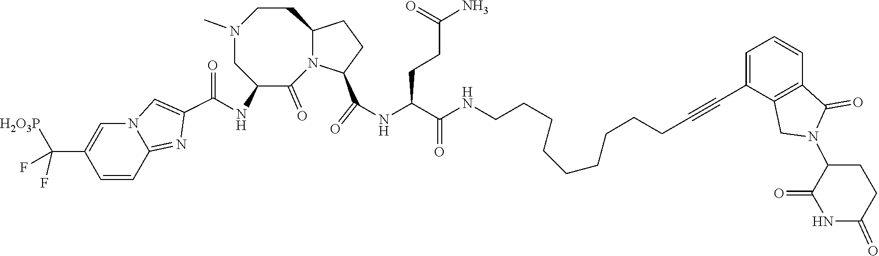 | ((2-(((5S,8S,10aR)-8-(((2S)-5-amino-1-((11-(2-(2,6- dioxopiperidin-3-yl)-1-oxoisoindolin-4-yl)undec-10-yn- 1-yl)amino)-1,5-dioxopentan-2-yl)carbamoyl)-3-methyl- 6-oxodecahydropyrrolo[1,2-a][1,5]diazocin-5- yl)carbamoyl)imidazo[1,2-a]pyridin-6- yl)difluoromethyl)phosphonic acid |
| 112 | 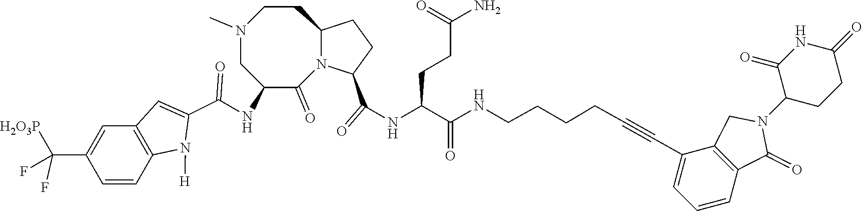 | ((2-(((5S,8S,10aR)-8-(((2S)-5-amino-1-((6-(2-(2,6- dioxopiperidin-3-yl)-1-oxoisoindolin-4-yl)hex-5-yn-1- yl)amino)-1,5-dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 113 | 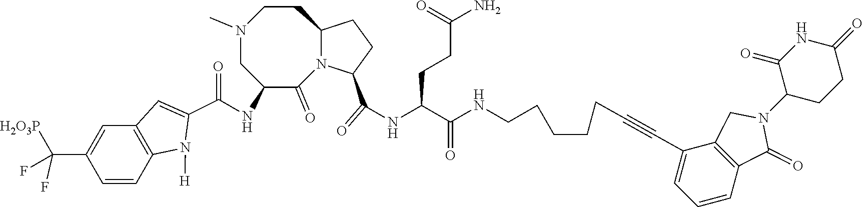 | ((2-(((5S,8S,10aR)-8-(((2S)-5-amino-1-((7-(2-(2,6- dioxopiperidin-3-yl)-1-oxoisoindolin-4-yl)hept-6-yn-1- yl)amino)-1,5-dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 114 | 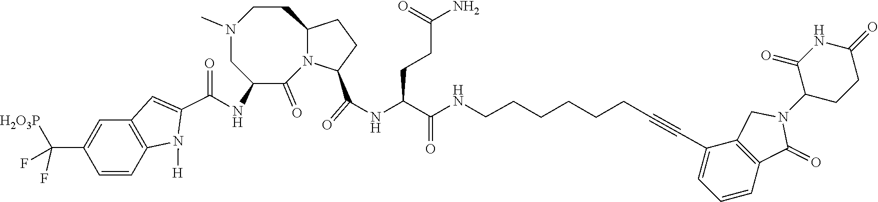 | ((2-(((5S,8S,10aR)-8-(((2S)-5-amino-1-((8-(2-(2,6- dioxopiperidin-3-yl)-1-oxoisoindolin-4-yl)oct-7-yn-1- yl)amino)-1,5-dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 115 | 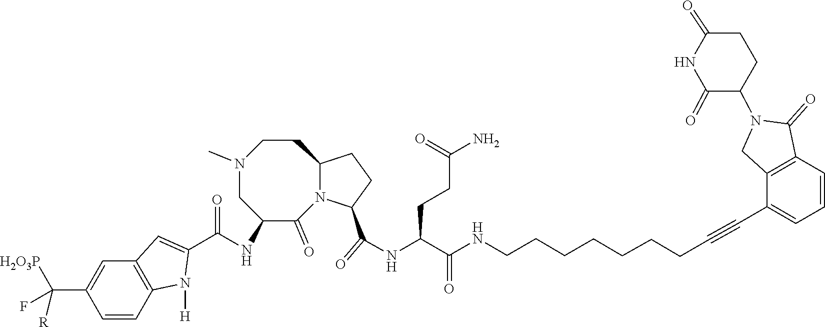 | ((2-(((5S,8S,10aR)-8-(((2S)-5-amino-1-((9-(2-(2,6- dioxopiperidin-3-yl)-1-oxoisoindolin-4-yl)non-8-yn-1- yl)amino)-1,5-dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 116 | 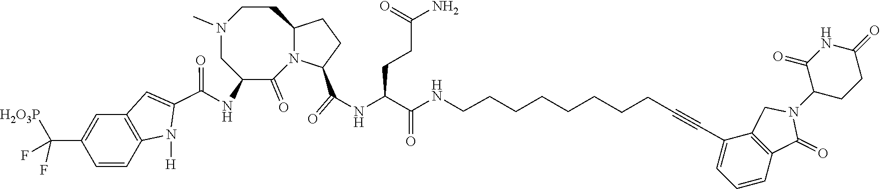 | ((2-(((5S,8S,10aR)-8-(((2S)-5-amino-1-((10-(2-(2,6- dioxopiperidin-3-yl)-1-oxoisoindolin-4-yl)dec-9-yn-1- yl)amino)-1,5-dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 117 | 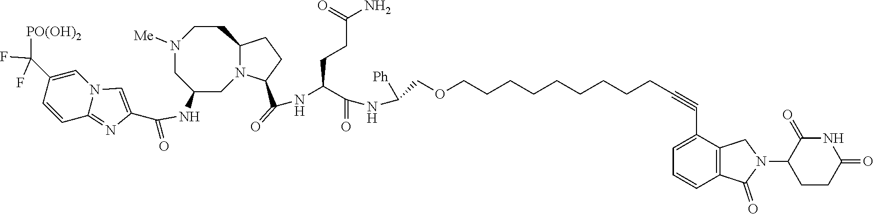 | ((2-(((5S,8S,10aR)-8-(((2S)-5-amino-1-((2-((11-(2-(2,6- dioxopiperidin-3-yl)-1-oxoisoindolin-4-yl)undec-10-yn- 1-yl)oxy)-1-phenylethyl)amino)-1,5-dioxopentan-2- yl)carbamoyl)-3-methyl-6-oxodecahydropyrrolo[1,2- a][1,5]diazocin-5-yl)carbamoyl)imidazo[1,2-a]pyridin-6- yl)difluoromethyl)phosphonic acid |
| 118 | 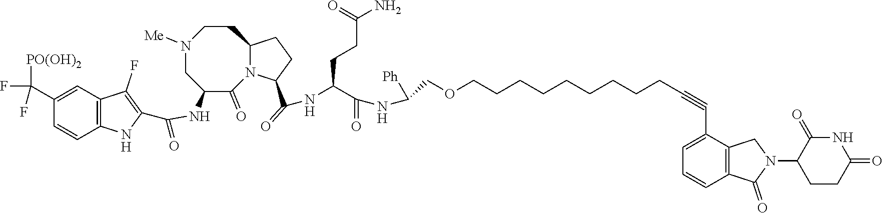 | ((2-(((5S,8S,10aR)-8-(((2S)-5-amino-1-((2-((11-(2-(2,6- dioxopiperidin-3-yl)-1-oxoisoindolin-4-yl)undec-10-yn- 1-yl)oxy)-1-phenylethyl)amino)-1,5-dioxopentan-2- yl)carbamoyl)-3-methyl-6-oxodecahydropyrrolo[1,2- a][1,5]diazocin-5-yl)carbamoyl)-3-fluoro-1H-indol-5- yl)difluoromethyl)phosphonic acid |
| 119 | 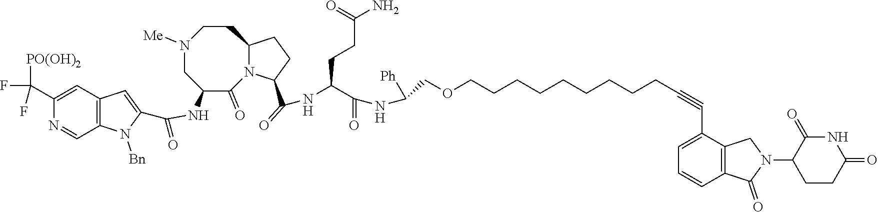 | ((2-(((5S,8S,10aR)-8-(((2S)-5-amino-1-((2-((11-(2-(2,6- dioxopiperidin-3-yl)-1-oxoisoindolin-4-yl)undec-10-yn- 1-yl)oxy)-1-phenylethyl)amino)-1,5-dioxopentan-2- yl)carbamoyl)-3-methyl-6-oxodecahydropyrrolo[1,2- a][1,5]diazocin-5-yl)carbamoyl)-1-benzyl-1H- pyrrolo[2,3-c]pyridin-5-yl)difluoromethyl)phosphonic acid |
| 120 | 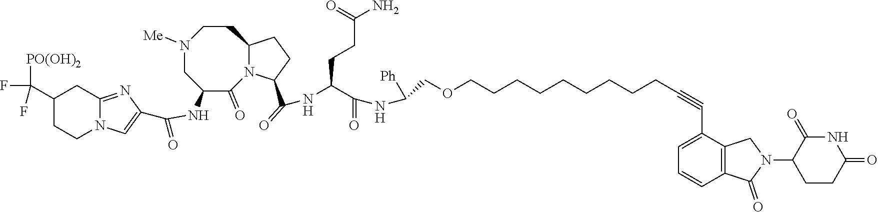 | ((2-(((5S,8S,10aR)-8-(((2S)-5-amino-1-((2-((11-(2-(2,6- dioxopiperidin-3-yl)-1-oxoisoindolin-4-yl)undec-10-yn- 1-yl)oxy)-1-phenylethyl)amino)-1,5-dioxopentan-2- yl)carbamoyl)-3-methyl-6-oxodecahydropyrrolo[1,2- a][1,5]diazocin-5-yl)carbamoyl)-5,6,7,8- tetrahydroimidazo[1,2-a]pyridin-7- yl)difluoromethyl)phosphonic acid |
| 121 | 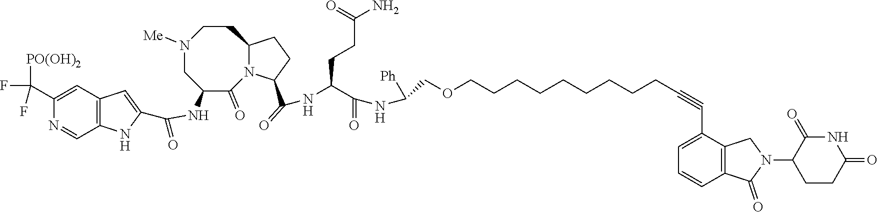 | ((2-(((5S,8S,10aR)-8-(((2S)-5-amino-1-((2-((11-(2-(2,6- dioxopiperidin-3-yl)-1-oxoisoindolin-4-yl)undec-10-yn- 1-yl)oxy)-1-phenylethyl)amino)-1,5-dioxopentan-2- yl)carbamoyl)-3-methyl-6-oxodecahydropyrrolo[1,2- a][1,5]diazocin-5-yl)carbamoyl)-1H-pyrrolo[2,3- c]pyridin-5-yl)difluoromethyl)phosphonic acid |
| 122 | 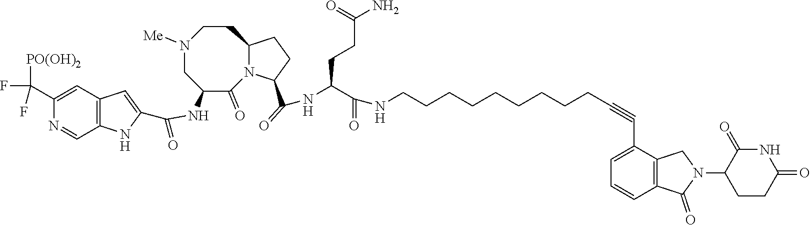 | ((2-(((5S,8S,10aR)-8-(((2S)-5-amino-1-((11-(2-(2,6- dioxopiperidin-3-yl)-1-oxoisoindolin-4-yl)undec-10-yn- 1-yl)amino)-1,5-dioxopentan-2-yl)carbamoyl)-3-methyl- 6-oxodecahydropyrrolo[1,2-a][1,5]diazocin-5- yl)carbamoyl)-1H-pyrrolo[2,3-c]pyridin-5- yl)difluoromethyl)phosphonic acid |
| 123 | 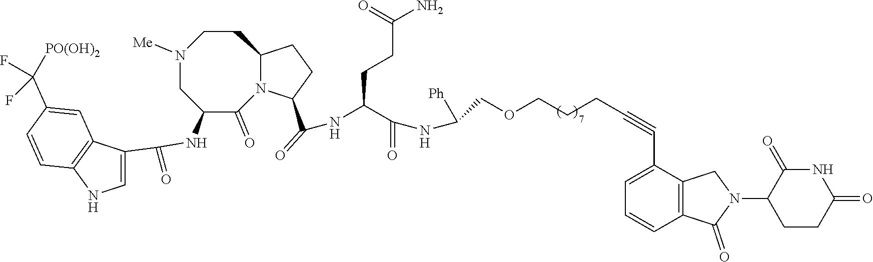 | ((3-(((5S,8S,10aR)-8-(((2S)-5-amino-1-((2-((5-(2-(2,6- dioxopiperidin-3-yl)-1-oxoisoindolin-4-yl)pent-4-yn-1- yl)oxy)-1-phenylethyl)amino)-1,5-dioxopentan-2- yl)carbamoyl)-3-methyl-6-oxodecahydropyrrolo[1,2- a][1,5]diazocin-5-yl)carbamoyl)-1H-indol-5- yl)difluoromethyl)phosphonic acid |
| 124 | 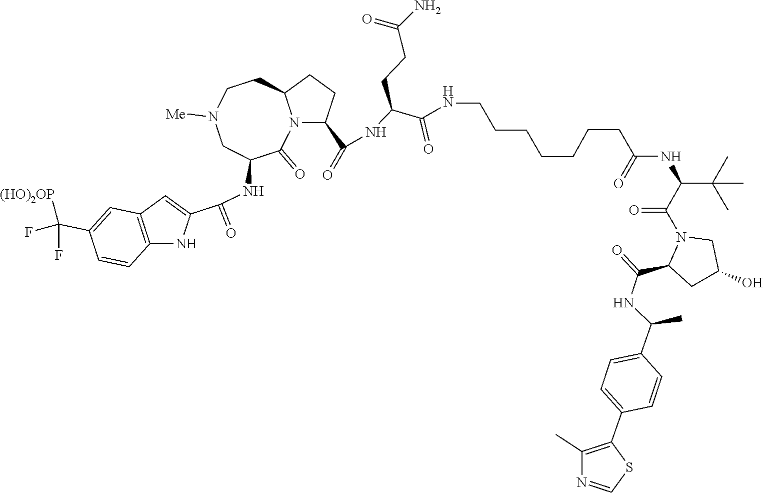 | ((2-(((5S,8S,10aR)-8-(((S)-5-amino-1-((8-(((S)-1- ((2S,4R)-4-hydroxy-2-(((S)-1-(4-(4-methylthiazol-5- yl)phenyl)ethyl)carbamoyl)pyrrolidin-1-yl)-3,3-dimethyl- 1-oxobutan-2-yl)amino)-8-oxooctyl)amino)-1,5- dioxopentan-2-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 125 | 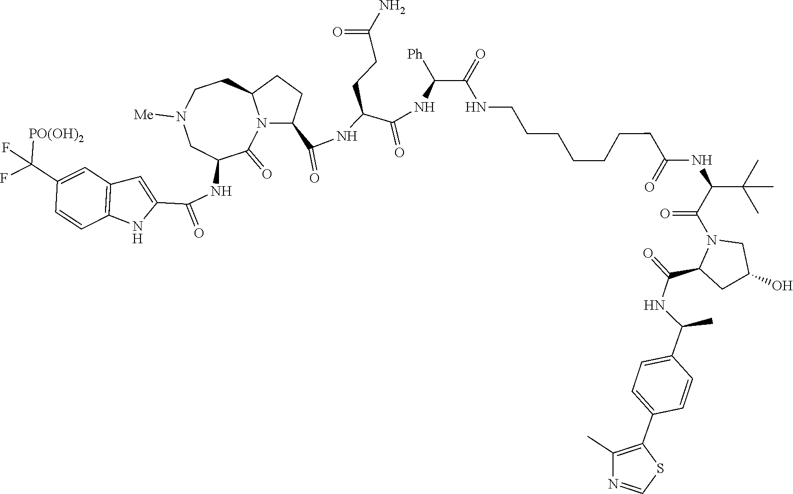 | ((2-(((5S,8S,10aR)-8-(((S)-5-amino-1-(((S)-2-((8-(((S)-1- ((2S,4R)-4-hydroxy-2-(((S)-1-(4-(4-methylthiazol-5- yl)phenyl)ethyl)carbamoyl)pyrrolidin-1-yl)-3,3-dimethyl- 1-oxobutan-2-yl)amino)-8-oxooctyl)amino)-2-oxo-1- phenylethyl)amino)-1,5-dioxopentan-2-yl)carbamoyl)-3- methyl-6-oxodecahydropyrrolo[1,2-a][1,5]diazocin-5- yl)carbamoyl)-1H-indol-5-yl)difluoromethyl)phosphonic acid |
| 126 | 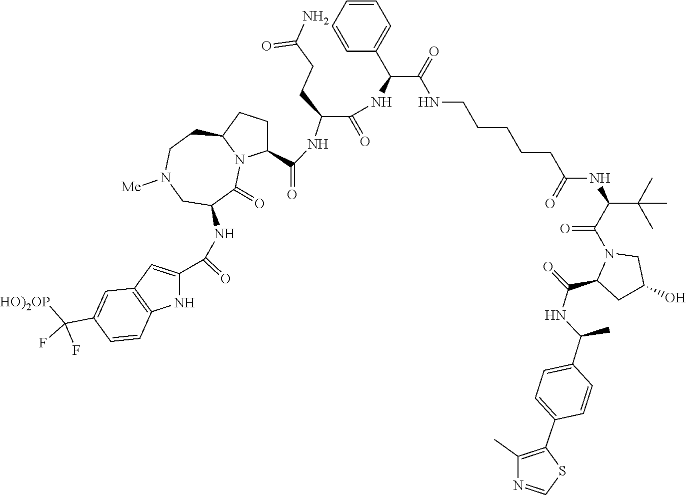 | ((2-(((5S,8S,10aR)-8-(((S)-5-amino-1-(((S)-2-((6-(((S)-1- ((2S,4R)-4-hydroxy-2-(((S)-1-(4-(4-methylthiazol-5- yl)phenyl)ethyl)carbamoyl)pyrrolidin-1-yl)-3,3-dimethyl- 1-oxobutan-2-yl)amino)-6-oxohexyl)amino)-2-oxo-1- phenylethyl)amino)-1,5-dioxopentan-2-yl)carbamoyl)-3- methyl-6-oxodecahydropyrrolo[1,2-a][1,5]diazocin-5- yl)carbamoyl)-1H-indol-5-yl)difluoromethyl)phosphonic acid |
| 127 | 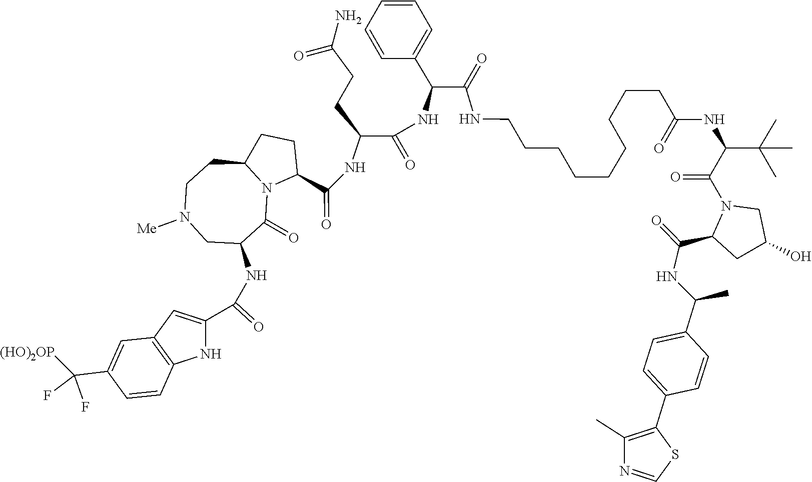 | ((2-(((5S,8S,10aR)-8-(((S)-5-amino-1-(((S)-2-((10-(((S)- 1-((2S,4R)-4-hydroxy-2-(((S)-1-(4-(4-methylthiazol-5- yl)phenyl)ethyl)carbamoyl)pyrrolidin-1-yl)-3,3-dimethyl- 1-oxobutan-2-yl)amino)-10-oxodecyl)amino)-2-oxo-1- phenylethyl)amino)-1,5-dioxopentan-2-yl)carbamoyl)-3- methyl-6-oxodecahydropyrrolo[1,2-a][1,5]diazocin-5- yl)carbamoyl)-1H-indol-5-yl)difluoromethyl)phosphonic acid |
| 128 | 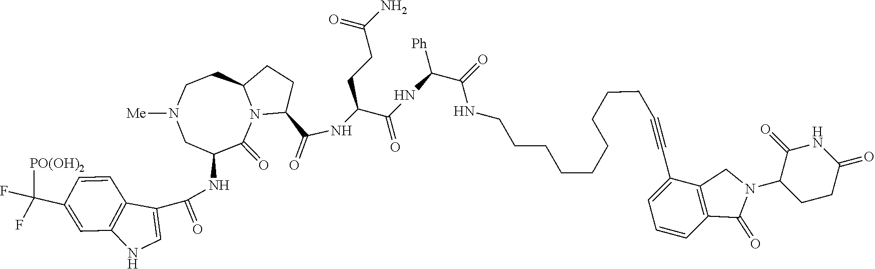 | ((3-(((5S,8S,10aR)-8-(((2S)-5-amino-1-(((1S)-2-((11-(2- (2,6-dioxopiperidin-3-yl)-1-oxoisoindolin-4-yl)undec-10- yn-1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)-1H-indol-6-yl)difluoromethyl)phosphonic acid |
| 129 | 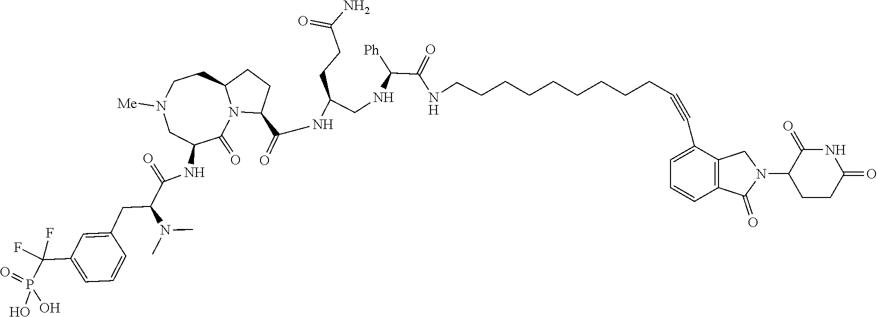 [SMILES: CN1CC[C@H]2CC[C@@H](C(=O)N[C@@H](CCC(N)=O)CN[C@H](C(=O)NCCCCCCCCCC#Cc3cccc4c3CN(C3CCC(=O)NC3=O)C4=O)c3ccccc3)N2C(=O)[C@@H](NC(=O)[C@H](Cc2cccc(C(F)(F)P(=O)(O)O)c2)N(C)C)C1] | ((3-((2S)-3-(((5S,8S,10aR)-8-(((2S)-5-amino-1-(((1S)-2- ((11-(2-(2,6-dioxopiperidin-3-yl)-1-oxoisoindolin-4- yl)undec-10-yn-1-yl)amino)-2-oxo-1- phenylethyl)amino)-5-oxopentan-2-yl)carbamoyl)-3- methyl-6-oxodecahydropyrrolo[1,2-a][1,5]diazocin-5- yl)amino)-2-(dimethylamino)-3- oxopropyl)phenyl)difluoromethyl)phosphonic acid |
| 130 | 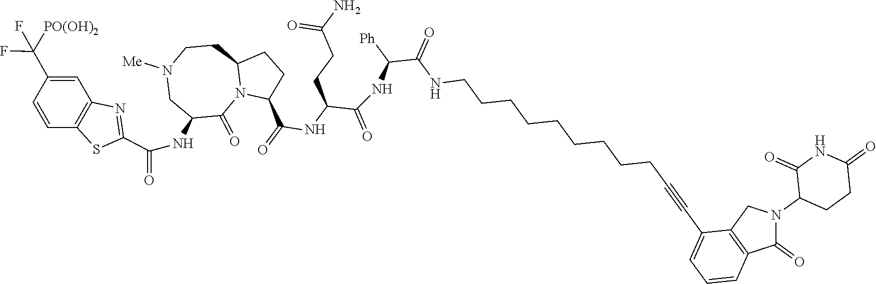 | ((2-(((5S,8S,10aR)-8-(((2S)-5-amino-1-(((1S)-2-((11-(2- (2,6-dioxopiperidin-3-yl)-1-oxoisoindolin-4-yl)undec-10- yn-1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)benzo[d]thiazol-5- yl)difluoromethyl)phosphonic acid |
| 131 | 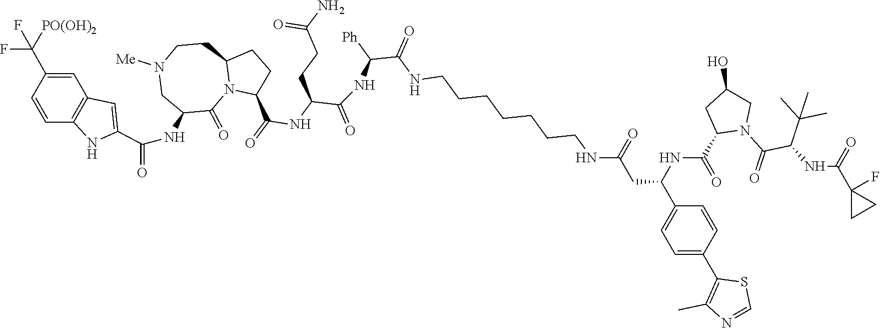 | ((2-(((5S,8S,10aR)-8-(((3S,16S,19S)-22-amino-1- ((2S,4R)-1-((S)-2-(1-fluorocyclopropane-1- carboxamido)-3,3-dimethylbutanoyl)-4- hydroxypyrrolidin-2-yl)-3-(4-(4-methylthiazol-5- yl)phenyl)-1,5,15,18,22-pentaoxo-16-phenyl-2,6,14,17- tetraazadocosan-19-yl)carbamoyl)-3-methyl-6- oxodecahydropyrrolo[1,2-a][1,5]diazocin-5- yl)carbamoyl)-1H-indol-5-yl)difluoromethyl)phosphonic acid |
| 132 | 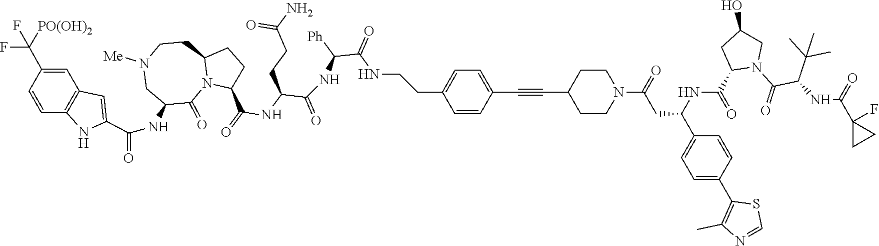 | ((2-(((5S,8S,10aR)-8-(((S)-5-amino-1-(((S)-2-((4-((1- ((S)-3-((2S,4R)-1-((S)-2-(1-fluorocyclopropane-1- carboxamido)-3,3-dimethylbutanoyl)-4- hydroxypyrrolidine-2-carboxamido)-3-(4-(4- methylthiazol-5-yl)phenyl)propanoyl)piperidin-4- yl)ethynyl)phenethyl)amino)-2-oxo-1- phenylethyl)amino)-1,5-dioxopentan-2-yl)carbamoyl)-3- methyl-6-oxodecahydropyrrolo[1,2-a][1,5]diazocin-5- yl)carbamoyl)-1H-indol-5-yl)difluoromethyl)phosphonic acid |
| 133 | 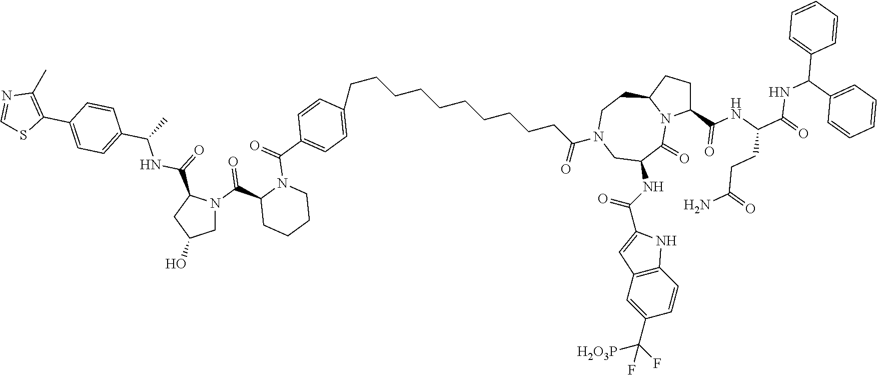 | ((2-(((5S,8S,10aR)-8-(((S)-5-amino-1- (benzhydrylamino)-1,5-dioxopentan-2-yl)carbamoyl)-3- (11-(4-((S)-2-((2S,4R)-4-hydroxy-2-(((S)-1-(4-(4- methylthiazol-5-yl)phenyl)ethyl)carbamoyl)pyrrolidine- 1-carbonyl)piperidine-1-carbonyl)phenyl)undecanoyl)-6- oxodecahydropyrrolo[1,2-a][1,5]diazocin-5- yl)carbamoyl)-1H-indol-5-yl)difluoromethyl)phosphonic acid |
| 134 | 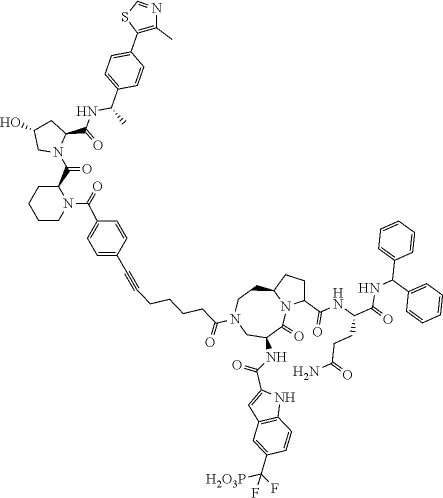 | ((2-(((5S,8S,10aR)-8-(((S)-5-amino-1- (benzhydrylamino)-1,5-dioxopentan-2-yl)carbamoyl)-3- (7-(4-((S)-2-((2S,4R)-4-hydroxy-2-(((S)-1-(4-(4- methylthiazol-5-yl)phenyl)ethyl)carbamoyl)pyrrolidine- 1-carbonyl)piperidine-1-carbonyl)phenyl)hept-6-ynoyl)- 6-oxodecahydropyrrolo[1,2-a][1,5]diazocin-5- yl)carbamoyl)-1H-indol-5-yl)difluoromethyl)phosphonic acid |
| 135 | 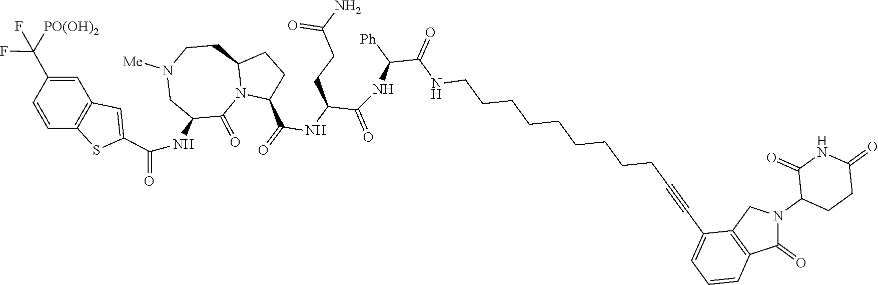 | ((2-(((5S,8S,10aR)-8-(((2S)-5-amino-1-(((1S)-2-((11-(2- (2,6-dioxopiperidin-3-yl)-1-oxoisoindolin-4-yl)undec-10- yn-1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 136 | 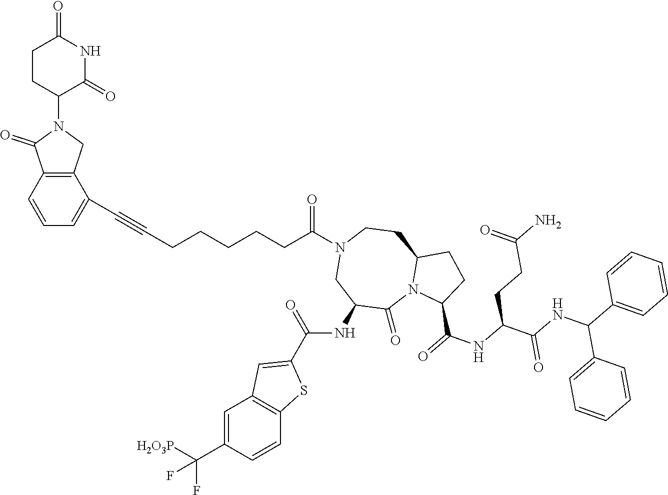 | ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)benzo[b]thiophen-5- yl)difluoromethyl)phosphonic acid |
| 137 | 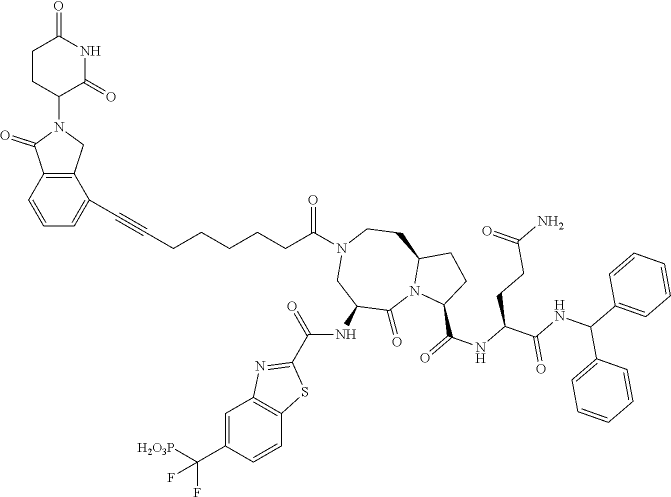 | ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)benzo[d] thiazol-5- yl)difluoromethyl)phosphonic acid |
| 138 | 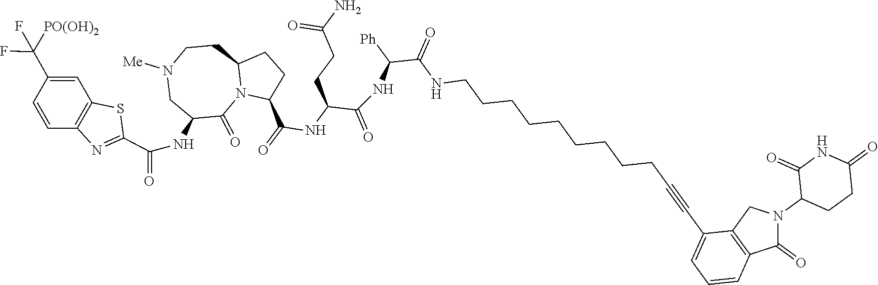 | ((2-(((5S,8S,10aR)-8-(((2S)-5-amino-1-(((1S)-2-((11-(2- (2,6-dioxopiperidin-3-yl)-1-oxoisoindolin-4-yl)undec-10- yn-1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)benzo[d]thiazol-6- yl)difluoromethyl)phosphonic acid |
| 139 | 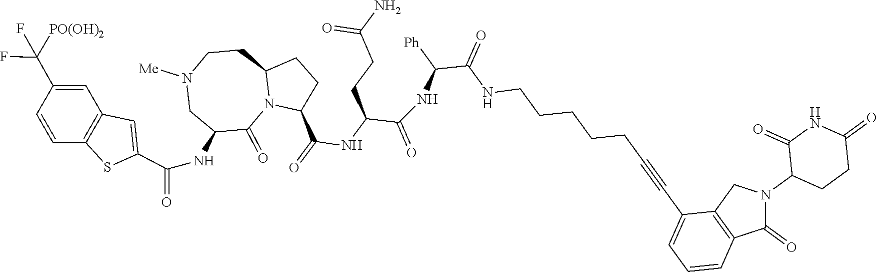 | ((2-(((5S,8S,10aR)-8-(((2S)-5-amino-1-(((1S)-2-((7-(2- (2,6-dioxopiperidin-3-yl)-1-oxoisoindolin-4-yl)hept-6- yn-1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 140 | 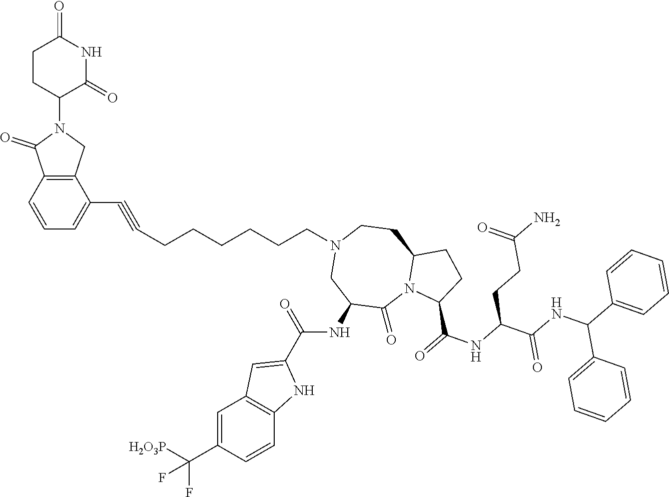 | ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-yn-1-yl)-6-oxodecahydropyrrolo[1,2-a][1,5]diazocin-5- yl)carbamoyl)-1H-indol-5-yl)difluoromethyl)phosphonic acid |
| 141 | 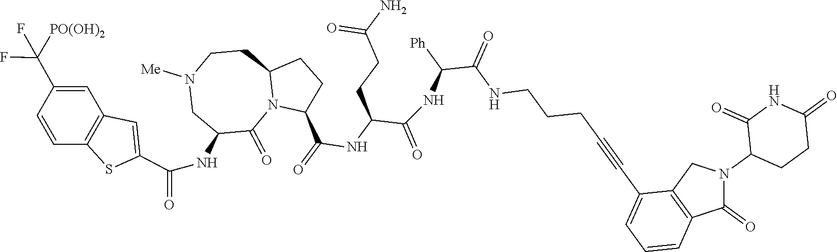 | ((2-(((5S,8S,10aR)-8-(((2S)-5-amino-1-(((1S)-2-((5-(2- (2,6-dioxopiperidin-3-yl)-1-oxoisoindolin-4-yl)pent-4- yn-1-yl)amino)-2-oxo-1-phenylethyl)amino)-1,5- dioxopentan-2-yl)carbamoyl)-3-methyl-6- oxodecahydropyrrolo[1,2-a][1,5] diazocin-5- yl)carbamoyl)benzo[b]thiophen-5- yl)difluoromethyl)phosphonic acid |
| 142 | 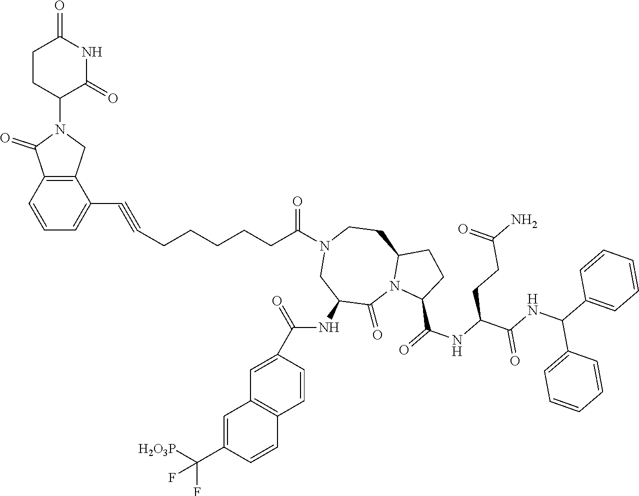 | ((7-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)naphthalen-2- yl)difluoromethyl)phosphonic acid |
| 143 | 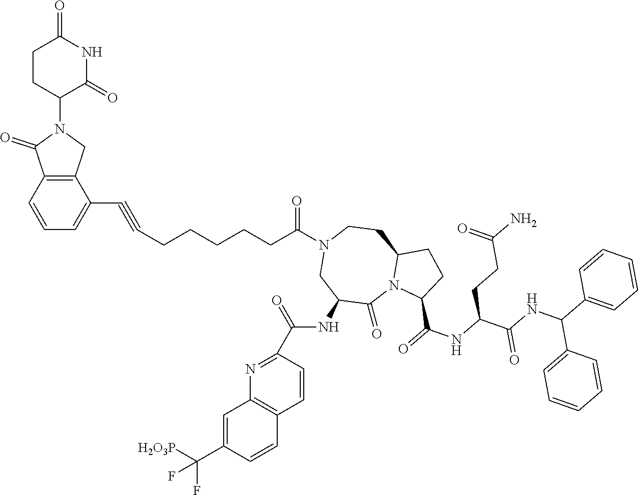 | ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)quinolin-7-yl)difluoromethyl)phosphonic acid |
| 144 | 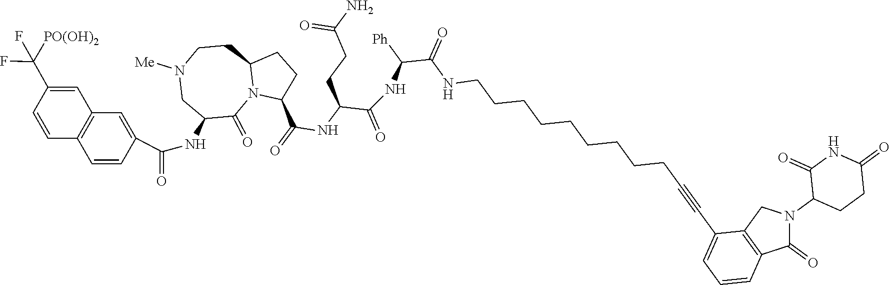 | ((7-(((5S,8S,10aR)-8-(((2S)-5-amino-1-(((1S)-2-((11-(2- (2,6-dioxopiperidin-3-yl)-1-oxoisoindolin-4-yl)undec-10- yn-1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)naphthalen-2- yl)difluoromethyl)phosphonic acid |
| 145 | 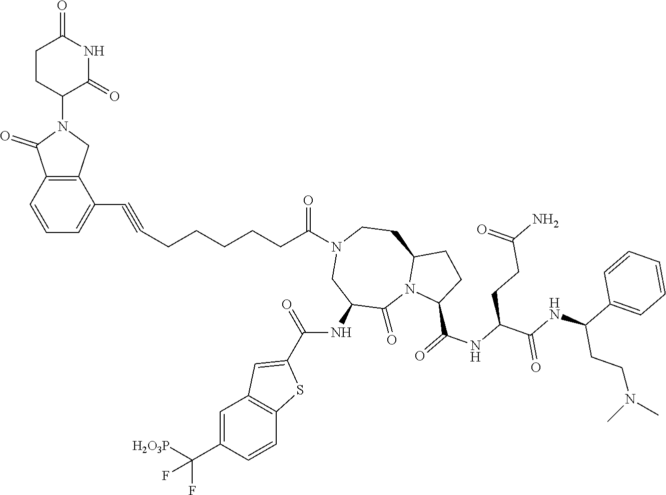 | ((2-(((5S,8S,10aR)-8-(((S)-5-amino-1-(((R)-3- (dimethylamino)-1-phenylpropyl)amino)-1,5- dioxopentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)benzo[b]thiophen-5- yl)difluoromethyl)phosphonic acid |
| 146 | 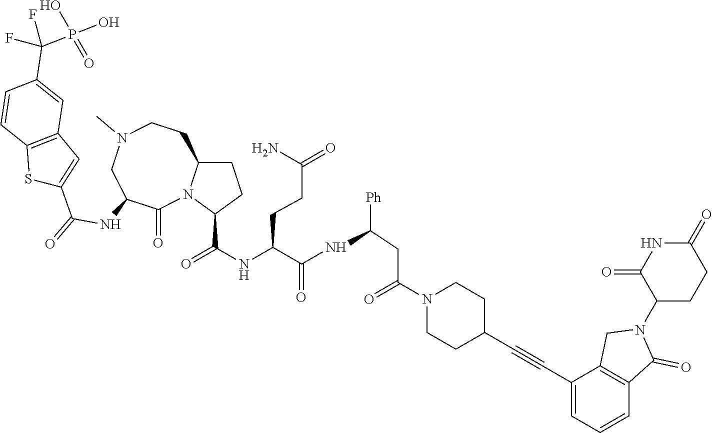 [SMILES: CN1CC[C@H]2CC[C@@H](C(=O)N[C@@H](CCC(N)=O)C(=O)N[C@H](CC(=O)N3CCC(C#Cc4cccc5c4CN(C4CCC(=O)NC4=O)C5=O)CC3)c3ccccc3)N2C(=O)[C@@H](NC(=O)c2cc3cc(C(F)(F)P(=O)(O)O)ccc3s2)C1] | ((2-(((5S,8S,10aR)-8-(((2S)-5-amino-1-(((1R)-3-(4-((2- (2,6-dioxopiperidin-3-yl)-1-oxoisoindolin-4- yl)ethynyl)piperidin-1-yl)-3-oxo-1-phenylpropyl)amino)- 1,5-dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 147 | 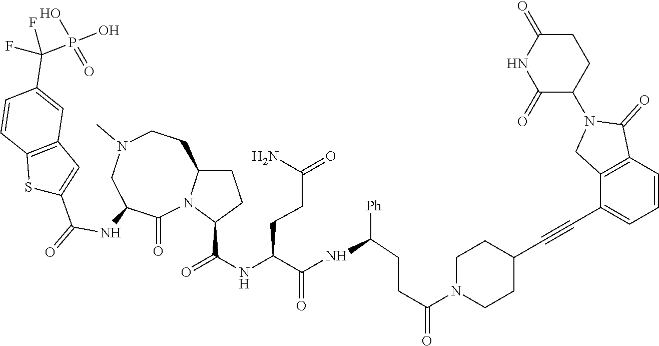 [SMILES: CN1CC[C@H]2CC[C@@H](C(=O)N[C@@H](CCC(N)=O)C(=O)N[C@H](CCC(=O)N3CCC(C#Cc4cccc5c4CN(C4CCC(=O)NC4=O)C5=O)CC3)c3ccccc3)N2C(=O)[C@@H](NC(=O)c2cc3cc(C(F)(F)P(=O)(O)O)ccc3s2)C1] | ((2-(((5S,8S,10aR)-8-(((2S)-5-amino-1-(((1R)-4-(4-((2- (2,6-dioxopiperidin-3-yl)-1-oxoisoindolin-4- yl)ethynyl)piperidin-1-yl)-4-oxo-1-phenylbutyl)amino)- 1,5-dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 148 | 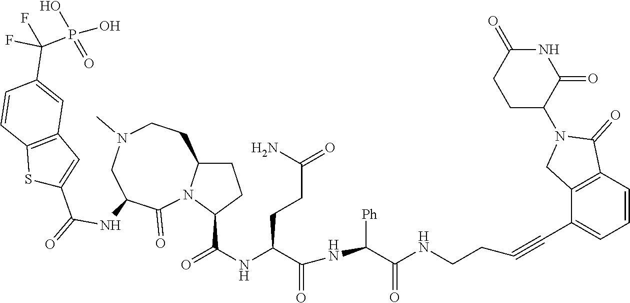 [SMILES: CN1CC[C@H]2CC[C@@H](C(=O)N[C@@H](CCC(N)=O)C(=O)N[C@H](C(=O)NCCC#Cc3cccc4c3CN(C3CCC(=O)NC3=O)C4=O)c3ccccc3)N2C(=O)[C@@H](NC(=O)c2cc3cc(C(F)(F)P(=O)(O)O)ccc3s2)C1] | ((2-(((5S,8S,10aR)-8-(((2S)-5-amino-1-(((1S)-2-((6-(2- (2,6-dioxopiperidin-3-yl)-1-oxoisoindolin-4-yl)hex-5-yn- 1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 149 | 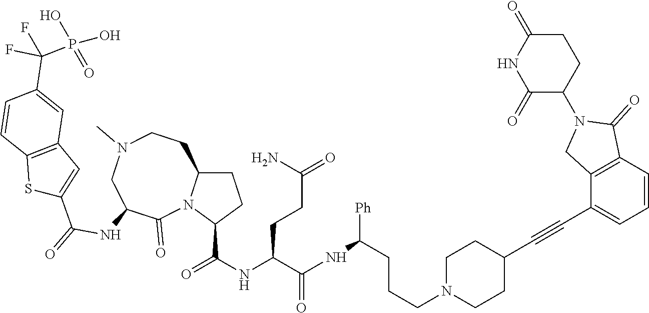 [SMILES: CN1CC[C@H]2CC[C@@H](C(=O)N[C@@H](CCC(N)=O)C(=O)N[C@H](CCCN3CCC(C#Cc4cccc5c4CN(C4CCC(=O)NC4=O)C5=O)CC3)c3ccccc3)N2C(=O)[C@@H](NC(=O)c2cc3cc(C(F)(F)P(=O)(O)O)ccc3s2)C1] | ((2-(((5S,8S,10aR)-8-(((2S)-5-amino-1-(((1R)-4-(4-((2- (2,6-dioxopiperidin-3-yl)-1-oxoisoindolin-4- yl)ethynyl)piperidin-1-yl)-1-phenylbutyl)amino)-1,5- dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 150 | 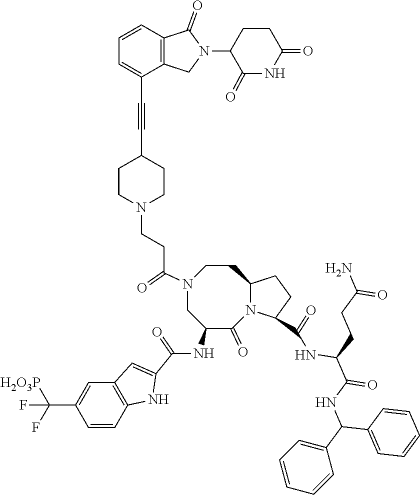 | ((2-(((5S,8S,10aR)-8-(((S)-5-amino-1- (benzhydrylamino)-1,5-dioxopentan-2-yl)carbamoyl)-3- (3-(4-((2-(2,6-dioxopiperidin-3-yl)-1-oxoisoindolin-4- yl)ethynyl)piperidin-1-yl)propanoyl)-6- oxodecahydropyrrolo[1,2-a][1,5]diazocin-5- yl)carbamoyl)-1H-indol-5-yl)difluoromethyl)phosphonic acid |
| 151 | 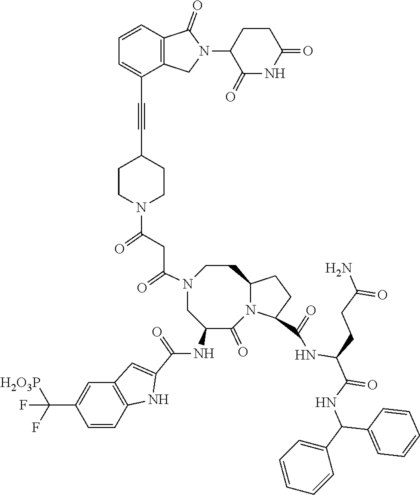 | ((2-(((5S,8S,10aR)-8-(((S)-5-amino-1- (benzhydrylamino)-1,5-dioxopentan-2-yl)carbamoyl)-3- (3-(4-((2-(2,6-dioxopiperidin-3-yl)-1-oxoisoindolin-4- yl)ethynyl)piperidin-1-yl)-3-oxopropanoyl)-6- oxodecahydropyrrolo[1,2-a][1,5]diazocin-5- yl)carbamoyl)-1H-indol-5-yl)difluoromethyl)phosphonic acid |
| 152 | 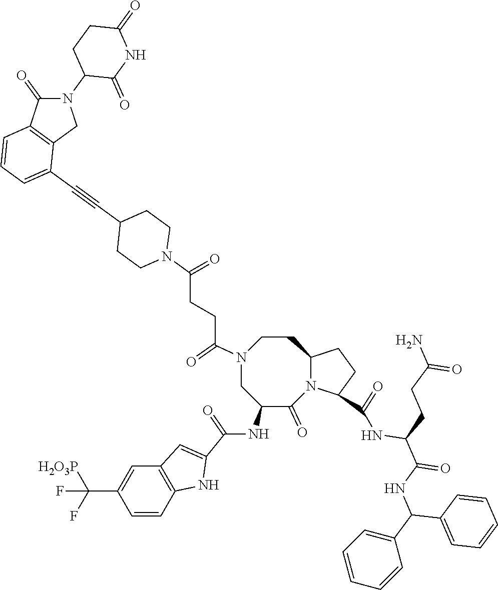 | ((2-(((5S,8S,10aR)-8-(((S)-5-amino-1- (benzhydrylamino)-1,5-dioxopentan-2-yl)carbamoyl)-3- (4-(4-((2-(2,6-dioxopiperidin-3-yl)-1-oxoisoindolin-4- yl)ethynyl)piperidin-1-yl)-4-oxobutanoyl)-6- oxodecahydropyrrolo[1,2-a][1,5]diazocin-5- yl)carbamoyl)-1H-indol-5-yl)difluoromethyl)phosphonic acid |
| 153 | 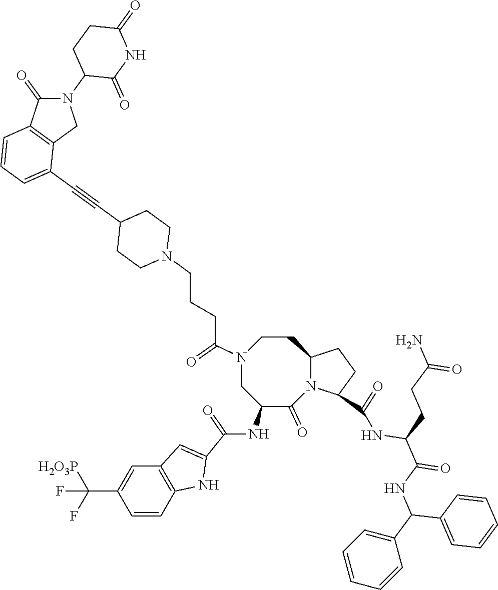 | ((2-(((5S,8S,10aR)-8-(((S)-5-amino-1- (benzhydrylamino)-1,5-dioxopentan-2-yl)carbamoyl)-3- (4-(4-((2-(2,6-dioxopiperidin-3-yl)-1-oxoisoindolin-4- yl)ethynyl)piperidin-1-yl)butanoyl)-6- oxodecahydropyrrolo[1,2-a][1,5]diazocin-5- yl)carbamoyl)-1H-indol-5-yl)difluoromethyl)phosphonic acid |
| 154 | 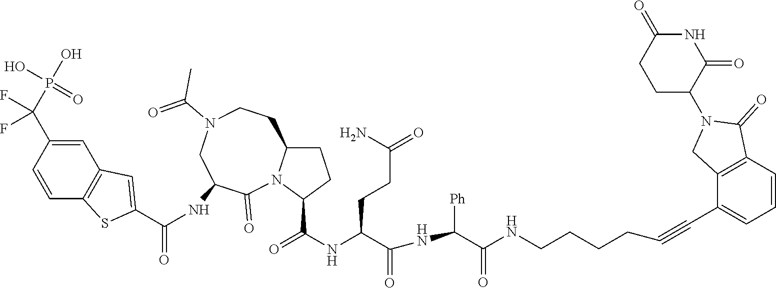 [SMILES: CC(=O)N1CC[C@H]2CC[C@@H](C(=O)N[C@@H](CCC(N)=O)C(=O)N[C@H](C(=O)NCCCCC#Cc3cccc4c3CN(C3CCC(=O)NC3=O)C4=O)c3ccccc3)N2C(=O)[C@@H](NC(=O)c2cc3cc(C(F)(F)P(=O)(O)O)ccc3s2)C1] | ((2-(((5S,8S,10aR)-3-acetyl-8-(((2S)-5-amino-1-(((1S)-2- ((6-(2-(2,6-dioxopiperidin-3-yl)-1-oxoisoindolin-4- yl)hex-5-yn-1-yl)amino)-2-oxo-1-phenylethyl)amino)- 1,5-dioxopentan-2-yl)carbamoyl)-6- oxodecahydropyrrolo[1,2-a][1,5]diazocin-5- yl)carbamoyl)benzo[b]thiophen-5- yl)difluoromethyl)phosphonic acid |
| 155 | 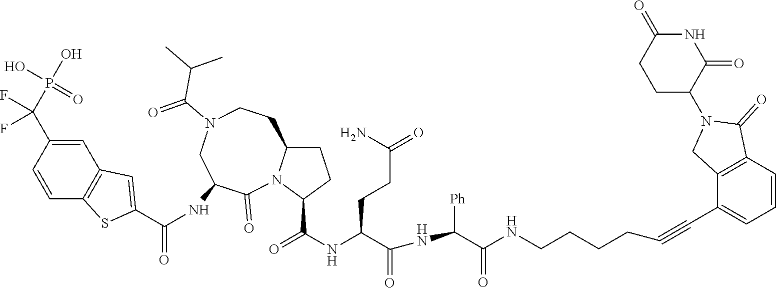 [SMILES: CC(C)C(=O)N1CC[C@H]2CC[C@@H](C(=O)N[C@@H](CCC(N)=O)C(=O)N[C@H](C(=O)NCCCCC#Cc3cccc4c3CN(C3CCC(=O)NC3=O)C4=O)c3ccccc3)N2C(=O)[C@@H](NC(=O)c2cc3cc(C(F)(F)P(=O)(O)O)ccc3s2)C1] | ((2-(((5S,8S,10aR)-8-(((2S)-5-amino-1-(((1S)-2-((6-(2- (2,6-dioxopiperidin-3-yl)-1-oxoisoindolin-4-yl)hex-5-yn- 1-yl)amino)-2-oxo-1-phenylethyl)amino)-1,5- dioxopentan-2-yl)carbamoyl)-3-isobutyryl-6- oxodecahydropyrrolo[1,2-a][1,5]diazocin-5- yl)carbamoyl)benzo[b]thiophen-5- yl)difluoromethyl)phosphonic acid |
| 156 | 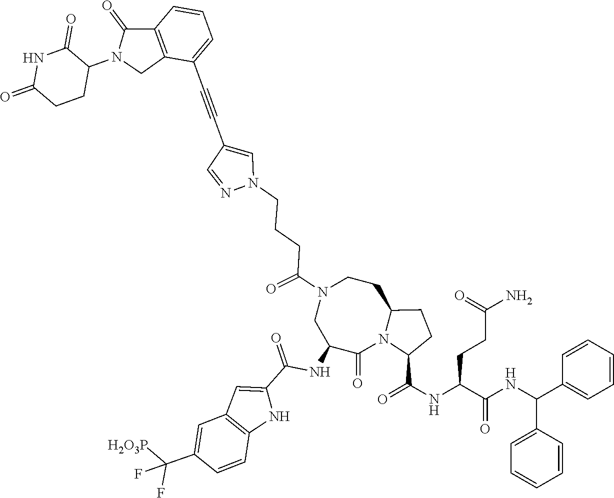 | ((2-(((5S,8S,10aR)-8-(((S)-5-amino-1- (benzhydrylamino)-1,5-dioxopentan-2-yl)carbamoyl)-3- (4-(4-((2-(2,6-dioxopiperidin-3-yl)-1-oxoisoindolin-4- yl)ethynyl)-1H-pyrazol-1-yl)butanoyl)-6- oxodecahydropyrrolo[1,2-a][1,5]diazocin-5- yl)carbamoyl)-1H-indol-5-yl)difluoromethyl)phosphonic acid |
| 157 | 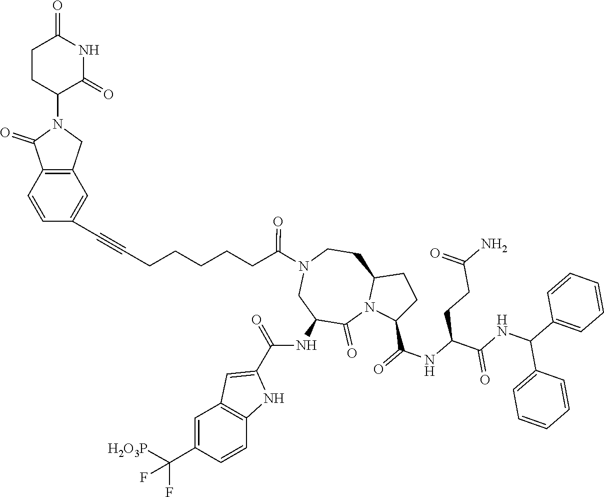 | ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-5-yl)oct- 7-ynoyl)-6-oxodecahydropyrrolo[1,2-a][1,5]diazocin-5- yl)carbamoyl)-1H-indol-5-yl)difluoromethyl)phosphonic acid |
| 158 | 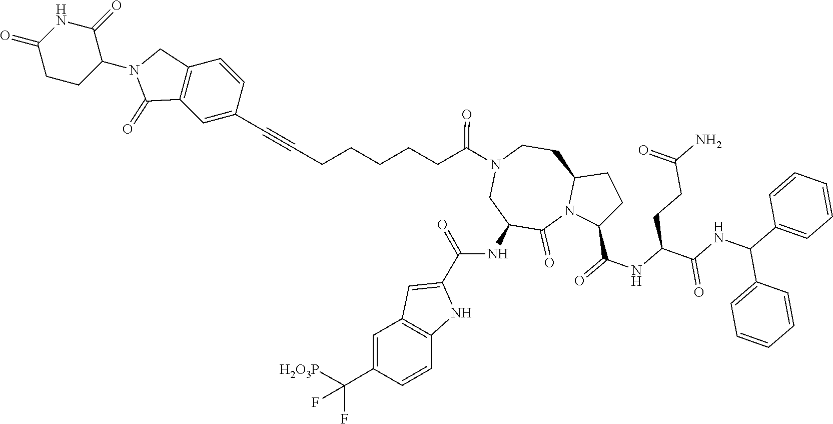 | ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-3-oxoisoindolin-5-yl)oct- 7-ynoyl)-6-oxodecahydropyrrolo[1,2-a][1,5]diazocin-5- yl)carbamoyl)-1H-indol-5-yl)difluoromethyl)phosphonic acid |
| 159 | 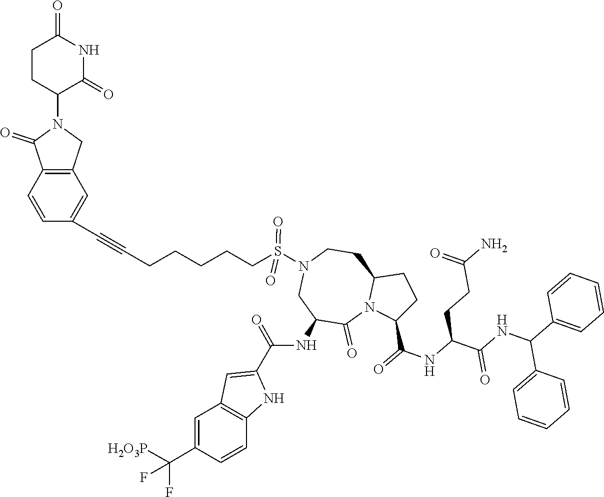 | ((2-(((5S,8S,10aR)-8-(((S)-5-amino-1- (benzhydrylamino)-1,5-dioxopentan-2-yl)carbamoyl)-3- ((7-(2-(2,6-dioxopiperidin-3-yl)-1-oxoisoindolin-4- yl)hept-6-yn-1-yl)sulfonyl)-6-oxodecahydropyrrolo[1,2- a][1,5]diazocin-5-yl)carbamoyl)-1H-indol-5- yl)difluoromethyl)phosphonic acid |
| 160 | 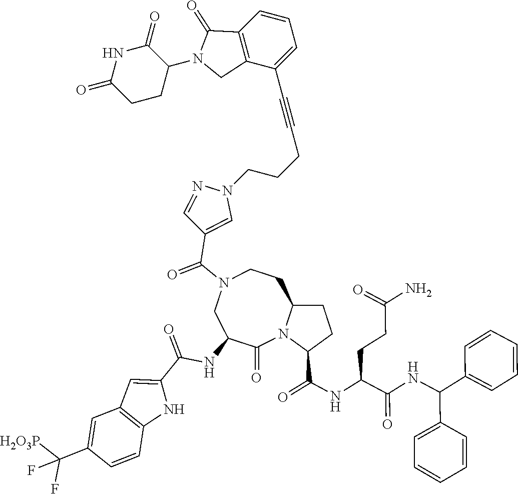 | ((2-(((5S,8S,10aR)-8-(((S)-5-amino-1- (benzhydrylamino)-1,5-dioxopentan-2-yl)carbamoyl)-3- (1-(5-(2-(2,6-dioxopiperidin-3-yl)-1-oxoisoindolin-4- yl)pent-4-yn-1-yl)-1H-pyrazole-4-carbonyl)-6- oxodecahydropyrrolo[1,2-a][1,5]diazocin-5- yl)carbamoyl)-1H-indol-5-yl)difluoromethyl)phosphonic acid |
| 161 | 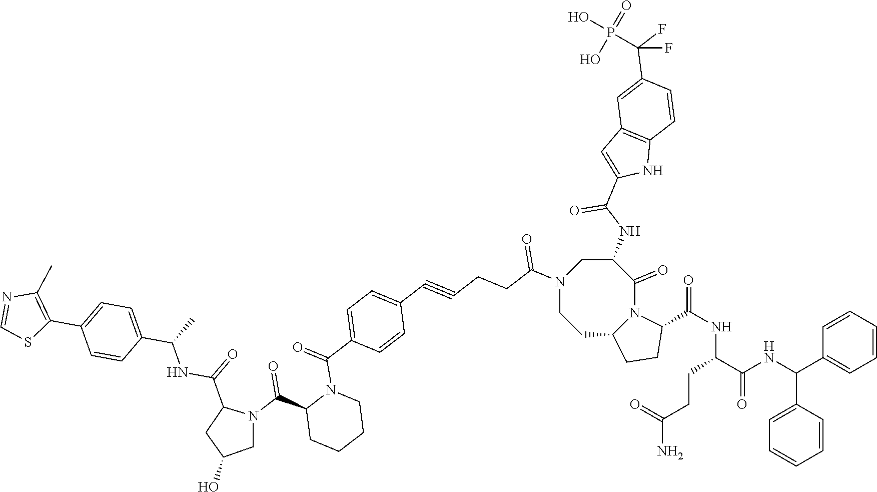 [SMILES: Cc1ncsc1-c1ccc([C@H](C)NC(=O)C2C[C@@H](O)CN2C(=O)[C@@H]2CCCCN2C(=O)c2ccc(C#CCCC(=O)N3CC[C@H]4CC[C@@H](C(=O)N[C@@H](CCC(N)=O)C(=O)NC(c5ccccc5)c5ccccc5)N4C(=O)[C@@H](NC(=O)c4cc5cc(C(F)(F)P(=O)(O)O)ccc5[nH]4)C3)cc2)cc1] | ((2-(((5S,8S,10aR)-8-(((S)-5-amino-1- (benzhydrylamino)-1,5-dioxopentan-2-yl)carbamoyl)-3- (5-(4-((S)-2-((2S,4R)-4-hydroxy-2-(((S)-1-(4-(4- methylthiazol-5-yl)phenyl)ethyl)carbamoyl)pyrrolidine- 1-carbonyl)piperidine-1-carbonyl)phenyl)pent-4-ynoyl)- 6-oxodecahydropyrrolo[1,2-a][1,5]diazocin-5- yl)carbamoyl)-1H-indol-5-yl)difluoromethyl)phosphonic acid |
| 162 | 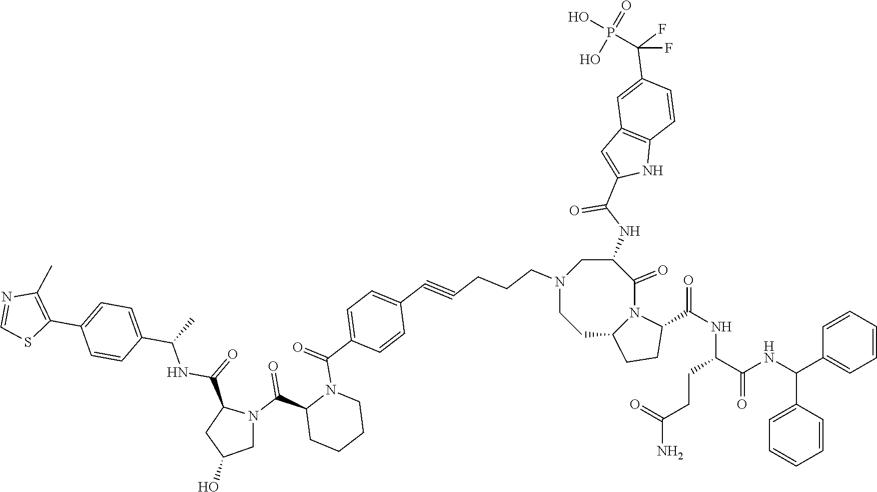 [SMILES: Cc1ncsc1-c1ccc([C@H](C)NC(=O)[C@@H]2C[C@@H](O)CN2C(=O)[C@@H]2CCCCN2C(=O)c2ccc(C#CCCCN3CC[C@H]4CC[C@@H](C(=O)N[C@@H](CCC(N)=O)C(=O)NC(c5ccccc5)c5ccccc5)N4C(=O)[C@@H](NC(=O)c4cc5cc(C(F)(F)P(=O)(O)O)ccc5[nH]4)C3)cc2)cc1] | ((2-(((5S,8S,10aR)-8-(((S)-5-amino-1- (benzhydrylamino)-1,5-dioxopentan-2-yl)carbamoyl)-3- (5-(4-((S)-2-((2S,4R)-4-hydroxy-2-(((S)-1-(4-(4- methylthiazol-5-yl)phenyl)ethyl)carbamoyl)pyrrolidine- 1-carbonyl)piperidine-1-carbonyl)phenyl)pent-4-yn-1- yl)-6-oxodecahydropyrrolo[1,2-a][1,5]diazocin-5- yl)carbamoyl)-1H-indol-5-yl)difluoromethyl)phosphonic acid |
| 163 | 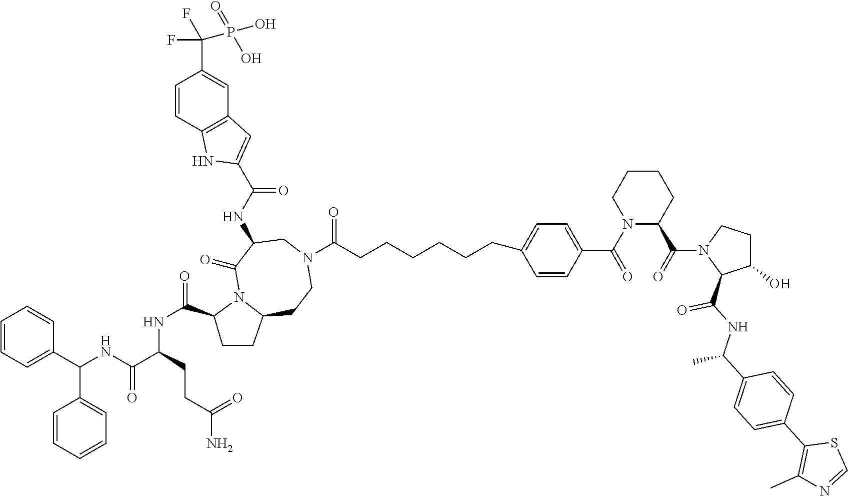 [SMILES: Cc1ncsc1-c1ccc([C@H](C)NC(=O)[C@@H]2[C@@H](O)CCN2C(=O)[C@@H]2CCCCN2C(=O)c2ccc(CCCCCCC(=O)N3CC[C@H]4CC[C@@H](C(=O)N[C@@H](CCC(N)=O)C(=O)NC(c5ccccc5)c5ccccc5)N4C(=O)[C@@H](NC(=O)c4cc5cc(C(F)(F)P(=O)(O)O)ccc5[nH]4)C3)cc2)cc1] | ((2-(((5S,8S,10aR)-8-(((S)-5-amino-1- (benzhydrylamino)-1,5-dioxopentan-2-yl)carbamoyl)-3- (7-(4-((S)-2-((2S,3S)-3-hydroxy-2-(((S)-1-(4-(4- methylthiazol-5-yl)phenyl)ethyl)carbamoyl)pyrrolidine- 1-carbonyl)piperidine-1-carbonyl)phenyl)heptanoyl)-6- oxodecahydropyrrolo[1,2-a][1,5]diazocin-5- yl)carbamoyl)-1H-indol-5-yl)difluoromethyl)phosphonic acid |
| 164 | 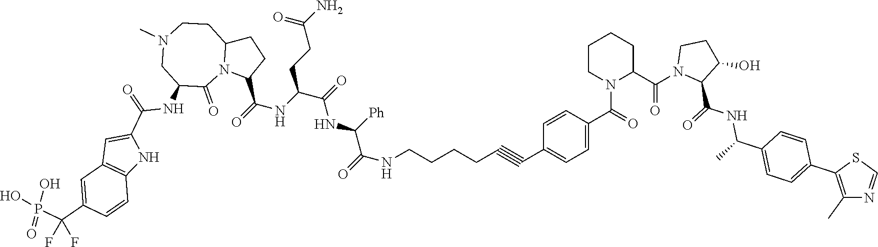 | ((2-(((5S,8S,10aR)-8-(((S)-5-amino-1-(((S)-2-((6-(4-((S)- 2-((2S,3S)-3-hydroxy-2-(((S)-1-(4-(4-methylthiazol-5- yl)phenyl)ethyl)carbamoyl)pyrrolidine-1- carbonyl)piperidine-1-carbonyl)phenyl)hex-5-yn-1- yl)amino)-2-oxo-1-phenylethyl)amino)-1,5-dioxopentan- 2-yl)carbamoyl)-3-methyl-6-oxodecahydropyrrolo[1,2- a][1,5]diazocin-5-yl)carbamoyl)-1H-indol-5- yl)difluoromethyl)phosphonic acid |
| 165 | 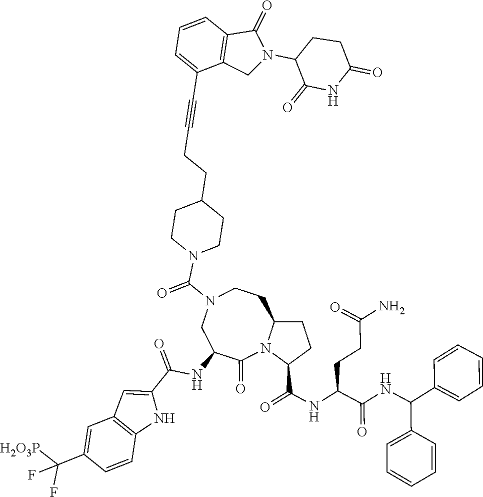 | ((2-(((5S,8S,10aR)-8-(((S)-5-amino-1- (benzhydrylamino)-1,5-dioxopentan-2-yl)carbamoyl)-3- (4-(4-(2-(2,6-dioxopiperidin-3-yl)-1-oxoisoindolin-4- yl)but-3-yn-1-yl)piperidine-1-carbonyl)-6- oxodecahydropyrrolo[1,2-a][1,5]diazocin-5- yl)carbamoyl)-1H-indol-5-yl)difluoromethyl)phosphonic acid |
| 166 | 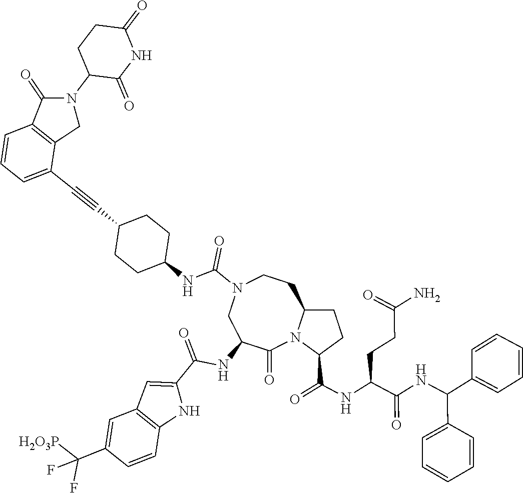 | ((2-(((5S,8S,10aR)-8-(((S)-5-amino-1- (benzhydrylamino)-1,5-dioxopentan-2-yl)carbamoyl)-3- (((1r,4S)-4-((2-(2,6-dioxopiperidin-3-yl)-1- oxoisoindolin-4-yl)ethynyl)cyclohexyl)carbamoyl)-6- oxodecahydropyrrolo[1,2-a][1,5]diazocin-5- yl)carbamoyl)-1H-indol-5-yl)difluoromethyl)phosphonic acid |
| 167 | 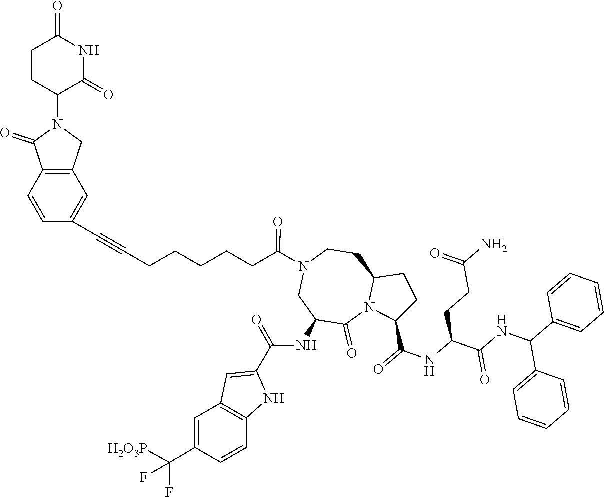 | ((2-(((5S,8S,10aR)-8-(((S)-5-amino-1- (benzhydrylamino)-1,5-dioxopentan-2-yl)carbamoyl)-3- (7-(2-(2,6-dioxopiperidin-3-yl)-1-oxoisoindolin-5- yl)hept-6-ynoyl)-6-oxodecahydropyrrolo[1,2- a][1,5]diazocin-5-yl)carbamoyl)-1H-indol-5- yl)difluoromethyl)phosphonic acid |
| 168 | 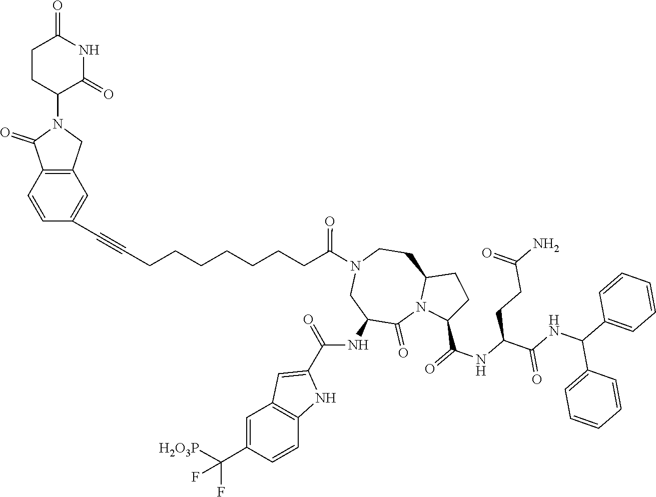 | ((2-(((5S,8S,10aR)-8-(((S)-5-amino-1- (benzhydrylamino)-1,5-dioxopentan-2-yl)carbamoyl)-3- (9-(2-(2,6-dioxopiperidin-3-yl)-1-oxoisoindolin-5- yl)non-8-ynoyl)-6-oxodecahydropyrrolo[1,2- a][1,5]diazocin-5-yl)carbamoyl)-1H-indol-5- yl)difluoromethyl)phosphonic acid |
| 169 | 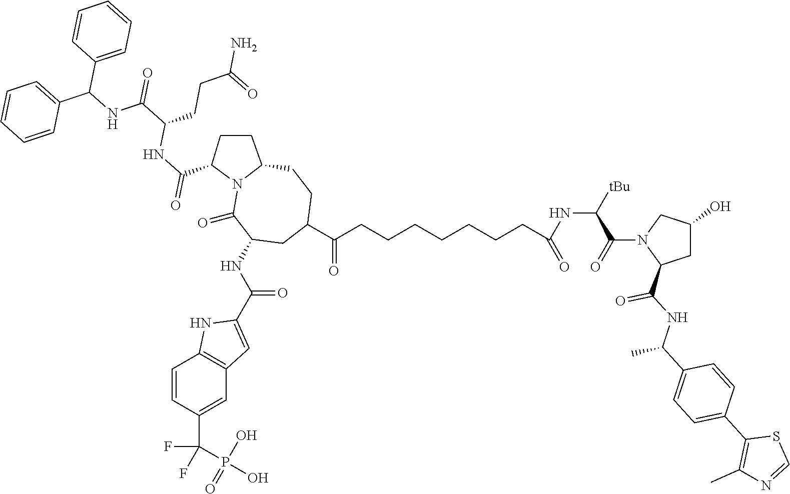 | ((2-(((5S,8S,10aR)-8-(((S)-5-amino-1- (benzhydrylamino)-1,5-dioxopentan-2-yl)carbamoyl)-3- (9-(((S)-1-((2S,4R)-4-hydroxy-2-(((S)-1-(4-(4- methylthiazol-5-yl)phenyl)ethyl)carbamoyl)pyrrolidin-1- yl)-3,3-dimethyl-1-oxobutan-2-yl)amino)-9- oxononanoyl)-6-oxodecahydropyrrolo[1,2- a][1,5]diazocin-5-yl)carbamoyl)-1H-indol-5- yl)difluoromethyl)phosphonic acid |
| 170 | 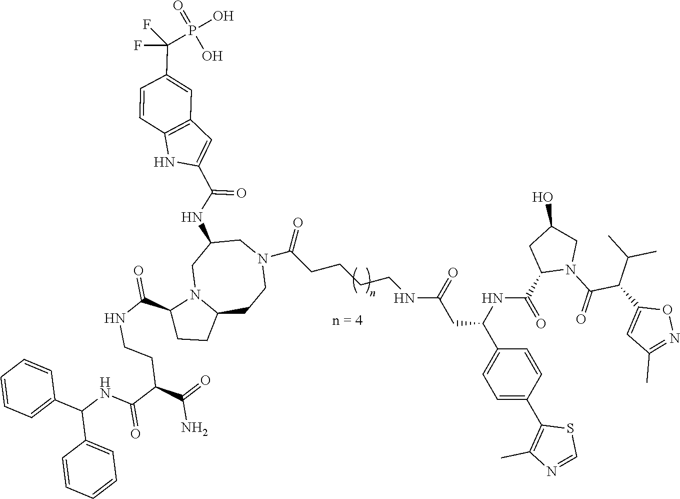 | ((2-(((5S,8S,10aR)-8-(((S)-5-amino-1- (benzhydrylamino)-1,5-dioxopentan-2-yl)carbamoyl)-3- (5-((S)-3-((2S,4R)-4-hydroxy-1-((R)-3-methyl-2-(3- methylisoxazol-5-yl)butanoyl)pyrrolidine-2- carboxamido)-3-(4-(4-methylthiazol-5- yl)phenyl)propanamido)pentanoyl)-6- oxodecahydropyrrolo[1,2-a][1,5]diazocin-5- yl)carbamoyl)-1H-indol-5-yl)difluoromethyl)phosphonic acid |
| 171 | 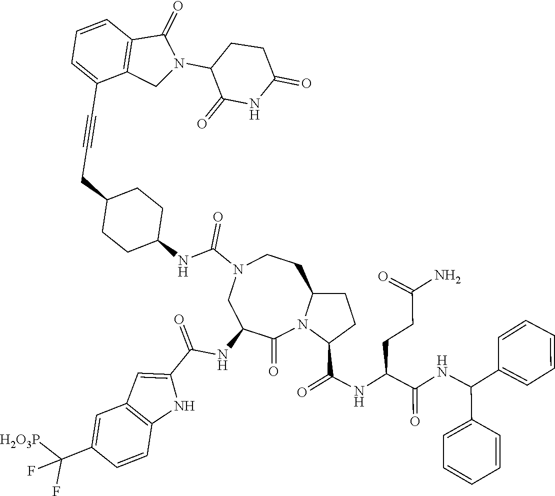 | (benzhydrylamino)-1,5-dioxopentan-2-yl)carbamoyl)-3- (((1s,4R)-4-(3-(2-(2,6-dioxopiperidin-3-yl)-1- oxoisoindolin-4-yl)prop-2-yn-1- yl)cyclohexyl)carbamoyl)-6-oxodecahydropyrrolo[1,2- a][1,5]diazocin-5-yl)carbamoyl)-1H-indol-5- yl)difluoromethyl)phosphonic acid |
| 172 | 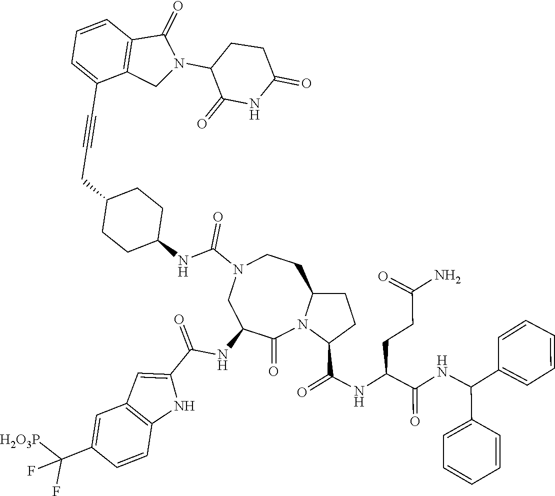 | ((2-(((5S,8S,10aR)-8-(((S)-5-amino-1- (benzhydrylamino)-1,5-dioxopentan-2-yl)carbamoyl)-3- (((1r,4S)-4-(3-(2-(2,6-dioxopiperidin-3-yl)-1- oxoisoindolin-4-yl)prop-2-yn-1- yl)cyclohexyl)carbamoyl)-6-oxodecahydropyrrolo[1,2- a][1,5]diazocin-5-yl)carbamoyl)-1H-indol-5- yl)difluoromethyl)phosphonic acid |
| 173 | 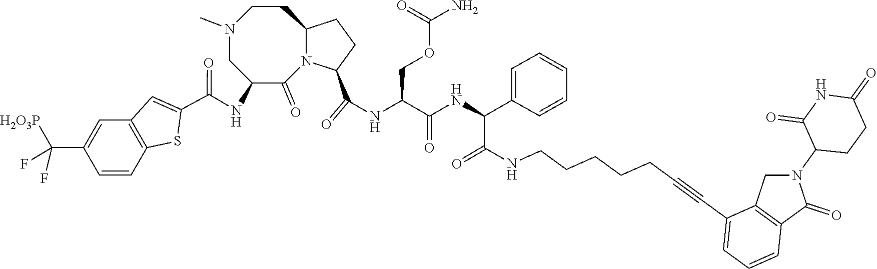 | ((2-(((5S,8S,10aR)-8-(((2S)-3-(carbamoyloxy)-1-(((1S)- 2-((7-(2-(2,6-dioxopiperidin-3-yl)-1-oxoisoindolin-4- yl)hept-6-yn-1-yl)amino)-2-oxo-1-phenylethyl)amino)-1- oxopropan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 174 | 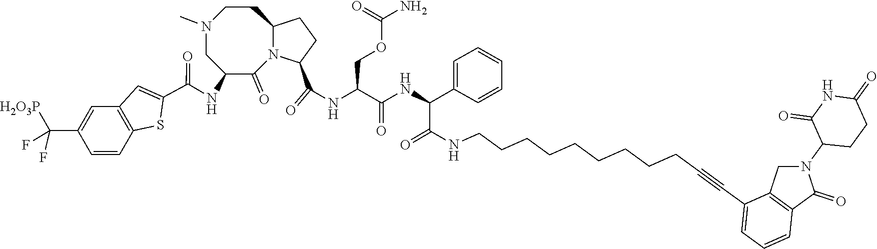 | ((2-(((5S,8S,10aR)-8-(((2S)-3-(carbamoyloxy)-1-(((1S)- 2-((11-(2-(2,6-dioxopiperidin-3-yl)-1-oxoisoindolin-4- yl)undec-10-yn-1-yl)amino)-2-oxo-1- phenylethyl)amino)-1-oxopropan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 175 | 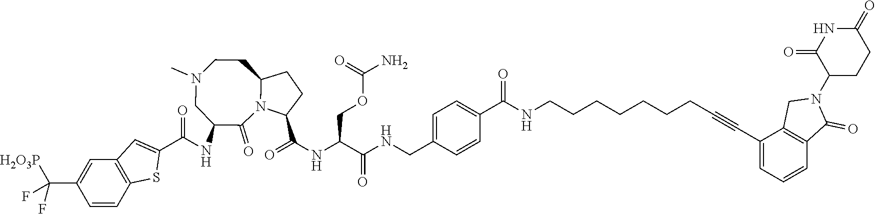 | ((2-(((5S,8S,10aR)-8-(((2S)-3-(carbamoyloxy)-1-((4- ((11-(2-(2,6-dioxopiperidin-3-yl)-1-oxoisoindolin-4- yl)undec-10-yn-1-yl)carbamoyl)benzyl)amino)-1- oxopropan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 176 | 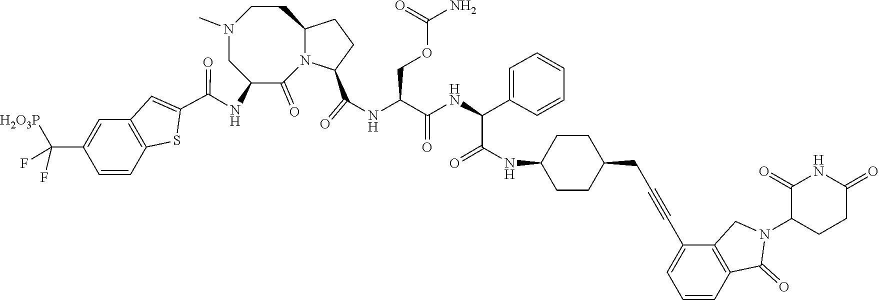 | ((2-(((5S,8S,10aR)-8-(((2S)-3-(carbamoyloxy)-1-(((1S)- 2-(((1s,4R)-4-(3-(2-(2,6-dioxopiperidin-3-yl)-1- oxoisoindolin-4-yl)prop-2-yn-1-yl)cyclohexyl)amino)-2- oxo-1-phenylethyl)amino)-1-oxopropan-2-yl)carbamoyl)- 3-methyl-6-oxodecahydropyrrolo[1,2-a][1,5]diazocin-5- yl)carbamoyl)benzo[b]thiophen-5- yl)difluoromethyl)phosphonic acid |
| 177 | 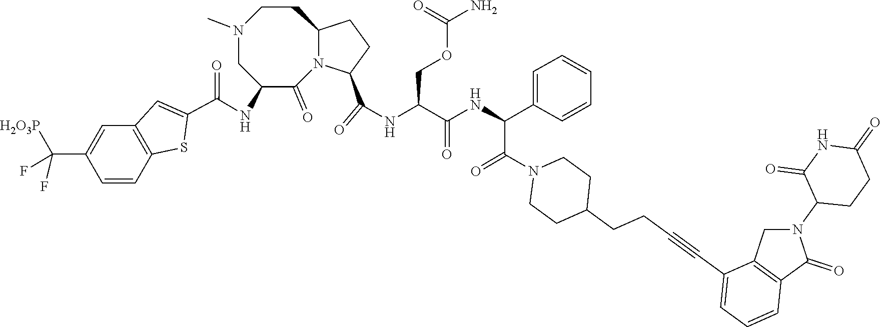 | ((2-(((5S,8S,10aR)-8-(((2S)-3-(carbamoyloxy)-1-(((1S)- 2-(4-(4-(2-(2,6-dioxopiperidin-3-yl)-1-oxoisoindolin-4- yl)but-3-yn-1-yl)piperidin-1-yl)-2-oxo-1- phenylethyl)amino)-1-oxopropan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 178 | 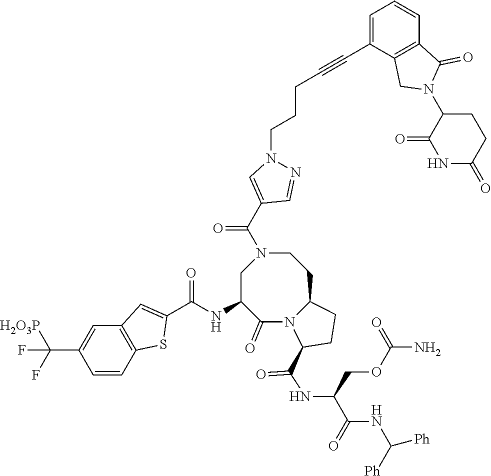 | ((2-(((5S,8S,10aR)-8-(((S)-1-(benzhydrylamino)-3- (carbamoyloxy)-1-oxopropan-2-yl)carbamoyl)-3-(1-(5- (2-(2,6-dioxopiperidin-3-yl)-1-oxoisoindolin-4-yl)pent-4- yn-1-yl)-1H-pyrazole-4-carbonyl)-6- oxodecahydropyrrolo[1,2-a][1,5]diazocin-5- yl)carbamoyl)benzo[b]thiophen-5- yl)difluoromethyl)phosphonic acid |
| 179 | 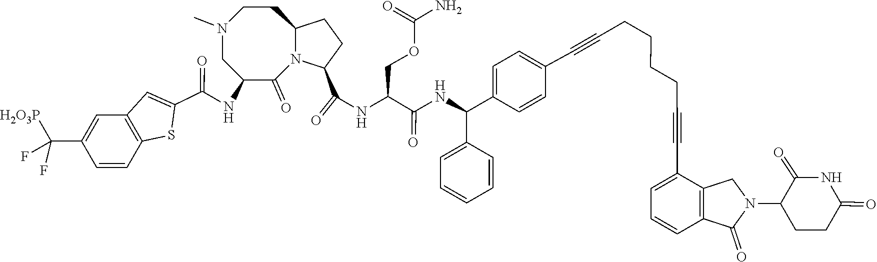 | ((2-(((5S,8S,10aR)-8-(((2S)-3-(carbamoyloxy)-1-(((1R)- (4-(8-(2-(2,6-dioxopiperidin-3-yl)-1-oxoisoindolin-4- yl)octa-1,7-diyn-1-yl)phenyl)(phenyl)methyl)amino)-1- oxopropan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 180 | 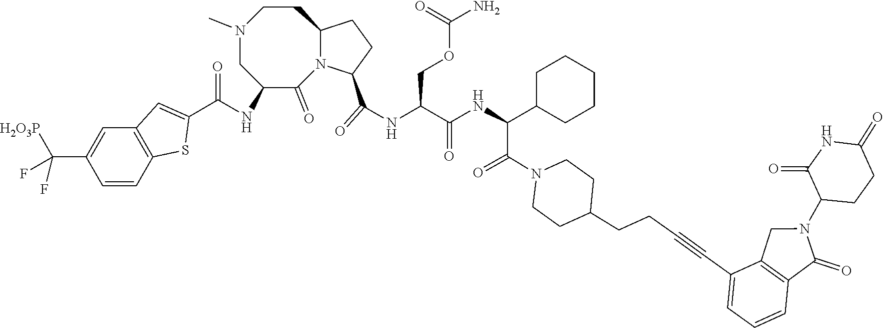 | ((2-(((5S,8S,10aR)-8-(((2S)-3-(carbamoyloxy)-1-(((1S)- 1-cyclohexyl-2-(4-(4-(2-(2,6-dioxopiperidin-3-yl)-1- oxoisoindolin-4-yl)but-3-yn-1-yl)piperidin-1-yl)-2- oxoethyl)amino)-1-oxopropan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 181 | 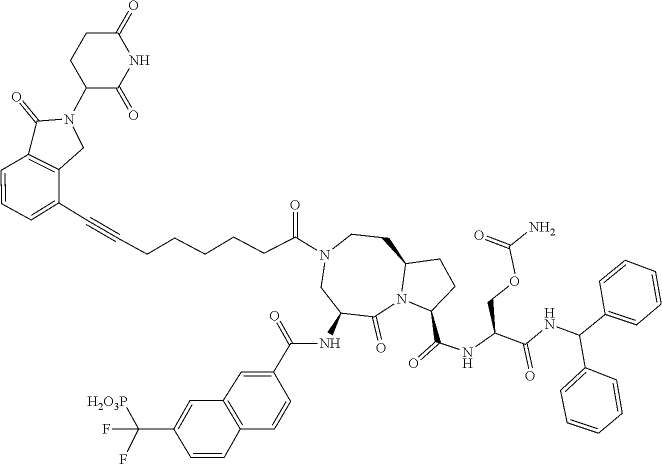 | ((7-(((5S,8S,10aR)-8-(((S)-1-(benzhydrylamino)-3- (carbamoyloxy)-1-oxopropan-2-yl)carbamoyl)-3-(8-(2- (2,6-dioxopiperidin-3-yl)-1-oxoisoindolin-4-yl)oct-7- ynoyl)-6-oxodecahydropyrrolo[1,2-a][1,5]diazocin-5- yl)carbamoyl)naphthalen-2- yl)difluoromethyl)phosphonic acid |
| 182 | 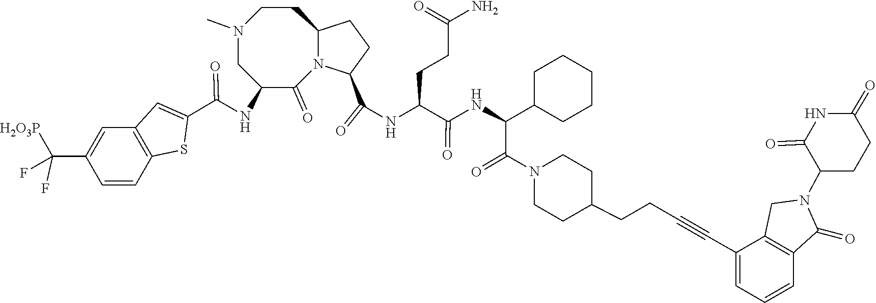 | ((2-(((5S,8S,10aR)-8-(((2S)-5-amino-1-(((1S)-1- cyclohexyl-2-(4-(4-(2-(2,6-dioxopiperidin-3-yl)-1- oxoisoindolin-4-yl)but-3-yn-1-yl)piperidin-1-yl)-2- oxoethyl)amino)-1,5-dioxopentan-2-yl)carbamoyl)-3- methyl-6-oxodecahydropyrrolo[1,2-a][1,5]diazocin-5- yl)carbamoyl)benzo[b] thiophen-5- yl)difluoromethyl)phosphonic acid |
| 183 | 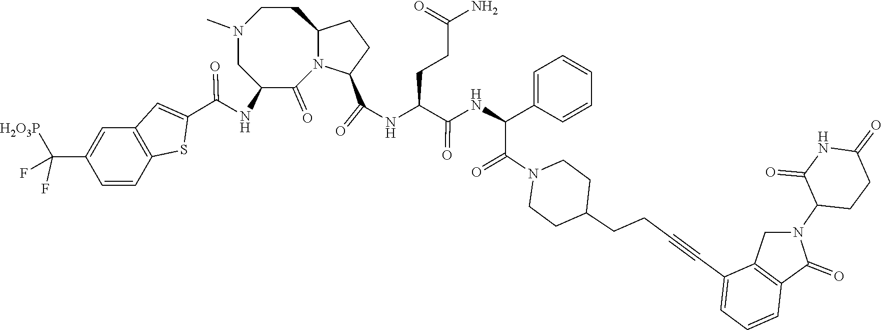 | ((2-(((5S,8S,10aR)-8-(((2S)-5-amino-1-(((1S)-2-(4-(4-(2- (2,6-dioxopiperidin-3-yl)-1-oxoisoindolin-4-yl)but-3-yn- 1-yl)piperidin-1-yl)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 184 | 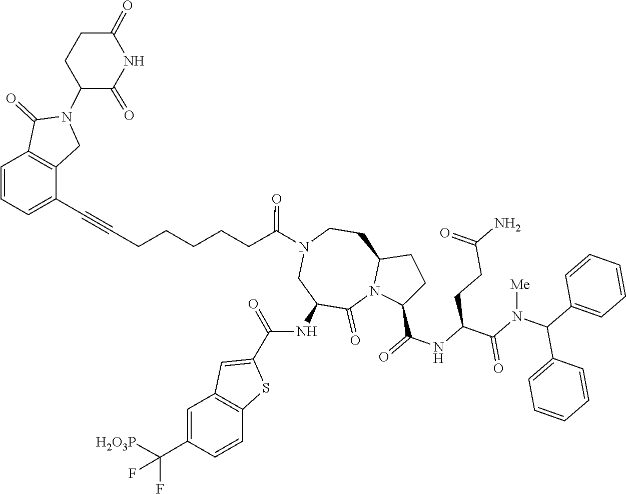 | ((2-(((5S,8S,10aR)-8-(((S)-5-amino-1- (benzhydryl(methyl)amino)-1,5-dioxopentan-2- yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)benzo[b]thiophen-5- yl)difluoromethyl)phosphonic acid |
| 185 | 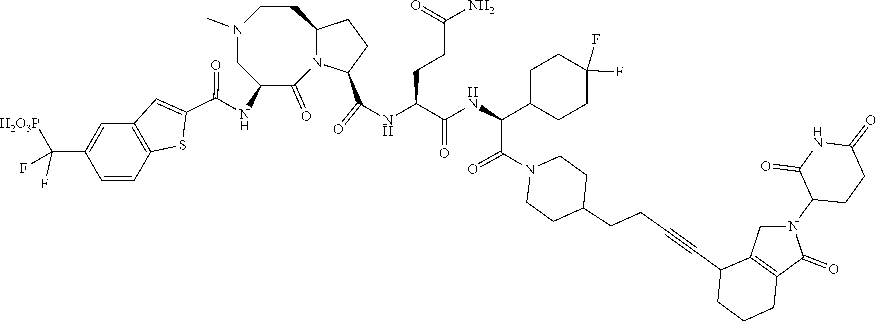 | ((2-(((5S,8S,10aR)-8-(((2S)-5-amino-1-(((1S)-1-(4,4- difluorocyclohexyl)-2-(4-(4-(2-(2,6-dioxopiperidin-3-yl)- 1-oxoisoindolin-4-yl)but-3-yn-1-yl)piperidin-1-yl)-2- oxoethyl)amino)-1,5-dioxopentan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 186 | 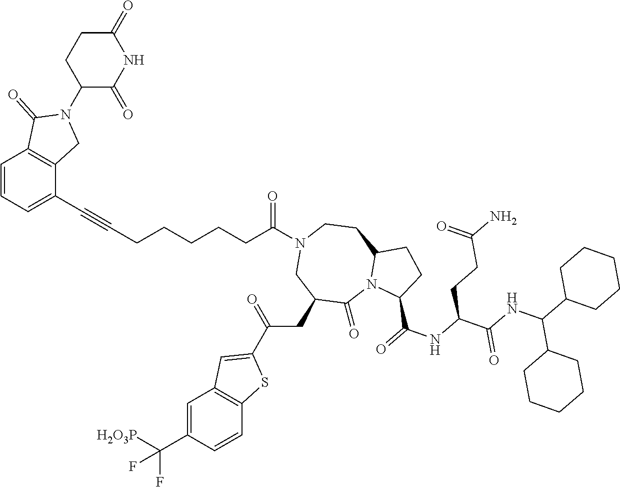 | ((2-(((5S,8S,10aR)-8-(((S)-5-amino-1- ((dicyclohexylmethyl)amino)-1,5-dioxopentan-2- yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)benzo[b]thiophen-5- yl)difluoromethyl)phosphonic acid |
| 187 | 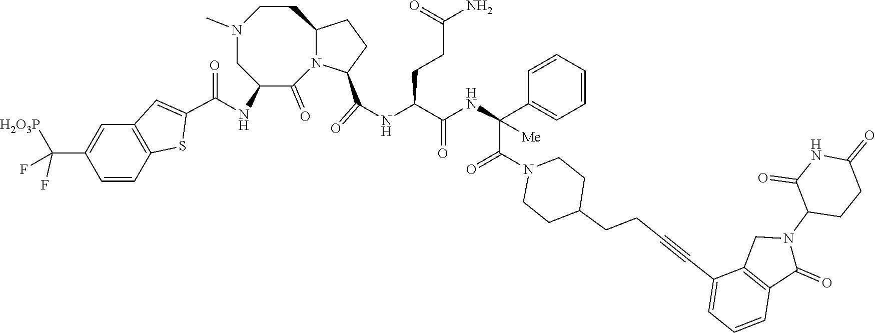 | ((2-(((5S,8S,10aR)-8-(((2S)-5-amino-1-((1-(4-(4-(2-(2,6- dioxopiperidin-3-yl)-1-oxoisoindolin-4-yl)but-3-yn-1- yl)piperidin-1-yl)-1-oxo-2-phenylpropan-2-yl)amino)- 1,5-dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 188 | 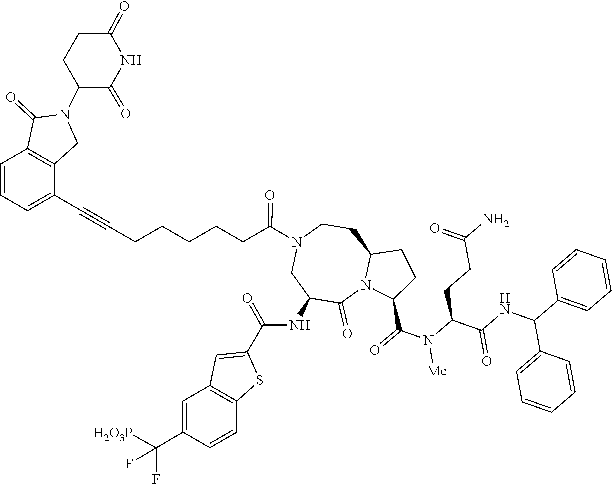 | ((2-(((5S,8S,10aR)-8-(((S)-5-amino-1- (benzhydrylamino)-1,5-dioxopentan-2- yl)(methyl)carbamoyl)-3-(8-(2-(2,6-dioxopiperidin-3-yl)- 1-oxoisoindolin-4-yl)oct-7-ynoyl)-6- oxodecahydropyrrolo[1,2-a][1,5]diazocin-5- yl)carbamoyl)benzo[b]thiophen-5- yl)difluoromethyl)phosphonic acid |
| 189 | 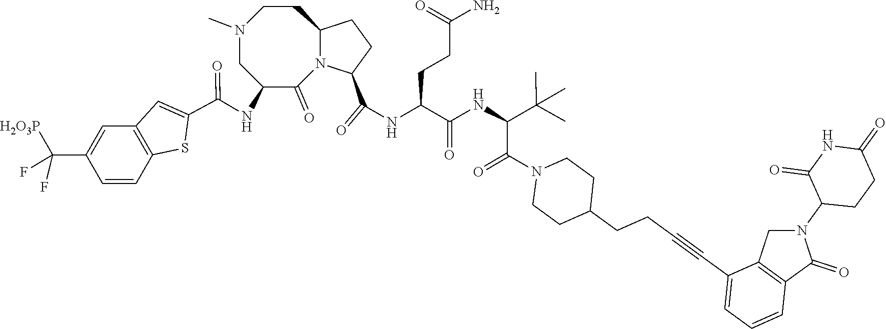 | ((2-(((5S,8S,10aR)-8-(((2S)-5-amino-1-(((2S)-1-(4-(4-(2- (2,6-dioxopiperidin-3-yl)-1-oxoisoindolin-4-yl)but-3-yn- 1-yl)piperidin-1-yl)-3,3-dimethyl-1-oxobutan-2- yl)amino)-1,5-dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 190 | 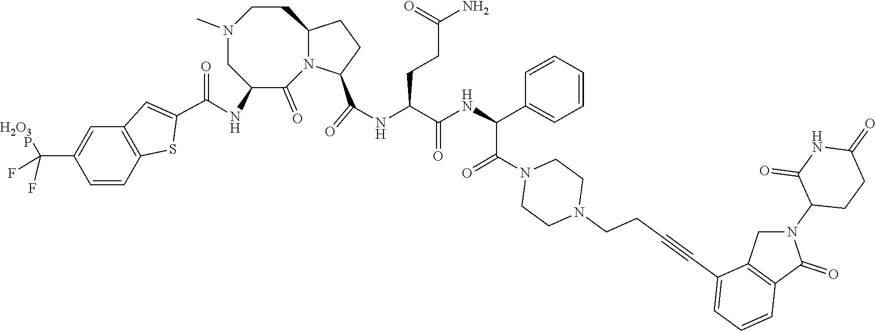 | ((2-(((5S,8S,10aR)-8-(((2S)-5-amino-1-(((1S)-2-(4-(4-(2- (2,6-dioxopiperidin-3-yl)-1-oxoisoindolin-4-yl)but-3-yn- 1-yl)piperazin-1-yl)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 191 | 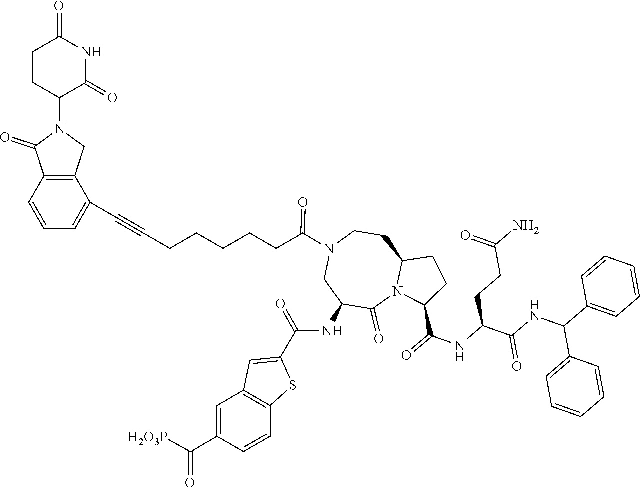 | (2-(((5S,8S,10aR)-8-(((S)-5-amino-1-(benzhydrylamino)- 1,5-dioxopentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)benzo[b]thiophene-5-carbonyl)phosphonic acid |
| 191E | 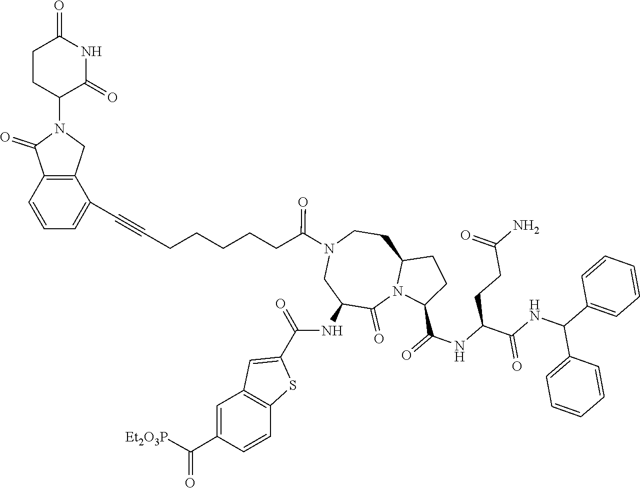 | (2S)-N1-benzhydryl-2-((5S,8S,10aR)-5-(5-(((diethyl-13- oxidaneyl)(11- oxidaneyl)phosphoryl)carbonyl)benzo[b]thiophene-2- carboxamido)-3-(8-(2-(2,6-dioxopiperidin-3-yl)-1- oxoisoindolin-4-yl)oct-7-ynoyl)-6- oxodecahydropyrrolo[1,2-a][1,5]diazocine-8- carboxamido)pentanediamide |
| 192 | 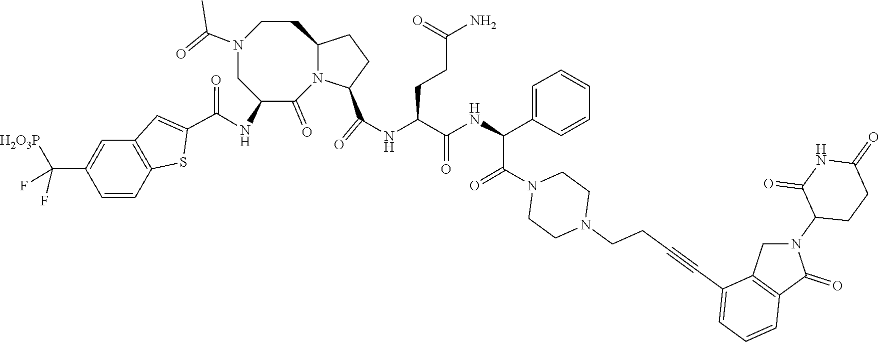 | ((2-(((5S,8S,10aR)-3-acetyl-8-(((2S)-5-amino-1-(((1S)-2- (4-(4-(2-(2,6-dioxopiperidin-3-yl)-1-oxoisoindolin-4- yl)but-3-yn-1-yl)piperidin-1-yl)-2-oxo-1- phenylethyl)amino)-1,5-dioxopentan-2-yl)carbamoyl)-6- oxodecahydropyrrolo[1,2-a][1,5]diazocin-5- yl)carbamoyl)benzo[b] thiophen-5- yl)difluoromethyl)phosphonic acid |
| 193 | 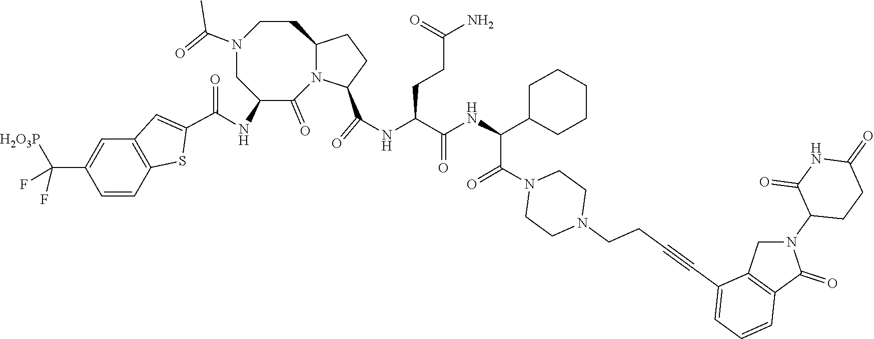 | ((2-(((5S,8S,10aR)-3-acetyl-8-(((2S)-5-amino-1-(((1S)-1- cyclohexyl-2-(4-(4-(2-(2,6-dioxopiperidin-3-yl)-1- oxoisoindolin-4-yl)but-3-yn-1-yl)piperidin-1-yl)-2- oxoethyl)amino)-1,5-dioxopentan-2-yl)carbamoyl)-6- oxodecahydropyrrolo[1,2-a][1,5]diazocin-5- yl)carbamoyl)benzo[b]thiophen-5- yl)difluoromethyl)phosphonic acid |
| 194 | 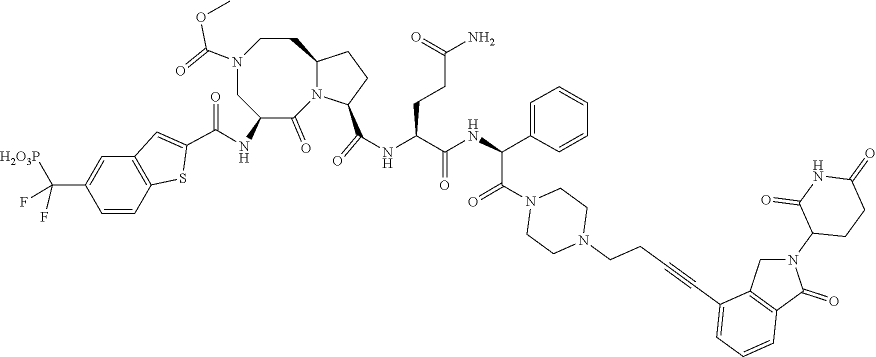 | ((2-(((5S,8S,10aR)-8-(((2S)-5-amino-1-(((1S)-2-(4-(4-(2- (2,6-dioxopiperidin-3-yl)-1-oxoisoindolin-4-yl)but-3-yn- 1-yl)piperidin-1-yl)-2-oxo-1-phenylethyl)amino)-1,5- dioxopentan-2-yl)carbamoyl)-3-(methoxycarbonyl)-6- oxodecahydropyrrolo[1,2-a][1,5]diazocin-5- yl)carbamoyl)benzo[b]thiophen-5- yl)difluoromethyl)phosphonic acid |
| 195 | 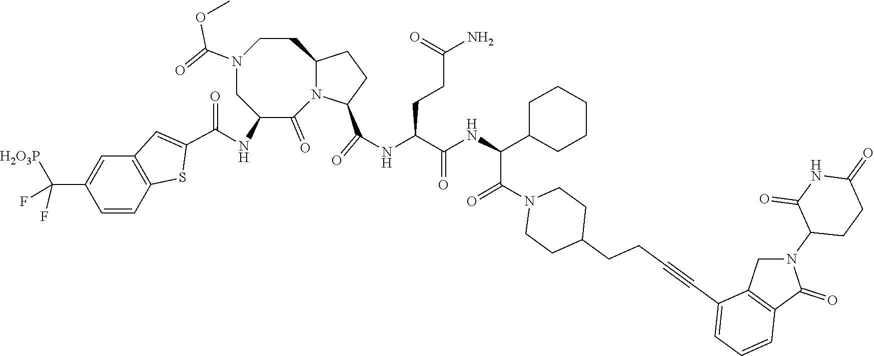 | ((2-(((5S,8S,10aR)-8-(((2S)-5-amino-1-(((1S)-1- cyclohexyl-2-(4-(4-(2-(2,6-dioxopiperidin-3-yl)-1- oxoisoindolin-4-yl)but-3-yn-1-yl)piperidin-1-yl)-2- oxoethyl)amino)-1,5-dioxopentan-2-yl)carbamoyl)-3- (methoxycarbonyl)-6-oxodecahydropyrrolo[1,2- a][1,5]diazocin-5-yl)carbamoyl)benzo[b]thiophen-5- yl)difluoromethyl)phosphonic acid |
| 196 | 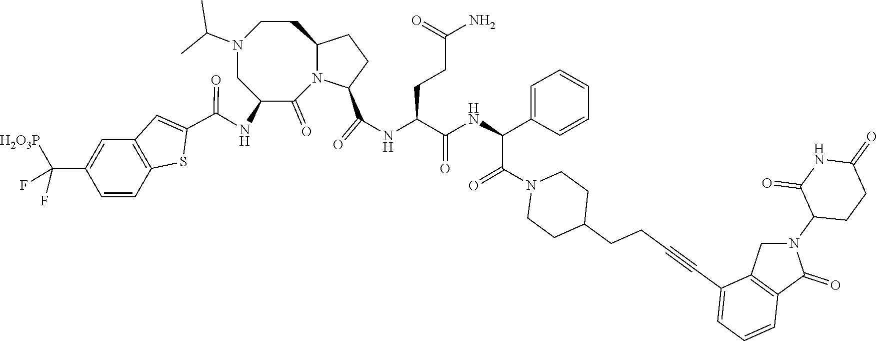 | ((2-(((5S,8S,10aR)-8-(((2S)-5-amino-1-(((1S)-2-(4-(4-(2- (2,6-dioxopiperidin-3-yl)-1-oxoisoindolin-4-yl)but-3-yn- 1-yl)piperidin-1-yl)-2-oxo-1-phenylethyl)amino)-1,5- dioxopentan-2-yl)carbamoyl)-3-isopropyl-6- oxodecahydropyrrolo[1,2-a][1,5]diazocin-5- yl)carbamoyl)benzo[b]thiophen-5- yl)difluoromethyl)phosphonic acid |
| 197 | 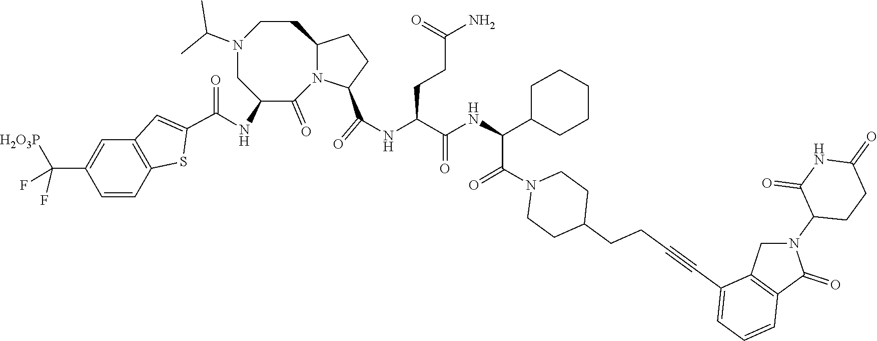 | ((2-(((5S,8S,10aR)-8-(((2S)-5-amino-1-(((1S)-1- cyclohexyl-2-(4-(4-(2-(2,6-dioxopiperidin-3-yl)-1- oxoisoindolin-4-yl)but-3-yn-1-yl)piperidin-1-yl)-2- oxoethyl)amino)-1,5-dioxopentan-2-yl)carbamoyl)-3- isopropyl-6-oxodecahydropyrrolo[1,2-a][1,5]diazocin-5- yl)carbamoyl)benzo[b]thiophen-5- yl)difluoromethyl)phosphonic acid |
| 198 | 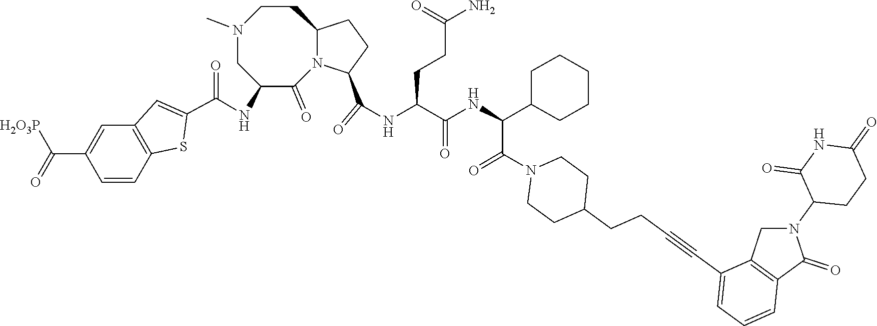 | (2-(((5S,8S,10aR)-8-(((2S)-5-amino-1-(((1S)-1- cyclohexyl-2-(4-(4-(2-(2,6-dioxopiperidin-3-yl)-1- oxoisoindolin-4-yl)but-3-yn-1-yl)piperidin-1-yl)-2- oxoethyl)amino)-1,5-dioxopentan-2-yl)carbamoyl)-3- methyl-6-oxodecahydropyrrolo[1,2-a][1,5]diazocin-5- yl)carbamoyl)benzo[b]thiophene-5-carbonyl)phosphonic acid |
| 199 | 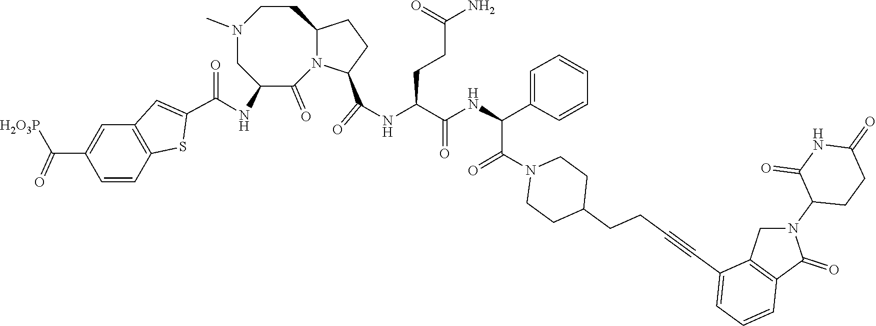 | (2-(((5S,8S,10aR)-8-(((2S)-5-amino-1-(((1S)-2-(4-(4-(2- (2,6-dioxopiperidin-3-yl)-1-oxoisoindolin-4-yl)but-3-yn- 1-yl)piperidin-1-yl)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)benzo[b]thiophene-5-carbonyl)phosphonic acid |
| 243 | 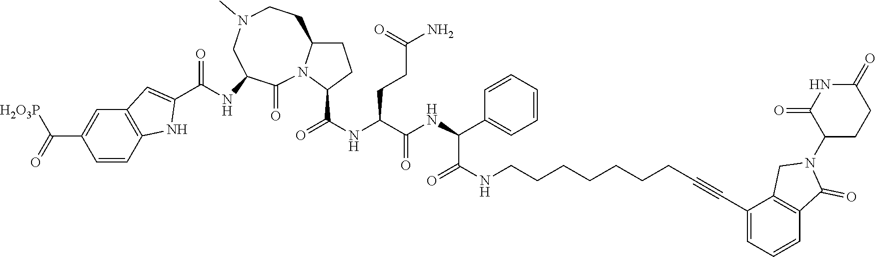 | (2-(((5S,8S,10aR)-8-(((2S)-5-amino-1-(((1S)-2-((9-(2- (2,6-dioxopiperidin-3-yl)-1-oxoisoindolin-4-yl)non-8-yn- 1-yl)amino)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)-1H-indole-5-carbonyl)phosphonic acid |
| 244 | 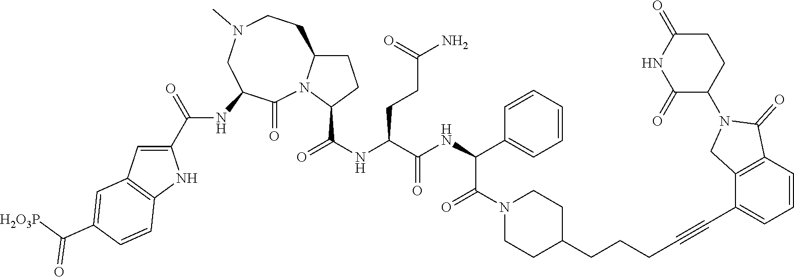 | (2-(((5S,8S,10aR)-8-(((2S)-5-amino-1-(((1S)-2-(4-(5-(2- (2,6-dioxopiperidin-3-yl)-1-oxoisoindolin-4-yl)pent-4- yn-1-yl)piperidin-1-yl)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)-1H-indole-5-carbonyl)phosphonic acid |
| 245 | 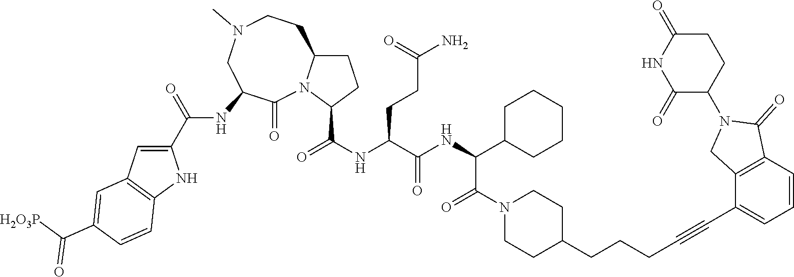 | (2-(((5S,8S,10aR)-8-(((2S)-5-amino-1-(((1S)-1- cyclohexyl-2-(4-(5-(2-(2,6-dioxopiperidin-3-yl)-1- oxoisoindolin-4-yl)pent-4-yn-1-yl)piperidin-1-yl)-2- oxoethyl)amino)-1,5-dioxopentan-2-yl)carbamoyl)-3- methyl-6-oxodecahydropyrrolo[1,2-a][1,5]diazocin-5- yl)carbamoyl)-1H-indole-5-carbonyl)phosphonic acid |
| 246 | 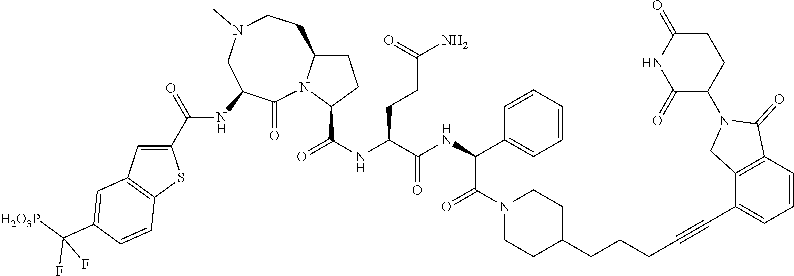 | ((2-(((5S,8S,10aR)-8-(((2S)-5-amino-1-(((1S)-2-(4-(5-(2- (2,6-dioxopiperidin-3-yl)-1-oxoisoindolin-4-yl)pent-4- yn-1-yl)piperidin-1-yl)-2-oxo-1-phenylethyl)amino)-1,5- dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 247 | 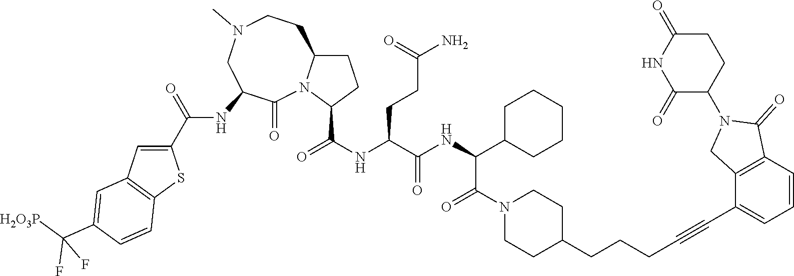 | ((2-(((5S,8S,10aR)-8-(((2S)-5-amino-1-(((1S)-1- cyclohexyl-2-(4-(5-(2-(2,6-dioxopiperidin-3-yl)-1- oxoisoindolin-4-yl)pent-4-yn-1-yl)piperidin-1-yl)-2- oxoethyl)amino)-1,5-dioxopentan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 248 | 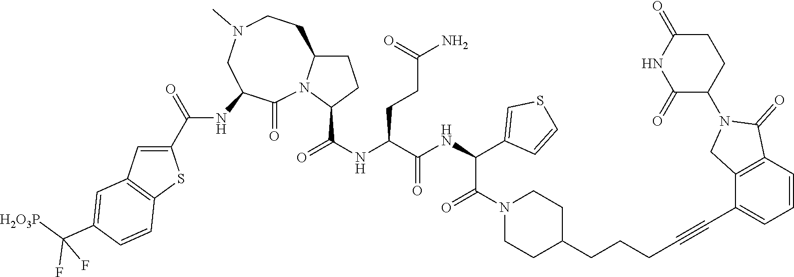 | ((2-(((5S,8S,10aR)-8-(((2S)-5-amino-1-(((1S)-2-(4-(5-(2- (2,6-dioxopiperidin-3-yl)-1-oxoisoindolin-4-yl)pent-4- yn-1-yl)piperidin-1-yl)-2-oxo-1-(thiophen-3- yl)ethyl)amino)-1,5-dioxopentan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 249 | 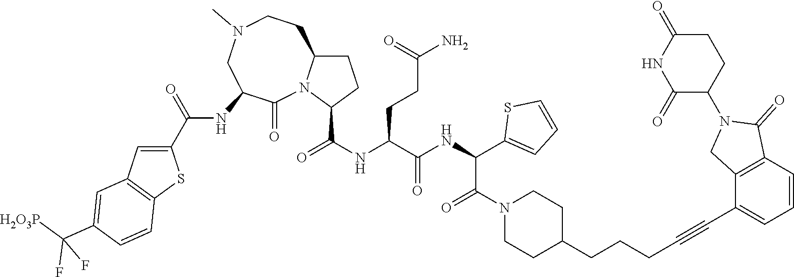 | ((2-(((5S,8S,10aR)-8-(((2S)-5-amino-1-(((1R)-2-(4-(5-(2- (2,6-dioxopiperidin-3-yl)-1-oxoisoindolin-4-yl)pent-4- yn-1-yl)piperidin-1-yl)-2-oxo-1-(thiophen-2- yl)ethyl)amino)-1,5-dioxopentan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 250 | 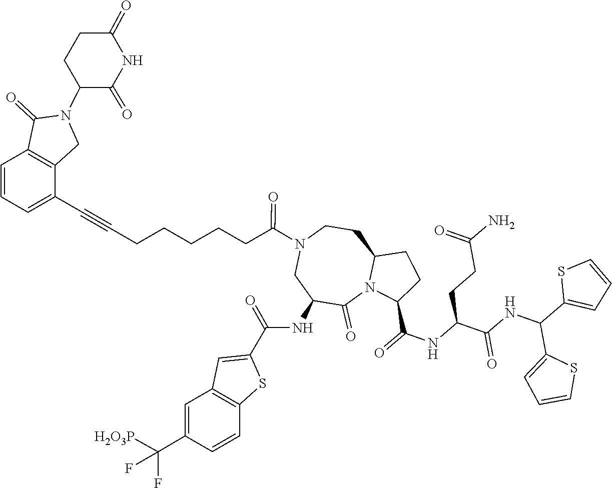 | ((2-(((5S,8S,10aR)-8-(((S)-5-amino-1-((di(thiophen-2- yl)methyl)amino)-1,5-dioxopentan-2-yl)carbamoyl)-3-(8- (2-(2,6-dioxopiperidin-3-yl)-1-oxoisoindolin-4-yl)oct-7- ynoyl)-6-oxodecahydropyrrolo[1,2-a][1,5]diazocin-5- yl)carbamoyl)benzo[b]thiophen-5- yl)difluoromethyl)phosphonic acid |
| 251 | 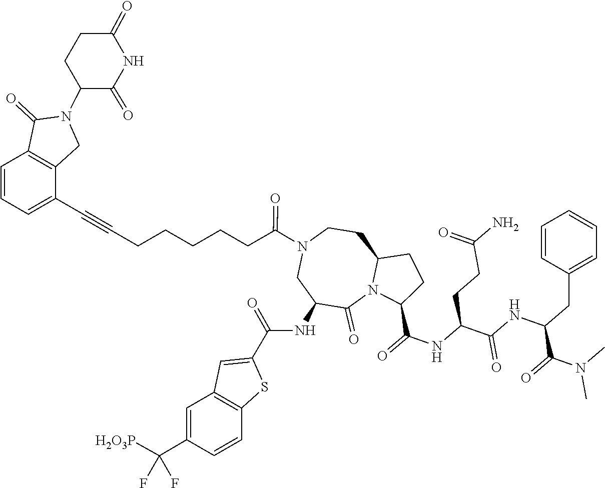 | ((2-(((5S,8S,10aR)-8-(((S)-5-amino-1-(((S)-1- (dimethylamino)-1-oxo-3-phenylpropan-2-yl)amino)-1,5- dioxopentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)benzo[b]thiophen-5- yl)difluoromethyl)phosphonic acid |
| 252 | 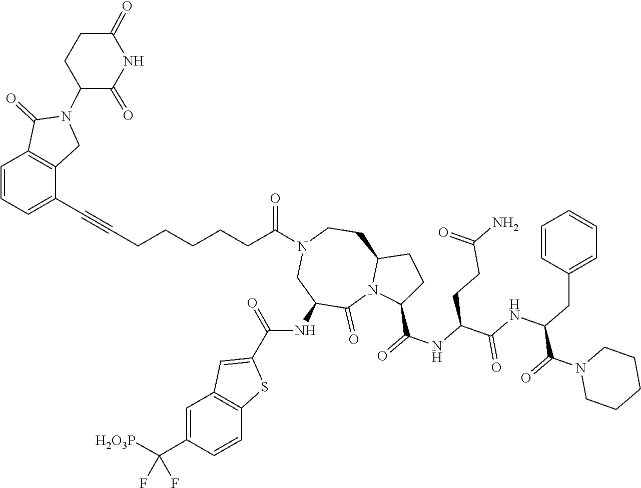 | ((2-(((5S,8S,10aR)-8-(((S)-5-amino-1,5-dioxo-1-(((S)-1- oxo-3-phenyl-iperidinedin-1-yl)propan-2- yl)amino)pentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)benzo[b]thiophen-5- yl)difluoromethyl)phosphonic acid |
| 253 | 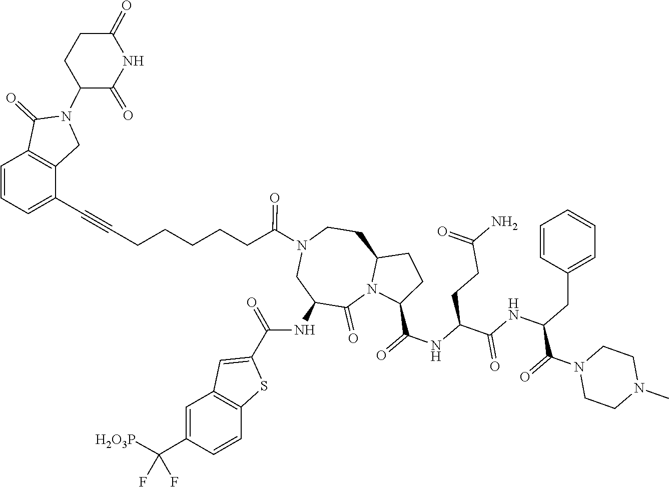 | ((2-(((5S,8S,10aR)-8-(((S)-5-amino-1-(((S)-1-(4- methylpiperazin-1-yl)-1-oxo-3-phenylpropan-2- yl)amino)-1,5-dioxopentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)benzo[b]thiophen-5- yl)difluoromethyl)phosphonic acid |
| 254 | 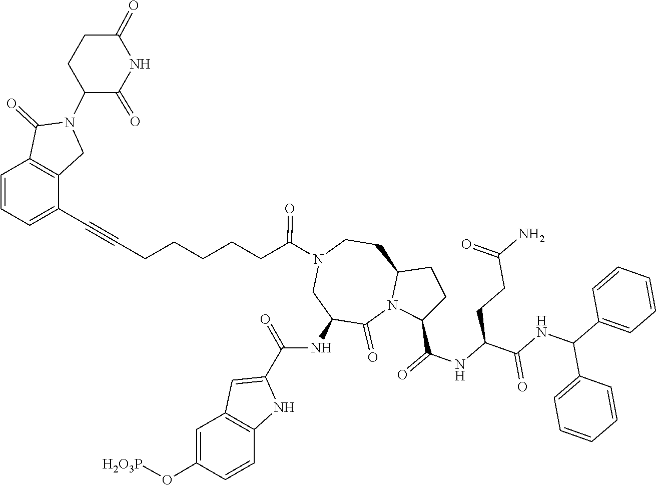 | 2-(((5S,8S,10aR)-8-(((S)-5-amino-1-(benzhydrylamino)- 1,5-dioxopentan-2-yl)carbamoyl)-3-(8-(2-(2,6- dioxopiperidin-3-yl)-1-oxoisoindolin-4-yl)oct-7-ynoyl)- 6-oxodecahydropyrrolo[1,2-a][1,5]diazocin-5- yl)carbamoyl)-1H-indol-5-yl dihydrogen phosphate |
| 255 | 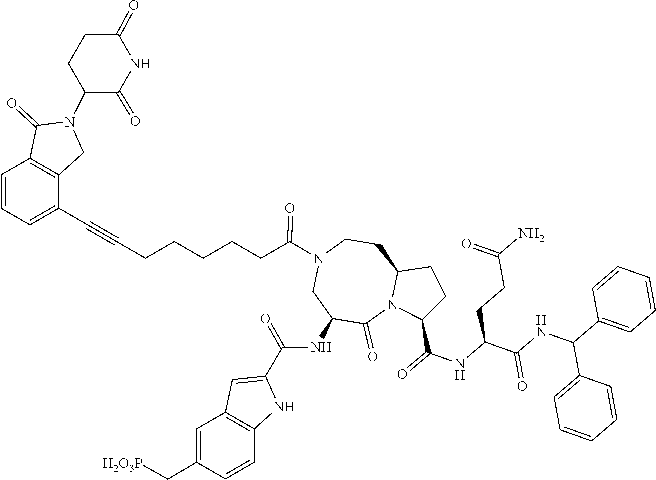 | ((2-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)carbamoyl)-1H-indol-5-yl)methyl)phosphonic acid |
| 256 | 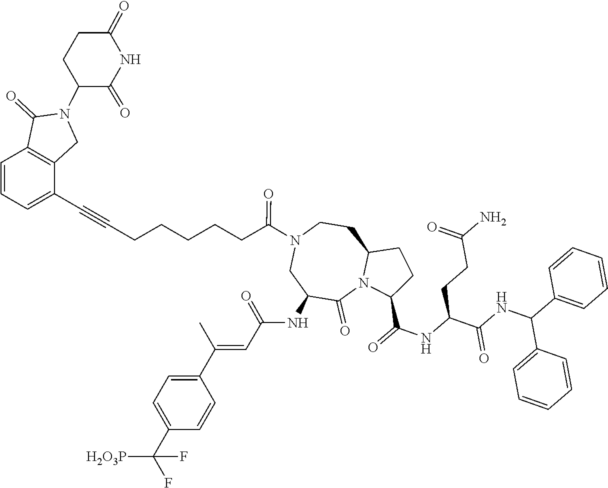 | ((4-((E)-4-(((5S,8S,10aR)-8-(((S)-5-amino-1- (benzhydrylamino)-1,5-dioxopentan-2-yl)carbamoyl)-3- (8-(2-(2,6-dioxopiperidin-3-yl)-1-oxoisoindolin-4-yl)oct- 7-ynoyl)-6-oxodecahydropyrrolo[1,2-a][1,5]diazocin-5- yl)amino)-4-oxobut-2-en-2- yl)phenyl)difluoromethyl)phosphonic acid |
| 257 | 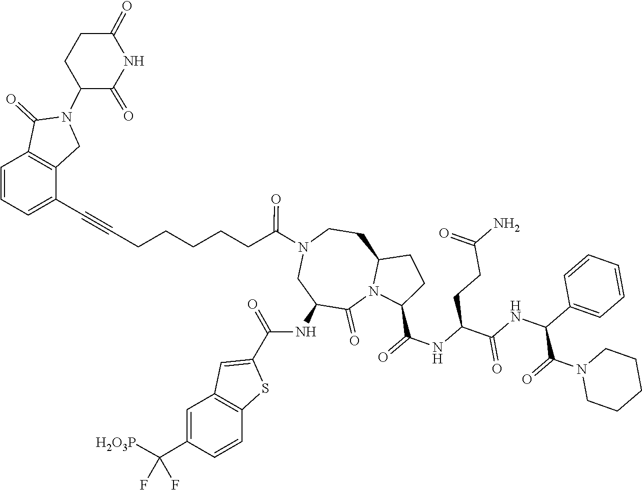 | ((2-(((5S,8S,10aR)-8-(((S)-5-amino-1,5-dioxo-1-(((S)-2- oxo-1-phenyl-iperidinedin-1-yl)ethyl)amino)pentan-2- yl)carbamoyl)-3-(8-(2-(2,6-dioxopiperidin-3-yl)-1- oxoisoindolin-4-yl)oct-7-ynoyl)-6- oxodecahydropyrrolo[1,2-a][1,5]diazocin-5- yl)carbamoyl)benzo[b]thiophen-5- yl)difluoromethyl)phosphonic acid |
| 258 | 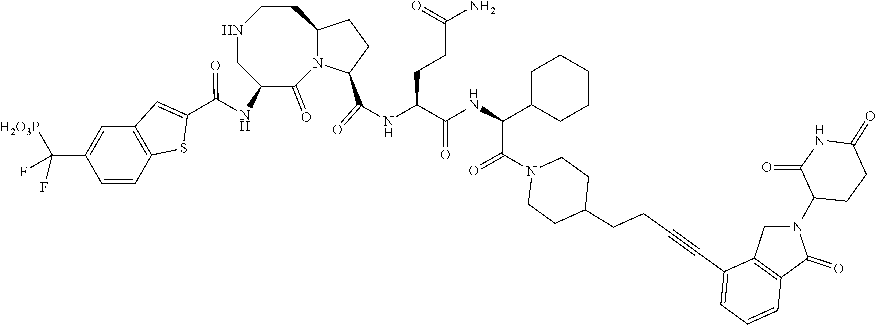 | ((2-(((5S,8S,10aR)-8-(((2S)-5-amino-1-(((1S)-1- cyclohexyl-2-(4-(4-(2-(2,6-dioxopiperidin-3-yl)-1- oxoisoindolin-4-yl)but-3-yn-1-yl)piperidin-1-yl)-2- oxoethyl)amino)-1,5-dioxopentan-2-yl)carbamoyl)-6- oxodecahydropyrrolo[1,2-a][1,5]diazocin-5- yl)carbamoyl)benzo[b]thiophen-5- yl)difluoromethyl)phosphonic acid |
| 259 | 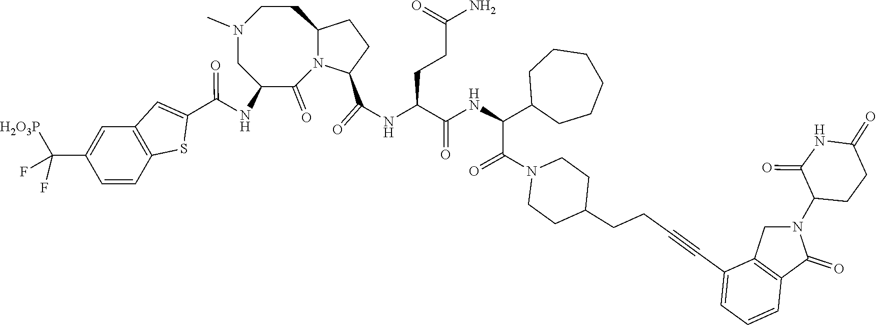 | ((2-(((5S,8S,10aR)-8-(((2S)-5-amino-1-(((1S)-1- cycloheptyl-2-(4-(4-(2-(2,6-dioxopiperidin-3-yl)-1- oxoisoindolin-4-yl)but-3-yn-1-yl)piperidin-1-yl)-2- oxoethyl)amino)-1,5-dioxopentan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 260 | 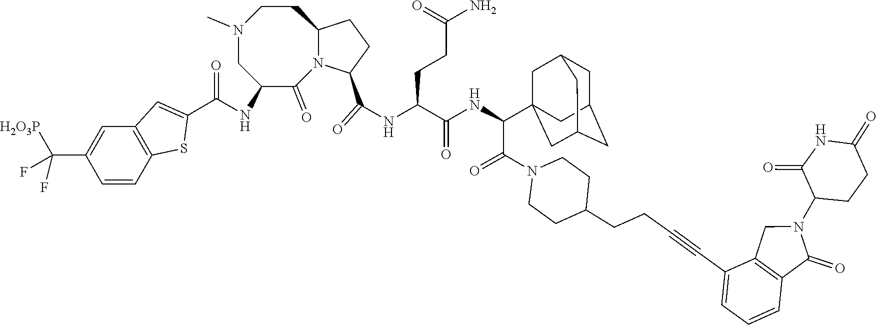 | ((2-(((5S,8S,10aR)-8-(((2S)-1-(((1S)-1-((1s,3R)- adamantan-1-yl)-2-(4-(4-(2-(2,6-dioxopiperidin-3-yl)-1- oxoisoindolin-4-yl)but-3-yn-1-yl)piperidin-1-yl)-2- oxoethyl)amino)-5-amino-1,5-dioxopentan-2- yl)carbamoyl)-3-methyl-6-oxodecahydropyrrolo[1,2- a][1,5]diazocin-5-yl)carbamoyl)benzo[b]thiophen-5- yl)difluoromelhyl)phosphonic acid |
| 261 | 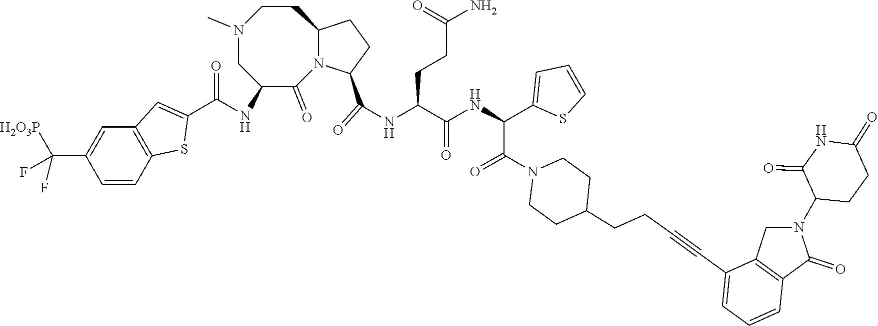 | ((2-(((5S,8S,10aR)-8-(((2S)-5-amino-1-(((1R)-2-(4-(4-(2- (2,6-dioxopiperidin-3-yl)-1-oxoisoindolin-4-yl)but-3-yn- 1-yl)piperidin-1-yl)-2-oxo-1-(thiophen-2- yl)ethyl)amino)-1,5-dioxopentan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 262 | 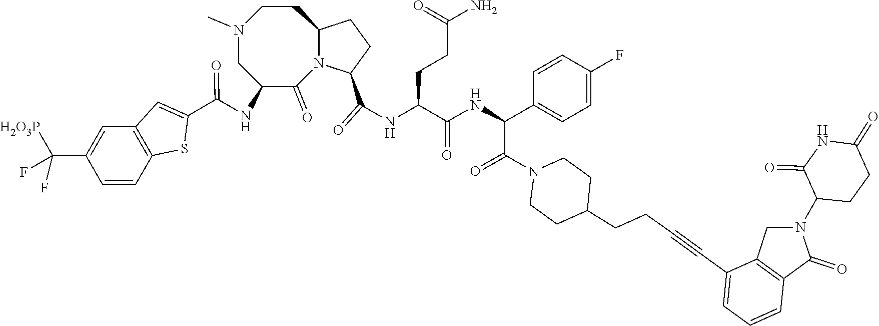 | ((2-(((5S,8S,10aR)-8-(((2S)-5-amino-1-(((1S)-2-(4-(4-(2- (2,6-dioxopiperidin-3-yl)-1-oxoisoindolin-4-yl)but-3-yn- 1-yl)piperidin-1-yl)-1-(4-fluorophenyl)-2- oxoethyl)amino)-1,5-dioxopentan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 263 | 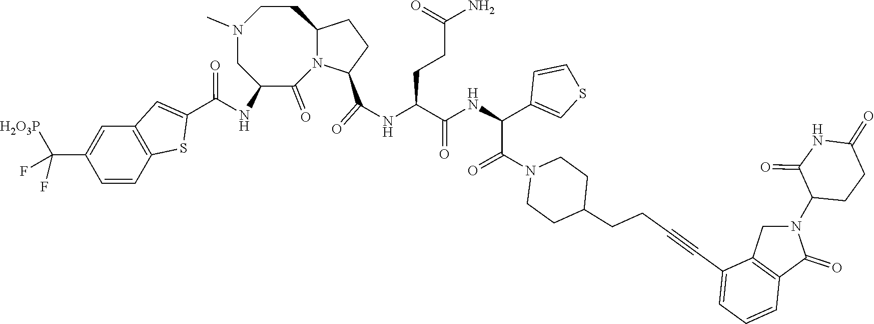 | ((2-(((5S,8S,10aR)-8-(((2S)-5-amino-1-(((1S)-2-(4-(4-(2- (2,6-dioxopiperidin-3-yl)-1-oxoisoindolin-4-yl)but-3-yn- 1-yl)piperidin-1-yl)-2-oxo-1-(thiophen-3- yl)ethyl)amino)-1,5-dioxopentan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 264 | 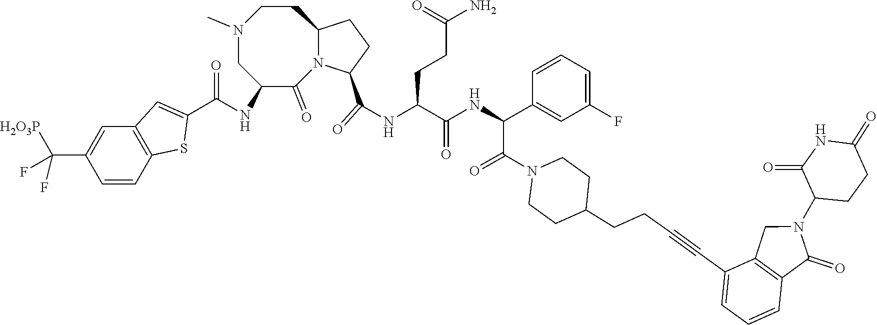 | ((2-(((5S,8S,10aR)-8-(((2S)-5-amino-1-(((1S)-2-(4-(4-(2- (2,6-dioxopiperidin-3-yl)-1-oxoisoindolin-4-yl)but-3-yn- 1-yl)piperidin-1-yl)-1-(3-fluorophenyl)-2- oxoethyl)amino)-1,5-dioxopentan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 265 | 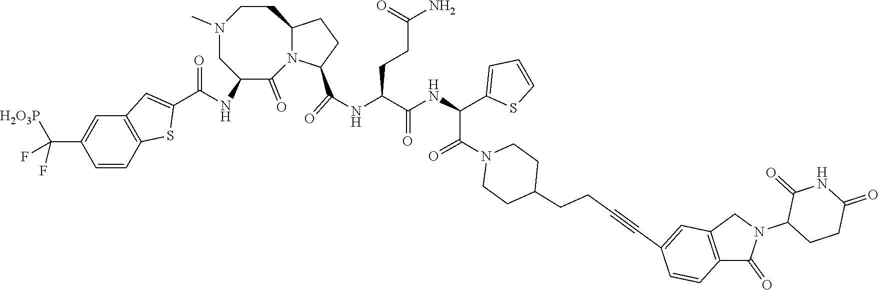 | ((2-(((5S,8S,10aR)-8-(((2S)-5-amino-1-(((1R)-2-(4-(4-(2- (2,6-dioxopiperidin-3-yl)-1-oxoisoindolin-5-yl)but-3-yn- 1-yl)piperidin-1-yl)-2-oxo-1-(thiophen-2- yl)ethyl)amino)-1,5-dioxopentan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 266 | 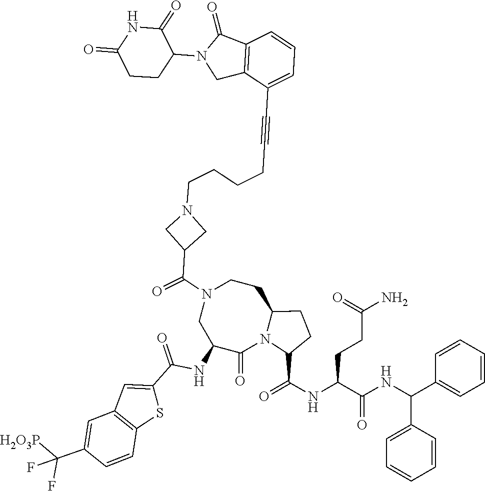 | ((2-(((5S,8S,10aR)-8-(((S)-5-amino-1- (benzhydrylamino)1,5-dioxopentan-2-yl)carbamoyl)-3- (1-(6-(2-(2,6-dioxopiperidin-3-yl)-1-oxoisoindolin-4- yl)hex-5-yn-1-yl)azetidine-3-carbonyl)-6- oxodecahydropyrrolo[1,2-a][1,5]diazocin-5- yl)carbamoyl)benzo[b]thiophen-5- yl)difluoromethyl)phosphonic acid |
| 267 | 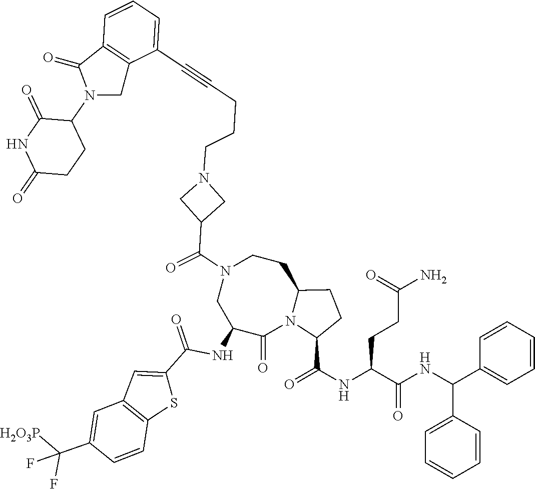 | ((2-(((5S,8S,10aR)-8-(((S)-5-amino-1- (benzhydrylamino)-1,5-dioxopentan-2-yl)carbamoyl)-3- (1-(5-(2-(2,6-dioxopiperidin-3-yl)-1-oxoisoindolin-4- yl)pent-4-yn-1-yl)azetidine-3-carbonyl)-6- oxodecahydropyrrolo[1,2-a][1,5]diazocin-5- yl)carbamoyl)benzo[b]thiophen-5- yl)difluoromethyl)phosphonic acid |
| 268 | 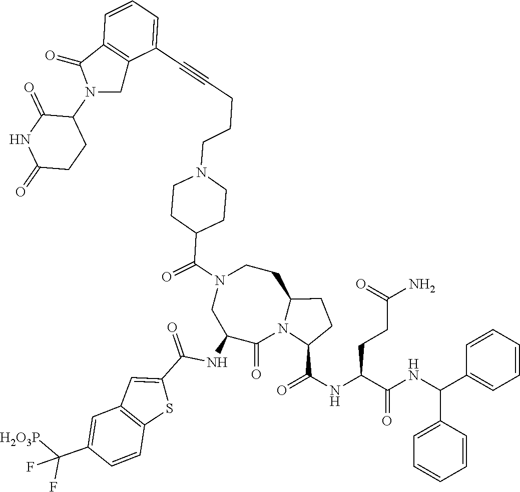 | ((2-(((5S,8S,10aR)-8-(((S)-5-amino-1- (benzhydrylamino)-1,5-dioxopentan-2-yl)carbamoyl)-3- (1-(5-(2-(2,6-dioxopiperidin-3-yl)-1-oxoisoindolin-4- yl)pent-4-yn-1-yl)piperidine-4-carbonyl)-6- oxodecahydropyrrolo[1,2-a][1,5]diazocin-5- yl)carbamoyl)benzo[b]thiophen-5- yl)difluoromethyl)phosphonic acid |
| 269 | 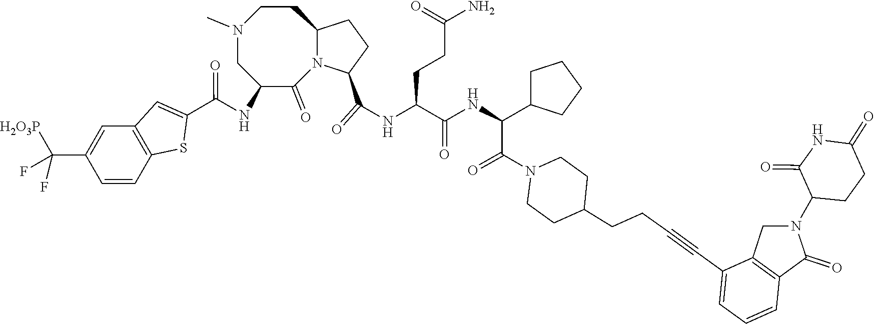 | ((2-(((5S,8S,10aR)-8-(((2S)-5-amino-1-(((1S)-1- cyclopentyl-2-(4-(4-(2-(2,6-dioxopiperidin-3-yl)-1- oxoisoindolin-4-yl)but-3-yn-1-yl)piperidin-1-yl)-2- oxoethyl)amino)-1,5-dioxopentan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 270 | 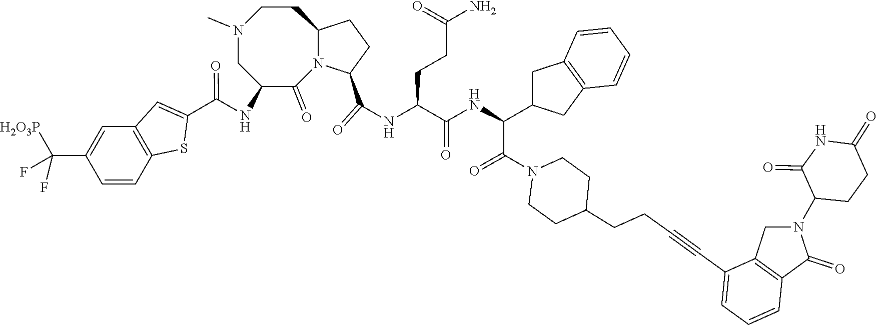 | ((2-(((5S,8S,10aR)-8-(((2S)-5-amino-1-(((1S)-1-(2,3- dihydro-1H-inden-2-yl)-2-(4-(4-(2-(2,6-dioxopiperidin-3- yl)-1-oxoisoindolin-4-yl)but-3-yn-1-yl)piperidin-1-yl)-2- oxoethyl)amino)-1,5-dioxopentan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 271 | 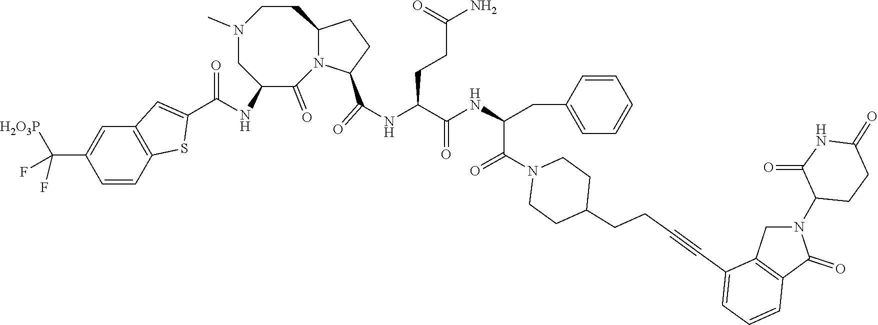 | ((2-(((5S,8S,10aR)-8-(((2S)-5-amino-1-(((2S)-1-(4-(4-(2- (2,6-dioxopiperidin-3-yl)-1-oxoisoindolin-4-yl)but-3-yn- 1-yl)piperidin-1-yl)-1-oxo-3-phenylpropan-2-yl)amino)- 1,5-dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 272 | 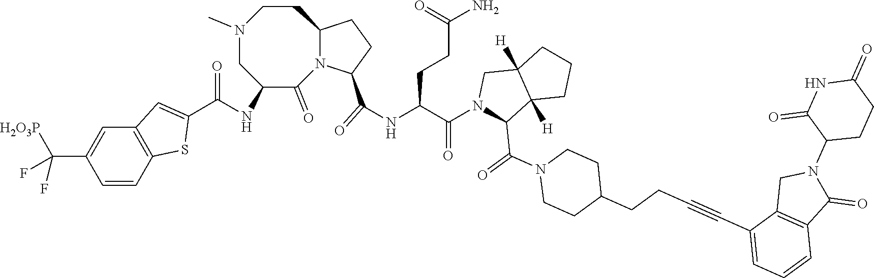 | ((2-(((5S,8S,10aR)-8-(((2S)-5-amino-1-((1S,3aR,6aS)-1- (4-(4-(2-(2,6-dioxopiperidin-3-yl)-1-oxoisoindolin-4- yl)but-3-yn-1-yl)piperidine-1- carbonyl)hexahydrocyclopenta[c]pyrrol-2(1H)-yl)-1,5- dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| F273 | 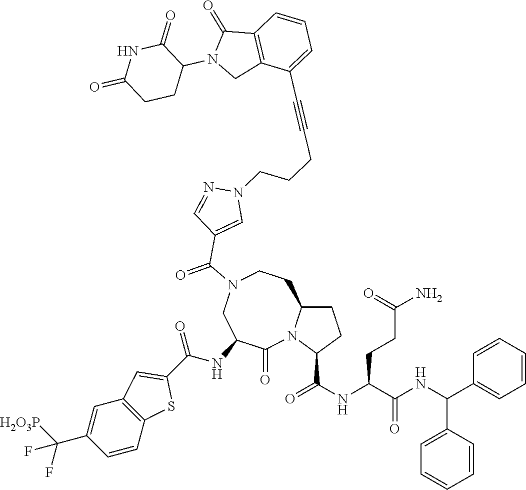 | ((2-(((5S,8S,10aR)-8-(((S)-5-amino-1- (benzhydrylamino)-1,5-dioxopentan-2-yl)carbamoyl)-3- (1-(5-(2-(2,6-dioxopiperidin-3-yl)-1-oxoisoindolin-4- yl)pent-4-yn-1-yl)-1H-pyrazole-4-carbonyl)-6- oxodecahydropyrrolo[1,2-a][1,5]diazocin-5- yl)carbamoyl)benzo[b]thiophen-5- yl)difluoromethyl)phosphonic acid |
| 274 | 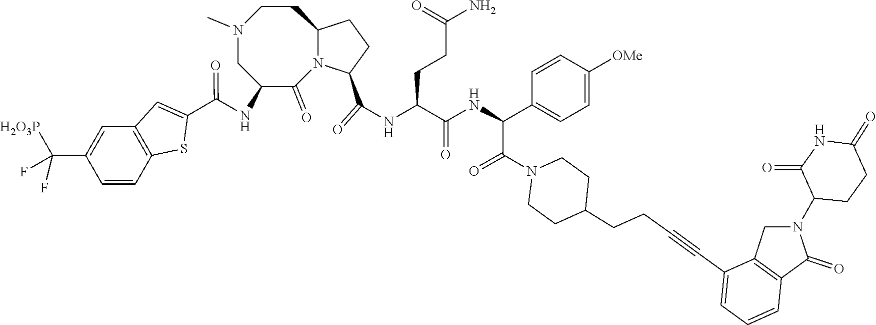 | ((2-(((5S,8S,10aR)-8-(((2S)-5-amino-1-(((1S)-2-(4-(4-(2- (2,6-dioxopiperidin-3-yl)-1-oxoisoindolin-4-yl)but-3-yn- 1-yl)piperidin-1-yl)-1-(4-methoxyphenyl)-2- oxoethyl)amino)-1,5-dioxopentan-2-yl)carbamoyl)-3- methyl-6-oxodecahydropyrrolo[1,2-a][1,5]diazocin-5- yl)carbamoyl)benzo[b]thiophen-5- yl)difluoromethyl)phosphonic acid |
| 275 | 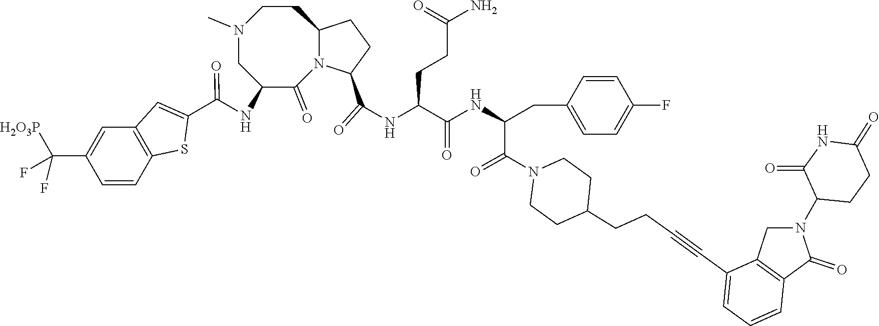 | ((2-(((5S,8S,10aR)-8-(((2S)-5-amino-1-(((2S)-1-(4-(4-(2- (2,6-dioxopiperidin-3-yl)-1-oxoisoindolin-4-yl)but-3-yn- 1-yl)piperidin-1-yl)-3-(4-fluorophenyl)-1-oxopropan-2- yl)amino)-1,5-dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 276 | 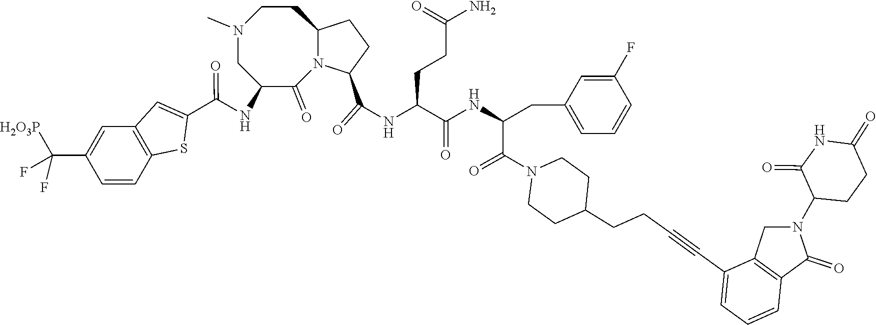 | ((2-(((5S,8S,10aR)-8-(((2S)-5-amino-1-(((2S)-1-(4-(4-(2- (2,6-dioxopiperidin-3-yl)-1-oxoisoindolin-4-yl)but-3-yn- 1-yl)piperidin-1-yl)-3-(3-fluorophenyl)-1-oxopropan-2- yl)amino)-1,5-dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 277 | 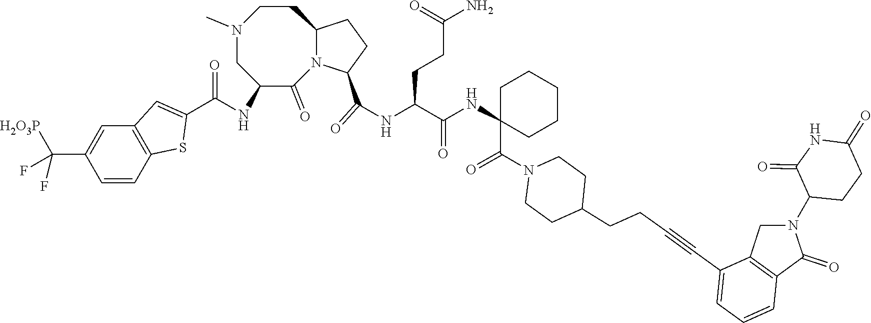 | ((2-(((5S,8S,10aR)-8-(((2S)-5-amino-1-((1-(4-(4-(2-(2,6- dioxopiperidin-3-yl)-1-oxoisoindolin-4-yl)but-3-yn-1- yl)piperidine-1-carbonyl)cyclohexyl)amino)-1,5- dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 278 | 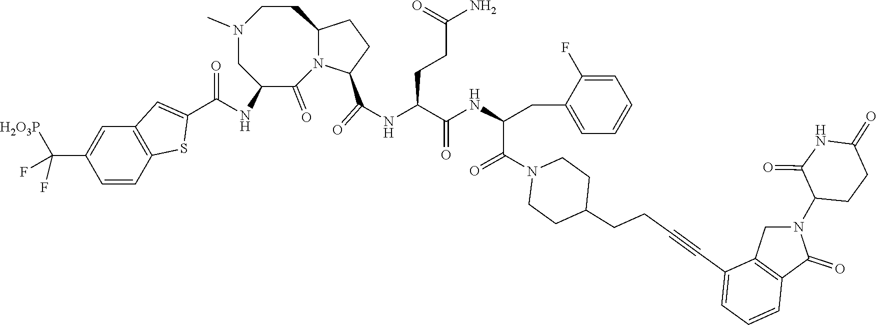 | ((2-(((5S,8S,10aR)-8-(((2S)-5-amino-1-(((2S)-1-(4-(4-(2- (2,6-dioxopiperidin-3-yl)-1-oxoisoindolin-4-yl)but-3-yn- 1-yl)piperidin-1-yl)-3-(2-fluorophenyl)-1-oxopropan-2- yl)amino)-1,5-dioxopentan-2-yl)carbamoyl)-3-methyl-6- oxodecahydropyrrolo[1,2-a][1,5]diazocin-5- yl)carbamoyl)benzo[b]thiophen-5- yl)difluoromethyl)phosphonic acid |
| 279 | 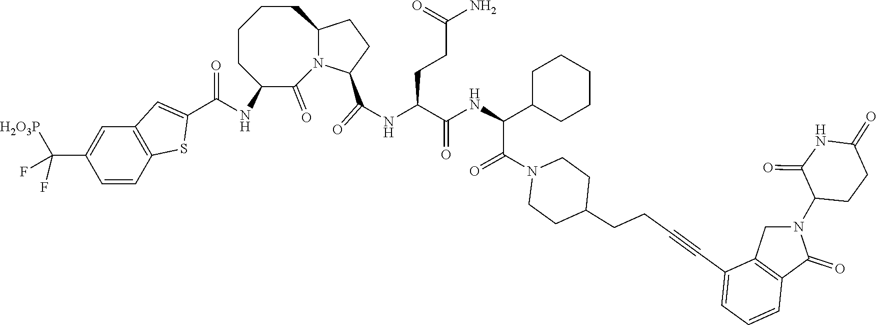 | ((2-(((3S,6S,10aS)-3-(((2S)-5-amino-1-(((1S)-1- cyclohexyl-2-(4-(4-(2-(2,6-dioxopiperidin-3-yl)-1- oxoisoindolin-4-yl)but-3-yn-1-yl)piperidin-1-yl)-2- oxoethyl)amino)-1,5-dioxopentan-2-yl)carbamoyl)-5- oxodecahydropyrrolo[1,2-a]azocin-6- yl)carbamoyl)benzo[b]thiophen-5- yl)difluoromethyl)phosphonic acid |
| 280 | 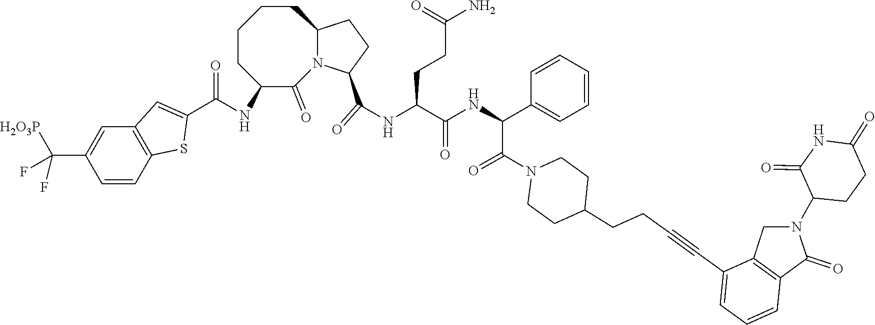 | ((2-(((3S,6S,10aS)-3-(((2S)-5-amino-1-(((1S)-2-(4-(4-(2- (2,6-dioxopiperidin-3-yl)-1-oxoisoindolin-4-yl)but-3-yn- 1-yl)piperidin-1-yl)-2-oxo-1-phenylethyl)amino)-1,5- dioxopentan-2-yl)carbamoyl)-5- oxodecahydropyrrolo[1,2-a]azocin-6- yl)carbamoyl)benzo[b]thiophen-5- yl)difluoromethyl)phosphonic acid |
| 281 | 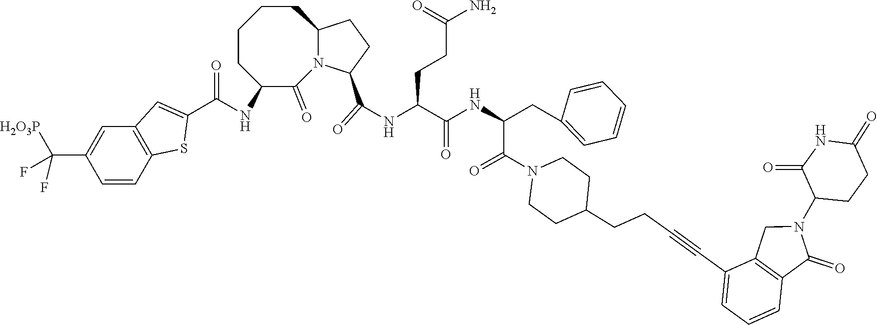 | ((2-(((3S,6S,10aS)-3-(((2S)-5-amino-1-(((2S)-1-(4-(4-(2- (2,6-dioxopiperidin-3-yl)-1-oxoisoindolin-4-yl)but-3-yn- 1-yl)piperidin-1-yl)-1-oxo-3-phenylpropan-2-yl)amino)- 1,5-dioxopentan-2-yl)carbamoyl)-5- oxodecahydropyrrolo[1,2-a]azocin-6- yl)carbamoyl)benzo[b]thiophen-5- yl)difluoromethyl)phosphonic acid |
| 282 | 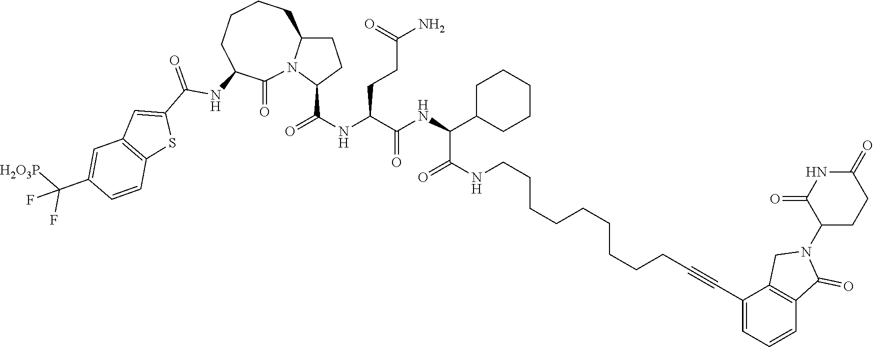 | ((2-(((3S,6S,10aS)-3-(((2S)-5-amino-1-(((1S)-1- cyclohexyl-2-((11-(2-(2,6-dioxopiperidin-3-yl)-1- oxoisoindolin-4-yl)undec-10-yn-1-yl)amino)-2- oxoethyl)amino)-1,5-dioxopentan-2-yl)carbamoyl)-5- oxodecahydropyrrolo[1,2-a] azocin-6- yl)carbamoyl)benzo[b]thiophen-5- yl)difluoromethyl)phosphonic acid |
| 283 | 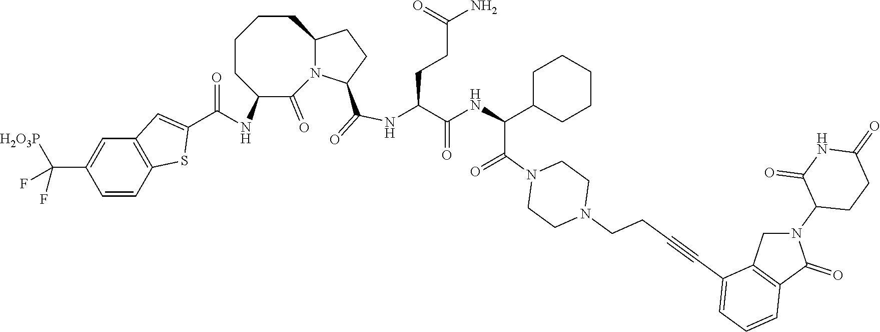 | ((2-(((3S,6S,10aS)-3-(((2S)-5-amino-1-(((1S)-1- cyclohexyl-2-(4-(4-(2-(2,6-dioxopiperidin-3-yl)-1- oxoisoindolin-4-yl)but-3-yn-1-yl)piperazin-1-yl)-2- oxoethyl)amino)-1,5-dioxopentan-2-yl)carbamoyl)-5- oxodecahydropyrrolo[1,2-a]azocin-6- yl)carbamoyl)benzo[b]thiophen-5- yl)difluoromethyl)phosphonic acid |

TABLE 1A

| Cpd. | | |
|---|---|---|
| No. | Structure | Name |
| 284 | 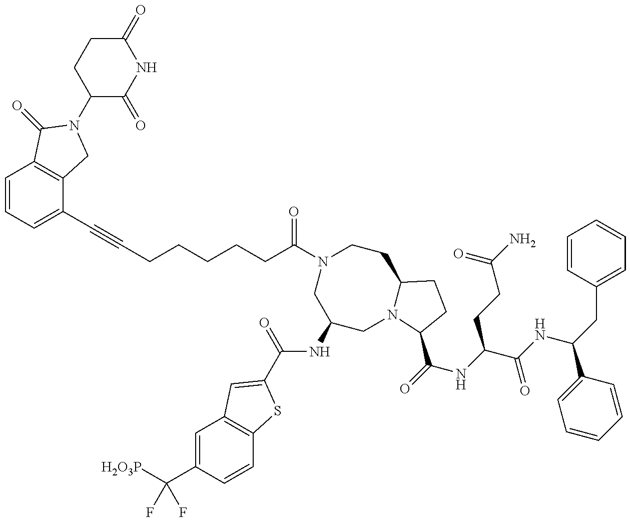 | ((2-(((5S,8S,10aR)-8-(((S)-5- amino-1-(((S)-1,2- diphenylethyl)amino)-1,5-dioxopentan- 2-yl)carbamoyl)-3- (8-(2-(2,6-dioxopiperidin-3-yl)-1- oxoisoindolin-4-yl)oct-7- ynoyl)-6-oxodecahydropyrrolo[1,2- a][1,5]diazocin-5- yl)carbamoyl)benzo[b]thiophen-5- yl)difluoromethyl)phosphonic acid |
| 285 | 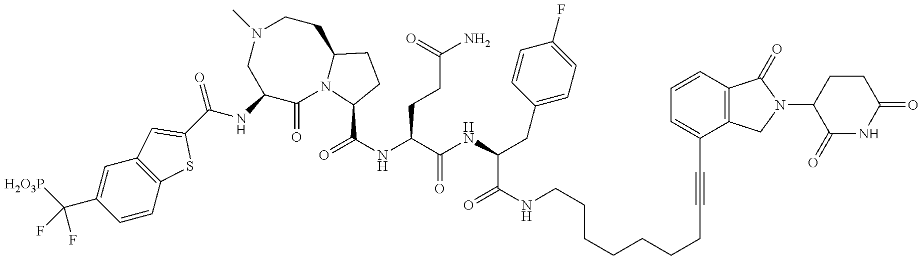 | ((2-(((5S,8S,10aR)-8-(((2S)-5-amino- 1-(((2S)-1-((9-(2-(2,6- dioxopiperidin-3-yl)-1-oxoisoindolin- 4-yl)non-8-yn-1- yl)amino)-3-(4-fluorophenyl)-1- oxopropan-2-yl)amino)- 1,5-dioxopentan-2-yl)carbamoyl)- 3-methyl-6- oxodecahydropyrrolo[1,2-a][1,5]diazocin- 5-yl)carbamoyl)benzo[b]thiophen-5- yl)difluoromethyl)phosphonic acid |
| 286 | 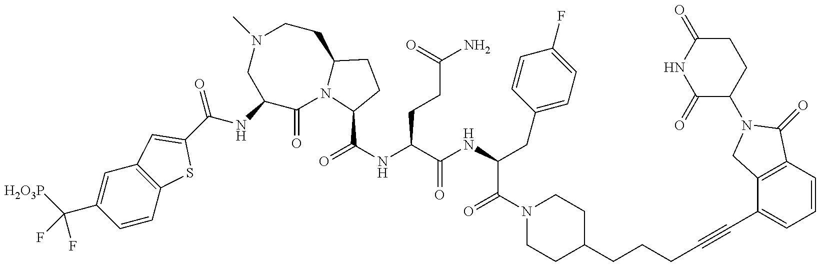 | ((2-(((5S,8S,10aR)-8-(((2S)-5-amino-1- (((2S)-1-(4-(5-(2- (2,6-dioxopiperidin-3-yl)-1-oxoisoindolin- 4-yl)pent-4-yn-1- yl)piperidin-1-yl)-3-(4-fluorophenyl)-1- oxopropan-2-yl)amino)- 1,5-dioxopentan-2-yl)carbamoyl)- 3-methyl-6- oxodecahydropyrrolo[1,2- a][1,5]diazocin-5- yl)carbamoyl)benzo[b]thiophen-5- yl)difluoromethyl)phosphonic acid |
| 287 | 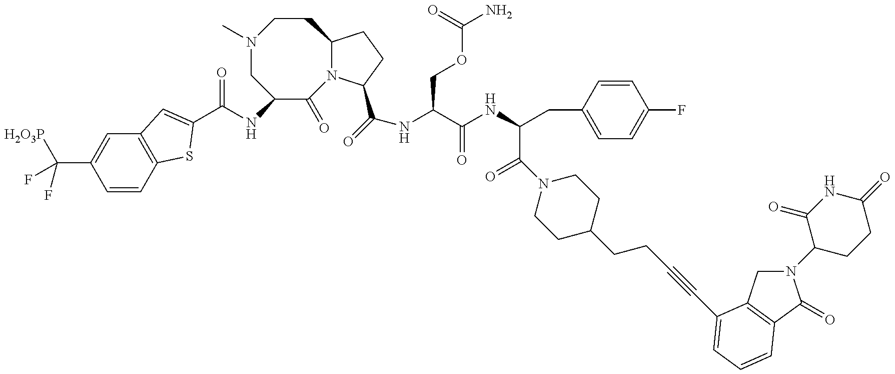 | ((2-(((5S,8S,10aR)-8-(((2S)-3- (carbamoyloxy)-1-(((2S)-1- (4-(4-(2-(2,6-dioxopiperidin-3-yl)- 1-oxoisoindolin-4- yl)but-3-yn-1-yl)piperidin-1-yl)-3- (4-fluorophenyl)-1- oxopropan-2-yl)amino)-1-oxopropan- 2-yl)carbamoyl)-3- methyl-6-oxodecahydropyrrolo[1,2- a][1,5]diazocin-5- yl)carbamoyl)benzo[b]thiophen-5- yl)difluoromethyl)phosphonic acid |
| 288 | 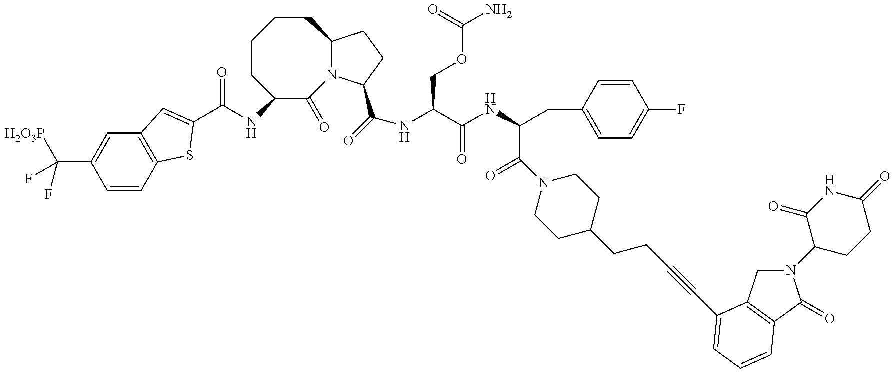 | ((2-(((3S,6S,10aS)-3-(((2S)-5-amino- 1-(((2S)-1-(4-(4-(2- (2,6-dioxopiperidin-3-yl)-1- oxoisoindolin-4-yl)but-3-yn-1- yl)piperidin-1-yl)-3-(4-fluorophenyl)- 1-oxopropan-2-yl)amino)- 1,5-dioxopentan2-yl)carbamoyl)-5- oxodecahydropyrrolo[1,2-a]azocin-6- yl)carbamoyl)benzo[b]thiophen-5- yl)difluoromethyl)phosphonic acid |
| 289 | 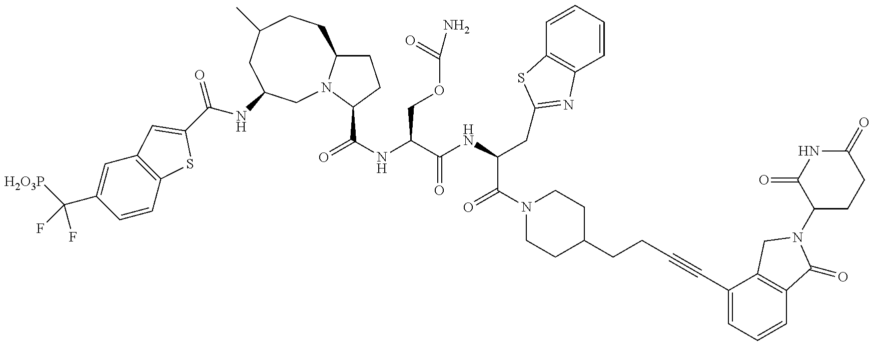 | ((2-(((5S,8S,10aR)-8-(((2S)-1-(((2S)- 3-(benzo[d]thiazol-2- yl)-1-(4-(4-(2-(2,6-dioxopiperidin-3- yl)-1-oxoisoindolin-4- yl)but-3-yn-1-yl)piperidin-1-yl)-1- oxopropan-2-yl)amino)- 3-(carbamoyloxy)-1-oxopropan- 2-yl)carbamoyl)-3-methyl-6- oxodecahydropyrrolo[1,2-a][1,5]diazocin- 5-yl)carbamoyl)benzo[b]thiophen-5- yl)difluoromethyl)phosphonic acid |
| 290 | 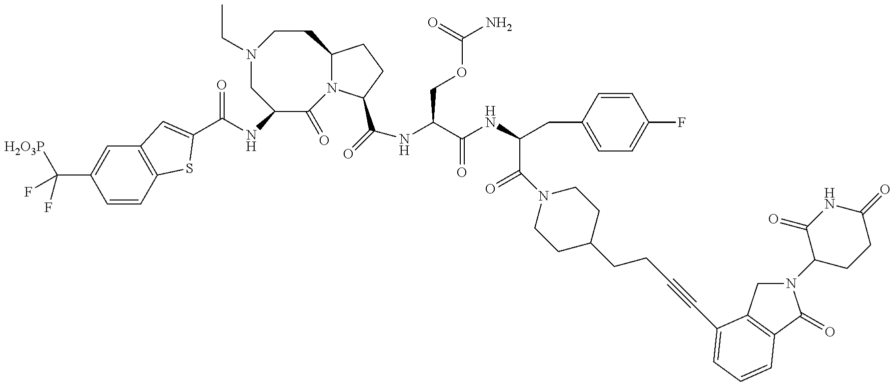 | ((2-(((5S,8S,10aR)-8-(((2S)-5-amino- 1-(((2S)-1-(4-(4-(2- (2,6-dioxopiperidin-3-yl)-1-oxoisoindolin- 4-yl)but-3-yn-1- yl)piperidin-1-yl)-3-(4-fluorophenyl)- 1-oxopropan-2- yl)amino)-1,5-dioxopentan-2- yl)carbamoyl)-3-ethyl-6- oxodecahydropyrrolo[1,2-a][1,5]diazocin- 5-yl)carbamoyl)benzo[b]thiophen-5- yl)difluoromethyl)phosphonic acid |
| 291 | 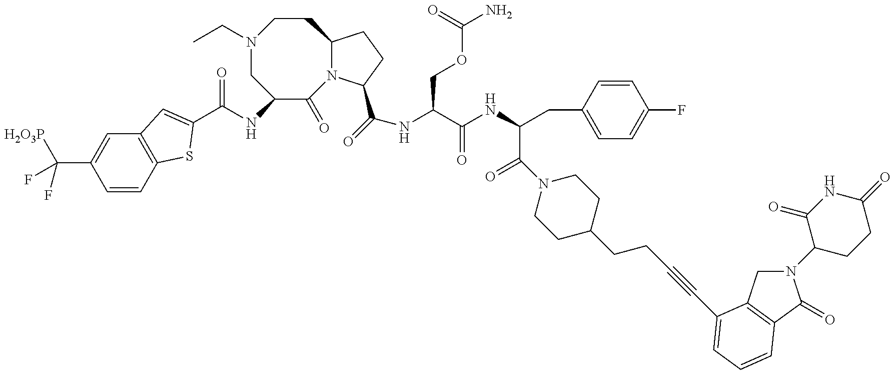 | ((2-(((5S,8S,10aR)-8-(((2S)-3- (carbamoyloxy)-1-(((2S)-1- (4-(4-(2-(2,6-dioxopiperidin-3-yl)- 1-oxoisoindolin-4- yl)but-3-yn-1-yl)piperidin-1-yl)- 3-(4-fluorophenyl)-1- oxopropan-2-yl)amino)-1-oxopropan- 2-yl)carbamoyl)-3- ethyl-6-oxodecahydropyrrolo[1,2 -a][1,5]diazocin-5- yl)carbamoyl)benzo[b]thiophen-5- yl)difluoromethyl)phosphonic acid |
| 293 | 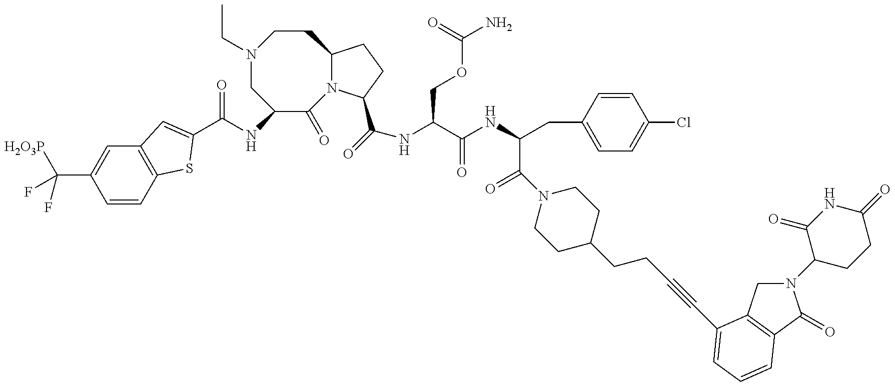 | ((2-(((5S,8S,10aR)-8-(((2S)-5- amino-1-(((2S)-3-(4- chlorophenyl)-1-(4-(4-(2-(2,6- dioxopiperidin-3-yl)-1- oxoisoindolin-4-yl)but-3-yn-1- yl)piperidin-1-yl)-1-oxopropan- 2-yl)amino)-1,5-dioxopentan- 2-yl)carbamoyl)- 3-ethyl-6-oxodecahydropyrrolo[1,2- a][1,5]diazocin-5- yl)carbamoyl)benzo[b]thiophen-5- yl)difluoromethyl)phosphonic acid |
| 294 | 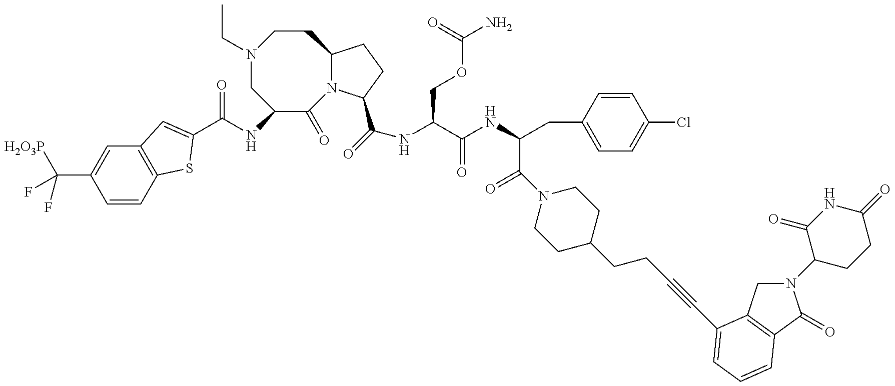 | ((2-(((5S,8S,10aR)-8-(((2S)-3- (carbamoyloxy)-1-(((2S)-3- (4-chlorophenyl)-1-(4-(4-(2- (2,6-dioxopiperidin-3-yl)-1- oxoisoindolin-4-yl)but-3-yn-1- yl)piperidin-1-yl)-1- oxopropan-2-yl)amino)-1- oxopropan-2-yl)carbamoyl)-3- ethyl-6-oxodecahydro- pyrrolo[1,2-a][1,5]diazocin-5- yl)carbamoyl)benzo[b]thiophen-5- yl)difluoromethyl)phosphonic acid |
| 295 | 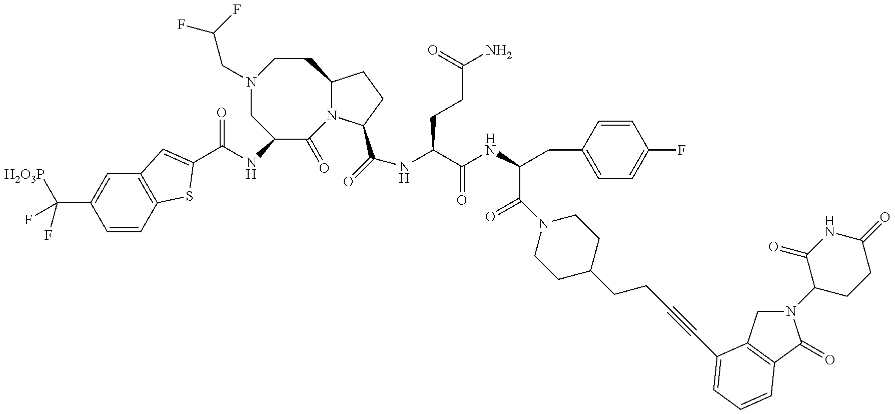 | ((2-(((5S,8S,10aR)-8-(((2S)- 5-amino-1-(((2S)-1-(4-(4-(2- (2,6-dioxopiperidin-3-yl)-1- oxoisoindolin-4-yl)but-3-yn-1- yl)piperidin-1-yl)-3-(4-fluorophenyl)- 1-oxopropan-2- yl)amino)-1,5-dioxopentan-2- yl)carbamoyl)-3-(2,2-difluoroethyl)- 6-oxodecahydropyrrolo[1,2- a][1,5]diazocin- 5-yl)carbamoyl)benzo[b]thiophen-5- yl)difluoromethyl)phosphonic acid |
| 296 | 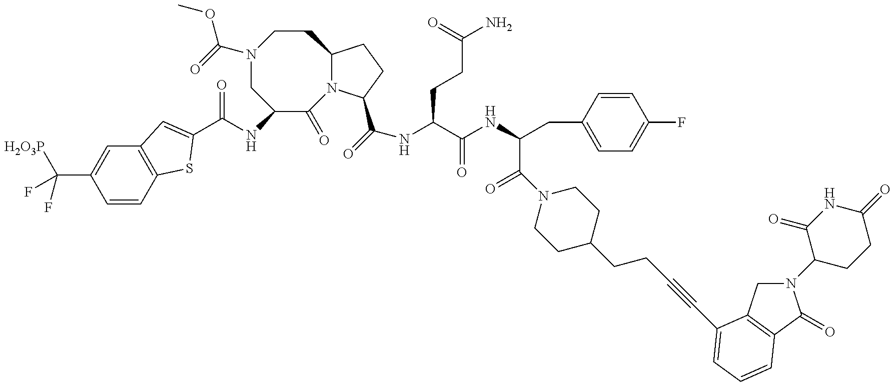 | ((2-(((5S,8S,10aR)-8-(((2S)-5- amino-1-(((2S)-1-(4-(4-(2- (2,6-dioxopiperidin-3-yl)-1- oxoisoindolin-4-yl)but-3-yn-1- yl)piperidin-1-yl)-3-(4-fluorophenyl)- 1-oxopropan-2-yl)amino)- 1,5-dioxopentan-2-yl)carbamoyl)-3- (methoxycarbonyl)-6-oxo- decahydropyrrolo[1,2- a][1,5]diazocin-5- yl)carbamoyl)benzo[b]thiophen-5- yl)difluoromethyl)phosphonic acid |
| 297 | 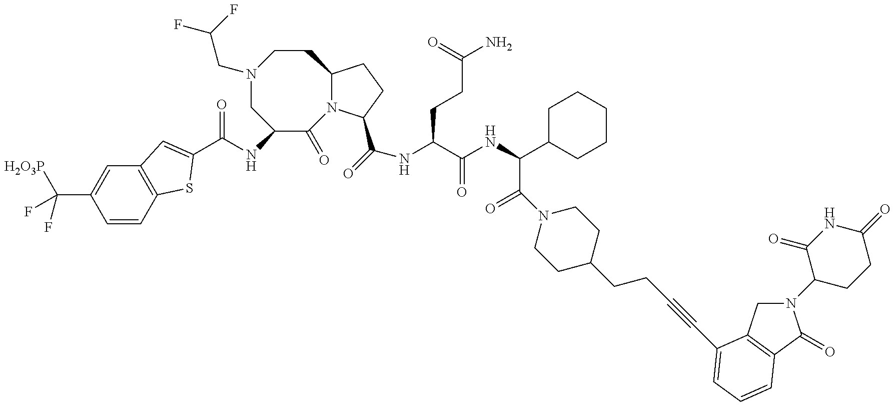 | ((2-(((5S,8S,10aR)-8- (((2S)-5-amino-1-(((1S)-1- cyclohexyl-2-(4-(4-(2-(2,6- dioxopiperidin-3-yl)-1- oxoisoindolin-4-yl)but-3-yn- 1-yl)piperidin-1-yl)-2- oxoethyl)amino)-1,5-dioxopentan- 2-yl)carbamoyl)-3-(2,2- difluoroethyl)-6-oxodecahydro- pyrrolo[1,2-a][1,5]diazocin- 5-yl)carbamoyl)benzo[b]thiophen-5- yl)difluoromethyl)phosphonic acid |
| 298 | 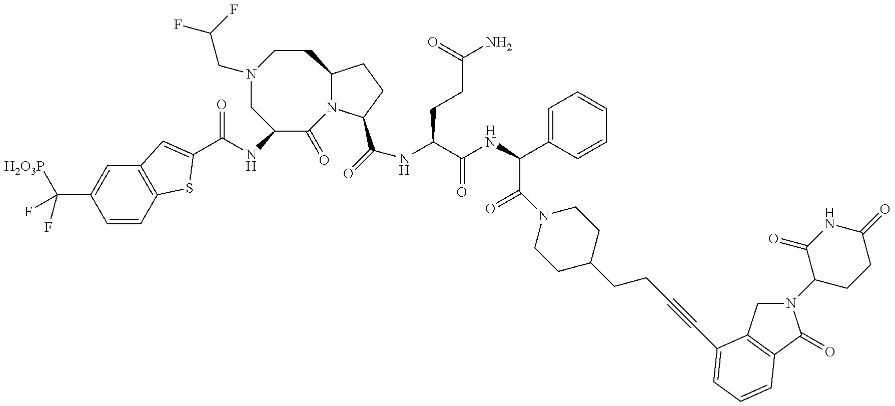 | ((2-(((5S,8S,10aR)-8-(((2S)- 5-amino-1-(((1S)-2-(4-(4-(2- (2,6-dioxopiperidin-3-yl)-1- oxoisoindolin-4-yl)but-3-yn-1- yl)piperidin-1-yl)-2-oxo-1- phenylethyl)amino)-1,5- dioxopentan-2-yl)carbamoyl)- 3-(2,2-difluoroethyl)-6-oxo- decahydropyrrolo[1,2-a][1,5]diazocin- 5-yl)carbamoyl)benzo[b]thiophen-5- yl)difluoromethyl)phosphonic acid |
| 299 | 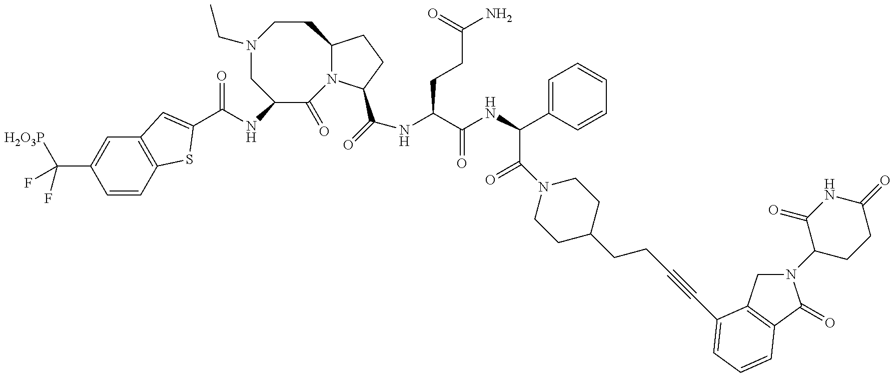 | ((2-(((5S,8S,10aR)-8-(((2S)-5-amino- 1-(((1S)-2-(4-(4-(2- (2,6-dioxopiperidin-3-yl)-1- oxoisoindolin-4-yl)but-3-yn-1- yl)piperidin-1-yl)-2-oxo-1-phenyl- ethyl)amino)-1,5-dioxopentan- 2-yl)carbamoyl)-3-ethyl-6-oxo- decahydropyrrolo[1,2-a][1,5]diazocin-5- yl)carbamoyl)benzo[b]thiophen-5- yl)difluroomethyl)phosphonic acid |
| 300 | 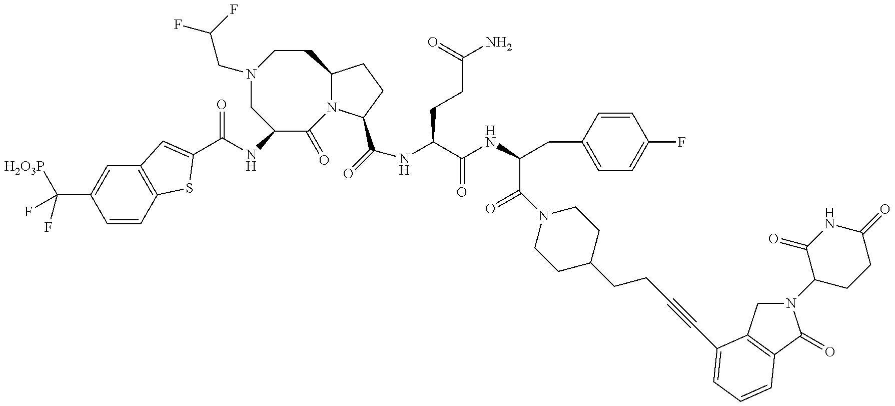 | ((2-(((5S,8S,10aR)-8-(((2S)-3- (carbamoyloxy)-1-(((2S)-1- (4-(4-(2-(2,6-dioxopiperidin-3-yl)- 1-oxoisoindolin-4- yl)but-3-yn-1-yl)piperidin-1-yl)- 3-(4-fluorophenyl)-1- oxopropan-2-yl)amino)-1- oxopropan-2-yl)carbamoyl)-3- (2,2-difluoroethyl)-6-oxo- decahydropyrrolo[1,2- a][1,5]diazocin-5-yl)carba- moyl)benzo[b]thiophen-5- y)difluoromethyl)phosphonic acid |
| 301 | 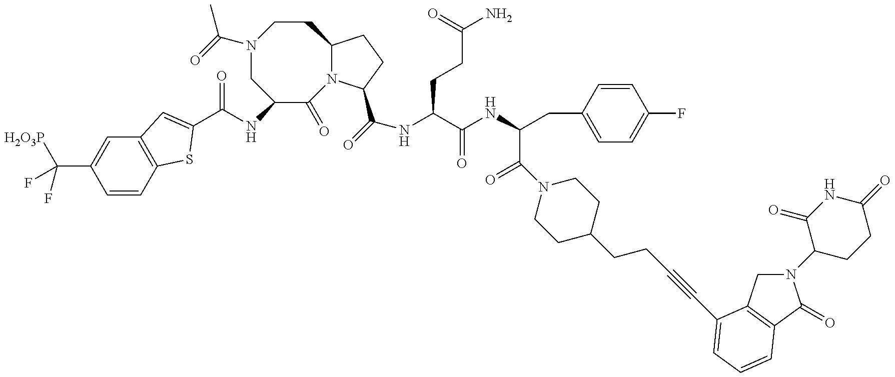 | ((2-(((5S,8S,10aR)-3-acetyl- 8-(((2S)-5-amino-1-(((2S)-1- (4-(4-(2-(2,6-dioxopiperidin- 3-yl)-1-oxoisoindolin-4- yl)but-3-yn-1-yl)piperidin-1-yl)- 3-(4-fluorophenyl)-1- oxopropan-2-yl)amino)-1,5- dioxopentan-2-yl)carbamoyl)- 6-oxodecahydorpyrrolo[1,2- a][1,5]diazocin-5- yl)carbamoyl)benzo[b]thiophen-5- yl)difluoromethyl)phosphonic acid |
| 302 | 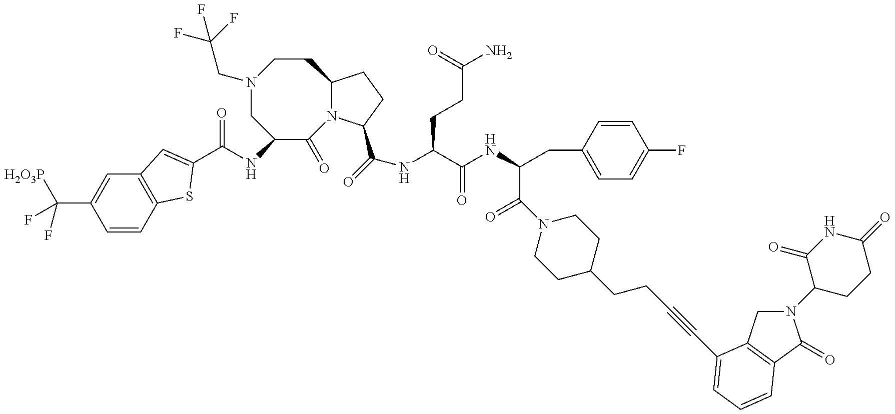 | ((2-(((5S,8S,10aR)-8-(((2S)-5-amino- 1-(((2S)-1-(4-(4-(2- (2,6-dioxopiperidin-3-yl)-1- oxoisoindolin-4-yl)but-3-yn-1- yl)piperidin-1-yl)-3-(4-fluorophenyl)- 1-oxopropan-2- yl)amino)-1,5-dioxopentan-2- yl)carbamoyl)-6-oxo-3- (2,2,2,-trifluoromethyl)deca- hydropyrrolo[1,2-a][1,5]diazocin- 5-yl)carbamoyl)benzo[b]thiophne-5- yl)difluoromethyl)phosphonic acid |
| 303 | 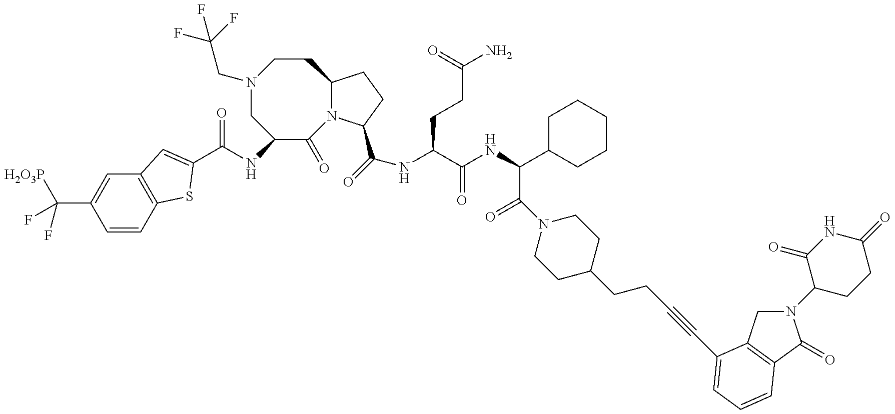 | ((2-(((5S,8S,10aR)-8-(((2S)- 5-amino-1-(((1S)-1- cyclohexyl-2-(4-(4-(2-(2,6-dioxo- piperidin-3-yl)-1-oxoisoindolin-4- yl)but-3-yn-1-yl)piperidin-1-yl)-2- oxoethyl)amino)-1,5-dioxopentan-2- yl)carbamoyl)-6-oxo-3-(2,2,2- trifluoroethyl)decahydropyrrolo[1,2- a][1,5]diazocin-5- yl)carbamoyl)benzo[b]thiophen-5- yl)difluoromethyl)phosphonic acid |
| 304 | 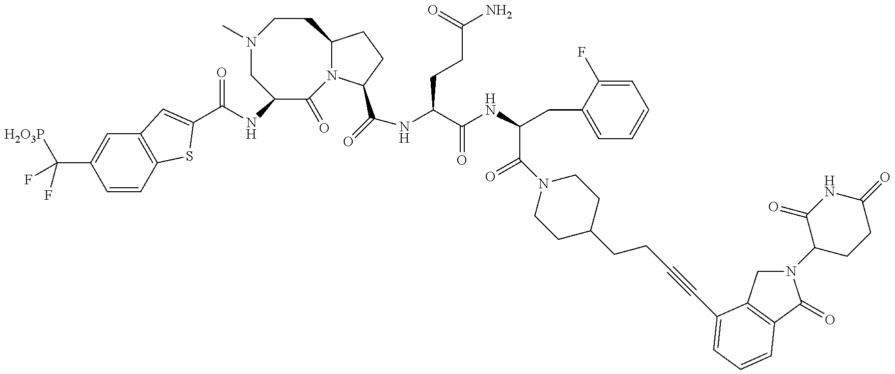 | ((2-(((5S,8S,10aR)-8-(((2S)- 5-amino-1-(((2S)-1-(4-(4-(2- (2,6-dioxopiperidin-3-yl)-1- oxoisoindolin-4-yl)but-3-yn-1- yl)piperidin-1-yl)-3-(2-fluoro- phenyl)-1-oxopropan-2- yl)amino)-1,5-dioxopentan-2- yl)carbamoyl)-3-methyl-6- oxodecahydropyrrolo[1,2- a][1,5]diazocin-5- yl)carbamoyl)benzo[b]thiophen-5- yl)difluoromethyl)phosphonic acid |
| 305 | 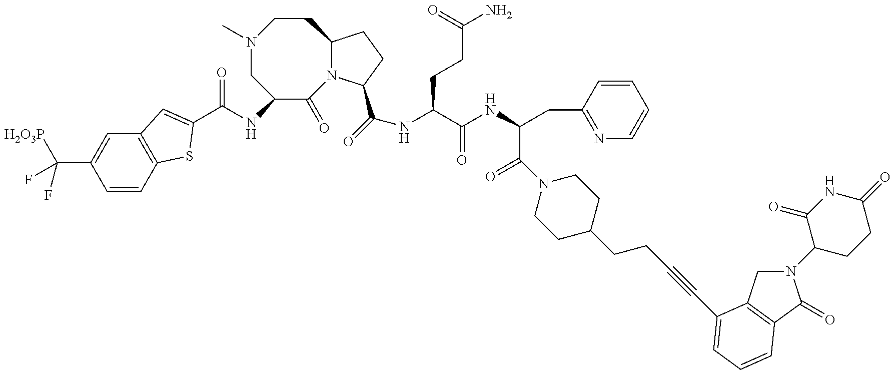 | ((2-(((5S,8S,10aR)-8-(((2S)-5- amino-1-(((2S)-1-(4-(4-(2- (2,6-dioxopiperidin-3-yl)-1- oxoisoindolin-4-yl)but-3-yn-1- yl)piperidin-1-yl)-1-oxo-3- (pyridin-2-yl)propan-2- yl)amino)-1,5-dioxopentan-2- yl)carbamoyl)-3-methyl-6- oxodecahydropyrrolo[1,2- a][1,5]diazocin-5- yl)carbamoyl)benzo[b]thiophen-5- yl)difluoromethyl)phosphonic acid |
| 306 | 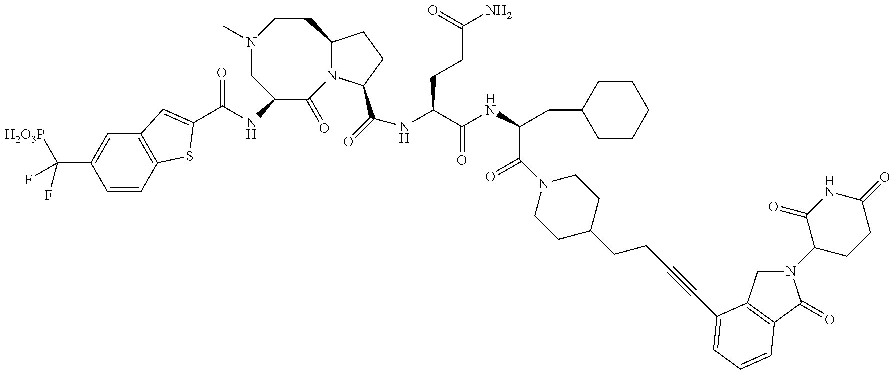 | ((2-(((5S,8S,10aR)-8-(((2S)-5- amino-1-(((2S)-3- cyclohexyl-1-(4-(4-(2-(2,6- dioxopiperidin-3-yl)-1- oxoisoindolin-4-yl)but-3-yn-1- yl)piperidin-1-yl)-1- oxopropan-2-yl)amino)-1,5- dioxopentan-2-yl)carbamoyl)- 3-methyl-6-oxodecahydro- pyrrolo[1,2-a][1,5]diazocin-5- yl)carbamoyl)benzo[b]thiophen-5- yl)difluoromethyl)phosphonic acid |
| 307 | 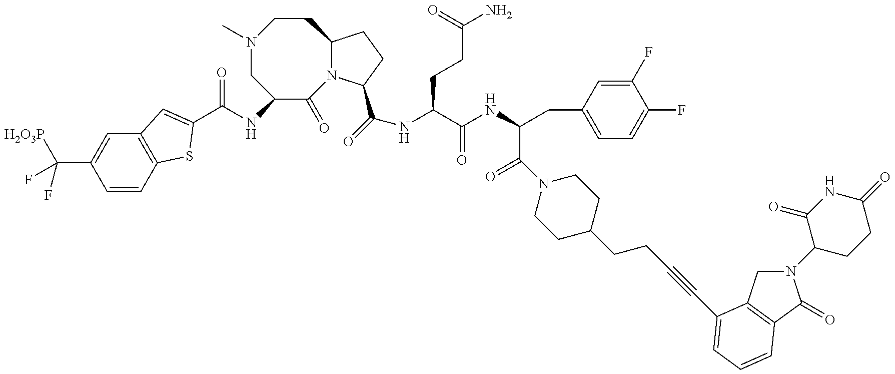 | ((2-(((5S,8S,10aR)-8-(((2S)- 5-amino-1-(((2S)-3-(3,4- difluorophenyl)-1-(4-(4-(2-(2,6- dioxopiperidin-3-yl)-1- oxoisoindolin-4-yl)but-3-yn-1- yl)piperidin-1-yl)-1- oxopropan-2-yl)amino)-1,5- dioxopentan-2-yl)carbamoyl)- 3-methyl-6-oxodecahydro- pyrrolo[1,2-a][1,5]diazocin-5- yl)carbamoyl)benzo[b]thiophen-5- yl)difluoromethyl)phosphonic acid |
| 308 | 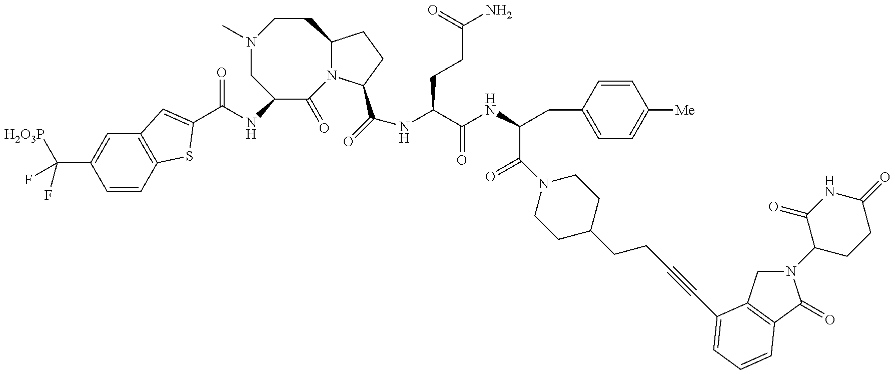 | ((2-(((5S,8S,10aR)-8-(((2S)-5- amino-1-(((2S)-1-(4-(4-(2- (2,6-dioxopiperidin-3-yl)-1- oxoisoindolin-4-yl)but-3-yn-1- yl)piperidin-1-yl)-1-oxo-3-(p- tolyl)propan-2-yl)amino)-1,5- dioxopentan-2-yl)carbamoyl)- 3-methyl-6-oxodecahydro- pyrrolo[1,2-a][1,5]diazocin-5- yl)carbamoyl)benzo[b]thiophen-5- yl)difluoromethyl)phosphonic acid |
| 309 | 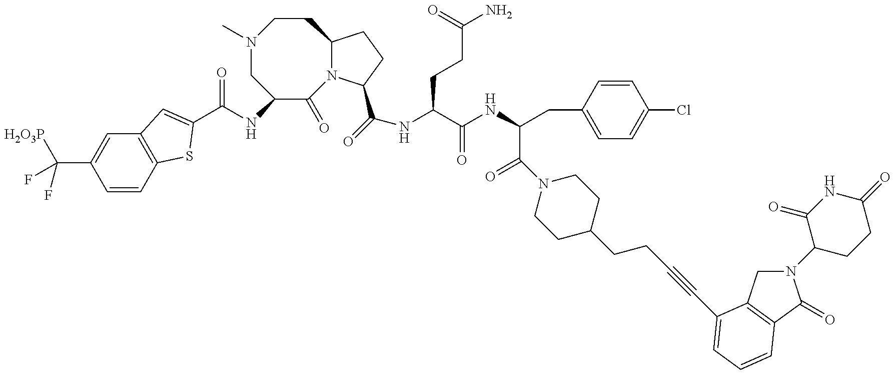 | ((2-(((5S,8S,10aR)-8-(((2S)-5-amino- 1-(((2S)-3-(4- chlorophenyl)-1-(4-(4-(2-(2,6- dioxopiperidin-3-yl)-1- oxoisoindolin-4-yl)but-3-yn-1- yl)piperidin-1-yl)-1- oxopropan-2-yl)amino)-1,5-dioxo- pentan-2-yl)carbamoyl)- 3-methyl-6-oxodecahydro- pyrrolo[1,2-a][1,5]diazocin-5- yl)carbamoyl)benzo[b]thiophen-5- yl)difluoromethyl)phosphonic acid |
| 310 | 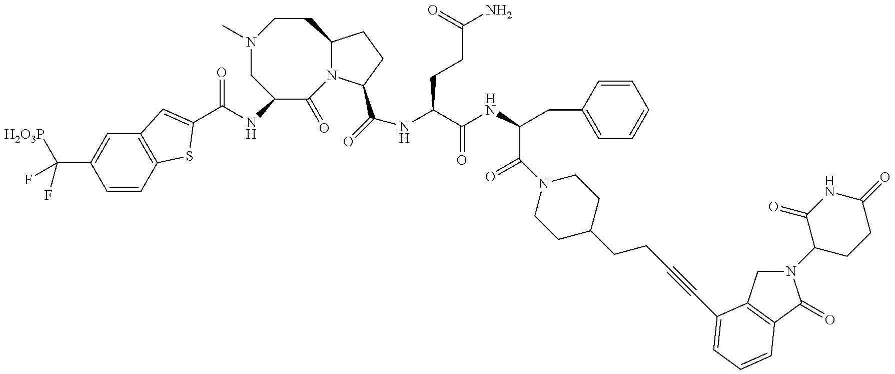 | ((2-(((5S,8S,10aR)-8-(((2S)- 3-(carbamoyloxy)-1-(((2S)-1- (4-(4-(2-(2,6-dioxopiperidin-3- yl)-1-oxoisoindolin-4- yl)but-3-yn-1-yl)piperidin-1- yl)-1-oxo-3-phenylpropan-2- yl)amino)-1-oxopropan-2- yl)carbamoyl)-3-methyl-6- oxodecahydropyrrolo[1,2- a][1,5]diazocin-5- yl)carbamoyl)benzo[b]thiopen-5- yl)difluoromethyl)phosphonic acid |
| 311 | 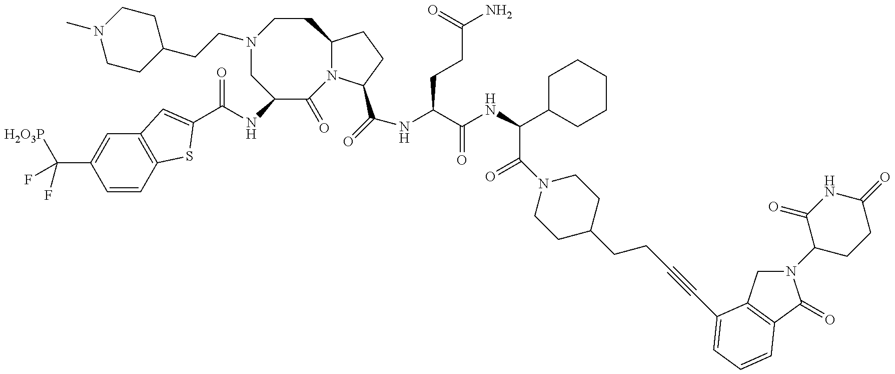 | ((2-(((5S,8S,10aR)-8-(((2S)- 5-amino-1-(((1S)-1- cyclohexyl-2-(4-(4-(2-(2,6- dioxopiperidin-3-yl)-1- oxoisoindolin-4-yl)but-3-yn- 1-yl)piperidin-1-yl)-2- oxoethyl)amino)-1,5-dioxopentan- 2-yl)carbamoyl)-3-(2-(1- methylpiperidin-4-yl)ethyl)-6- oxodecahydropyrrolo[1,2- a][1,5]diazocin-5- yl)carbamoyl)benzo[b]thiophen-5- yl)difluoromethyl)phosphonic acid |
| 312 | 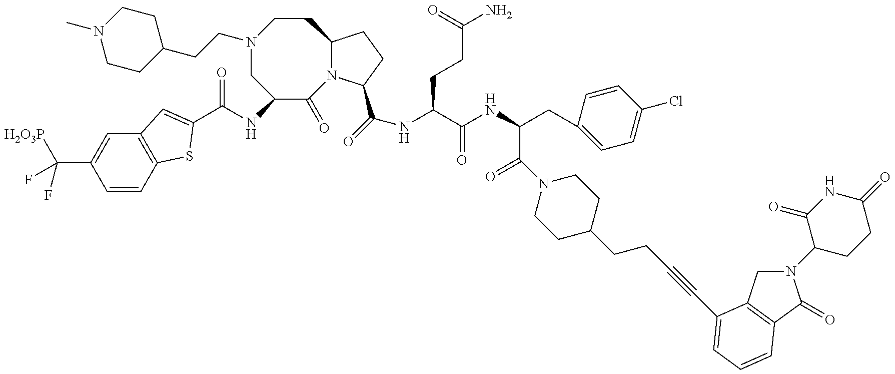 | ((2-(((5S,8S,10aR)-8-(((2S)- 5-amino-1-(((2S)-3-(4- chlorophenyl)-1-(4-(4-(2-(2,6- dioxopiperidin-3-yl)-1- oxoisoindolin-4-yl)but-3-yn- 1-yl)piperidin-1-yl)-1- oxopropan-2-yl)amino)-1,5- dioxopentan-2-yl)carbamoyl)- 3-(2-(1-methylpiperidin- 4-yl)ethyl)-6-oxodecahydro- pyrrolo[1,2-a][1,5]diazocin-5- yl)carbamoyl)benzo[b]thiophen-5- yl)difluoromethyl)phosphonic acid |
| 313 | 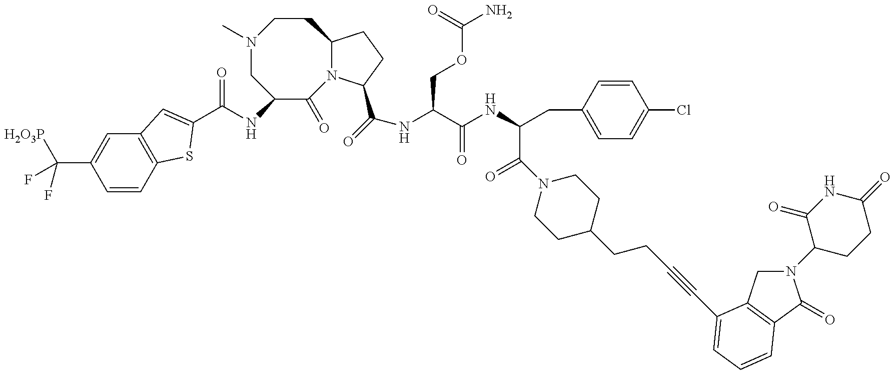 | ((2-(((5S,8S,10aR)-8-(((2S)- 3-(carbamoyloxy)-1-(((2S)-3- (4-chlorophenyl)-1-(4-(4-(2- (2,6-dioxopiperidin-3-yl)-1- oxoisoindolin-4-yl)but-3-yn-1- yl)piperidin-1-yl)-1- oxopropan-2-yl)amino)-1- oxopropan-2-yl)carbamoyl)-3- methyl-6-oxodecahydro- pyrrolo[1,2-a][1,5]diazocin-5- yl)carbamoyl)benzo[b]thiophen-5- yl)difluoromethyl)phosphonic acid |
| 314 | 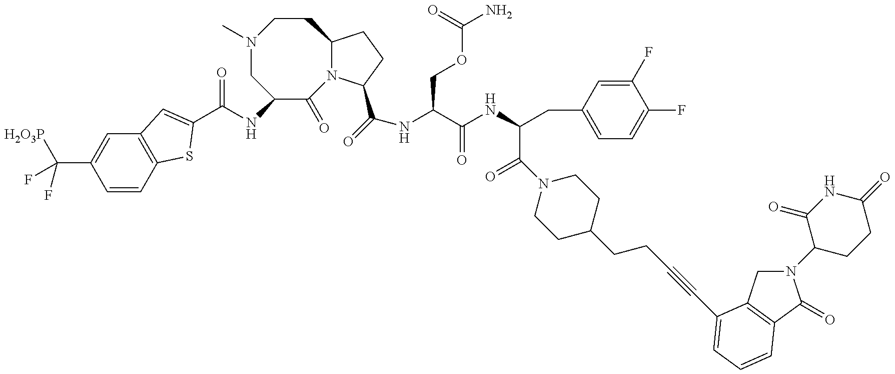 | ((2-(((5S,8S,10aR)-8-(((2S)- 3-(carbamoyloxy)-1-(((2S)-3- (3,4-difluorophenyl)-1-(4-(4- (2-(2,6-dioxopiperidin-3-yl)-1- oxoisoindolin-4-yl)but-3-yn-1- yl)piperidin-1-yl)-1- oxopropan-2-yl)amino)-1- oxopropan-2-yl)carbamoyl)-3- methyl-6-oxodecahydro- pyrrolo[1,2-a][1,5]diazocin-5- yl)carbamoyl)benzo[b]thiophen-5- yl)difluoromethyl)phosphonic acid |
| 315 | 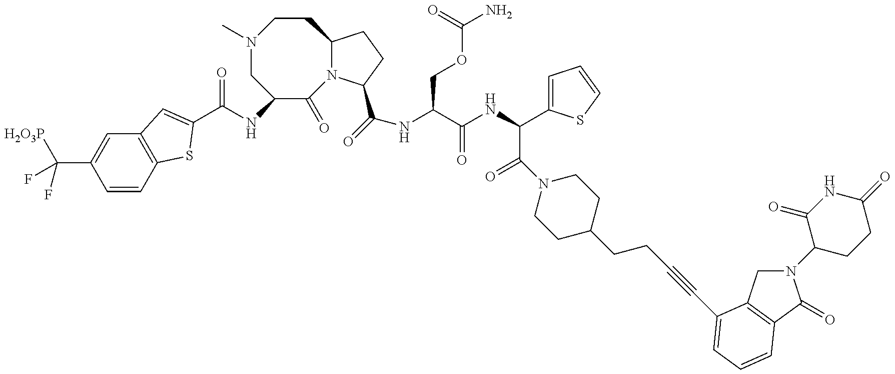 | ((2-(((5S,8S,10aR)-8-(((2S)- 3-(carbamoyloxy)-1-(((1R)-2- (4-(4-(2-(2,6-dioxopiperidin- 3-yl)-1-oxoisoindolin-4- yl)but-3-yn-1-yl)piperidin- 1-yl)-2-oxo-1-(thiophen-2- yl)ethyl)amino)-1-oxopropan- 2-yl)carbamoyl)-3-methyl-6- oxodecahydropyrrolo[1,2- a][1,5]diazocin-5- yl)carbamoyl)benzo[b]thiophen-5- yl)difluroomethyl)phosphonic acid |
| 316 | 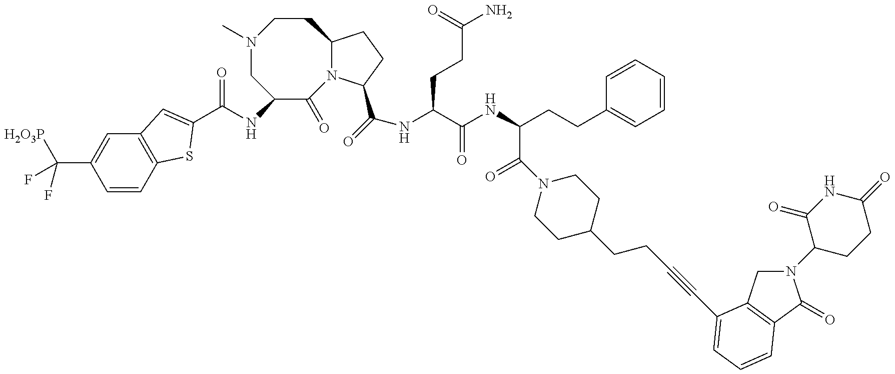 | ((2-(((5S,8S,10aR)-8-(((2S)- 5-amino-1-(((2S)-1-(4-(4-(2- (2,6-dioxopiperidin-3-yl)-1- oxoisoindolin-4-yl)but-3-yn-1- yl)piperidin-1-yl)-1-oxo-4- phenylbutan-2-yl)amino)-1,5- dioxopentan-2-yl)carbamoyl)- 3-methyl-6-oxodecahydro- pyrrolo[1,2-a][1,5]diazocin-5- yl)carbamoyl)benzo[b]thiophen-5- yl)difluoromethyl)phosphonic acid |
| 317 | 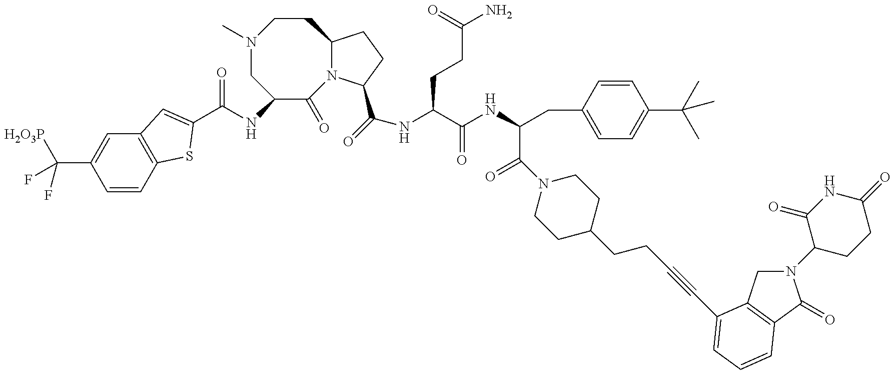 | ((2-(((5S,8S,10aR)-8-(((2S)- 5-amino-1-(((2S)-3-(4-(tert- butyl)phenyl)-1-(4-(4-(2-(2,6- dioxopiperidin-3-yl)-1- oxoisoindolin-4-yl)but-3-yn- 1-yl)piperidin-1-yl)-1- oxopropan-2-yl)amino)-1,5- dioxopentan-2-yl)carbamoyl)- 3-methyl-6-oxodecahydro- pyrrolo[1,2-a][1,5]diazocin-5- yl)carbamoyl)benzo[b]thiophen-5- yl)difluoromethyl)phosphonic acid |
| 318 | 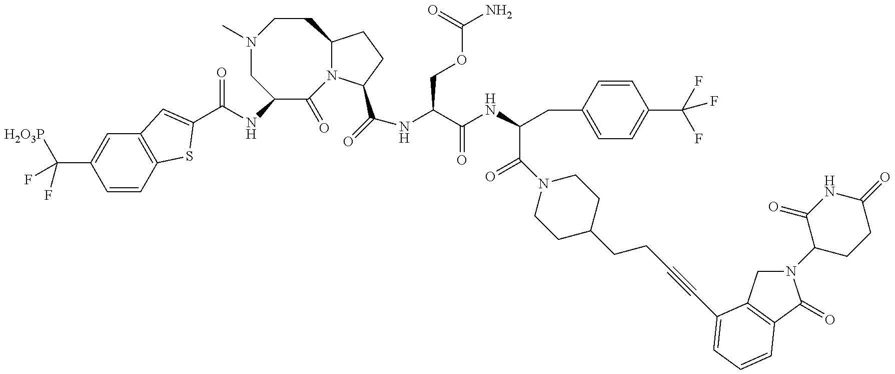 | ((2-(((5S,8S,10aR)-8-(((2S)-5- amino-1-(((2S)-1-(4-(4-(2- (2,6-dioxopiperidin-3-yl)-1- oxoisoindolin-4-yl)but-3-yn-1- yl)piperidin-1-yl)-1-oxo-3-(4- (trifluoromethyl)phenyl)propan- 2-yl)amino)-1,5- dioxopentan-2-yl)carbamoyl)- 3-methyl-6-oxodecahydro- pyrrolo[1,2-a][1,5]diazocin-5- yl)carbamoyl)benzo[b]thiophen-5- yl)difluoromethyl)phosphonic acid |
| 319 | 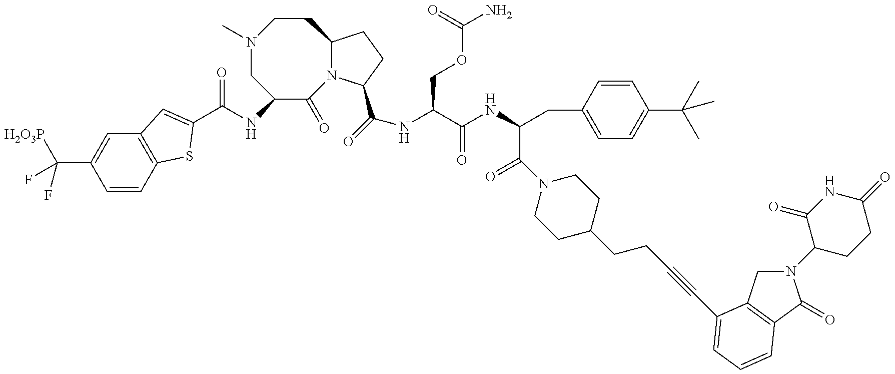 | ((2-(((5S,8S,10aR)-8-(((2S)- 1-(((2S)-3-(4-(tert- butyl)phenyl)-1-(4-(4-(2-(2,6- dioxopiperidin-3-yl)-1- oxoisoindolin-4-yl)but-3-yn-1- yl)piperidin-1-yl)-1- oxoproapn-2-yl)amino)-3- (carbamoyloxy)-1-oxopropan-2- yl)carbamoyl)-3-methyl-6- oxodecahydropyrrolo[1,2- a][1,5]diazocin-5- yl)carbamoyl)benzo[b]thiophen-5- yl)difluoromethyl)phosphonic acid |
| 320 | 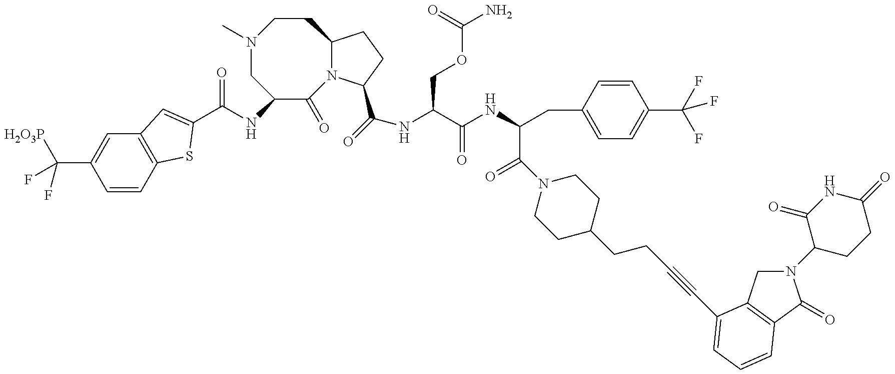 | ((2-(((5S,8S,10aR)-8-(((2S)- 3-(carbamoyloxy)-1-(((2S)-1- (4-(4-(2-(2,6-dioxopiperidin- 3-yl)-1-oxoisoindolin-4- yl)but-3-yn-1-yl)piperidin-1- yl)-1-oxo-3-(4- (trifluoromethyl)phenyl)propan- 2-yl)amino)-1-oxopropan- 2-yl)carbamoyl)-3-methyl-6- oxodecahydropyrrolo[1,2- a][1,5]diazocin-5- yl)carbamoyl)benzo[b]thiophen-5- yl)difluoromethyl)phosphonic acid |
| 321 | 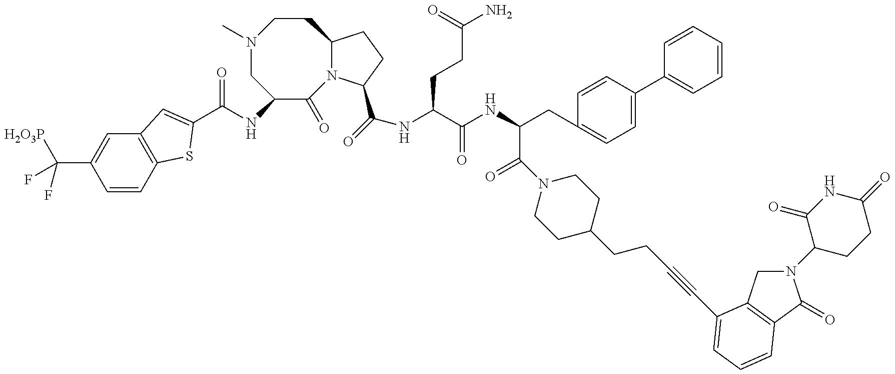 | ((2-(((5S,8S,10aR)-8-(((2S)- 1-(((2S)-3-([1,1′-biphenyl]-4- yl)-1-(4-(4-(2-(2,6-dioxopiperidin- 3-yl)-1-oxoisoindolin-4- yl)but-3-yn-1-yl)piperidin-1- yl)-1-oxopropan-2-yl)amino)- 5-amino-1,5-dioxopentan-2- yl)carbamoyl)-3-methyl-6- oxodecahydropyrrolo[1,2- a][1,5]diazocin-5- yl)carbamoyl)benzo[b]thiophen-5- yl)difluoromethyl)phosphonic acid |
| 322 | 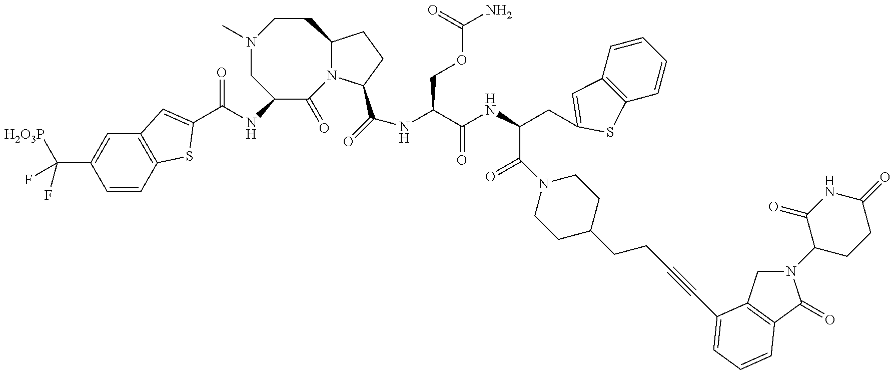 | ((2-(((5S,8S,10aR)-8-(((2S)-1- (((2S)-3-(benzo[b]thiophen- 2-yl)-1-(4-(4-(2-(2,6-dioxopiperidin- 3-yl)-1-oxoisoindolin- 4-yl)but-3-yn-1-yl)piperidin- 1-yl)-1-oxopropan-2- yl)amino)-3-(carbamoyloxy)- 1-oxopropan-2-yl)carbamoyl)- 3-methyl-6-oxodecahydro- pyrrolo[1,2-a][1,5]diazocin-5- yl)carbamoyl)benzo[b]thiophen-5- yl)difluoromethyl)phosphonic acid |
| 323 | 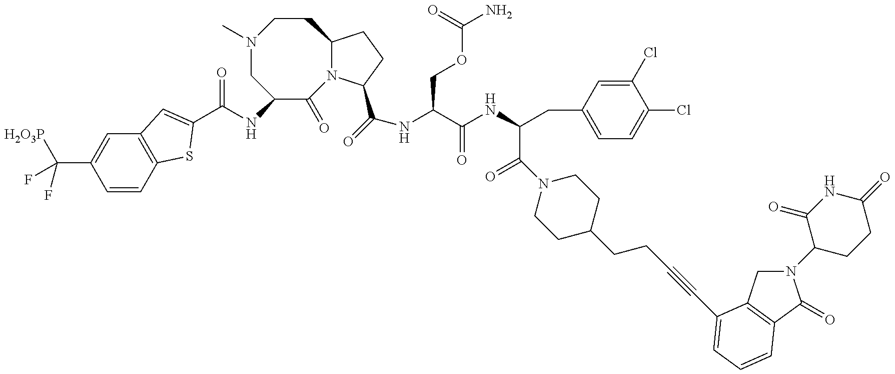 | ((2-(((5S,8S,10aR)-8-(((2S)-3- (carbamoyloxy)-1-(((2S)-3- (3,4-dichlorophenyl)-1-(4-(4- (2-(2,6-dioxopiperidin-3-yl)- 1-oxoisoindolin-4-yl)but-3-yn- 1-yl)piperidin-1-yl)-1- oxopropan-2-yl)amino)-1- oxopropan-2-yl)carbamoyl)-3- methyl-6-oxodecahydropyrrolo[1,2- a][1,5]diazocin-5- yl)carbamoyl)benzo[b]thiophen-5- yl)difluoromethyl)phosphonic acid |
| 324 | 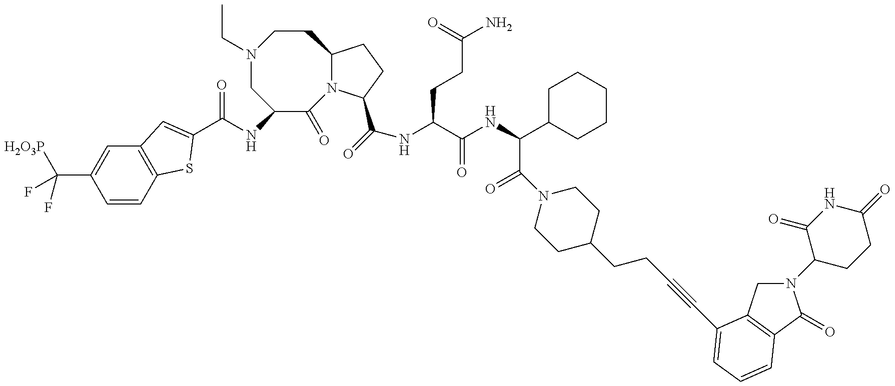 | ((2-(((5S,8S,10aR)-8-(((2S)- 5-amino-1-(((1S)-1- cyclohexyl-2-(4-(4-(2- (2,6-dioxopiperidin-3-yl)-1- oxoisoindolin-4-yl)but- 3-yn-1-yl)piperidin-1-yl)-2- oxoethyl)amino)-1,5-dioxo- pentan-2-yl)carbamoyl)-3-ethyl- 6-oxodecahydro- pyrrolo[1,2-a][1,5]diazocin-5- yl)carbamoyl)benzo[b]thiophen-5- yl)difluoromethyl)phosphonic acid |
| 325 | 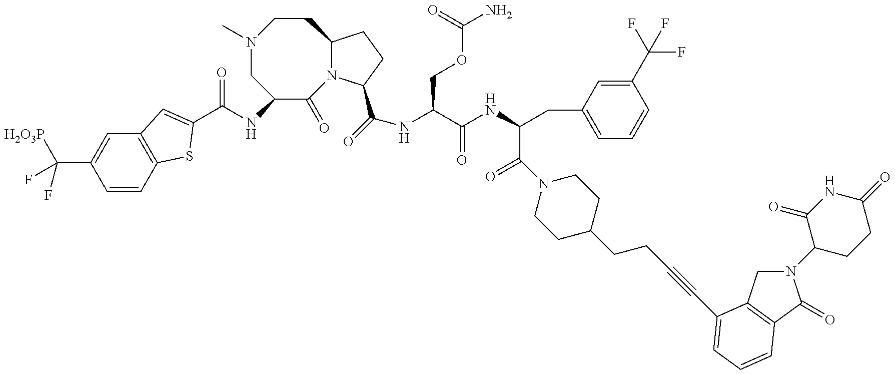 | ((2-(((5S,8S,10aR)-8-(((2S)- 3-(carbamoyloxy)-1-(((2S)-1- (4-(4-(2-(2,6-dioxopiperidin- 3-yl)-1-oxoisoindolin-4- yl)but-3-yn-1-yl)piperidin-1- yl)-1-oxo-3-(3- (trifluoromethyl)phenyl)propan- 2-yl)amino)-1-oxopropan- 2-yl)carbamoyl)-3-methyl-6- oxodecahydropyrrolo[1,2- a][1,5]diazocin-5- yl)carbamoyl)benzo[b]thiophen-5- yl)difluoromethyl)phosphonic acid |
| 326 | 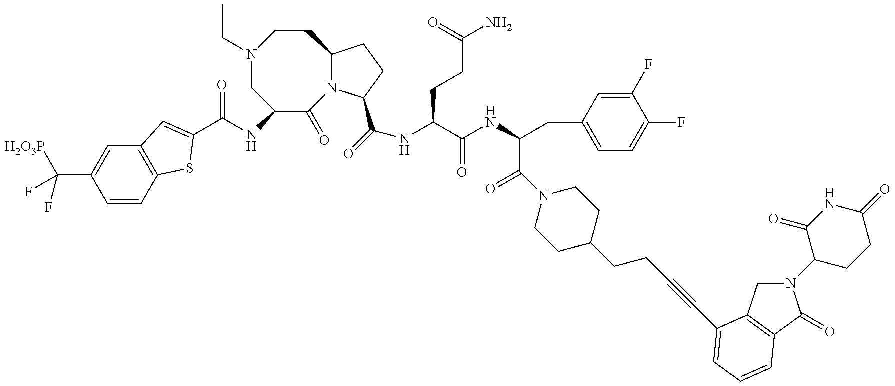 | ((2-(((5S,8S,10aR)-8-(((2S)- 5-amino-1-(((2S)-3-(3,4- difluorophenyl)-1-(4-(4-(2- (2,6-dioxopiperidin-3-yl)-1- oxoisoindolin-4-yl)but-3-yn- 1-yl)piperidin-1-yl)-1- oxopropan-2-yl)amino)-1,5- dioxopentan-2-yl)carbamoyl)- 3-ethyl-6-oxodecahydro- pyrrolo[1,2-a][1,5]diazocin-5- yl)carbamoyl)benzo[b]thiophen-5- yl)difluoromethyl)phosphonic acid |
| 327 | 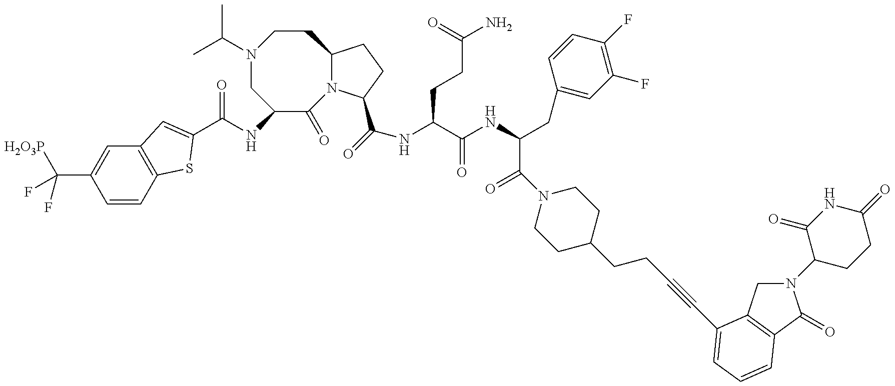 | ((2-(((5S,8S,10aR)-8-(((2S)- 5-amino-1-(((2S)-3-(3,4- difluorophenyl)-1-(4-(4-(2- (2,6-dioxopiperidin-3-yl)-1- oxoisoindolin-4-yl)but-3-yn- 1-yl)piperidin-1-yl)-1- oxopropan-2-yl)amino)- 1,5-dioxopentan-2-yl)carbamoyl)- 3-isopropyl-6-oxodeca- hydropyrrolo[1,2-a][1,5]diazocin-5- yl)carbamoyl)benzo[b]thiophen-5- yl)difluoromethyl)phosphonic acid |
| 328 | 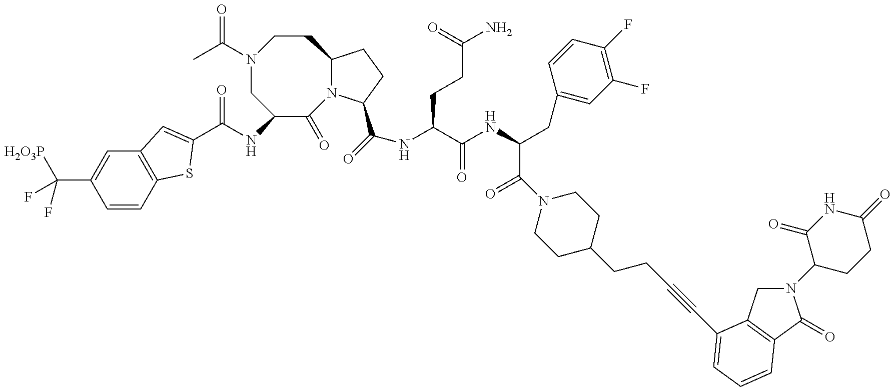 | ((2-(((5S,8S,10aR)-3-acetyl- 8-(((2S)-5-amino-1-(((2S)-3- (3,4-difluorophenyl)-1-(4-(4- (2-(2,6-dioxopiperidin-3-yl)-1- oxoisoindolin-4-yl)but-3-yn-1- yl)piperidin-1-yl)-1- oxopropan-2-yl)amino)-1,5- dioxopentan-2-yl)carbamoyl)- 6-oxodecahydropyrrolo[1,2- a][1,5]diazocin-5- yl)carbamoyl)benzo[b]thiophen-5- yl)difluoromethyl)phosphonic acid |
| 329 | 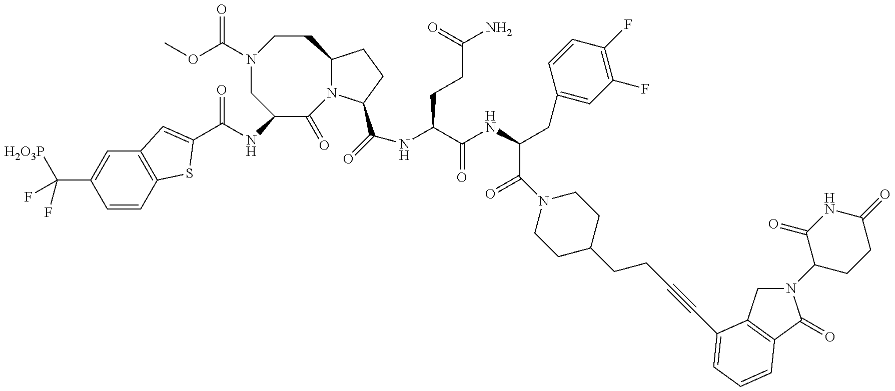 | ((2-(((5S,8S,10aR)-8-(((2S)- 5-amino-1-(((2S)-3-(3,4- difluorophenyl)-1-(4-(4-(2- (2,6-dioxopiperidin-3-yl)-1- oxoisoindolin-4-yl)but-3-yn- 1-yl)piperidin-1-yl)-1- oxopropan-2-yl)amino)-1,5- dioxopentan-2-yl)carbamoyl)- 3-(methoxycarbonyl)-6- oxodecahydropyrrolo[1,2- a][1,5]diazocin-5- yl)carbamoyl)benzo[b]thiophen-5- yl)difluoromethyl)phosphonic acid |
| 330 | 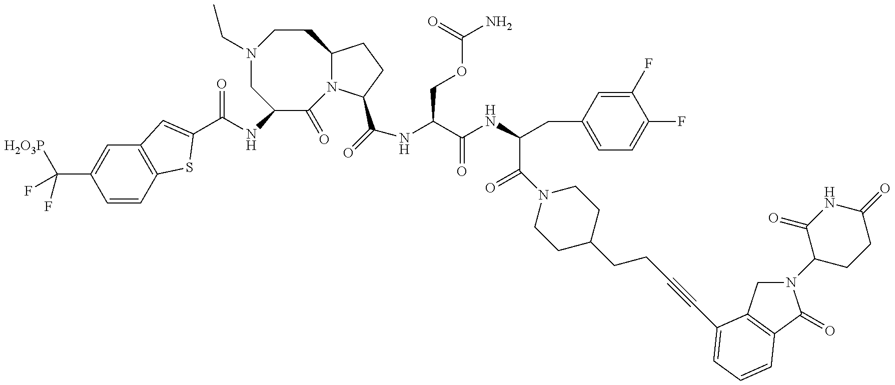 | ((2-(((5S,8S,10aR)-8-(((2S)- 3-(carbamoyloxy)-1-(((2S)-3- (3,4-difluorophenyl)-1-(4-(4- (2-(2,6-dioxopiperidin-3-yl)-1- oxoisoindolin-4-yl)but-3-yn- 1-yl)piperidin-1-yl)-1- oxopropan-2-yl)amino)-1- oxopropan-2-yl)carbamoyl)-3- ethyl-6-oxodecahydro- pyrrolo[1,2-a][1,5]tiazocin-5- yl)carbamoyl)benzo[b]thiophen-5- yl)difluoromethyl)phosphonic acid |
| 331 | 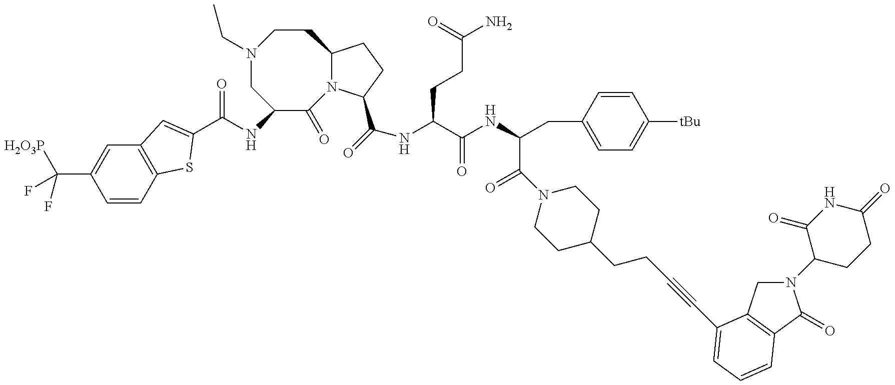 | ((2-(((5S,8S,10aR)-8-(((2S)- 5-amino-1-(((2S)-3-(4-(tert- butyl)phenyl)-1-(4-(4-(2-(2,6- dioxopiperidin-3-yl)-1- oxoisoindolin-4-yl)but-3-yn- 1-yl)piperidin-1-yl)-1- oxopropan-2-yl)amino)-1,5- dioxopentan-2-yl)carbamoyl)- 3-ethyl-6-oxodecahydro- pyrrolo[1,2-a]1,5]diazocin-5- yl)carbamoyl)benzo[b]thiophen-5- yl)difluoromethyl)phosphonic acid |
| 332 | 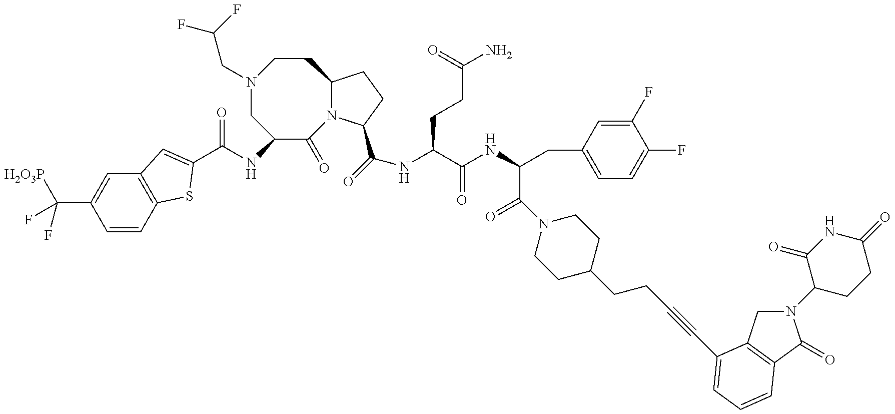 | ((2-(((5S,8S,10aR)-8-(((2S)- 5-amino-1-(((2S)-3-(3,4- difluorophenyl)-1-(4-(4-(2- (2,6-dioxopiperidin-3-yl)-1- oxoisoindolin-4-yl)but-3-yn- 1-yl)piperidin-1-yl)-1- oxopropan-2-yl)amino)-1,5- dioxopentan-2-yl)carbamoyl)- 3-(2,2-difluoroethyl)-6-oxo- decahydropyrrolo[1,2- a][1,5]diazocin-5- yl)carbamoyl)benzo[b]thiophen-5- yl)difluoromethyl)phosphonic acid |
| 333 | 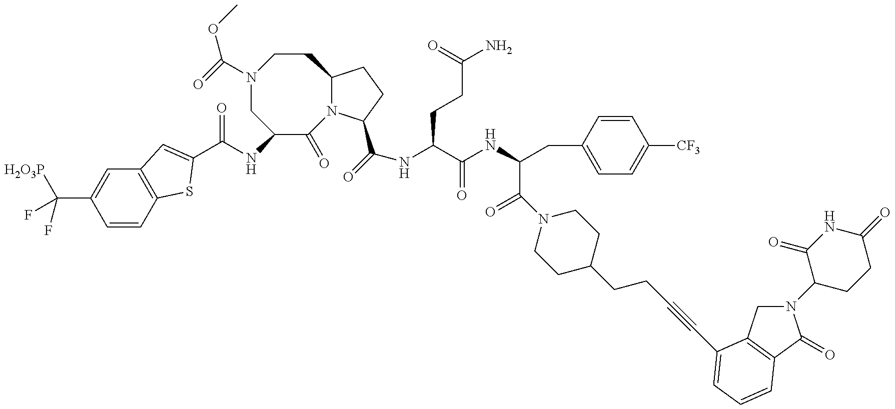 | ((2-(((5S,8S,10aR)-8- (((2S)-5-amino-1-(((2S)-1-(4-(4-(2- (2,6-dioxopiperidin-3-yl)- 1-oxoisoindolin-4-yl)but-3-yn-1- yl)piperidin-1-yl)-1-oxo-3-(4- (trifluoromethyl)phenyl)propan- 2-yl)amino)-1,5- dioxopentan-2-yl)carbamoyl)- 3-(methoxycarbonyl)-6- oxodecahydropyrrolo[1,2- a][1,5]diazocin-5- yl)carbamoyl)benzo[b]thiophen-5- yl)difluoromethyl)phosphonic acid |
| 334 | 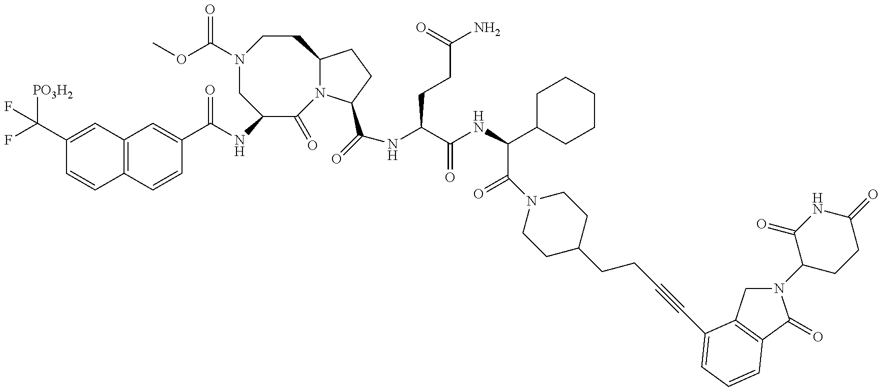 | ((7-(((5S,8S,10aR)-8- (((2S)-5-amino-1-(((1S)-1- cyclohexyl-2-(4-(4-(2- (2,6-dioxopiperidin-3-yl)-1- oxoisoindolin-4-yl)but-3- yn-1-yl)piperidin-1-yl)-2- oxoethyl)amino)-1,5-dioxo- pentan-2-yl)carbamoyl)-3- (methoxycarbonyl)-6- oxodecahydropyrrolo[1,2- a][1,5]diazocin-5- yl)carbamoyl)naphthalen-2- yl)difluoromethyl)phosphonic acid |
| 335 | 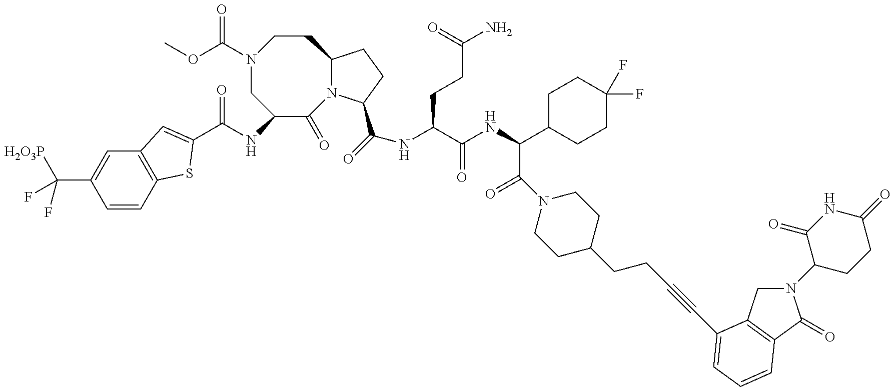 | ((2-(((5S,8S,10aR)-8-(((2S)- 5-amino-1-(((1S)-1-(4,4- difluorocyclohexyl)-2-(4-(4- (2-(2,6-dioxopiperidin-3-yl)-1- oxoisoindolin-4-yl)but-3-yn- 1-yl)piperidin-1-yl)-2- oxoethyl)amino)-1,5-dioxopentan- 2-yl)carbamoyl)-3- (methoxycarbonyl)-6-oxodeca- hydropyrrolo[1,2- a][1,5]diazocin-5- yl)carbamoyl)benzo[b]thiophen-5- yl)difluoromethyl)phosphonic acid |
| 336 | 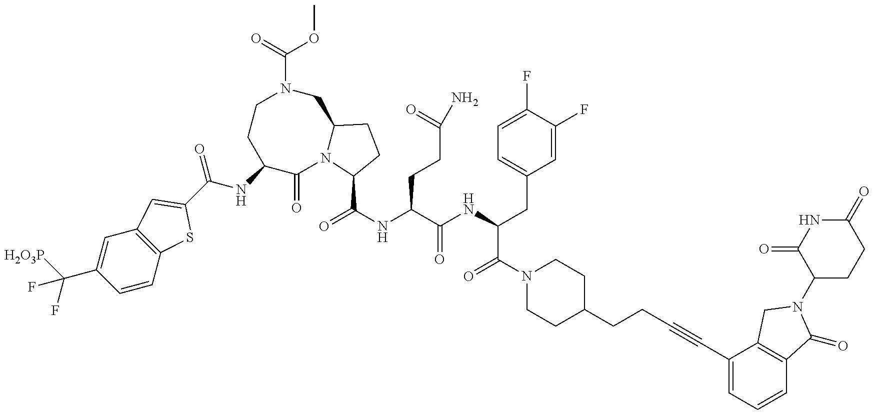 | ((2-(((5S,85S,10aR)-8- (((2S)-5-amino-1-(((2S)-3-(3,4- difluorophenyl)-1-(4-(4-(2- (2,6-dioxopiperidin-3-yl)-1- oxoisoindolin-4-yl)but-3-yn- 1-yl)piperidin-1-yl)-1- oxopropan-2-yl)amino)-1,5- dioxopentan-2-yl)carbamoyl)- 2-(methoxycarbonyl)-6- oxodecahydropyrrolo[1,2- a][1,4]diazocin-5- yl)carbamoyl)benzo[b]thipohen-5- yl)difluoromethyl)phosphonic acid |
| 337 | 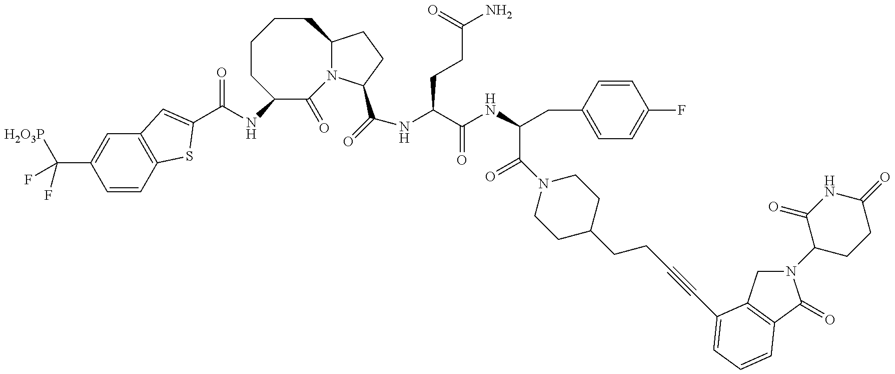 | ((2-(((3S,6S,10aS)-3-(((2S)- 5-amino-1-(((2S)-1-(4-(4-(2- (2,6-dioxopiperidin-3-yl)-1- oxoisoindolin-4-yl)but-3-yn-1- yl)piperidin-1-yl)-3-(4- fluorophenyl)-1-oxopropan-2- yl)amino)-1,5-dioxopentan- 2-yl)carbamoyl)-5- oxodecahydropyrrolo[1,2- a]azocin-6- yl)carbamoyl)benzo[b]thiophen-5- yl)difluoromethyl)phosphonic acid |
| 339 | 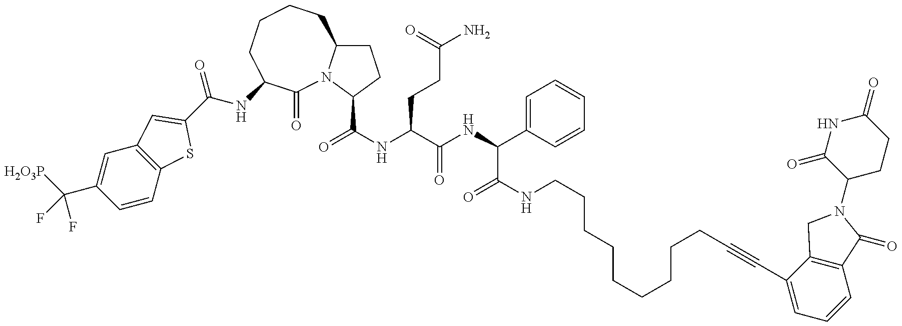 | ((2-(((3S,6S,10aS)-3-(((2S)-5- amino-1-(((1S)-2-((11-(2- (2,6-dioxopiperidin-3-yl)-1- oxoisoindolin-4-yl)undec-10- yn-1-yl)amino)-2-oxo-1- phenylethyl)amino)-1,5- dioxopentan-2-yl)carbamoyl)-5- oxodecahydropyrrolo[1,2- a]azocin-6-yl)carbamoyl)-1H-indol-5- yl)difluoromethyl)phosphonic acid |
| 340 | 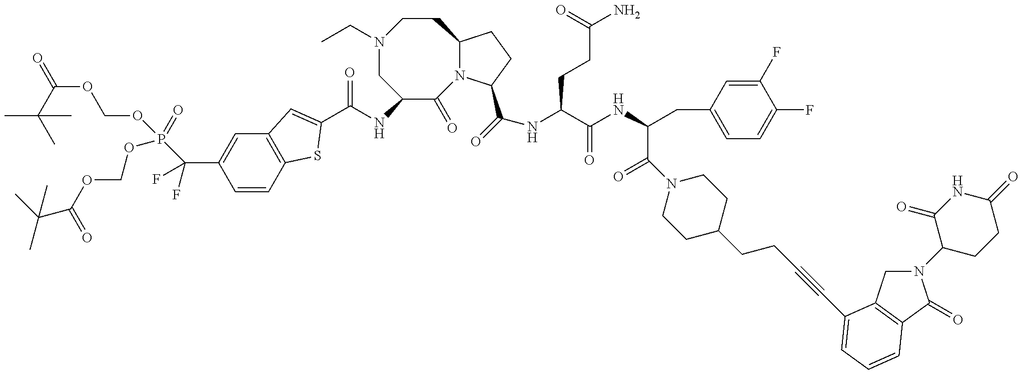 [SMILES: CCN1CC[C@H]2CC[C@@H](C(=O)N[C@@H](CCC(N)=O)C(=O)N[C@@H](Cc3ccc(F)c(F)c3)C(=O)N3CCC(CCC#Cc4cccc5c4CN(C4CCC(=O)NC4=O)C5=O)CC3)N2C(=O)[C@@H](NC(=O)c2cc3cc(C(F)(F)P(=O)(OCOC(=O)C(C)(C)C)OCOC(=O)C(C)(C)C)ccc3s2)C1] | (((2-(((5S,8S,10aR)-8-(((2S)- 5-amino-1-(((2S)-3-(3,4- difluorophenyl)-1-(4-(4-(2-(2,6- dioxopiperidin-3-yl)-1- oxoisoindolin-4-yl)but-3-yn-1- yl)piperidin-1-yl)-1- oxopropan-2-yl)amino)-1,5- dioxopentan-2-yl)carbamoyl)- 3-ethyl-6-oxodecahydropyrrolo[1,2- a][1,5]diazocin-5- yl)carbamoyl)benzo[b]thiophen-5- yl)difluoromethyl)phos- phoryl)bis(oxy))bis(methylene) bis(2,2-dimethylpropanoate |
| 341 | 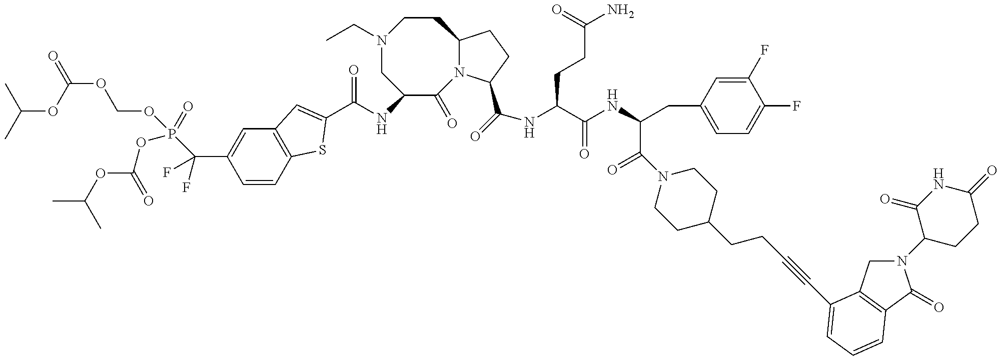 [SMILES: CCN1CC[C@H]2CC[C@@H](C(=O)N[C@@H](CCC(N)=O)C(=O)N[C@@H](Cc3ccc(F)c(F)c3)C(=O)N3CCC(CCC#Cc4cccc5c4CN(C4CCC(=O)NC4=O)C5=O)CC3)N2C(=O)[C@@H](NC(=O)c2cc3cc(C(F)(F)P(=O)(OCOC(=O)OC(C)C)OC(=O)OC(C)C)ccc3s2)C1] | ((((2-(((5S,8S,10aR)-8-(((2S)- 5-amino-1-(((2S)-3-(3,4- difluorophenyl)-1-(4-(4-(2-(2,6- dioxopiperidin-3-yl)-1- oxoisoindolin-4-yl)but-3-yn- 1-yl)piperidin-1-yl)-1- oxopropan-2-yl)amino)-1,5- dioxopentan-2-yl)carbamoyl)- 3-ethyl-6-oxodecahydropyrrolo[1,2- a][1,5]diazocin-5- yl)carbamoyl)benzo[b]thiophen-5- yl)difluoromethyl)phos- phoryl)bis(oxy))bis(methylene) diisopropyl bis(carbonate) |
| 342 | 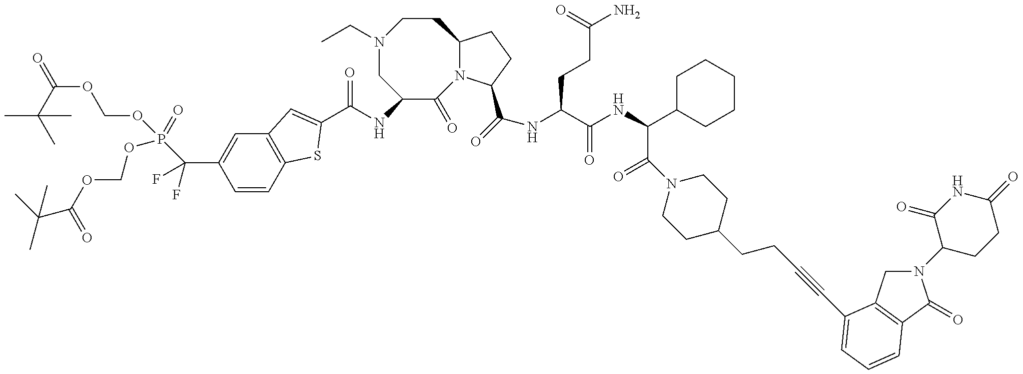 [SMILES: CCN1CC[C@H]2CC[C@@H](C(=O)N[C@@H](CCC(N)=O)C(=O)N[C@H](C(=O)N3CCC(CCC#Cc4cccc5c4CN(C4CCC(=O)NC4=O)C5=O)CC3)C3CCCCC3)N2C(=O)[C@@H](NC(=O)c2cc3cc(C(F)(F)P(=O)(OCOC(=O)C(C)(C)C)OCOC(=O)C(C)(C)C)ccc3s2)C1] | ((((2-(((5S,8S,10aR)-8- (((2S)-5-amino-1-(((1S)-1- cyclohexyl-2-(4-(4-(2-(2,6- dioxopiperidin-3-yl)-1- oxoisoindolin-4-yl)but-3-yn- 1-yl)piperidin-1-yl)-2- oxoethyl)amino)-1,5-dioxo- pentan-2-yl)carbamoyl)-3-ethyl- 6-oxodecahydropyrrolo[1,2-a][1,5]diazocin- 5-yl)carbamoyl)benzo[b]thiophen-5- yl)difluoromethyl)phos- phoryl)bis(oxy))bis(methylene) bis(2,2-dimethylpropanoate |
| 343 | 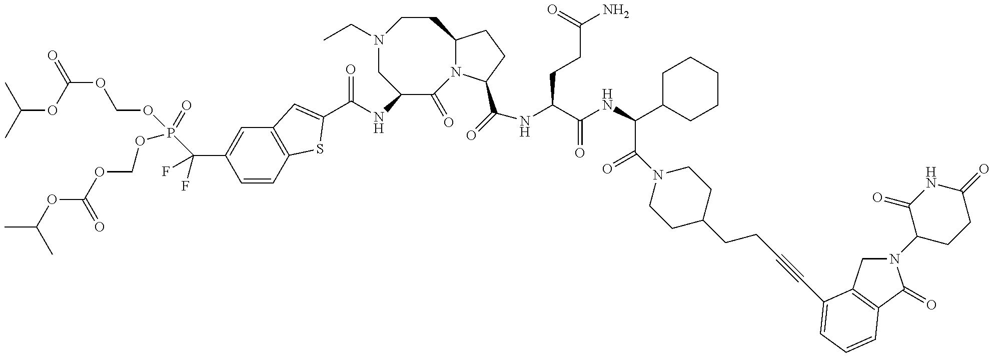 [SMILES: CCN1CC[C@H]2CC[C@@H](C(=O)N[C@@H](CCC(N)=O)C(=O)N[C@H](C(=O)N3CCC(CCC#Cc4cccc5c4CN(C4CCC(=O)NC4=O)C5=O)CC3)C3CCCCC3)N2C(=O)[C@@H](NC(=O)c2cc3cc(C(F)(F)P(=O)(OCOC(=O)OC(C)C)OCOC(=O)OC(C)C)ccc3s2)C1] | ((((2-(((5S,8S,10aR)-8-(((2S)-5- amino-1-(((1S)-1- cyclohexyl-2-(4-(4-(2-(2,6- dioxopiperidin-3-yl)-1- oxoisoindolin-4-yl)but-3-yn- 1-yl)piperidin-1-yl)-2- oxoethyl)amino)-1,5-dioxopentan- 2-yl)carbamoyl)-3-ethyl- 6-oxodecahydropyrrolo[1,2- a][1,5]diazocin-5- yl)carbamoyl)benzo[b]thiophen-5- yl)difluoromethyl)phos- phoryl)bis(oxy)bis(methylene) diisopropyl bis(carbonate) |
| 344 | 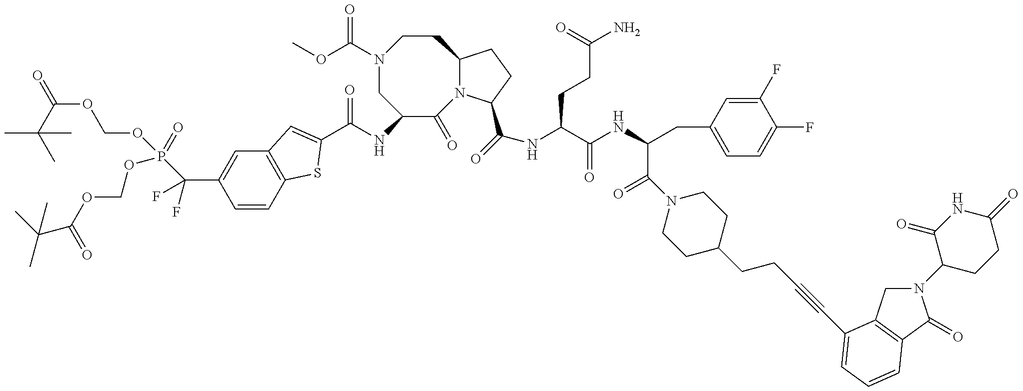 [SMILES: COC(=O)N1CC[C@H]2CC[C@@H](C(=O)N[C@@H](CCC(N)=O)C(=O)N[C@@H](Cc3ccc(F)c(F)c3)C(=O)N3CCC(CCC#Cc4cccc5c4CN(C4CCC(=O)NC4=O)C5=O)CC3)N2C(=O)[C@@H](NC(=O)c2cc3cc(C(F)(F)P(=O)(OCOC(=O)C(C)(C)C)OCOC(=O)C(C)(C)C)ccc3s2)C1] | ((((2-(((5S,8S,10aR)-8-(((2S)- 5-amino-1-(((2S)-3-(3,4- difluorophenyl)-1-(4-(4-(2- (2,6-dioxopiperidin-3-yl)-1- oxoisoindolin-4-yl)but-3-yn- 1-yl)piperidin-1-yl)-1- oxopropan-2-yl)amino)-1,5- dioxopentan-2-yl)carbamoyl)- 3-(methoxycarbonyl)-6-oxo- decahydropyrrolo[1,2- a][1,5]diazocin-5- yl)carbamoyl)benzo[b]thiophen-5- yl)difluoromethyl)phos- phoryl)bis(oxy))bis(methylene) bis(2,2-dimethylpropanoate) |
| 345 | 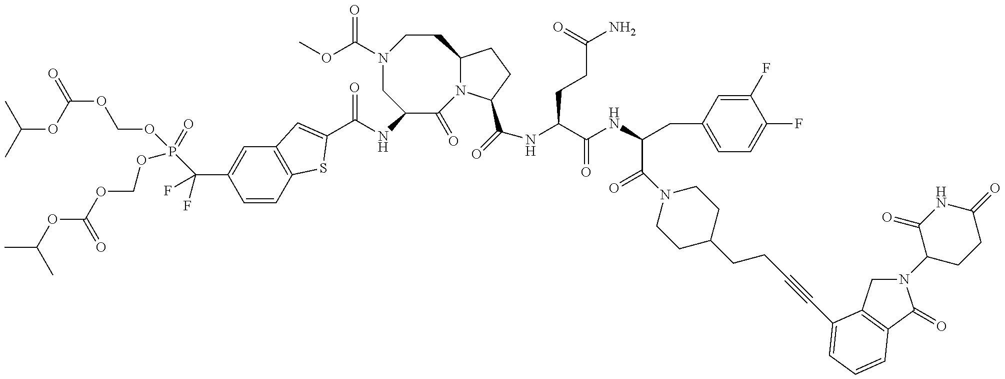 [SMILES: COC(=O)N1CC[C@H]2CC[C@@H](C(=O)N[C@@H](CCC(N)=O)C(=O)N[C@@H](Cc3ccc(F)c(F)c3)C(=O)N3CCC(CCC#Cc4cccc5c4CN(C4CCC(=O)NC4=O)C5=O)CC3)N2C(=O)[C@@H](NC(=O)c2cc3cc(C(F)(F)P(=O)(OCOC(=O)OC(C)C)OCOC(=O)OC(C)C)ccc3s2)C1] | methyl (5S,8S,10aR)-8- (((2S)-5-amino-1-(((2S)-3-(3,4- difluorophenyl)-1-(4-(4-(2-(2,6- dioxopiperidin-3-yl)-1- oxoisoindolin-4-yl)but-3-yn-1- yl)piperidin-1-yl)-1- oxopropan-2-yl)amino)-1,5- dioxopentan-2-yl)carbamoyl)- 5-(5- ((bis(((isopropoxycarbo- nyl)oxy)methoxy)phosphoryl)difluo- romethyl)benzo[b]thiophene- 2-carboxamideo)-6- oxooctahydropyrrolo[1,2- a][1,5]diazocine-3(4H)-carboxylate |
| 346 | 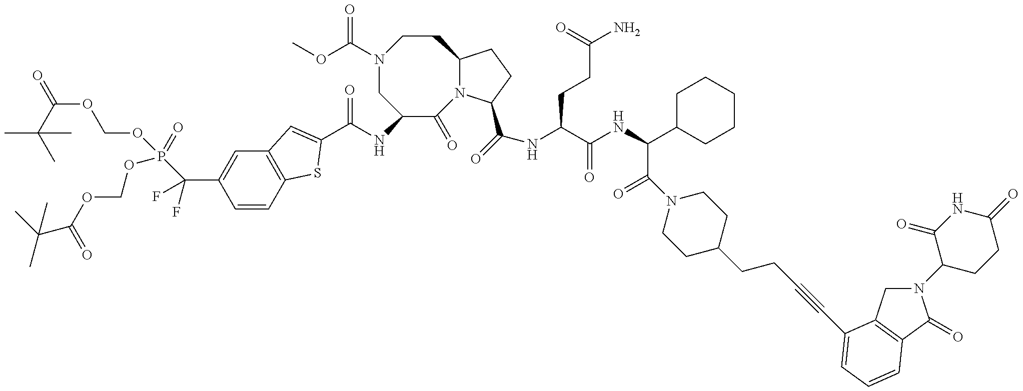 [SMILES: COC(=O)N1CC[C@H]2CC[C@@H](C(=O)N[C@@H](CCC(N)=O)C(=O)N[C@H](C(=O)N3CCC(CCC#Cc4cccc5c4CN(C4CCC(=O)NC4=O)C5=O)CC3)C3CCCCC3)N2C(=O)[C@@H](NC(=O)c2cc3cc(C(F)(F)P(=O)(OCOC(=O)C(C)(C)C)OCOC(=O)C(C)(C)C)ccc3s2)C1] | ((((2-(((5S,8S,10aR)-8-(((2S)- 5-amino-1-(((1S)-1- cyclohexyl-2-(4-(4-(2-(2,6- dioxopiperidin-3-yl)-1- oxoisoindolin-4-yl)but-3-yn- 1-yl)piperidin-1-yl)-2- oxoethyl)amino)-1,5-dioxopentan- 2-yl)carbamoyl)-3- (methoxycarbonyl)-6-oxodeca- hydropyrrolo[1,2- a][1,5]diazocin-5- yl)carbamoyl)benzo[b]thiophen-5- yl)difluoromethyl)phos- phoryl)bis(oxy))bis(methylene) bis(2,2-dimethylpropanoate |
| 347 | 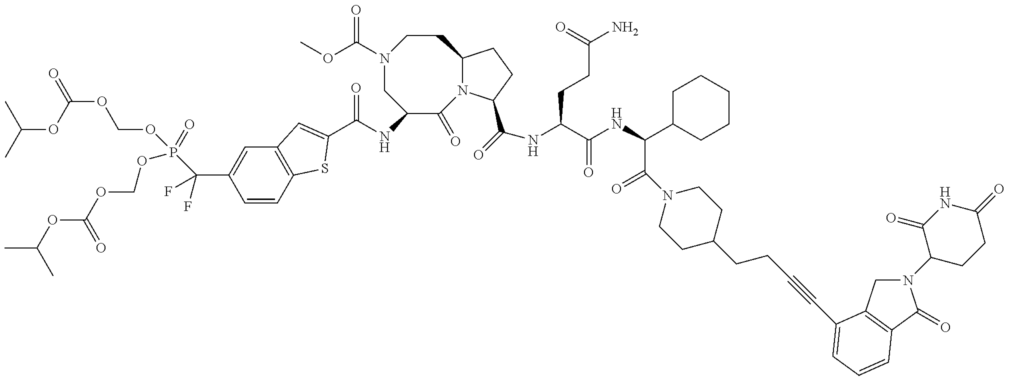 [SMILES: COC(=O)N1CC[C@H]2CC[C@@H](C(=O)N[C@@H](CCC(N)=O)C(=O)N[C@H](C(=O)N3CCC(CCC#Cc4cccc5c4CN(C4CCC(=O)NC4=O)C5=O)CC3)C3CCCCC3)N2C(=O)[C@@H](NC(=O)c2cc3cc(C(F)(F)P(=O)(OCOC(=O)OC(C)C)OCOC(=O)OC(C)C)ccc3s2)C1] | methyl (5S,8S,10aR)-8-(((2S)- 5-amino-1-(((1S)-1- cyclohexyl-2-(4-(4-(2-(2,6- dioxopiperidin-3-yl)-1- oxoisoindolin-4-yl)but-3-yn- 1-yl)piperidin-1-yl)-2- oxoethyl)amino)-1,5-dioxopentan- 2-yl)carbamoyl)-5-(5- ((bis(((isopropoxycarbo- nyl)oxy)methoxy)phosphoryl)difluoro- methyl)benzo[b]thiophen-2- carboxamido)-6-oxooctahydro- pyrrolo[1,2-a][1,5]diazocine-3(4H)- carboxylate |
| 348 | 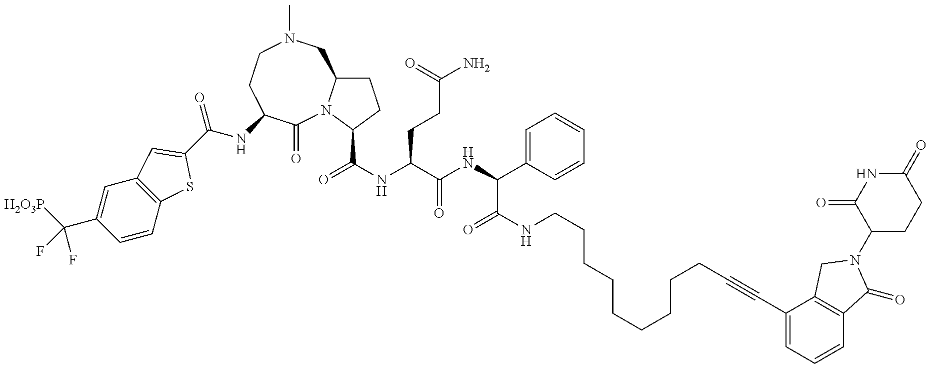 | ((2-(((5S,8S,10aR)-8-(((2S)-5- amino-1-(((1S)-2-((11-(2- (2,6-dioxopiperidin-3-yl)-1- oxoisoindolin-4-yl)undec-10- yn-1-yl)amino)-2-oxo-1- phenylethyl)amino)-1,5- dioxopentan-2-yl)carbamoyl)- 2-methyl-6- oxodecahydropyrrolo[1,2- a][1,4]diazocin-5-yl)carbamoyl)- 1H-indol-5-yl)difluoro- methyl)phosphonic acid |

TABLE 1B

| Cpd. | | |
|---|---|---|
| No. | Structure | Name |
| 349 | 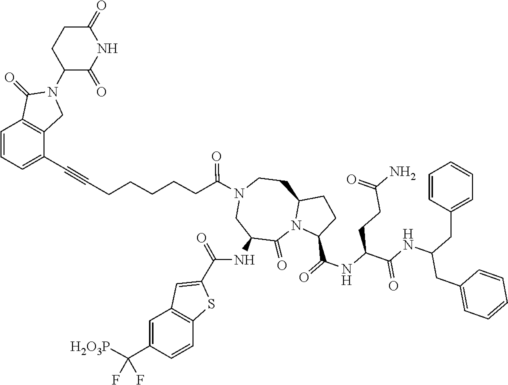 | ((2-(((5S,8S,10aR)-8-(((S)-5- amino-1-((1,3-diphenylpropan-2- yl)amino)-1,5-dioxopentan-2-yl) carbamoyl)-3-(8-(2-(2,6- dioxopiperidin-3-yl)-1- oxoisoindolin-4-yl)oct-7-ynoyl)-6- oxodecahydropyrrolo[1,2-a][1,5] diazocin-5-yl)carbamoyl) benzo[b]thiophen-5-yl) difluoromethyl)phosphonic acid |
| 350 | 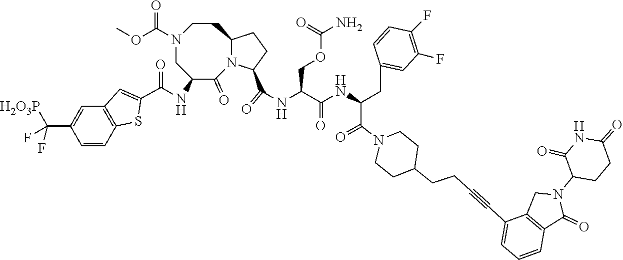 | ((2-(((5S,8S,10aR)-8-(((2S)- 5-(carbamoyloxy)-1-(((2S)-3-(3,4- difluorophenyl)-1-(4-(4-(2- (2,6-dioxopiperidin-3-yl)-1- oxoisoindolin-4-yl)but-3-yn-1-yl) piperidin-1-yl)-1-oxopropan-2- yl)amino)-1-oxopropan-2-yl) carbamoyl)-3-(methoxycarbonyl)- 6-oxodecahydropyrrolo [1,2-a][1,5]diazocin-5-yl) carbamoyl)benzo[b]thiophen-5-yl) difluoromethyl)phosphonic acid |
| 351 | 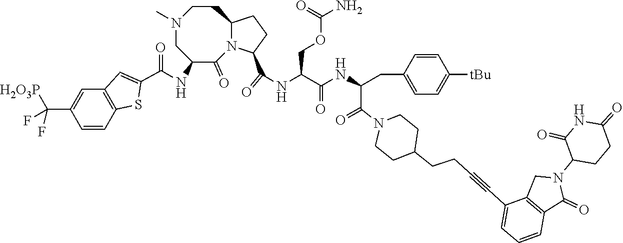 | ((2-(((5S,8S,10aR)-8-(((2S)-1- (((2S)-3-(4-(tert-butyl)phenyl)- 1-(4-(4-(2-(2,6-dioxopiperidin-3- yl)-1-oxoisoindolin-4-yl)but-3-yn-1- yl)piperidin-1-yl)-1-oxopropan-2-yl) amino)-3-(carbamoyloxy)-1- oxopropan-2-yl)carbamoyl)- 3-methyl-6-oxodecahydropyrrolo [1,2-a][1,5]diazocin-5-yl)carbamoyl) benzo[b]thiophen-5- yl)difluoromethyl)phosphonic acid |
| 352 | 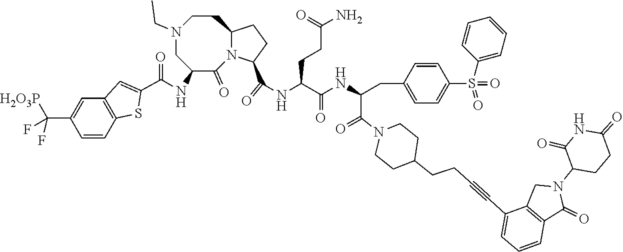 | ((2-(((5S,8S,10aR)-8-(((2S)- 5-amino-1-(((2S)-1-(4-(4-(2- (2,6-dioxopiperidin-3-yl)-1- oxoisoindolin-4-yl)but-3-yn-1-yl) piperidin-1-yl)-1-oxo-3-(4- (phenylsulfonyl)phenyl)propan- 2-yl)amino)-1,5-dioxopentan-2-yl) carbamoyl)-3-ethyl-6- oxodecahydropyrrolo[1,2-a][1,5] diazocin-5-yl)carbamoyl)benzo [b]thiophen-5-yl)difluoromethyl) phosphonic acid |
| 353 | 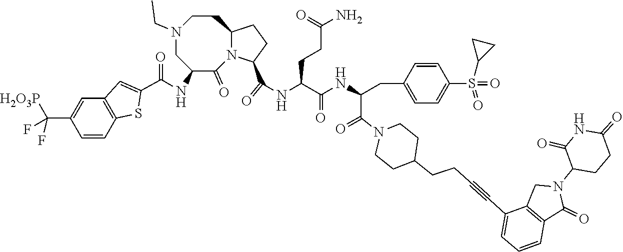 | ((2-(((5S,8S,10aR)-8-(((2S)- 5-amino-1-(((2S)-3-(4- (cyclopropylsulfonyl)phenyl)- 1-(4-(4-(2-(2,6-dioxopiperidin-3-yl)-1- oxoisoindolin-4-yl)but-3-yn-1-yl) piperidin-1-yl)-1-oxopropan-2- yl)amino)-1,5-dioxopentan-2-yl) carbamoyl)-3-ethyl-6- oxodecahydropyrrolo[1,2-a][1,5] diazocin-5-yl)carbamoyl) benzo[b]thiophen-5-yl) difluoromethyl)phosphonic acid |
| 354 | 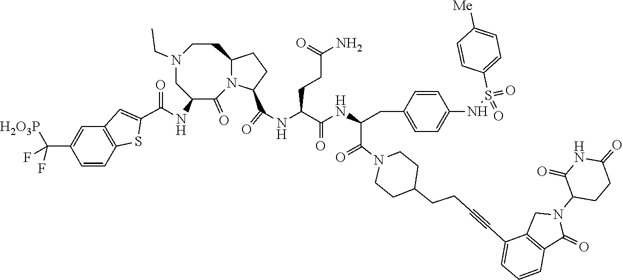 | ((2-(((5S,8S,10aR)-8-(((2S)-5- amino-1-(((2S)-1-(4-(4-(2-(2,6- dioxopiperidin-3-yl)-1-oxoisoindolin- 4-yl)but-3-yn-1-yl)piperidin-1- yl)-3-(4-((4-methylphenyl) sulfonamido)phenyl)-1-oxopropan-2- yl)amino)-1,5-dioxopentan-2-yl) carbamoyl)-3-ethyl-6- oxodecahydropyrrolo[1,2-a] [1,5]diazocin-5- yl)carbamoyl)benzo[b]thiophen-5-yl) difluoromethyl)phosphonic acid |
| 355 | 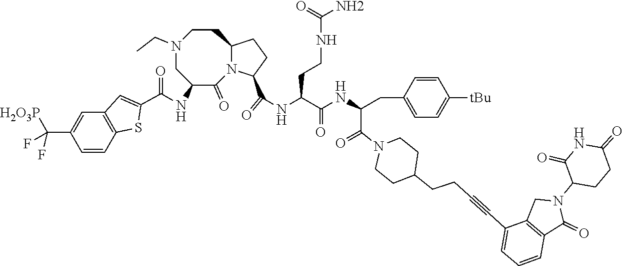 | ((2-(((5S,8S,10aR)-8-(((2S)-1- (((2S)-3-(4-(tert-butyl)phenyl)-1- (4-(4-(2-(2,6-dioxopiperidin- 3-yl)-1-oxoisoindolin-4-yl)but- 3-yn-1-yl)piperidin-1-yl)- 1-oxopropan-2-yl)amino)-1- oxo-4-ureidobutan-2-yl)carbamoyl)- 3-ethyl-6-oxodecahydropyrrolo[1,2- a][1,5]diazocin-5-yl)carbamoyl) benzo[b]thiophen-5- yl)difluoromethyl)phosphonic acid |
| 356 | 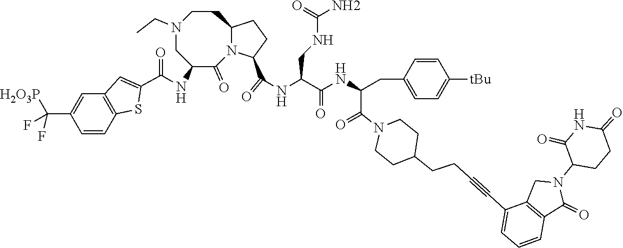 | ((2-(((5S,8S,10aR)-8-(((2S)-1- (((2S)-3-(4-(tert-butyl)phenyl)- 1-(4-(4-(2-(2,6-dioxopiperidin- 3-yl)-1-oxoisoindolin-4-yl)but- 3-yn-1-yl)piperidin-1-yl)-1- oxopropan-2-yl)amino)-1- oxo-3-ureidopropan-2-yl) carbamoyl)-3-ethyl-6- oxodecahydropyrrolo[1,2-a][1,5] diazocin-5-yl)carbamoyl) benzo[b]thiophen-5- yl)difluoromethyl)phosphonic acid |
| 357 | 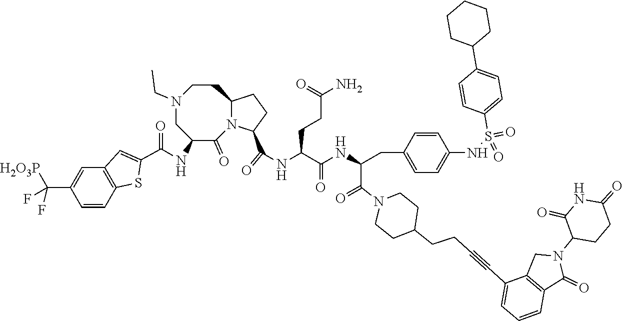 | ((2-(((5S,8S,10aR)-8-(((2S)- 5-amino-1-(((2S)-3-(4- ((4-cyclohexylphenyl)sulfonamido) phenyl)-1-(4-(4-(2-(2,6- dioxopiperidin-3-yl)-1- oxoisoindolin-4-yl)but-3- yn-1-yl)piperidin- 1-yl)-1-oxopropan-2-yl) amino)-1,5-dioxopentan- 2-yl)carbamoyl)-3-ethyl-6- oxodecahydropyrrolo[1,2-a] [1,5]diazocin-5-yl)carbamoyl) benzo[b]thiophen-5-yl) difluoromethyl)phosphonic acid |
| 358 | 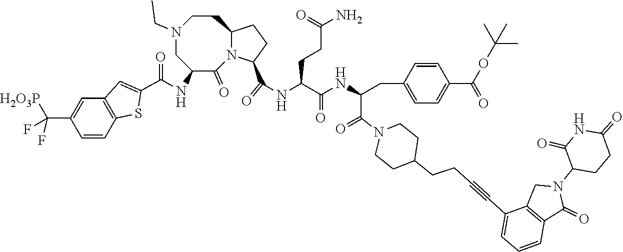 | ((2-(((5S,8S,10aR)-8-(((2S)- 5-amino-1-(((2S)-3-(4- (tert-butoxycarbonyl)phenyl)-1- (4-(4-(2-(2,6-dioxopiperidin-3-yl)-1- oxoisoindolin-4-yl)but-3-yn-1-yl) piperidin-1-yl)-1-oxopropan-2-yl) amino)-1,5-dioxopentan- 2-yl)carbamoyl)-3- ethyl-6-oxodecahydropyrrolo [1,2-a][1,5]diazocin-5- yl)carbamoyl)benzo[b] thiophen-5-yl)difluoromethyl) phosphonic acid |
| 359 | 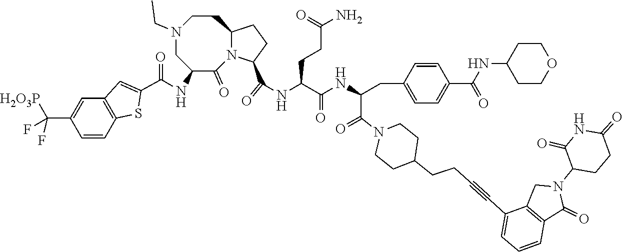 | ((2-(((5S,8S,10aR)-8-(((2S)- 5-amino-1-(((2S)-1-(4-(4- (2-(2,6-dioxopiperidin-3-yl)- 1-oxoisoindolin-4-yl)but-3- yn-1-yl)piperidin-1-yl)-1-oxo- 3-(4-((tetrahydro-2H-pyran-4- yl)carbamoyl)phenyl)propan- 2-yl)amino)-1,5-dioxopentan- 2-yl)carbamoyl)-3-ethyl-6- oxodecahydropyrrolo[1,2-a] [1,5]diazocin-5-yl)carbamoyl) benzo[b]thiophen-5-yl) difluoromethyl)phosphonic acid |
| 360 | 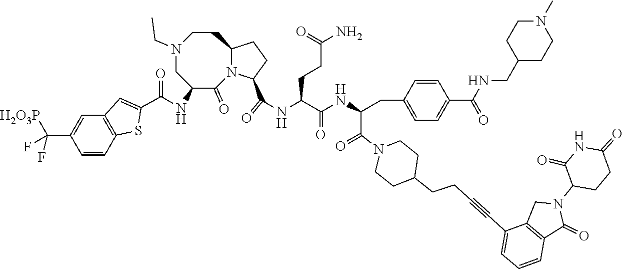 | ((2-(((5S,8S,10aR)-8-(((2S)- 5-amino-1-(((2S)-1-(4-(4-(2- (2,6-dioxopiperidin-3-yl)- 1-oxoisoindolin-4-yl)but-3- yn-1-yl)piperidin-1-yl)-3-(4- (((1-methylpiperidin-4-yl)methyl) carbamoyl)phenyl)-1-oxopropan- 2-yl)amino)-1,5-dioxopentan- 2-yl)carbamoyl)-3-ethyl- 6-oxodecahydropyrrolo[1,2-a] [1,5]diazocin-5-yl)carbamoyl) benzo[b]thiophen-5-yl) difluoromethyl)phosphonic acid |
| 361 | 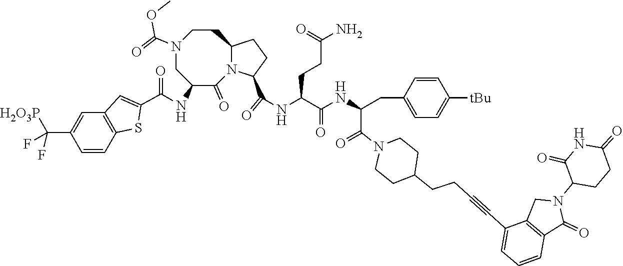 | ((2-(((5S,8S,10aR)-8-(((2S)- 5-amino-1-(((2S)-3-(4-(tert- butyl)phenyl)-1-(4-(4- (2-(2,6-dioxopiperidin-3-yl)- 1-oxoisoindolin-4-yl)but-3-yn- 1-yl)piperidin-1-yl)-1-oxopropan- 2-yl)amino)-1,5-dioxopentan- 2-yl)carbamoyl)- 3-(methoxycarbonyl)-6- oxodecahydropyrrolo[1,2-a] [1,5]diazocin-5-yl)carbamoyl) benzo[b]thiophen-5-yl) difluoromethyl)phosphonic acid |
| 362 | 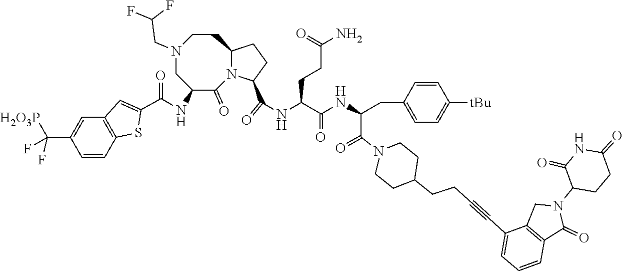 | ((2-(((5S,8S,10aR)-8-(((2S)- 5-amino-1-(((2S)-3-(4-(tert- butyl)phenyl)-1-(4-(4-(2-(2,6- dioxopiperidin-3-yl)-1- oxoisoindolin-4-yl)but-3-yn- 1-yl)piperidin-1-yl)-1-oxopropan- 2-yl)amino)-1,5-dioxopentan- 2-yl)carbamoyl)-3-(2,2- difluoroethyl)-6- oxodecahydropyrrolo[1,2-a] [1,5]diazocin-5-yl)carbamoyl) benzo[b]thiophen-5-yl) difluoromethyl)phosphonic acid |
| 363 | 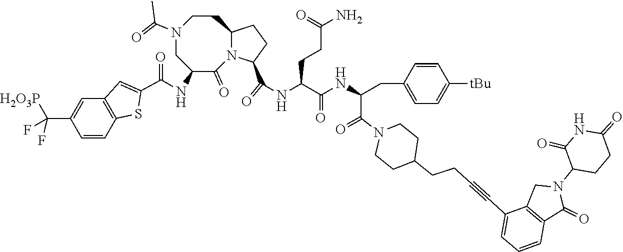 | ((2-(((5S,8S,10aR)-3-acetyl-8- (((2S)-5-amino-1-(((2S)-3-(4- (tert-butyl)phenyl)-1-(4-(4-(2- (2,6-dioxopiperidin-3-yl)- 1-oxoisoindolin-4-yl)but-3- yn-1-yl)piperidin-1-yl)-1- oxopropan-2-yl)amino)- 1,5-dioxopentan-2-yl)carbamoyl)- 6-oxodecahydropyrrolo[1,2-a] [1,5]diazocin-5-yl) carbamoyl)benzo[b]thiophen- 5-yl)difluoromethyl)phosphonic acid |
| 364 | 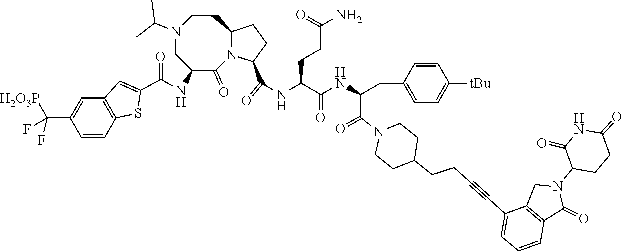 | ((2-(((5S,8S,10aR)-8-(((2S)- 5-amino-1-(((2S)-3-(4- (tert-butyl)phenyl)-1-(4-(4-(2- (2,6-dioxopiperidin-3-yl)-1- oxoisoindolin-4-yl)but-3- yn-1-yl)piperidin-1-yl)-1- oxopropan-2-yl)amino)-1,5- dioxopentan-2-yl)carbamoyl)- 3-isopropyl-6-oxodecahydropyrrolo [1,2-a][1,5]diazocin-5-yl) carbamoyl)benzo[b]thiophen- 5-yl)difluoromethyl)phosphonic acid |
| 365 | 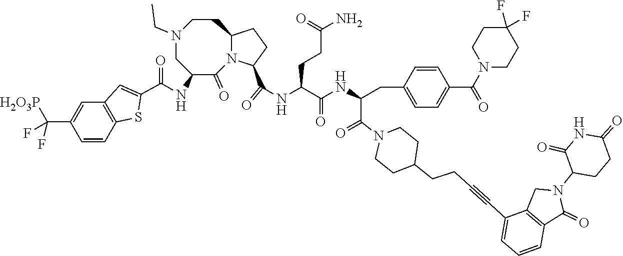 | ((2-(((5S,8S,10aR)-8-(((2S)- 5-amino-1-(((2S)-3-(4-(4,4- difluoropiperidine-1-carbonyl) phenyl)-1-(4-(4-(2-(2,6- dioxopiperidin-3-yl)-1- oxoisoindolin-4-yl)but-3- yn-1-yl)piperidin-1-yl)- 1-oxopropan-2-yl)amino)-1,5- dioxopentan-2-yl)carbamoyl)- 3-ethyl-6-oxodecahydropyrrolo [1,2-a][1,5]diazocin- 5-yl)carbamoyl)benzo[b] thiophen-5-yl)difluoromethyl) phosphonic acid |
| 366 | 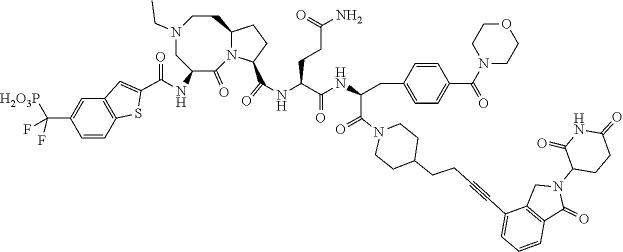 | ((2-(((5S,8S,10aR)-8-(((2S)- 5-amino-1-(((2S)-1-(4-(4-(2- (2,6-dioxopiperidin-3-yl)-1- oxoisoindolin-4-yl)but-3-yn-1- yl)piperidin-1-yl)-3-(4- (morpholine-4-carbonyl)phenyl)- 1-oxopropan-2-yl)amino)- 1,5-dioxopentan-2-yl) carbamoyl)-3-ethyl-6- oxodecahydropyrrolo[1,2-a] [1,5]diazocin-5-yl)carbamoyl) benzo[b]thiophen-5-yl) difluoromethyl)phosphonic acid |
| 367 | 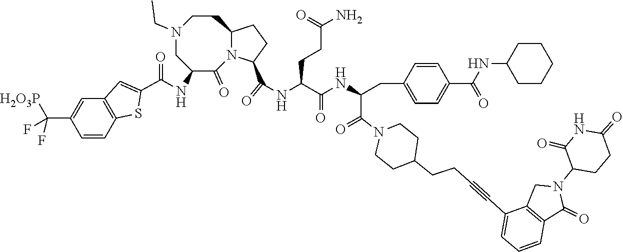 | ((2-(((5S,8S,10aR)-8-(((2S)- 5-amino-1-(((2S)-3-(4- (cyclohexylcarbamoyl)phenyl)- 1-(4-(4-(2-(2,6-dioxopiperidin- 3-yl)-1-oxoisoindolin-4-yl)but- 3-yn-1-yl)piperidin-1- yl)-1-oxopropan-2-yl)amino)- 1,5-dioxopentan-2-yl)carbamoyl)- 3-ethyl-6-oxodecahydropyrrolo [1,2-a][1,5]diazocin-5-yl) carbamoyl)benzo[b]thiophen- 5-yl)difluoromethyl) phosphonic acid |
| 368 | 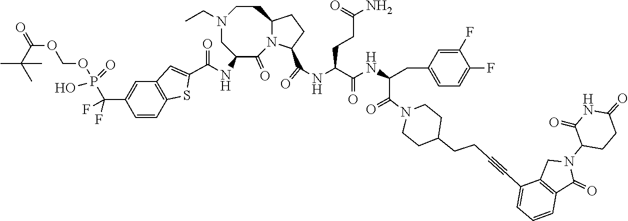 [SMILES: CCN1CC[C@H]2CC[C@@H](C(=O)N[C@@H](CCC(N)=O)C(=O)N[C@@H](Cc3ccc(F)c(F)c3)C(=O)N3CCC(CCC#Cc4cccc5c4CN(C4CCC(=O)NC4=O)C5=O)CC3)N2C(=O)[C@@H](NC(=O)c2cc3cc(C(F)(F)P(=O)(O)OCOC(=O)C(C)(C)C)ccc3s2)C1] | ((((2-(((5S,8S,10aR)-8-(((2S)- 5-amino-1-(((2S)-3-(3,4- difluorophenyl)-1-(4-(4-(2- (2,6-dioxopiperidin-3-yl)- 1-oxoisoindolin-4-yl)but- 3-yn-1-yl)piperidin-1-yl)-1- oxopropan-2-yl)amino)-1,5- dioxopentan-2-yl)carbamoyl)-3- ethyl-6-oxodecahydropyrrolo [1,2-a][1,5]diazocin-5- yl)carbamoyl)benzo[b]thiophen- 5-yl)difluoromethyl)(hydroxy) phosphoryl)oxy)methyl pivalate |
| 369 | 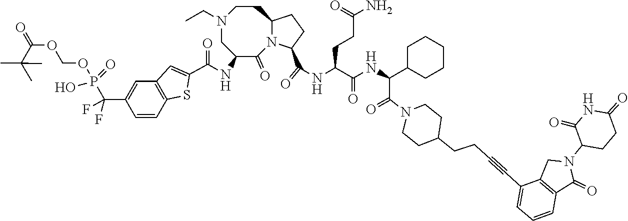 [SMILES: CCN1CC[C@H]2CC[C@@H](C(=O)N[C@@H](CCC(N)=O)C(=O)N[C@H](C(=O)N3CCC(CCC#Cc4cccc5c4CN(C4CCC(=O)NC4=O)C5=O)CC3)C3CCCCC3)N2C(=O)[C@@H](NC(=O)c2cc3cc(C(F)(F)P(=O)(O)OCOC(=O)C(C)(C)C)ccc3s2)C1] | ((((2-(((5S,8S,10aR)-8-(((2S)- 5-amino-1-(((1S)-1- cyclohexyl-2-(4-(4-(2-(2,6- dioxopiperidin-3-yl)-1- oxoisoindolin-4-yl)but- 3-yn-1-yl)piperidin-1-yl)-2- oxoethyl)amino)-1,5- dioxopentan-2-yl)carbamoyl)- 3-ethyl-6-oxodecahydropyrrolo [1,2-a][1,5]diazocin-5-yl) carbamoyl)benzo[b]thiophen- 5-yl)difluoromethyl)(hydroxy) phosphoryl)oxy)methyl pivalate |
| 370 | 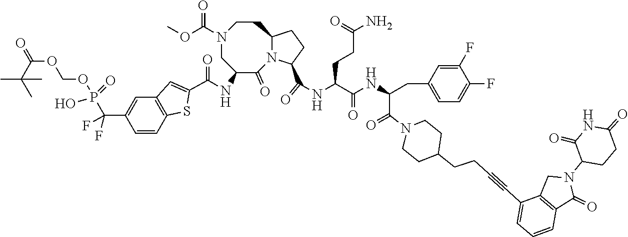 [SMILES: COC(=O)N1CC[C@H]2CC[C@@H](C(=O)N[C@@H](CCC(N)=O)C(=O)N[C@@H](Cc3ccc(F)c(F)c3)C(=O)N3CCC(CCC#Cc4cccc5c4CN(C4CCC(=O)NC4=O)C5=O)CC3)N2C(=O)[C@@H](NC(=O)c2cc3cc(C(F)(F)P(=O)(O)OCOC(=O)C(C)(C)C)ccc3s2)C1] | methyl (5S,8S,10aR)-8-(((2S)- 5-amino-1-(((2S)-3-(3,4- difluorophenyl)-1-(4-(4-(2- (2,6-dioxopiperidin-3-yl)-1- oxoisoindolin-4-yl)but-3-yn-1- yl)piperidin-1-yl)-1-oxopropan-2- yl)amino)-1,5-dioxopentan-2-yl) carbamoyl)-5-(5-(difluoro(hydroxy ((pivaloyloxy)methoxy)phosphoryl) methyl)benzo[b]thiophene-2- carboxamido)-6-oxooctahydropyrrolo [1,2-a][1,5]diazocine- 3(4H)-carboxylate |
| 394 | 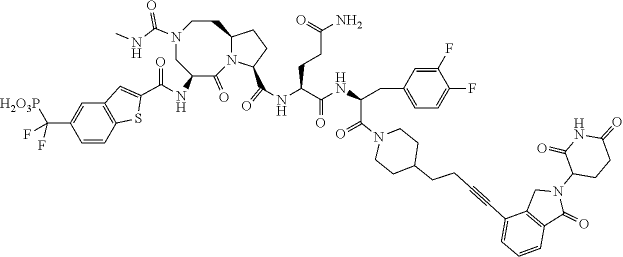 | ((2-(((5S,8S,10aR)-8-(((2S)- 5-amino-1-(((2S)-3-(3,4- difluorophenyl)-1-(4-(4-(2- (2,6-dioxopiperidin-3-yl)-1- oxoisoindolin-4-yl)but-3-yn- 1-yl)piperidin-1-yl)-1- oxopropan-2-yl)amino)-1,5- dioxopentan-2-yl)carbamoyl)- 3-(methylcarbamoyl)-6- oxodecahydropyrrolo[1,2-a] [1,5]diazocin-5-yl)carbamoyl) benzo[b]thiophen-5-yl) difluoromethyl)phosphonic acid |
| 395 | 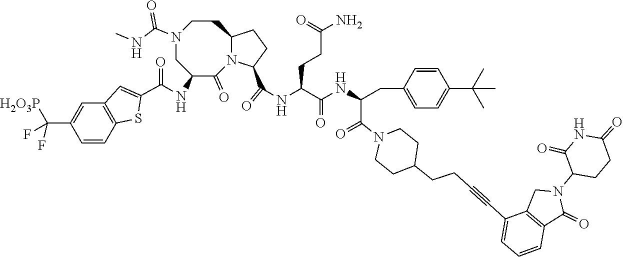 | ((2-(((5S,8S,10aR)-8-(((2S)- 5-amino-1-(((2S)-3-(4-(tert- butyl)phenyl)-1-(4-(4-(2- (2,6-dioxopiperidin-3-yl)-1- oxoisoindolin-4-yl)but- 3-yn-1-yl)piperidin-1-yl)-1- oxopropan-2-yl)amino)-1,5- dioxopentan-2-yl)carbamoyl)- 3-(methylcarbamoyl)-6- oxodecahydropyrrolo[1,2-a][1,5] diazocin-5-yl)carbamoyl) benzo[b]thiophen-5-yl) difluoromethyl)phosphonic acid |
| 396 | 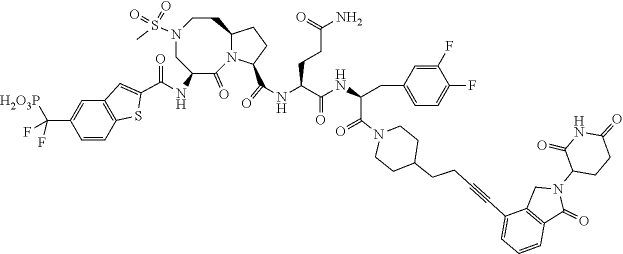 | ((2-(((5S,8S,10aR)-8-(((2S)- 5-amino-1-(((2S)-3-(3,4- difluorophenyl)-1-(4-(4-(2- (2,6-dioxopiperidin-3-yl)-1- oxoisoindolin-4-yl)but-3- yn-1-yl)piperidin-1-yl)-1- oxopropan-2-yl)amino)-1,5- dioxopentan-2-yl)carbamoyl)- 3-(methylsulfonyl)-6- oxodecahydropyrrolo[1,2-a] [1,5]diazocin-5-yl)carbamoyl) benzo[b]thiophen-5-yl) difluoromethyl)phosphonic acid |
| 397 | 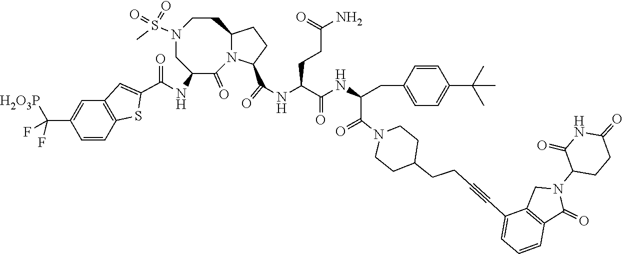 | ((2-(((5S,8S,10aR)-8-(((2S)- 5-amino-1-(((2S)-3-(4-(tert- butyl)phenyl)-1-(4-(4-(2- (2,6-dioxopiperidin-3-yl)-1- oxoisoindolin-4-yl)but-3- yn-1-yl)piperidin-1-yl)-1- oxopropan-2-yl)amino)-1,5- dioxopentan-2-yl) carbamoyl)-3-(methylsulfonyl)- 6-oxodecahydropyrrolo [1,2-a][1,5]diazocin-5- yl)carbamoyl)benzo[b]thiophen- 5-yl)difluoromethyl) phosphonic acid |

In some embodiments, the compound is selected from group consisting of:
((2-(((5S,8S,10aR)-8-(((S)-5-amino-1-(benzhydrylamino)-1,5-dioxopentan-2-yl)carbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)-1H-indol-5-yl)difluoromethyl)phosphonic acid;
(2-(((5S,8S,10aR)-8-(((S)-5-amino-1-(benzhydrylamino)-1,5-dioxopentan-2-yl)carbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)-1H-indole-5-carbonyl)phosphonic acid;
(2-(((5S,8S,10aR)-8-(((2S)-5-amino-1-(((1S)-2-(4-(4-(2-(2,6-dioxopiperidin-3-yl)-1-oxoisoindolin-4-yl)but-3-yn-1-yl)piperidin-1-yl)-2-oxo-1-phenylethyl)amino)-1,5-dioxopentan-2-yl)carbamoyl)-3-methyl-6-oxodecahydropyrrolo[1,2-a][1,5]diazocin-5-yl)carbamoyl)-1H-indole-5-carbonyl)phosphonic acid;
(2-(((5S,8S,10aR)-8-(((2S)-5-amino-1-(((1S)-1-cyclohexyl-2-(4-(4-(2-(2,6-dioxopiperidin-3-yl)-1-oxoisoindolin-4-yl)but-3-yn-1-yl)piperidin-1-yl)-2-oxoethyl)amino)-1,5-dioxopentan-2-yl)carbamoyl)-3-methyl-6-oxodecahydropyrrolo[1,2-a][1,5]diazocin-5-yl)carbamoyl)-1H-indole-5-carbonyl)phosphonic acid;
((2-(((5S,8S,10aR)-8-(((S)-5-amino-1-(benzhydrylamino)-1,5-dioxopentan-2-yl)carbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)benzo[b]thiophen-5-yl)difluoromethyl)phosphonic acid;
((2-(((5S,8S,10aR)-8-(((2S)-5-amino-1-(((1S)-1-cyclohexyl-2-(4-(4-(2-(2,6-dioxopiperidin-3-yl)-1-oxoisoindolin-4-yl)but-3-yn-1-yl)piperidin-1-yl)-2-oxoethyl)amino)-1,5-dioxopentan-2-yl)carbamoyl)-3-methyl-6-oxodecahydropyrrolo[1,2-a][1,5]diazocin-5-yl)carbamoyl)benzo[b]thiophen-5-yl)difluoromethyl)phosphonic acid;
((2-(((5S,8S,10aR)-8-(((2S)-5-amino-1-(((1S)-2-(4-(4-(2-(2,6-dioxopiperidin-3-yl)-1-oxoisoindolin-4-yl)but-3-yn-1-yl)piperidin-1-yl)-2-oxo-1-phenylethyl)amino)-1,5-dioxopentan-2-yl)carbamoyl)-3-methyl-6-oxodecahydropyrrolo[1,2-a][1,5]diazocin-5-yl)carbamoyl)benzo[b]thiophen-5-yl)difluoromethyl)phosphonic acid
(2-(((5S,8S,10aR)-8-(((S)-5-amino-1-(benzhydrylamino)-1,5-dioxopentan-2-yl)carbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)benzo[b]thiophene-5-carbonyl)phosphonic acid;
(2-(((5S,8S,10aR)-8-(((2S)-5-amino-1-(((1S)-1-cyclohexyl-2-(4-(4-(2-(2,6-dioxopiperidin-3-yl)-1-oxoisoindolin-4-yl)but-3-yn-1-yl)piperidin-1-yl)-2-oxoethyl)amino)-1,5-dioxopentan-2-yl)carbamoyl)-3-methyl-6-oxodecahydropyrrolo[1,2-a][1,5]diazocin-5-yl)carbamoyl)benzo[b]thiophene-5-carbonyl)phosphonic acid; and
(2-(((5S,8S,10aR)-8-(((2S)-5-amino-1-(((1S)-2-(4-(4-(2-(2,6-dioxopiperidin-3-yl)-1-oxoisoindolin-4-yl)but-3-yn-1-yl)piperidin-1-yl)-2-oxo-1-phenylethyl)amino)-1,5-dioxopentan-2-yl)carbamoyl)-3-methyl-6-oxodecahydropyrrolo[1,2-a][1,5]diazocin-5-yl)carbamoyl)benzo[b]thiophene-5-carbonyl)phosphonic acid,
   and pharmaceutically acceptable salts and solvates thereof.

In some embodiments, the compound is selected from group consisting of:
((2-(((5S,8S,10aR)-8-(((2S)-5-amino-1-(((2S)-3-(3,4-difluorophenyl)-1-(4-(4-(2-(2,6-dioxopiperidin-3-yl)-1-oxoisoindolin-4-yl)but-3-yn-1-yl)piperidin-1-yl)-1-oxopropan-2-yl)amino)-1,5-dioxopentan-2-yl)carbamoyl)-3-(methoxycarbonyl)-6-oxodecahydropyrrolo[1,2-a][1,5]diazocin-5-yl)carbamoyl)benzo[b]thiophen-5-yl)difluoromethyl)phosphonic acid; and
((2-(((5S,8S,10aR)-8-(((2S)-5-amino-1-(((1S)-1-cyclohexyl-2-(4-(4-(2-(2,6-dioxopiperidin-3-yl)-1-oxoisoindolin-4-yl)but-3-yn-1-yl)piperidin-1-yl)-2-oxoethyl)amino)-1,5-dioxopentan-2-yl)carbamoyl)-3-(methoxycarbonyl)-6-oxodecahydropyrrolo[1,2-a][1,5]diazocin-5-yl)carbamoyl)benzo[b]thiophen-5-yl)difluoromethyl)phosphonic acid, and pharmaceutically acceptable salts and solvates thereof.

In some embodiments, the compound is ((2-(((5S,8S,10aR)-8-(((2S)-5-amino-1-(((2S)-3-(3,4-difluorophenyl)-1-(4-(4-(2-(2,6-dioxopiperidin-3-yl)-1-oxoisoindolin-4-yl)but-3-yn-1-yl)piperidin-1-yl)-1-oxopropan-2-yl)amino)-1,5-dioxopentan-2-yl)carbamoyl)-3-(methoxycarbonyl)-6-oxodecahydropyrrolo[1,2-a][1,5]diazocin-5-yl)carbamoyl)benzo[b]thiophen-5-yl)difluoromethyl)phosphonic acid, or a pharmaceutically acceptable salt thereof.

In some embodiments, the compound is:

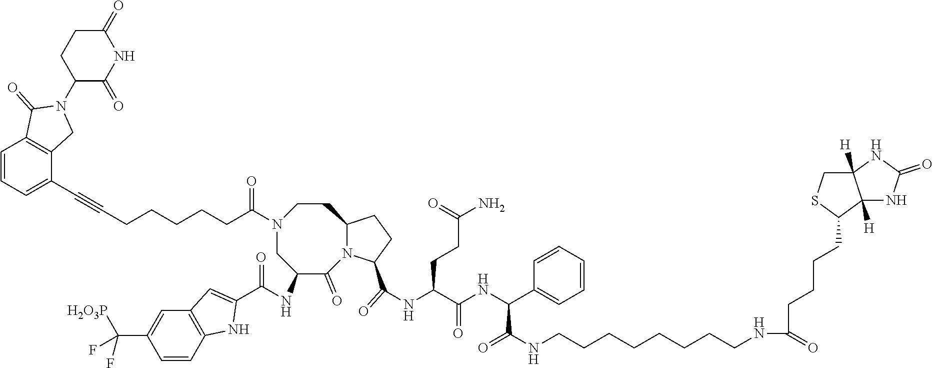

(chemical name: ((2-(((5S,8S,10aR)-8-(((S)-5-amino-1,5-dioxo-1-(((S)-2-oxo-2-((8-(5-((3aS,4S,6aR)-2-oxohexahydro-1H-thieno[3,4-d]imidazol-4-yl)pentanamido)octyl)amino)-1-phenylethyl)amino)pentan-2-yl)carbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)-1H-indol-5-yl)difluoromethyl)phosphonic acid).

In some embodiments, the compound is:

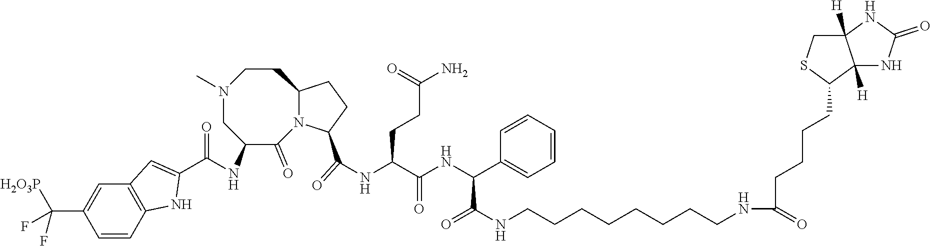

(chemical name: ((2-(((5S,8S,10aR)-8-(((S)-5-amino-1,5-dioxo-1-(((S)-2-oxo-2-((8-(5-((3aS,4S,6aR)-2-oxohexahydro-1H-thieno[3,4-d]imidazol-4-yl)pentanamido)octyl)amino)-1-phenylethyl)amino)pentan-2-yl)carbamoyl)-3-methyl-6-oxodecahydropyrrolo[1,2-a][1,5]diazocin-5-yl)carbamoyl)-1H-indol-5-yl)difluoromethyl)phosphonic acid).

In some embodiments, the compound is:

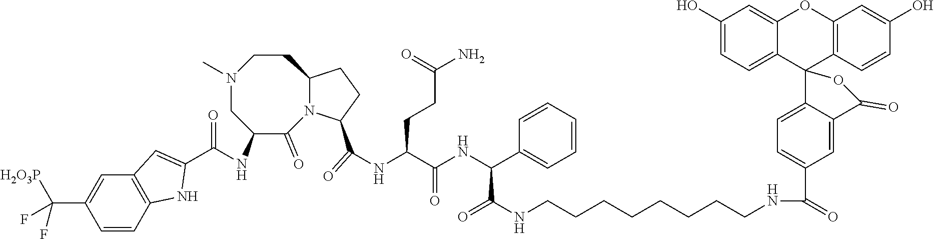

(chemical name: ((2-(((5S,8S,10aR)-8-(((S)-5-amino-1-(((S)-2-((8-(3′,6′-dihydroxy-3-oxo-3H-spiro[isobenzofuran-1,9′-xanthene]-5-carboxamido)octyl)amino)-2-oxo-1-phenylethyl)amino)-1,5-dioxopentan-2-yl)carbamoyl)-3-methyl-6-oxodecahydropyrrolo[1,2-a][1,5]diazocin-5-yl)carbamoyl)-1H-indol-5-yl)difluoromethyl)phosphonic acid).

In some embodiments, the compound is of Formula (VIII):

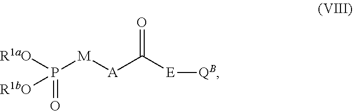

or a pharmaceutically acceptable salt or solvate thereof, wherein:

R^{1a} and R^{1b} are independently selected from the group consisting of hydrogen, C₁-C₆ alkyl, and aralkyl;

R^{2a}, R^{2b}, M, A, and E are as defined in connection with Formula I, except that in A the bond designated with an “*” is attached to —C(═O)-E-Q^{B} and that in E the bond designated with an “*” is attached to Q^{B};

R⁸ is selected from the group consisting of hydrogen, C₁-C₆ alkyl, C₁-C₆ haloalkyl, (heterocyclo)alkyl, alkylsulfonyl, and —C(═O)R⁹; and

Q^{B} is selected from the group consisting of Q-1 and Q-2.

In some embodiments, the compound is of Formula (VIII) or a pharmaceutically acceptable salt or solvate thereof, wherein:

(1) when X_{A} is —CH₂CH₂—, then:

(i) A is selected from the group consisting of A-2, A-5, A-6, A-7, A-8, A-9, A-10, A-11, A-12, A-13, A-14, A-15, A-16, A-17, A-18, A-19, and A-20;

(ii) A is A-4 and G¹ is —S—; or

(iii) R^{2a} and R^{2b} taken together with the carbon atom to which they are attached form a —C(═O)— group; (2) when X_{A} is —N(R⁸)CH₂—, then:

(i) A is selected from the group consisting of A-1, A-2, A-4, A-5, A-6, A-7, A-8, A-9, A-10, A-11, A-12, A-13, A-14, A-15, A-16, A-17, A-18, A-19, and A-20; or

(ii) R^{2a} and R^{2b} taken together with the carbon atom to which they are attached form a —C(═O)— group; or (3) when X_{A} is —CH₂N(R⁸)—, then:

(i) A is selected from the group consisting of A-1, A-2, A-4, A-5, A-6, A-7, A-8, A-9, A-10, A-11, A-12, A-13, A-14, A-15, A-16, A-17, A-18, A-19, and A-20; or

(ii) R^{2a} and R^{2b} taken together with the carbon atom to which they are attached form a —C(═O)— group.

In some embodiments, the compound is of Formula (VIII) or a pharmaceutically acceptable salt or solvate thereof, wherein M is —O—.

In some embodiments, the compound is of Formula (VIII) or a pharmaceutically acceptable salt or solvate thereof, wherein M is —C(R^{2a})(R^{2b})—.

In some embodiments, the compound is of Formula (IX):

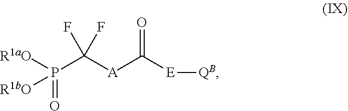

or a pharmaceutically acceptable salt or solvate thereof.

In some embodiments, the compound is of Formula (X):

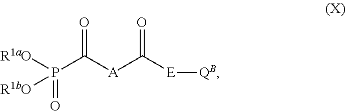

or a pharmaceutically acceptable salt or solvate thereof.

In some embodiments, Q^{B} is Q-1, or a pharmaceutically acceptable salt or solvate thereof. In some embodiments, E is E-1-1. In some embodiments, E is E-2-1. In some embodiments, E is E-3-1. In some embodiments, X¹ is selected from the group consisting of —CH₂— and —N(H)—. In some embodiments, X¹ is —CH₂—. In some embodiments, s is 0 or 1. In some embodiments, s is 0.

In some embodiments, Q^{B} is Q-2, or a pharmaceutically acceptable salt or solvate thereof. In some embodiments, E is E-1-1. In some embodiments, E is E-2-1. In some embodiments, E is E-3-1. In some embodiments, Q-2 is Q-2-1 (wherein Q-2-1 is as defined in connection with Formula I). In some embodiments, X² is selected from the group consisting of —CH₂—, —O—, and —N(H)—. In some embodiments, X² is —CH₂—. In some embodiments, t is 0 or 1. In some embodiments, t is 0. In some embodiments, R^{12b} is hydrogen. In some embodiments, R^{12a} is aralkyl.

In some embodiments, the compound is of Formula (XI):

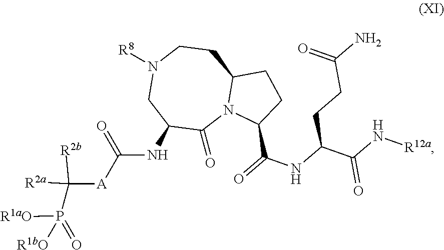

or a pharmaceutically acceptable salt or solvate thereof.

In some embodiments, the compound is of Formula (XII):

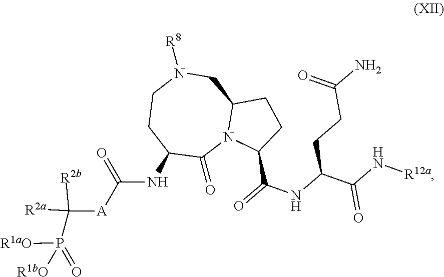

or a pharmaceutically acceptable salt or solvate thereof.

In some embodiments, R^{2a} and R^{2b} are fluoro.

In some embodiments, R^{2a} and R^{2b} taken together with the carbon to which they are attached form a —C(═O)— group.

In some embodiments, A is A-4. In some embodiments, G¹ is —S—.

In some embodiments, A is A-8. In some embodiments, G, G⁴, and G⁶ are —C(H)═; and R³ is hydrogen.

In some embodiments, R⁸ is hydrogen.

In some embodiments, R⁸ is C₁-C₄ alkyl.

In some embodiments, R^{12a} is aralkyl.

In some embodiments, R^{12a} is:

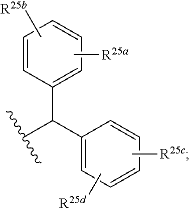

wherein R^{25a}, R^{25b}, R^{25c}, and R^{25d} are each independently selected from the group consisting of hydrogen, halo, C₁-C₄ alkyl, and C₁-C₄ alkoxy.

In some embodiments, R^{1a} and R^{1b} are C₁-C₄ alkyl.

In some embodiments, R^{1a} and R^{1b} hydrogen.

In some embodiments, the compound is selected from the compounds described in Table 2, and pharmaceutically acceptable salts and solvates thereof.

TABLE 2

| Cpd. No. | Structure |
|---|---|
| 200 | 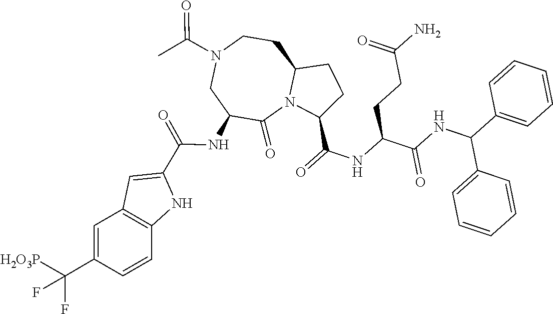 |
| 201 | 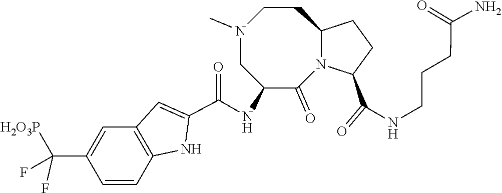 |
| 202 | 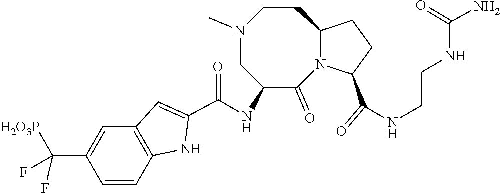 |
| 203 | 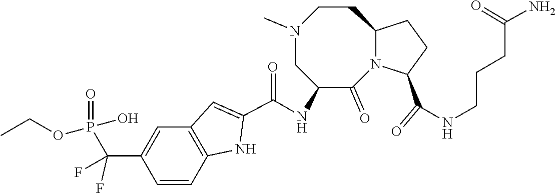 [SMILES: CCOP(=O)(O)C(F)(F)c1ccc2[nH]c(C(=O)N[C@H]3CN(C)CC[C@H]4CC[C@@H](C(=O)NCCCC(N)=O)N4C3=O)cc2c1] |
| 204 | 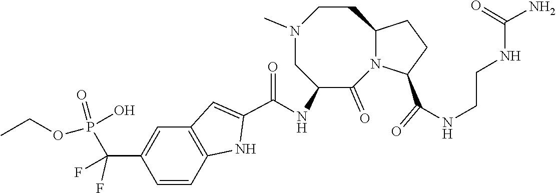 [SMILES: CCOP(=O)(O)C(F)(F)c1ccc2[nH]c(C(=O)N[C@H]3CN(C)CC[C@H]4CC[C@@H](C(=O)NCCNC(N)=O)N4C3=O)cc2c1] |
| 205 | 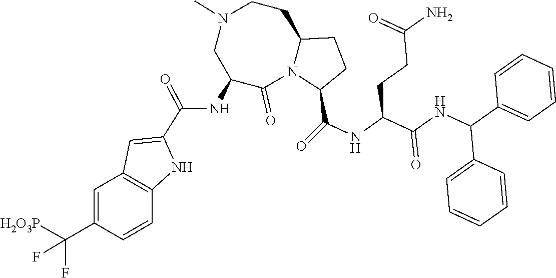 |
| 206 | 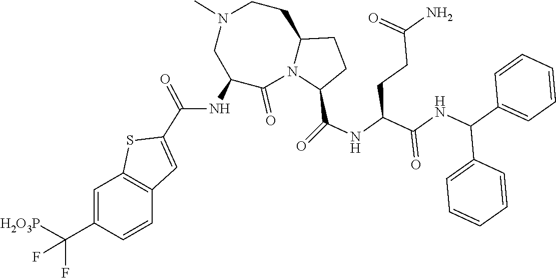 |
| 207 | 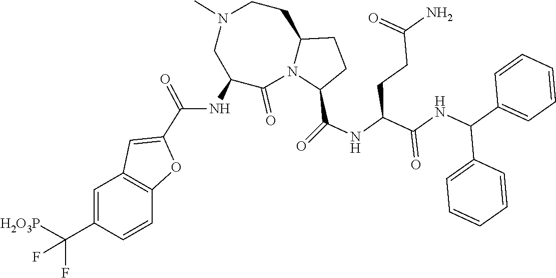 |
| 208 | 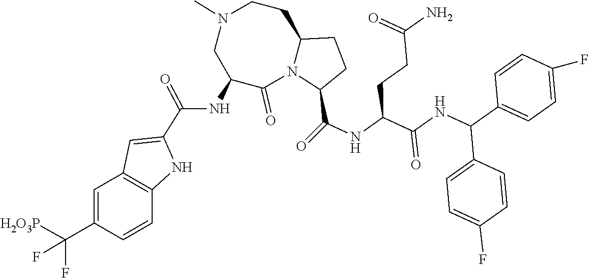 |
| 209 | 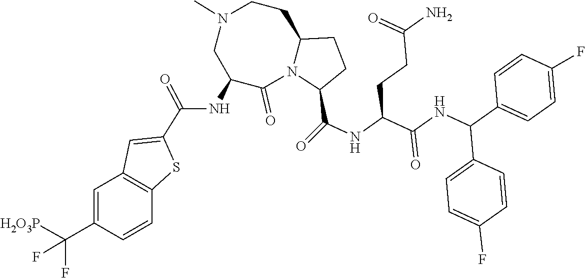 |
| 210 | 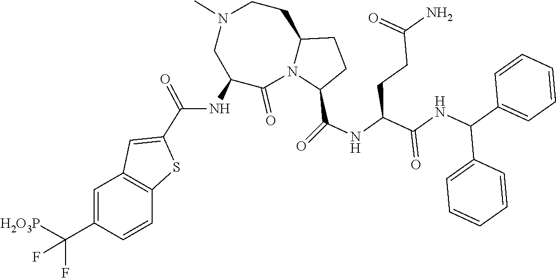 |
| 211 | 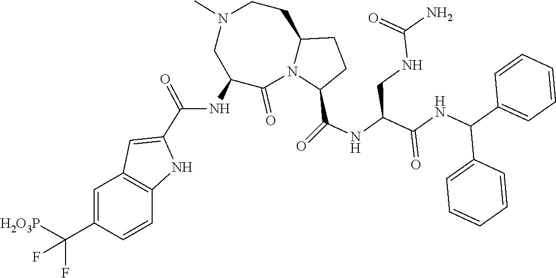 |
| 212 | 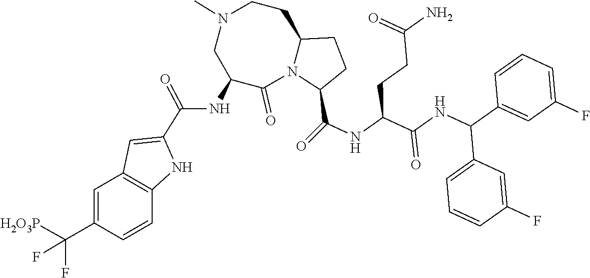 |
| 213 | 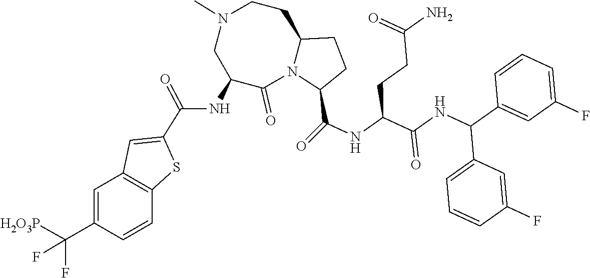 |
| 214 | 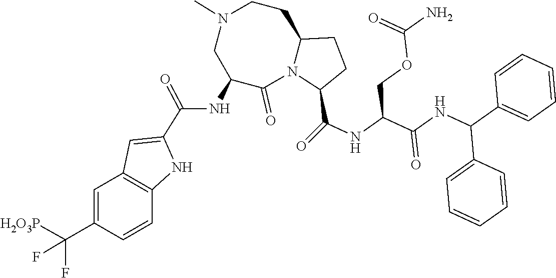 |
| 215 | 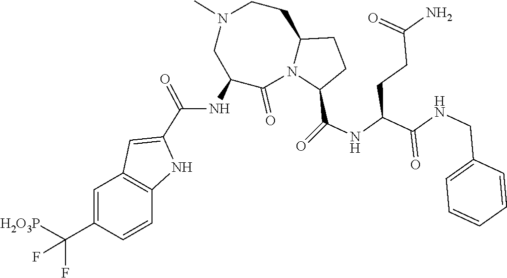 |
| 216 | 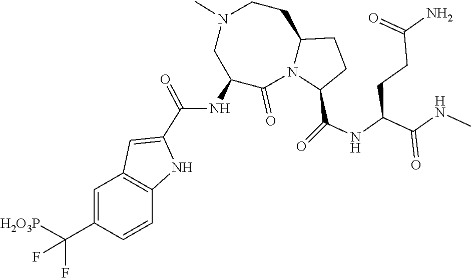 |
| 217 | 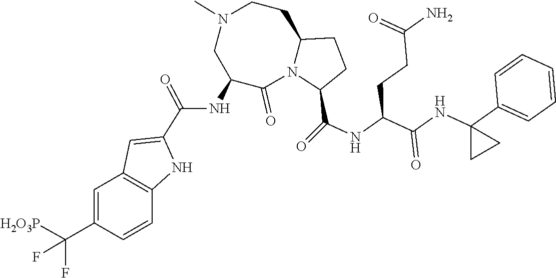 |
| 218 | 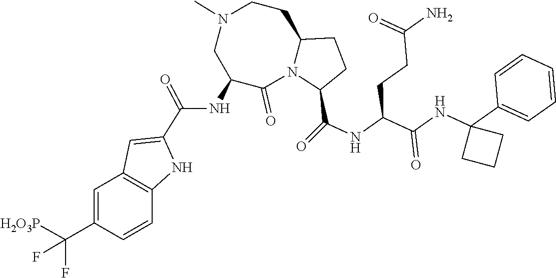 |
| 219 | 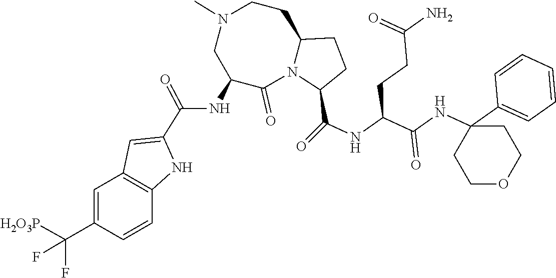 |
| 220 | 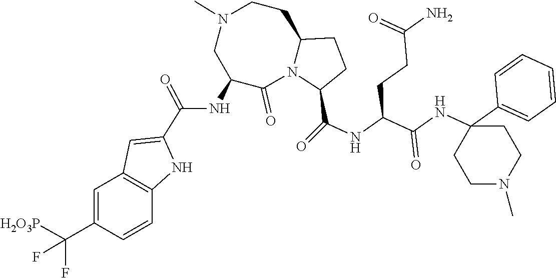 |
| 221 | 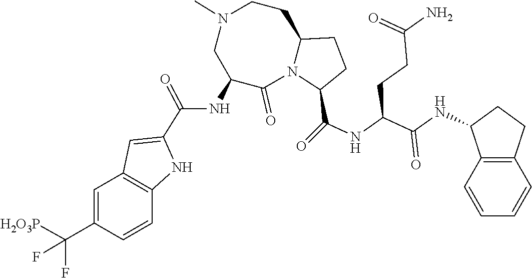 |
| 222 | 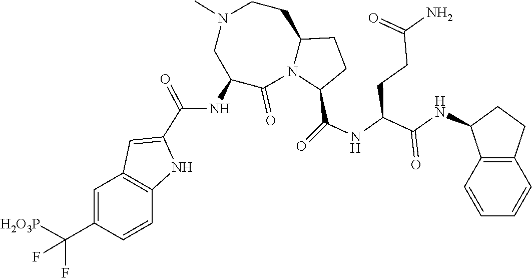 |
| 223 | 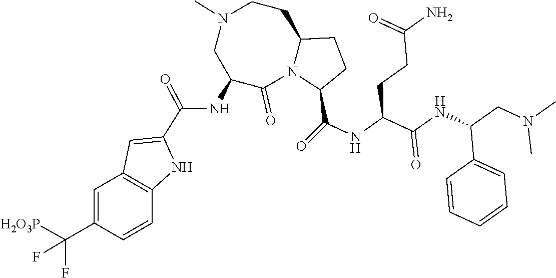 |
| 224 | 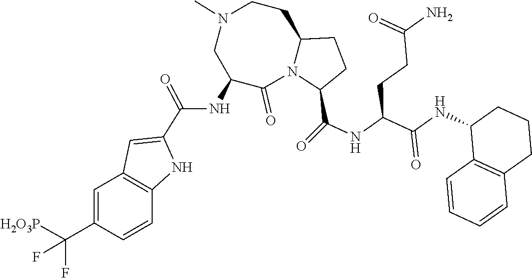 |
| 226 | 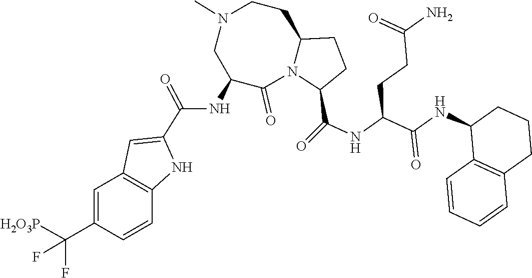 |
| 227 | 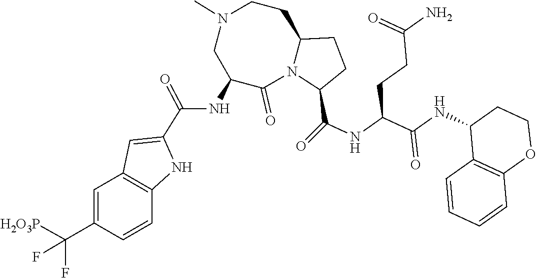 |
| 228 | 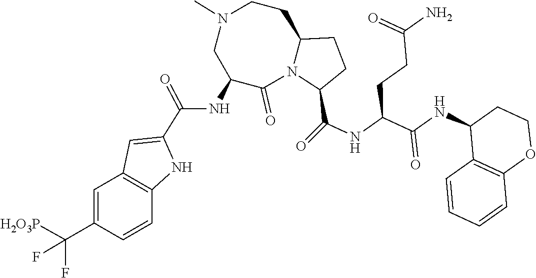 |
| 229 | 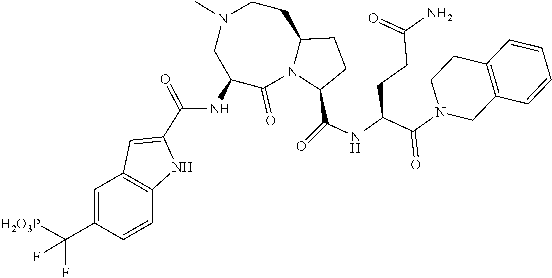 |
| 230 | 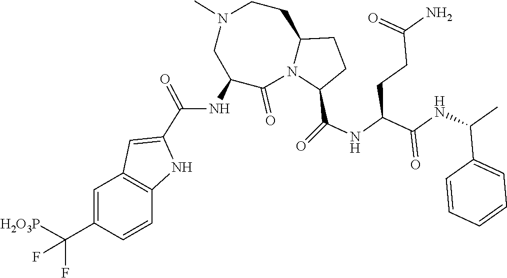 |
| 231 | 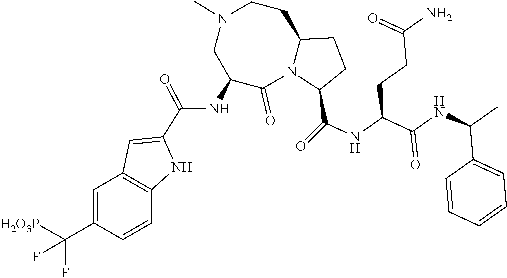 |
| 232 | 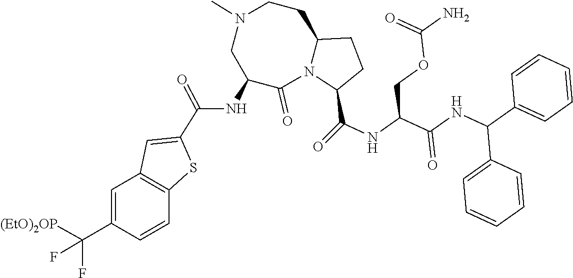 |
| 233 | 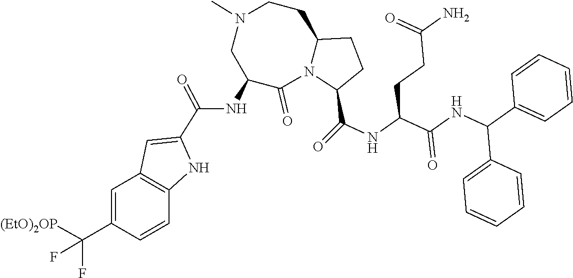 |
| 234 | 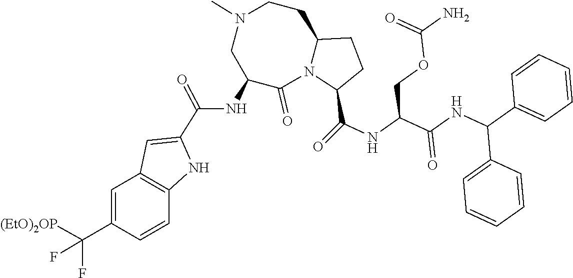 |
| 235 | 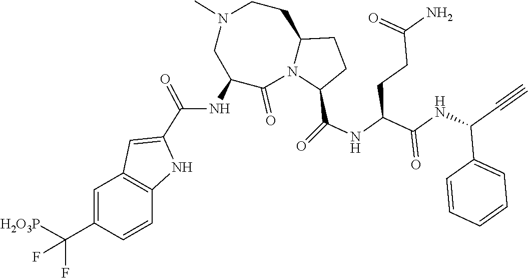 |
| 236 | 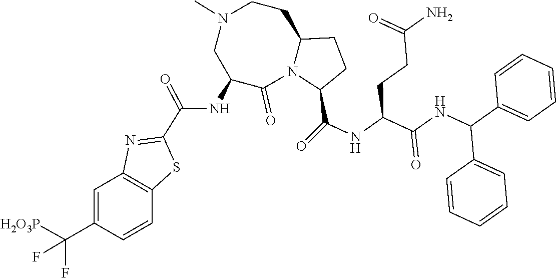 |
| 237 | 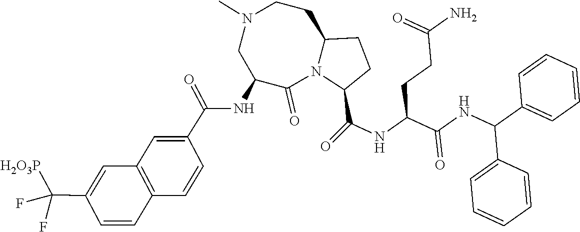 |
| 238 | 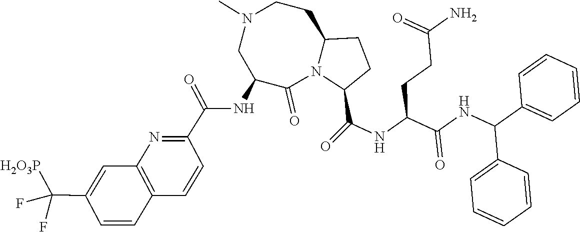 |
| 239 | 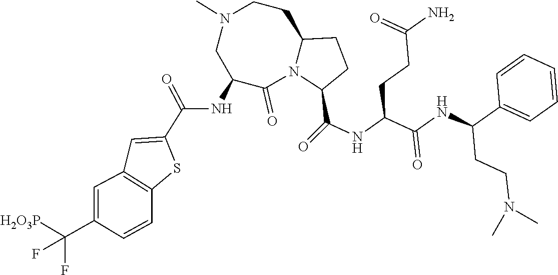 |
| 240 | 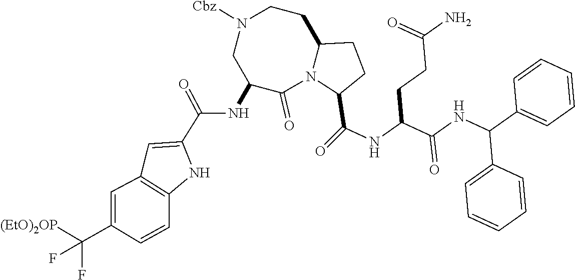 |
| 241 | 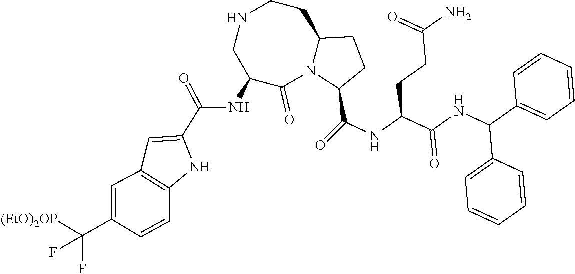 |
| 242 | 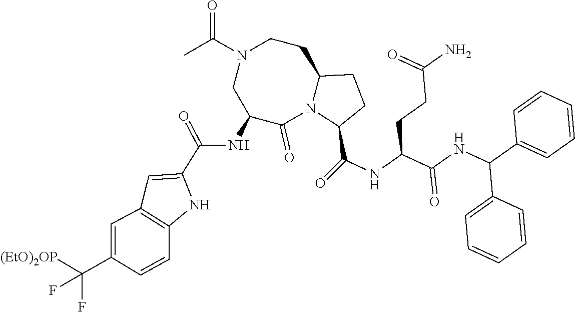 |

In some embodiments, the compound is enantiomerically enriched, e.g., the enantiomeric excess or “ee” of the compound is about 5% or more as measured by chiral HPLC. In some embodiments, the ee is about 10%. In some embodiments, the ee is about 20%. In some embodiments, the ee is about 30%. In some embodiments, the ee is about 40%. In some embodiments, the ee is about 50%. In some embodiments, the ee is about 60%. In some embodiments, the ee is about 70%. In some embodiments, the ee is about 80%. In some embodiments, the ee is about 85%. In some embodiments, the ee is about 90%. In some embodiments, the ee is about 91%. In some embodiments, the ee is about 92%. In some embodiments, the ee is about 93%. In some embodiments, the ee is about 94%. In some embodiments, the ee is about 95%. In some embodiments, the ee is about 96%. In some embodiments, the ee is about 97%. In some embodiments, the ee is about 98%. In some embodiments, the ee is about 99%.

In some embodiments, the compound is a heterobifunctional molecule. In some embodiments, the E portion of the compound

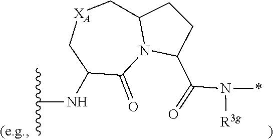

is enantiomerically enriched, e.g., the enantiomeric excess or “ee” of this part of the heterobifunctional compound is about 5% or more as measured by chiral HPLC. In some embodiments, the ee is about 10%. In some embodiments, the ee is about 20%. In some embodiments, the ee is about 30%. In some embodiments, the ee is about 40%. In some embodiments, the ee is about 50%. In some embodiments, the ee is about 60%. In some embodiments, the ee is about 70%. In some embodiments, the ee is about 80%. In some embodiments, the ee is about 85%. In some embodiments, the ee is about 90%. In some embodiments, the ee is about 91%. In some embodiments, the ee is about 92%. In some embodiments, the ee is about 93%. In some embodiments, the ee is about 94%. In some embodiments, the ee is about 95%. In some embodiments, the ee is about 96%. In some embodiments, the ee is about 97%. In some embodiments, the ee is about 98%. In some embodiments, the ee is about 99%.

In some embodiments, the cereblon binding portion of the compound (e.g., —B) is enantiomerically enriched. In some embodiments, the cereblon binding portion of the compound is racemic. The present disclosure encompasses all possible stereoisomeric, e.g., diastereomeric, forms of the compound of disclosed herein. For example, all possible stereoisomers are encompassed when E portion of the compound is enantiomerically enriched and the cereblon binding portion of the compound is racemic.

As used herein, the pharmaceutical “pharmaceutically acceptable salt” refers to salts or zwitterionic forms of the compound. Salts of the compound can be prepared during the final isolation and purification of the compounds or separately by reacting the compound with a suitable acid. The pharmaceutically acceptable salts of the compound can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Non-limiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerophosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference compound appearing herein is intended to include the compound as well as pharmaceutically acceptable salts, hydrates, or solvates thereof.

Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. The compound can be present as solvated forms with a pharmaceutically acceptable solvent, such as water, methanol, and ethanol, and it is intended that the disclosure includes both solvated and unsolvated forms of the compound. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1):Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvate in a crystal of the solvate.

It is understood that the compound of the present disclosure may be synthesized, insulated, and/or purified using techniques well known in the art. For examples, one or more of the compounds may be synthesized following the procedures described in PCT Appl'n Pub. No. WO 2020/198435 (incorporate herein by reference).

### Biological Assays

Compounds designed, selected and/or optimized, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays are may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological assay is described in the Examples herein.

In some embodiments, the effect of the compounds may be determined utilizing an acute GVHD model.

In some embodiments, the effect of the compounds on may be determined utilizing an acute GVHD model challenged with A20 tumor cells.

In some embodiments, the effect of the compounds may be determined utilizing an in vitro intestinal organoids culture.

### Pharmaceutical Compositions and Kits

In some aspects, the present disclosure provides a pharmaceutical composition comprising a compound disclosed herein.

In some aspects, the present disclosure provides a pharmaceutical composition for treating or preventing GVHD, comprising a compound disclosed herein.

In some aspects, the present disclosure provides a pharmaceutical kit comprising a compound disclosed herein.

In some aspects, the present disclosure provides a pharmaceutical kit for treating or preventing GVHD, comprising a compound disclosed herein.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease and also preferably causing complete regression of the disease.

It is understood that the pharmaceutical compositions can be included in a container, pack, or dispenser together with instructions for administration.

### Definitions

The term “STAT3” refers to a protein encoded by the STAT3 gene. STAT3 is a member of the STAT protein family. In response to cytokines and growth factors, STAT3 is phosphorylated by receptor-associated Janus kinases (JAK), form homo- or heterodimers, and translocate to the cell nucleus where they act as transcription activators.

The term “STAT1” refers to a protein encoded by the STAT1 gene. STAT1 is a member of the STAT protein family. STAT1 plays a role in many gene expressions that cause survival of the cell, viability or pathogen response.

The term “STAT3 inhibitor” or “STAT3 protein inhibitor” and the like refers to a Compound of the Disclosure that inhibits STAT3 protein. STAT3 inhibitors typically have a half maximal inhibitory concentration (IC₅₀) for inhibiting STAT3 of less than about 100 μM, e.g., less than about 50 μM, less than about 25 μM, and less than 5 μM, less than about 1 μM, less than about 0.5 μM, less than about 0.1 μM, less than about 0.05 μM, or less than about 0.01 μM. STAT3 inhibitors can be used as synthetic intermediates to prepare Compounds of the Disclosure that degrade STAT3. Representative Compounds of the Disclosure that inhibit STAT3 are disclosed in Table 2.

The term “STAT3 degrader” or “STAT3 protein degrader” and the like refer to a Compound of the Disclosure that degrades STAT3 protein. STAT3 degraders are heterobifunctional small molecules containing a first ligand which binds to STAT3 protein, a second ligand for an E3 ligase system, and a chemical linker that tethers the first and second ligands. Representative Compounds of the Disclosure that degrade STAT3 are disclosed in Tables 1 1A, and IB. Likewise, a “STAT1” degrader and the like refer to a Compound of the Disclosure that degrades STAT1 protein. Compounds of the Disclosure may preferentially degrade STAT3, or preferentially degrade STAT1, or degrade both STAT3 and STAT1.

The term “second therapeutic agent” refers to a therapeutic agent different from a Compound of the Disclosure and that is known to treat the disease or condition of interest.

The term “disease” or “condition” denotes disturbances and/or anomalies that as a rule are regarded as being pathological conditions or functions, and that can manifest themselves in the form of particular signs, symptoms, and/or malfunctions. Compounds of the Disclosure are inhibitors or degraders of STAT3 and can be used in treating or preventing diseases and conditions wherein inhibition or degradation of STAT3 provides a benefit. Compounds of the Disclosure may also degrade STAT3 and STAT1 and thus can be used in treating or preventing diseases and conditions wherein degradation of both STAT3 and STAT1 provides a benefit.

The term “Graft-versus-host disease” or “GVHD” refers to the condition that occurs in a subject following an allogeneic or autologous transplant (e.g. a HCT) in which immune cells presented in the allogeneic or autologous transplant material (referred to as the “graft”) attack the transplant recipient's own tissues. It is understood that the terms “Graft-versus-host disease” or “Graft versus Host Disease” or “GVHD” or “GvHD” can be used interchangeably.

The terms “acute Graft versus Host Disease”, “acute GVHD”, “classic acute Graft versus Host Disease” and “classic acute GVHD” refer to Graft versus Host Disease that develops in a transplant recipient within about 100 days following transplantation and manifests with clinical features that are typically associated with acute Graft versus Host Disease, including, but not limited to inflammation and tissue damage in the skin, oral and genital mucosa, eyes, gut, liver, lungs, joints, and muscle.

The terms “late acute Graft versus Host Disease” or “late acute GVHD” refer to the conditions of persistent acute Graft versus Host Disease, recurrent acute Graft versus Host Disease, and/or new-onset acute Graft versus Host Disease, which are forms of Graft versus Host Disease that manifest with the clinical features of acute Graft versus Host Disease, but more than 100 days after the transplantation.

The term “chronic Graft versus Host Disease” or “chronic GVHD” refers to Graft versus Host Disease that develop more than 100 days after the transplantation and manifests with clinical features that are typically associated with chronic Graft versus Host Disease, including, but not limited to, damage to connective tissue of exocrine glands, tissue fibrosis and limitation of joint motility, fibrosis of the lungs and liver, immune dysregulation and autoimmunity.

The term “graft-versus-leukemia” or “GVL” refers to the effect that results in a subject that results in reduced risk of underlying disease relapse (e.g. cancer, a blood cancer).

The term “allogeneic” refers to biological material isolated from a donor that is to be transplanted into a recipient, wherein the donor and the recipient are two different subjects.

The term “autologous” refers to biological material isolated from a donor that is to be transplanted into a recipient, wherein the donor and the recipient is the same subject.

The term “therapeutically effective amount” or “effective dose” as used herein refers to an amount of the active ingredient(s) that is(are) sufficient, when administered by a method of the disclosure, to efficaciously deliver the active ingredient(s) for the treatment of condition or disease of interest to a subject in need thereof. In the case of a cancer or other proliferation disorder, the therapeutically effective amount of the agent may reduce (i.e., retard to some extent or stop) unwanted cellular proliferation; reduce the number of cancer cells; reduce the tumor size; inhibit (i.e., retard to some extent or stop) cancer cell infiltration into peripheral organs; inhibit (i.e., retard to some extent or stop) tumor metastasis; inhibit, to some extent, tumor growth; and/or relieve, to some extent, one or more of the symptoms associated with the cancer. To the extent the administered compound or composition prevents growth and/or kills existing cancer cells, it may be cytostatic and/or cytotoxic.

As used herein, the term “therapeutically effective amount”, refers to an amount of a pharmaceutical agent to treat or ameliorate an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

“Concurrent administration,” “administered in combination,” “simultaneous administration,” (e.g., at the same time as, in temporal proximity to, or in overlapping time periods with the administration of) and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to a subject in a sequence and sufficiently close in time so as to provide the desired therapeutic effect and can act in concert. For example, a Compound of the Disclosure can be administered at the same time or sequentially in any order at different points in time as a second therapeutic agent. A Compound of the Disclosure and the second therapeutic agent can be administered separately, in any appropriate form and by any suitable route. When a Compound of the Disclosure and the second therapeutic agent are not administered concurrently, it is understood that they can be administered in any order to a subject in need thereof. For example, a Compound of the Disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent treatment modality (e.g., radiotherapy), to a subject in need thereof. In various embodiments, a Compound of the Disclosure and the second therapeutic agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the components of the combination therapies are administered at about 1 minute to about 24 hours apart.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “halo” as used herein by itself or as part of another group refers to —Cl, —F, —Br, or —I.

The term “nitro” as used herein by itself or as part of another group refers to —NO₂.

The term “cyano” as used herein by itself or as part of another group refers to —CN.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In one embodiment, the alkyl is a C₁-C₁₀ alkyl. In some embodiments, the alkyl is a C₁-C₆ alkyl. In some embodiments, the alkyl is a C₁-C₄ alkyl. In some embodiments, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “optionally substituted alkyl” as used herein by itself or as part of another group refers to an alkyl group that is either unsubstituted or substituted with one, two, or three substituents, wherein each substituent is independently nitro, haloalkoxy, aryloxy, aralkyloxy, alkylthio, sulfonamido, alkylcarbonyl, arylcarbonyl, alkylsulfonyl, arylsulfonyl, ureido, guanidino, carbamate, carboxy, alkoxycarbonyl, carboxyalkyl, —N(R^{56a})C(═O)R^{56b}, —N(R^{56c})S(═O)₂R^{56d}, —C(═O)R⁵⁷, —S(═O)R^{56e}, or —S(═O)₂R⁵⁸; wherein:

R^{56a} is hydrogen or alkyl;

R^{56b} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;

R^{56c} is hydrogen or alkyl;

R^{56d} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;

R^{56e} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;

R⁵⁷ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl; and

R⁵⁸ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl. Non-limiting exemplary optionally substituted alkyl groups include —CH(CO₂Me)CH₂CO₂Me and —CH(CH₃)CH₂N(H)C(═O)O(CH₃)₃.

The term “alkenyl” as used herein by itself or as part of another group refers to an alkyl group containing one, two, or three carbon-to-carbon double bonds. In one embodiment, the alkenyl group is a C₂-C₆ alkenyl group. In some embodiments, the alkenyl group is a C₂-C₄ alkenyl group. In some embodiments, the alkenyl group has one carbon-to-carbon double bond. Non-limiting exemplary alkenyl groups include ethenyl, propenyl, isopropenyl, butenyl, sec-butenyl, pentenyl, and hexenyl.

The term “optionally substituted alkenyl” as used herein by itself or as part of another refers to an alkenyl group that is either unsubstituted or substituted with one, two or three substituents, wherein each substituent is independently halo, nitro, cyano, hydroxy, amino (e.g., alkylamino, dialkylamino), haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkenyl groups include —CH═CHPh.

The term “alkynyl” as used herein by itself or as part of another group refers to an alkyl group containing one, two, or three carbon-to-carbon triple bonds. In one embodiment, the alkynyl is a C₂-C₆ alkynyl. In some embodiments, the alkynyl is a C₂-C₄ alkynyl. In some embodiments, the alkynyl has one carbon-to-carbon triple bond. Non-limiting exemplary alkynyl groups include ethynyl, propynyl, butynyl, 2-butynyl, pentynyl, and hexynyl groups.

The term “optionally substituted alkynyl” as used herein by itself or as part of another group refers to an alkynyl group that is either unsubstituted or substituted with one, two or three substituents, wherein each substituent is independently halo, nitro, cyano, hydroxy, amino, e.g., alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkynyl groups include —C≡CPh and —CH(Ph)C≡CH.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In some embodiments, the alkyl is substituted by one, two, or three fluorine atoms. In some embodiments, the alkyl is a C₁-C₆ alkyl. In some embodiments, the alkyl is a C₁-C₄ alkyl. In some embodiments, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The terms “hydroxyalkyl” or “(hydroxy)alkyl” as used herein by themselves or as part of another group refer to an alkyl group substituted with one, two, or three hydroxy groups. In one embodiment, the alkyl is a C₁-C₆ alkyl. In some embodiments, the alkyl is a C₁-C₄ alkyl. In some embodiments, the alkyl is a C₁ or C₂ alkyl. In some embodiments, the hydroxyalkyl is a monohydroxyalkyl group, i.e., substituted with one hydroxy group. In some embodiments, the hydroxyalkyl group is a dihydroxyalkyl group, i.e., substituted with two hydroxy groups. Non-limiting exemplary (hydroxyl)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “alkoxy” as used herein by itself or as part of another group refers to an alkyl group attached to a terminal oxygen atom. In one embodiment, the alkyl is a C₁-C₆ alkyl and resulting alkoxy is thus referred to as a “C₁-C₆ alkoxy.” In some embodiments, the alkyl is a C₁-C₄ alkyl group. Non-limiting exemplary alkoxy groups include methoxy, ethoxy, and tert-butoxy.

The term “haloalkoxy” as used herein by itself or as part of another group refers to a haloalkyl group attached to a terminal oxygen atom. In one embodiment, the haloalkyl group is a C₁-C₆ haloalkyl. In some embodiments, the haloalkyl group is a C₁-C₄ haloalkyl group. Non-limiting exemplary haloalkoxy groups include fluoromethoxy, difluoromethoxy, trifluoromethoxy, and 2,2,2-trifluoroethoxy.

The term “alkylthio” as used herein by itself or as part of another group refers to an alkyl group attached to a terminal sulfur atom. In one embodiment, the alkyl group is a C₁-C₄ alkyl group. Non-limiting exemplary alkylthio groups include —SCH₃, and —SCH₂CH₃.

The terms “alkoxyalkyl” or “(alkoxy)alkyl” as used herein by themselves or as part of another group refers to an alkyl group substituted with one alkoxy group. In one embodiment, the alkoxy is a C₁-C₆ alkoxy. In some embodiments, the alkoxy is a C₁-C₄ alkoxy. In some embodiments, the alkyl is a C₁-C₆ alkyl. In some embodiments, the alkyl is a C₁-C₄ alkyl. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

The term “heteroalkyl” as used by itself or part of another group refers to unsubstituted straight- or branched-chain aliphatic hydrocarbons containing from three to twenty chain atoms, i.e., 3- to 20-membered heteroalkyl, or the number of chain atoms designated, wherein at least one —CH₂— is replaced with at least one of —O—, —N(H)—, —N(C₁-C₄ alkyl)-, or —S—. The —O—, —N(H)—, —N(C₁-C₄ alkyl)-, or —S— can independently be placed at any interior position of the aliphatic hydrocarbon chain so long as each —O—, —N(H)—, —N(C₁-C₄ alkyl)-, and —S— group is separated by at least two —CH₂— groups. In one embodiment, one —CH₂— group is replaced with one —O— group. In some embodiments, two —CH₂— groups are replaced with two —O— groups. In some embodiments, three —CH₂— groups are replaced with three —O— groups. In some embodiments, four —CH₂— groups are replaced with four —O— groups. Non-limiting exemplary heteroalkyl groups include —CH₂OCH₃, —CH₂OCH₂CH₂CH₃, —CH₂CH₂CH—₂OCH₃, —CH₂CH₂OCH₂CH₂OCH₂CH₃, —CH₂CH₂OCH₂CH₂OCH₂CH₂OCH₂CH₃.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In one embodiment, the cycloalkyl is bicyclic, i.e., it has two rings. In some embodiments, the cycloalkyl is monocyclic, i.e., it has one ring. In some embodiments, the cycloalkyl is a C₃₋₈ cycloalkyl. In some embodiments, the cycloalkyl is a C₃₋₆ cycloalkyl, i.e., cyclopropyl, cyclobutyl, cyclopentyl, or cyclohexyl. In some embodiments, the cycloalkyl is a C₅ cycloalkyl, i.e., cyclopentyl. In some embodiments, the cycloalkyl is a C₆ cycloalkyl, i.e., cyclohexyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl” as used herein by itself or as part of another group refers to a cycloalkyl group that is either unsubstituted or substituted with one, two, or three substituents, wherein each substituent is independently halo, nitro, cyano, hydroxy, amino (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, and R⁵⁸ are as defined in connection with the term “optionally substituted alkyl” and R⁵⁹ is (hydroxy)alkyl or (amino)alkyl. The term optionally substituted cycloalkyl also includes cycloalkyl groups having fused optionally substituted aryl or optionally substituted heteroaryl groups such as

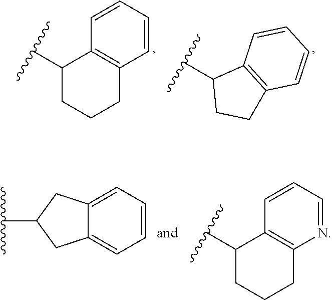

Non-limiting exemplary optionally substituted cycloalkyl groups include:

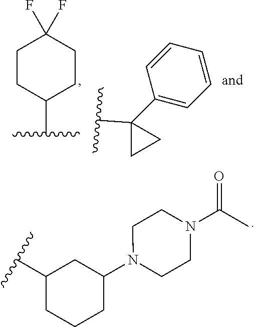

The term “heterocyclo” as used herein by itself or as part of another group refers to saturated and partially unsaturated, e.g., containing one or two double bonds, monocyclic, bicyclic, or tricyclic groups containing three to fourteen ring members, i.e., a 3- to 14-membered heterocyclo, comprising one, two, three, or four heteroatoms. Each heteroatom is independently oxygen, sulfur, or nitrogen. Each sulfur atom is independently oxidized to give a sulfoxide, i.e., S(═O), or sulfone, i.e., S(═O)₂.

The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one.

The term heterocyclo also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one.

In one embodiment, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In some embodiments, the heterocyclo group is a 5- to 8-membered cyclic group containing one ring and one or two nitrogen atoms and, optionally, one —CH₂— group is replaced with one —C(═O)— group. In some embodiments, the heterocyclo group is a 5- or 6-membered cyclic group containing one ring and one or two nitrogen atoms and, optionally, one —CH₂— group is replaced with one —C(═O)— group. In some embodiments, the heterocyclo group is a 8- to 12-membered cyclic group containing two rings and one or two nitrogen atoms. The heterocyclo can be linked to the rest of the molecule through any available carbon or nitrogen atom. Non-limiting exemplary heterocyclo groups include:

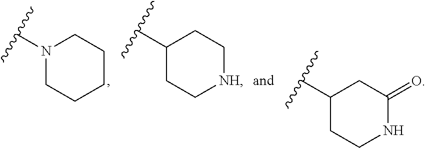

The term “optionally substituted heterocyclo” as used herein by itself or part of another group refers to a heterocyclo group that is either unsubstituted or substituted with one to four substituents, wherein each substituent is independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R⁵⁶a)C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.” Substitution may occur on any available carbon or nitrogen atom of the heterocyclo group. Non-limiting exemplary optionally substituted heterocyclo groups include:

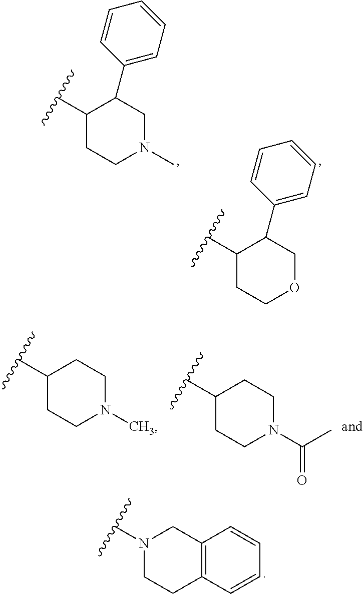

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In some embodiments, the aryl group is phenyl.

The term “optionally substituted aryl” as used herein by itself or as part of another group refers to aryl that is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R⁵⁶a)C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.”

In one embodiment, the optionally substituted aryl is an optionally substituted phenyl. In some embodiments, the optionally substituted phenyl has four substituents. In some embodiments, the optionally substituted phenyl has three substituents. In some embodiments, the optionally substituted phenyl has two substituents. In some embodiments, the optionally substituted phenyl has one substituent. Non-limiting exemplary optionally substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, and 2-phenylpropan-2-amine. The term optionally substituted aryl includes aryl groups having fused optionally substituted cycloalkyl groups and fused optionally substituted heterocyclo groups. Non-limiting examples include: 2,3-dihydro-1H-inden-1-yl, 1,2,3,4-tetrahydronaphthalen-1-yl, 1,3,4,5-tetrahydro-2H-benzo[c]azepin-2-yl, 1,2,3,4-tetrahydroisoquinolin-1-yl, and 2-oxo-2,3,4,5-tetrahydro-1H-benzo[d]azepin-1-yl.

The term “heteroaryl” as used herein by itself or as part of another group refers to monocyclic and bicyclic aromatic ring systems having five to 14 fourteen ring members, i.e., a 5- to 14-membered heteroaryl, comprising one, two, three, or four heteroatoms. Each heteroatom is independently oxygen, sulfur, or nitrogen. In one embodiment, the heteroaryl has three heteroatoms. In some embodiments, the heteroaryl has two heteroatoms. In some embodiments, the heteroaryl has one heteroatom. In some embodiments, the heteroaryl is a 5-to 10-membered heteroaryl. In some embodiments, the heteroaryl has 5 ring atoms, e.g., thienyl, a 5-membered heteroaryl having four carbon atoms and one sulfur atom. In some embodiments, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R⁵⁶a)C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.”

In one embodiment, the optionally substituted heteroaryl has two substituents. In some embodiments, the optionally substituted heteroaryl has one substituent. Any available carbon or nitrogen atom can be substituted.

The term “aryloxy” as used herein by itself or as part of another group refers to an optionally substituted aryl attached to a terminal oxygen atom. A non-limiting exemplary aryloxy group is PhO—.

The term “heteroaryloxy” as used herein by itself or as part of another group refers to an optionally substituted heteroaryl attached to a terminal oxygen atom. A non-limiting exemplary aryloxy group is pyridyl-O—.

The term “aralkyloxy” as used herein by itself or as part of another group refers to an aralkyl attached to a terminal oxygen atom. A non-limiting exemplary aralkyloxy group is PhCH₂O—.

The term “(cyano)alkyl” as used herein by itself or as part of another group refers to an alkyl substituted with one, two, or three cyano groups. In one embodiment, the alkyl is substituted with one cyano group. In some embodiments, the alkyl is a C₁-C₆ alkyl In some embodiments, the alkyl is a C₁-C₄ alkyl. Non-limiting exemplary (cyano)alkyl groups include —CH₂CH₂CN and —CH₂CH₂CH₂CN.

The term “(cycloalkyl)alkyl” as used herein by itself or as part of another group refers to an alkyl substituted with one or two optionally substituted cycloalkyl groups. In one embodiment, the cycloalkyl group(s) is an optionally substituted C₃-C₆ cycloalkyl. In some embodiments, the alkyl is a C₁-C₆ alkyl. In some embodiments, the alkyl is a C₁-C₄ alkyl. In some embodiments, the alkyl is a C₁ or C₂ alkyl. In some embodiments, the alkyl is substituted with one optionally substituted cycloalkyl group. In some embodiments, the alkyl is substituted with two optionally substituted cycloalkyl groups. Non-limiting exemplary (cycloalkyl)alkyl groups include:

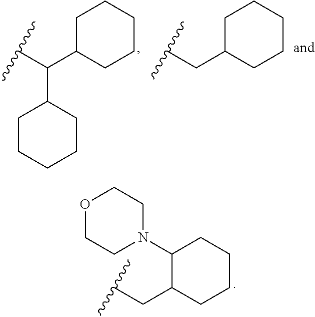

The term “sulfonamido” as used herein by itself or as part of another group refers to a radical of the formula —SO₂NR^{50a}R^{50b}, wherein R^{50a} and R^{50b} are each independently hydrogen, alkyl, optionally substituted cycloalkyl, optionally substituted heterocyclo, optionally substituted aryl, or optionally substituted heteroaryl; or R^{50a} and R^{50b} taken together with the nitrogen to which they are attached form a 3- to 8-membered optionally substituted heterocyclo group. Non-limiting exemplary sulfonamido groups include —SO₂NH₂, —SO₂N(H)CH₃, and —SO₂N(H)Ph.

The term “alkylcarbonyl” as used herein by itself or as part of another group refers to a carbonyl group, i.e., —C(═O)—, substituted by an alkyl group. In one embodiment, the alkyl is a C₁-C₄ alkyl. A non-limiting exemplary alkylcarbonyl group is —COCH₃.

The term “arylcarbonyl” as used herein by itself or as part of another group refers to a carbonyl group, i.e., —C(═O)—, substituted by an optionally substituted aryl group. A non-limiting exemplary arylcarbonyl group is —COPh.

The term “alkylsulfonyl” as used herein by itself or as part of another group refers to a sulfonyl group, i.e., —SO₂—, substituted by an alkyl group. A non-limiting exemplary alkylsulfonyl group is —SO₂CH₃.

The term “arylsulfonyl” as used herein by itself or as part of another group refers to a sulfonyl group, i.e., —SO₂—, substituted by an optionally substituted aryl group. A non-limiting exemplary arylsulfonyl group is —SO₂Ph.

The term “mercaptoalkyl” as used herein by itself or as part of another group refers to an alkyl substituted by a —SH group.

The term “carboxy” as used by itself or as part of another group refers to a radical of the formula —C(═O)OH.

The term “carboxamido” as used herein itself or as part of another group refers to a radical of the formula —C(═O)NR^{50c}R^{50d}, wherein R^{50c} and R^{50d} are each independently hydrogen, alkyl, optionally substituted cycloalkyl, optionally substituted heterocyclo, optionally substituted aryl, optionally substituted heteroaryl, aralkyl, (heteroaryl)alkyl, or (heterocyclo)alkyl; or R^{50c} and R^{50d} taken together with the nitrogen to which they are attached form a 3- to 8-membered optionally substituted heterocyclo group. Non-limiting exemplary carboxamido groups include —C(═O)NH₂, —C(═O)N(H)CH₃, and —C(═O)N(H)Ph

The term “ureido” as used herein by itself or as part of another group refers to a radical of the formula —NR^{51a}-C(═O)—NR^{51b}R^{51c}, wherein R^{51a} is hydrogen or alkyl; and R^{51b} and R^{51c} are each independently hydrogen, alkyl, optionally substituted cycloalkyl, optionally substituted heterocyclo, optionally substituted aryl, or optionally substituted heteroaryl, or R^{51b} and R^{51c} taken together with the nitrogen to which they are attached form a 4- to 8-membered optionally substituted heterocyclo group. Non-limiting exemplary ureido groups include —NH—C(C═O)—NH₂ and —NH—C(C═O)—NHCH₃.

The term “guanidino” as used herein by itself or as part of another group refers to a radical of the formula —NR^{52a}—C(═NR⁵³)—NR^{52b}R^{52c}, wherein R^{52a} is hydrogen or alkyl; R^{52b} and R^{53c} are each independently hydrogen, alkyl, optionally substituted cycloalkyl, optionally substituted heterocyclo, optionally substituted aryl, or optionally substituted heteroaryl; or R^{52b} and R^{52c} taken together with the nitrogen to which they are attached form a 4- to 8-membered optionally substituted heterocyclo group; and R⁵³ is hydrogen, alkyl, cyano, alkylsulfonyl, alkylcarbonyl, carboxamido, or sulfonamido. Non-limiting exemplary guanidino groups include —NH—C(C═NH)—NH₂, —NH—C(C═NCN)—NH₂, and —NH—C(C═NH)—NHCH₃.

The term “(heterocyclo)alkyl” as used herein by itself or as part of another group refers to an alkyl substituted with one, two, or three optionally substituted heterocyclo groups. In one embodiment, the alkyl is substituted with one optionally substituted 5- to 8-membered heterocyclo group. In some embodiments, alkyl is a C₁-C₆ alkyl. In some embodiments, alkyl is a C₁-C₄ alkyl. The heterocyclo group can be linked to the alkyl group through a carbon or nitrogen atom. Non-limiting exemplary (heterocyclo)alkyl groups include:

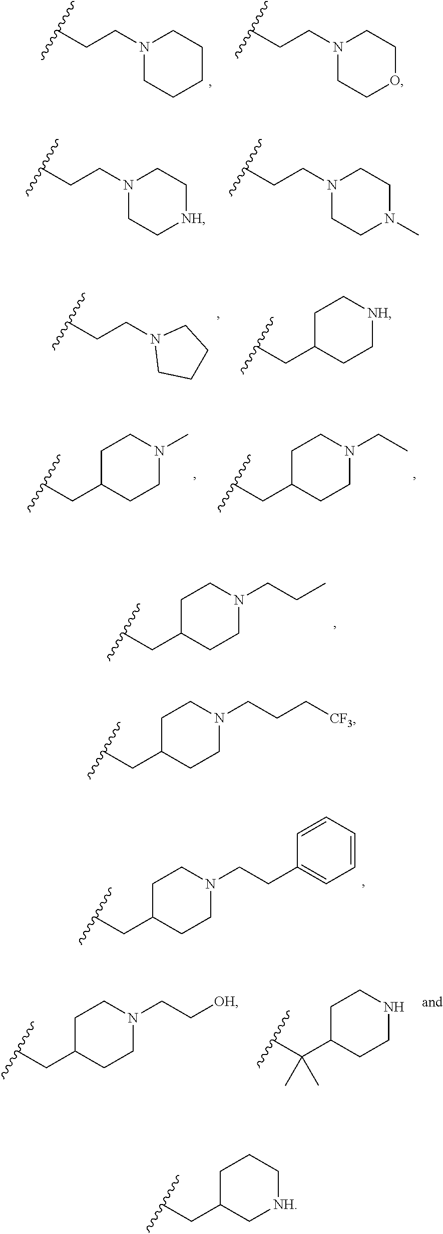

The term “carbamate” as used herein by itself or as part of another group refers to a radical of the formula —NR^{54a}—C(═O)—OR^{54b}, wherein R^{54a} is hydrogen or alkyl, and R^{54b} is hydrogen, alkyl, optionally substituted cycloalkyl, optionally substituted heterocyclo, optionally substituted aryl, or optionally substituted heteroaryl. A non-limiting exemplary carbamate group is —NH—(C═O)—OtBu.

The term “(heteroaryl)alkyl” as used herein by itself or as part of another group refers to an alkyl substituted with one or two optionally substituted heteroaryl groups. In one embodiment, the alkyl group is substituted with one optionally substituted 5- to 14-membered heteroaryl group. In some embodiments, the alkyl group is substituted with two optionally substituted 5- to 14-membered heteroaryl groups. In some embodiments, the alkyl group is substituted with one optionally substituted 5- to 9-membered heteroaryl group. In some embodiments, the alkyl group is substituted with two optionally substituted 5- to 9-membered heteroaryl groups. In some embodiments, the alkyl group is substituted with one optionally substituted 5- or 6-membered heteroaryl group. In some embodiments, the alkyl group is substituted with two optionally substituted 5- or 6-membered heteroaryl groups. In one embodiment, the alkyl group is a C₁-C₆ alkyl. In some embodiments, the alkyl group is a C₁-C₄ alkyl. In some embodiments, the alkyl group is a C₁ or C₂ alkyl. Non-limiting exemplary (heteroaryl)alkyl groups include:

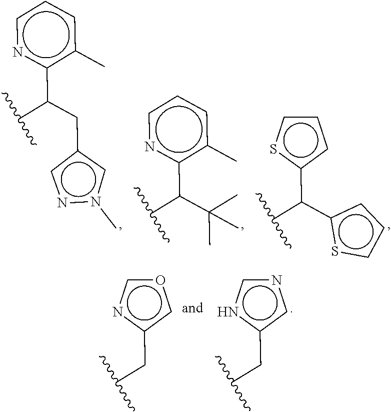

The term “(amino)(heteroaryl)alkyl” as used herein by itself or as part of another group refers to an alkyl group substituted with one optionally substituted heteroaryl group and one amino group. In one embodiment, the heteroaryl is an optionally substituted 5- to 9-membered heteroaryl group. In some embodiments, the heteroaryl is an optionally substituted 5- or 6-membered heteroaryl group. In one embodiment, the alkyl is a C₁-C₆ alkyl. In some embodiments, the alkyl is a C₁-C₄ alkyl. In some embodiments, the alkyl is a C₁ or C₂ alkyl. A non-limiting exemplary (amino)(heteroaryl)alkyl group is:

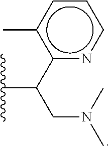

The terms “aralkyl” or “(aryl)alkyl” as used herein by themselves or as part of another group refers to an alkyl substituted with one, two, or three optionally substituted aryl groups. In one embodiment, the alkyl is substituted with one optionally substituted aryl group. In some embodiments, the alkyl is substituted with two optionally substituted aryl groups. In one embodiment, the aryl is an optionally substituted phenyl or optionally substituted naphthyl. In some embodiments, the aryl is an optionally substituted phenyl. In one embodiment, the alkyl is a C₁-C₆ alkyl. In some embodiments, the alkyl is a C₁-C₄ alkyl. In some embodiments, the alkyl is a C₁ or C₂ alkyl. Non-limiting exemplary (aryl)alkyl groups include benzyl, phenethyl, —CHPh₂, and —CH(4-F-Ph)₂.

The term “amido” as used herein by itself or as part of another group refers to a radical of formula —C(═O)NR^{60a}R^{60b}, wherein R^{60a} and R^{60b} are each independently hydrogen, optionally substituted alkyl, optionally substituted alkenyl, optionally substituted alkynyl, haloalkyl, (alkoxy)alkyl, (hydroxy)alkyl, (cyano)alkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl; or R^{60a} and R^{60b} taken together with the nitrogen to which they are attached from a 4- to 8-membered optionally substituted heterocyclo group. In one embodiment, R^{60a} and R^{60b} are each independently hydrogen or C₁-C₆ alkyl.

The term “(amido)(aryl)alkyl” as used herein by itself or as part of another group refers to an alkyl group substituted with one amido group and one optionally substituted aryl group. In one embodiment, the aryl group is an optionally substituted phenyl. In one embodiment, the alkyl is a C₁-C₆ alkyl. In some embodiments, the alkyl is a C₁-C₄ alkyl. Non-limiting exemplary (amido)(aryl)alkyl groups include:

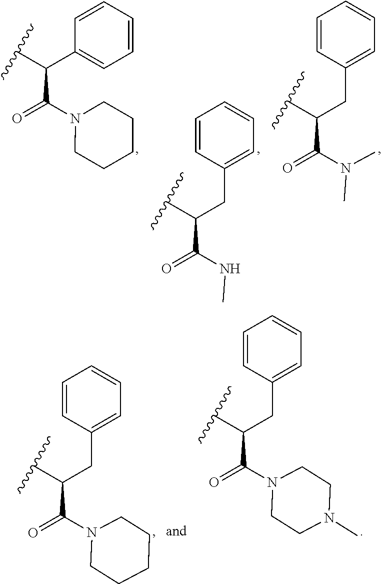

The term “(amino)(aryl)alkyl” as used herein by itself or as part of another group refers to an alkyl group substituted with one amino group and one optionally substituted aryl group. In one embodiment, the amino group is —NH₂, alkylamino, or dialkylamino. In one embodiment, the aryl group is an optionally substituted phenyl. In one embodiment, the alkyl is a C₁-C₆ alkyl. In some embodiments, the alkyl is a C₁-C₄ alkyl. Non-limiting exemplary (amino)(aryl)alkyl groups include:

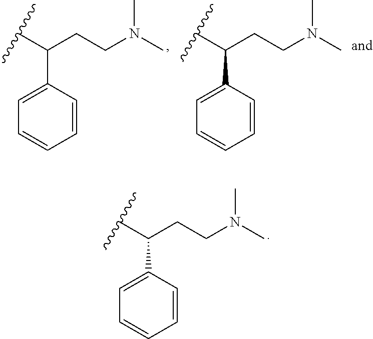

The term “amino” as used by itself or as part of another group refers to a radical of the formula —NR^{55a}R^{55b}, wherein R^{55a} and R^{55b} are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

In one embodiment, the amino is —NH₂.

In some embodiments, the amino is an “alkylamino,” i.e., an amino group wherein R^{55a} is C₁₋₆ alkyl and R^{55b} is hydrogen. In one embodiment, R^{55a} is C₁-C₄ alkyl. Non-limiting exemplary alkylamino groups include —N(H)CH₃ and —N(H)CH₂CH₃.

In some embodiments, the amino is a “dialkylamino,” i.e., an amino group wherein R^{55a} and R^{55b} are each independently C₁₋₆ alkyl. In one embodiment, R^{55a} and R^{55b} are each independently C₁-C₄ alkyl. Non-limiting exemplary dialkylamino groups include —N(CH₃)₂ and —N(CH₃)CH₂CH(CH₃)₂.

In some embodiments, the amino is a “hydroxyalkylamino,” i.e., an amino group wherein R^{55a} is (hydroxyl)alkyl and R^{55b} is hydrogen or C₁-C₄ alkyl.

In some embodiments, the amino is a “cycloalkylamino,” i.e., an amino group wherein R^{55a} is optionally substituted cycloalkyl and R^{55b} is hydrogen or C₁-C₄ alkyl.

In some embodiments, the amino is a “aralkylamino,” i.e., an amino group wherein R^{55a} is aralkyl and R^{55b} is hydrogen or C₁-C₄ alkyl. Non-limiting exemplary aralkylamino groups include —N(H)CH₂Ph, —N(H)CHPh₂, and —N(CH₃)CH₂Ph.

In some embodiments, the amino is a “(cycloalkyl)alkylamino,” i.e., an amino group wherein R^{55a} is (cycloalkyl)alkyl and R^{55b} is hydrogen or C₁-C₄ alkyl. Non-limiting exemplary (cycloalkyl)alkylamino groups include:

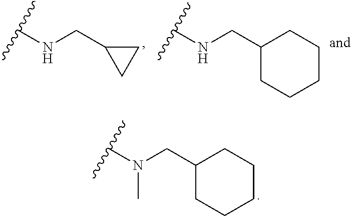

In some embodiments, the amino is a “(heterocyclo)alkylamino,” i.e., an amino group wherein R^{55a} is (heterocyclo)alkyl and R^{55b} is hydrogen or C₁-C₄ alkyl. Non-limiting exemplary (heterocyclo)alkylamino groups include:

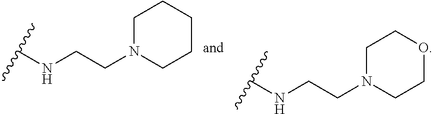 [SMILES: *NCCN1CCCCC1 ; *NCCN1CCOCC1]

The term “(amino)alkyl” as used herein by itself or as part of another group refers to an alkyl substituted with one amino group. In one embodiment, the amino group is —NH₂. In one embodiment, the amino group is an alkylamino. In some embodiments, the amino group is a dialkylamino. In some embodiments, the alkyl is a C₁-C₆ alkyl. In some embodiments, the alkyl is a C₁-C₄ alkyl. Non-limiting exemplary (amino)alkyl groups include —CH₂NH₂, CH₂CH₂N(H)CH₃, —CH₂CH₂N(CH₃)₂, CH₂N(H)cyclopropyl, —CH₂N(H)cyclobutyl, and —CH₂N(H)cyclohexyl, and —CH₂CH₂CH₂N(H)CH₂Ph and —CH₂CH₂CH₂N(H)CH₂(4-CF₃-Ph).

The term “heteroarylenyl” as used herein by itself or part of another group refers to a divalent form of an optionally substituted 5- to 9-membered heteroaryl group. In one embodiment, the heteroarylenyl is a bicyclic 9-membered heteroarylenyl. Exemplary non-limiting exemplary bicyclic 9-membered heteroarylenyl groups include:

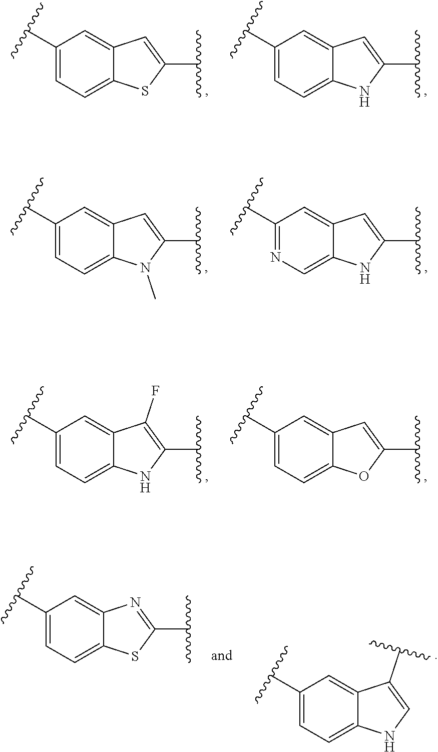

In the present disclosure, the term “alkylenyl” as used herein by itself or part of another group refers to a divalent form of an alkyl group, wherein the alkyl group is either unsubstituted or substituted with one or two groups independently selected from the group consisting of optionally substituted phenyl and optionally substituted 5- or 6-membered heteroaryl. In one embodiment, the alkylenyl is a divalent form of a C₁₋₁₂ alkyl. In one embodiment, the alkylenyl is a divalent form of a C₁₋₁₀ alkyl. In one embodiment, the alkylenyl is a divalent form of a C₁₋₆ alkyl. In one embodiment, the alkylenyl is a divalent form of an unsubstituted C₁₋₆ alkyl. In some embodiments, the alkylenyl is a divalent form of an unsubstituted C₁₋₄ alkyl. In some embodiments, the alkylenyl is a divalent form of a C₁₋₄ alkyl substituted with one or two optionally substituted phenyl groups. Non-limiting exemplary alkylenyl groups include —CH₂—, —CH₂CH₂—, —CH(Ph)-, —CH(Ph)CH₂—, —CH₂CH₂CH₂—, —CH(Ph)CH₂CH₂—, —CH₂(CH₂)₂CH₂—, —CH(CH₂)₃CH₂—, and —CH₂(CH₂)₄CH₂—.

The term “heteroalkylenyl” as used herein by itself or part of another group refers to a divalent form of a heteroalkyl group. In one embodiment, the heteroalkylenyl is a divalent form of a 3- to 20-membered heteroalkyl. In some embodiments, the heteroalkylenyl is a divalent form of a 3- to 10-membered heteroalkyl. In some embodiments, the heteroalkylenyl is a divalent form of a 3- to 8-membered heteroalkyl. In some embodiments, the heteroalkylenyl is a divalent form of a 3- to 6-membered heteroalkyl. In some embodiments, the heteroalkylenyl is a divalent form of a 3- to 4-membered heteroalkyl. In some embodiments, the heteroalkylenyl is a radical of the formula —(CH₂CH₂O)ᵤ₁— wherein u₁ is 1, 2, 3, 4, 5, or 6. Non-limiting exemplary heteroalkylenyl groups include —CH₂OCH₂—, —CH₂CH₂OCH₂CH₂O—, —CH₂OCH₂CH₂CH₂—, and —CH₂CH₂OCH₂CH₂OCH₂CH₂O—.

The term “heterocyclenyl” as used herein by itself or part of another group refers to a divalent form of an optionally substituted 4- to 8-membered heterocyclo group. In one embodiment, the heterocyclenyl is a divalent form of an optionally substituted azetidine. In one embodiment, the heterocyclenyl is a divalent form of an optionally substituted piperidinyl. Non-limiting exemplary heterocyclenyl groups include:

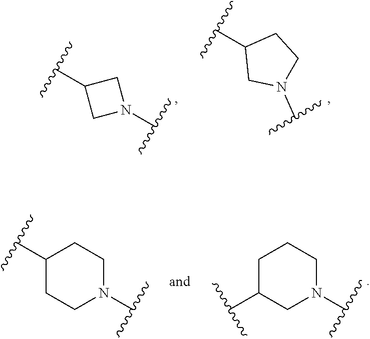 [SMILES: *C1CN(*)C1 ; *C1CCN(*)C1 ; *C1CCN(*)CC1 ; *C1CCCN(*)C1]

The term “cycloalkylenyl” as used herein by itself or part of another group refers to a divalent form of an optionally substituted C₄-C₆ cycloalkyl group. In one embodiment, the cycloalkylenyl is a 4-membered cycloalkylenyl. In some embodiments, the cycloalkylenyl is a 5-membered cycloalkylenyl. In some embodiments, the cycloalkylenyl is a 6-membered cycloalkylenyl. Non-limiting exemplary groups include:

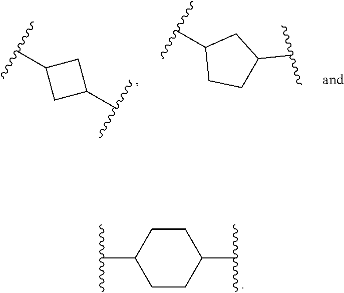 [SMILES: *C1CC(*)C1 ; *C1CCC(*)C1 ; *C1CCC(*)CC1]

The term “phenylenyl” as used herein by itself or part of another group refers to a divalent form of an optionally substituted phenyl group. Non-limiting examples include:

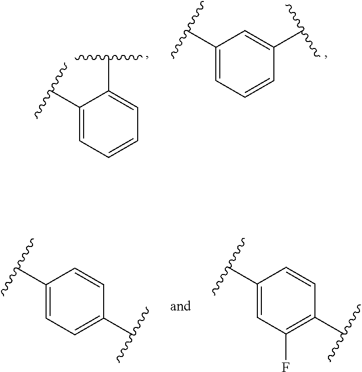 [SMILES: *c1ccccc1* ; *c1cccc(*)c1 ; *c1ccc(*)cc1 ; *c1ccc(*)c(F)c1]

The term “bicyclic 9- or 10-membered heteroarylenyl” as used herein by itself or part of another group refers to a divalent form of an optionally substituted bicyclic 9- or 10-membered heteroaryl group. In one embodiment, bicyclic 9- or 10-membered heteroarylenyl is a bicyclic 9-membered heteroarylenyl. In some embodiments, bicyclic 9- or 10-membered heteroarylenyl is a bicyclic 10-membered heteroarylenyl. Exemplary bicyclic 9-membered heteroarylenyl groups include, but are not limited to,

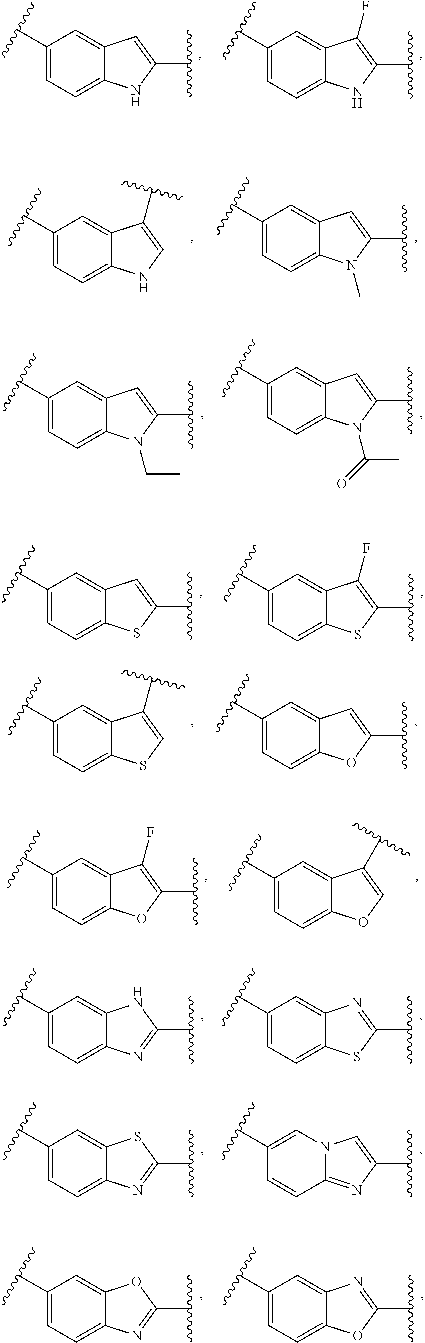

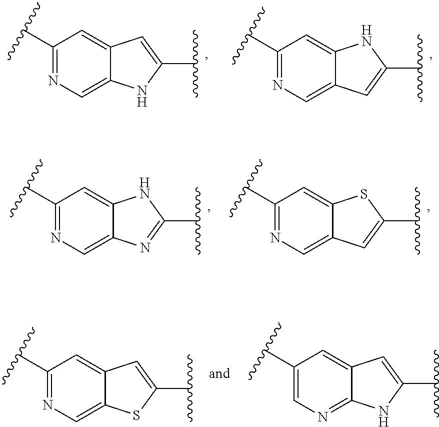 [SMILES: *c1cc2cc(*)[nH]c2cn1 ; *c1cc2[nH]c(*)cc2cn1 ; *c1cc2[nH]c(*)nc2cn1 ; *c1cc2sc(*)cc2cn1 ; *c1cc2cc(*)sc2cn1 ; *c1cnc2[nH]c(*)cc2c1]

Exemplary bicyclic 10-membered heteroarylenyl groups include, but are not limited to,

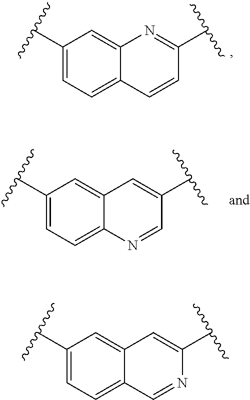 [SMILES: *c1ccc2ccc(*)nc2c1 ; *c1ccc2ncc(*)cc2c1 ; *c1ccc2cnc(*)cc2c1]

The term “naphthylenyl” as used herein by itself or part of another group refers to a divalent form of an optionally substituted naphthyl group. Exemplary naphthylenyl groups include, but are not limited to,

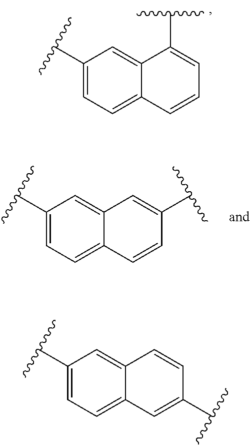 [SMILES: *c1ccc2cccc(*)c2c1 ; *c1ccc2ccc(*)cc2c1 ; *c1ccc2cc(*)ccc2c1]

The present disclosure encompasses any of the Compounds of the Disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C, ¹⁵N, ¹⁸O, ¹⁷O, ³¹P, ³²P, ³⁵S, ¹⁸F, and ³⁶Cl, respectively, e.g., ³H, ¹¹C, and ¹⁴C. In one embodiment, provided is a composition wherein substantially all of the atoms at a position within the Compound of the Disclosure are replaced by an atom having a different atomic mass or mass number. In some embodiments, provided is a composition wherein a portion of the atoms at a position within the Compound of the disclosure are replaced, i.e., the Compound of the Disclosure is enriched at a position with an atom having a different atomic mass or mass number.” Isotopically-labelled Compounds of the Disclosure can be prepared by methods known in the art.

Compounds of the Disclosure contain one or more asymmetric centers and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In one embodiment, Compounds of the Disclosure are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The term “coupling agent” as used herein refers to the reagent, e.g., activator, or combination of reagents, e.g., activator and base, or activator, base, and additive(s), used to form an amide bond between a carboxylic acid and an amine. Coupling agents are well known in the art. In one embodiment, the coupling agent comprises and activator, e.g., a carbodiimide (dicyclohexylcarbodiimide, diisopropylcarbodiimide, (N-(3-dimethylaminopropyl)-N′-ethylcarbodiimide.HCl) or (N-[(7-Aza-1H-benzotriazol-1-yl)(dimethylamino)-methylene]-N-methylmethanaminium hexafluorophosphate N-oxide (HATU). In some embodiments, the coupling agent comprises and activator, e.g., a carbodiimide or HATU, and a base, e.g., diisopropylethyl amine or 2,4,6-collidine. In some embodiments, the coupling agent comprises and activator, e.g., a carbodiimide, a base, e.g., 2,4,6-collidine, and at least one additive, e.g., 1-hydroxybenzotriazole or OxymaPure®. Solvents used in coupling reactions are also well known in the art. Exemplary solvents include, but are not limited to, dichloromethane, N,N-dimethylformamide, tetrahydrofuran, 2-methyltetrahydrofuran, and N-methyl-2-pyrrolidone.

As used herein, the term “about” refers to a range covering any normal fluctuations appreciated by one of ordinary skill in the relevant art. In some embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “pharmaceutically acceptable salt” refers to a derivative of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc. Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3. It is to be understood that all references to pharmaceutically acceptable salts include solvent addition forms (solvates) or crystal forms (polymorphs) as defined herein, of the same salt.

It is understood that the compounds described herein include the compounds themselves, as well as their pharmaceutically acceptable salts, and their solvates, if applicable. A pharmaceutically acceptable salt, for example, can be formed between a pharmaceutically acceptable anion and a positively charged group (e.g., amino) on a compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

It is understood that the compounds of the present disclosure, for example, the salts of the compounds, can exist in either hydrated or unhydrated (the anhydrous) form or as solvates with other solvent molecules. Nonlimiting examples of hydrates include monohydrates and dihydrates. Nonlimiting examples of solvates include ethanol solvates and acetone solvates.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art

As used herein, the term “subject” is interchangeable with the term “subject in need thereof”, both of which refer to a subject having a disease or having an increased risk of developing the disease.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

As used herein, the term “pharmaceutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Pharmaceutically effective amounts for a given situation can be determined by routine experimentation that is within the skill and judgment of the clinician.

It is understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. In some embodiments, the pharmaceutically acceptable salt of a compound is also a prodrug of the compound. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

It is understood that all references to pharmaceutically acceptable salts include solvent addition forms (solvates) or crystal forms (polymorphs) as defined herein, of the same salt.

As used herein, the term “prodrug” refers to any agent which, when administered to a mammal, is converted in whole or in part to a targeted compound. In some embodiments, the prodrug of a compound is also a pharmaceutically acceptable salt of the compound.

It is understood that the compounds of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1. Assessing Compound No. 36 on Established Parameters of Acute GVHD

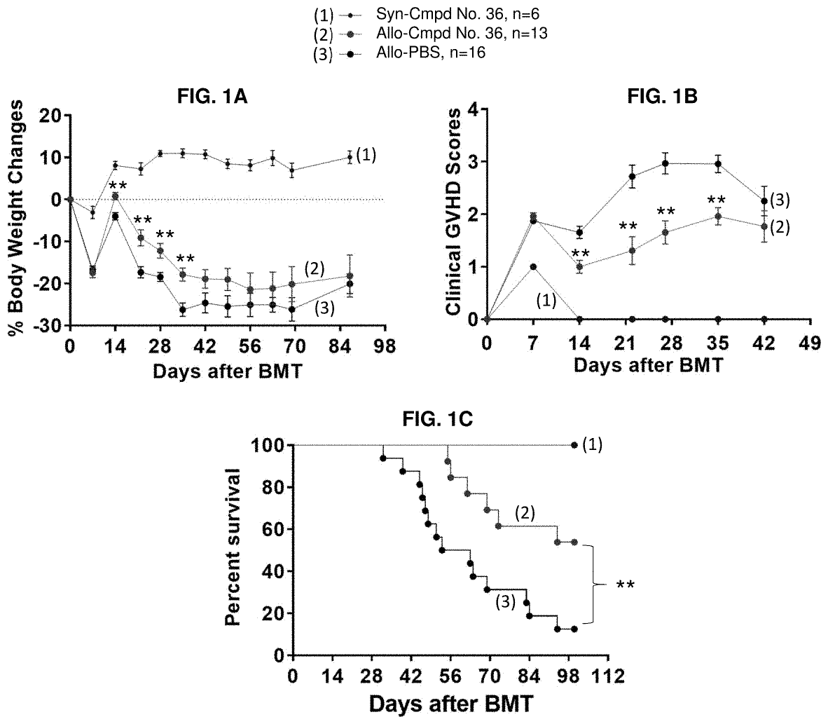

To determine whether Compound No. 36 affects established parameters of acute GVHD, a well-established C57BL/6 (H-2Kb) into a BALB/c (H-2Kd) major histocompatibility complex (MHC) mismatch model was used. On day 0, BALB/c recipient mice received 5×10⁶ of bone marrow cells and 1×10⁶ of purified splenic T cells from allogeneic C57BL/6 donor or syngeneic BALB/c donor following lethal irradiation with 800 centiGray (units of radiation) on day −1. Recipients received intraperitoneal injections of 25 mg/kg every other day of Compound No. 36 or vector from day −1 to day 21. Mice were monitored daily for survival and weekly for GVHD score. Compound No. 36 was found to significantly reduce acute GVHD severity by reducing body weight loss and clinical GVHD scores (FIG. 1A and FIG. 1B). Compound No. 36 was also determined to significantly improve survival compared to the group with vector only (day 50, 100% vs. 50%; day 100, 53% vs. 12%, p<0.001) (FIG. 1C). Syngeneic recipients of BALB/c to BALB/c which received the same doses of Compound No. 36 did not present any signs of toxicity from Compound No. 36, or GVHD, and their survival rate was 100% until the end point (FIG. 1C).

### Example 2. Assessing Compound No. 36 on Graft-Versus Tumor (GVT)

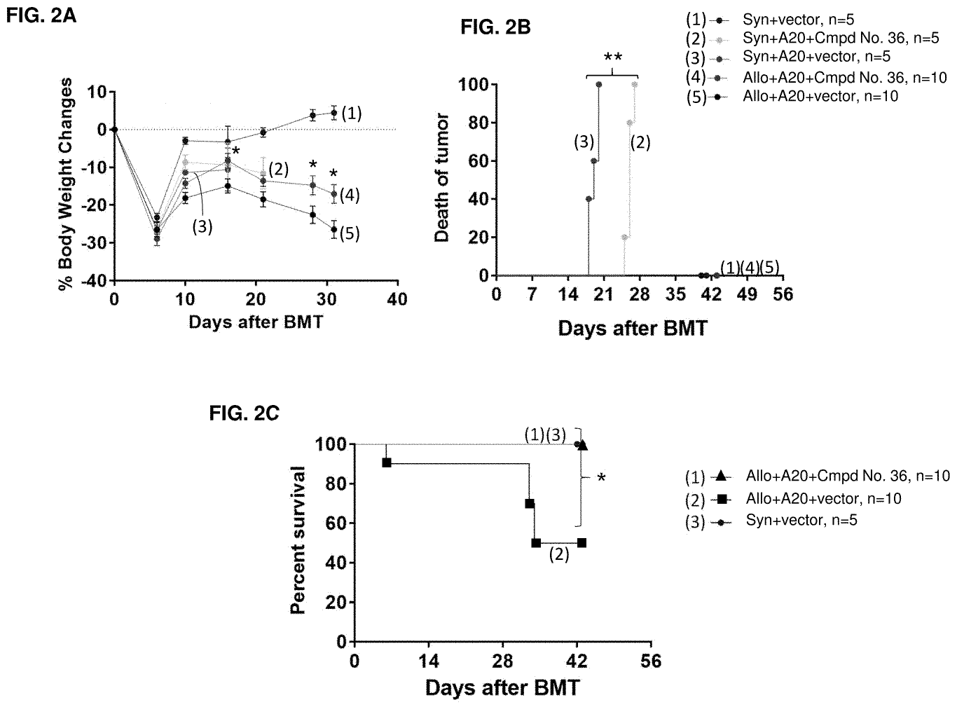

As GVHD is tightly linked to the graft-versus tumor (GVT) response, testing was undertaken to determine whether Compound No. 36 treatment reduces the effect of GVT in the same mouse model as for GVHD (Example 1). To that end, a mouse BALB/c lymphoma cell line, A20 (0.5×10⁶), thus syngeneic to the recipients, was incorporated to the bone marrow transplantation (BMT) cell inoculum on day 0. Recipients received the same doses of Compound No. 36 or vector from day −1 to day 21 as the above model. Syngeneic recipients of BALB/c to BALB/c were also setup with or without A20 cell inoculum and injected the same doses of Compound No. 36 or vector. Syngeneic recipients challenged with A20 cells got systemic tumor infiltration (lower-limb paralysis, hepatosplenomegaly) and 100% of recipients died within 3 weeks after BMT without Compound No. 36 treatment (FIG. 2A and FIG. 2B). Compound No. 36 treatment significantly extended the survival time in syngeneic recipients receiving A20 cells (FIG. 2A and FIG. 2B). In the allogeneic GVHD model challenged with A20 cells, no tumor-related death was observed both in Compound No. 36 treatment or vector group by 6 weeks after BMT (FIG. 2). By contrast, Compound No. 36 treatment significantly reduced GVHD severity and improved mice survival (100% vs. 50%, Compound No. 36 vs. vector, p<0.05) by 6 weeks after BMT (FIG. 2).

### Example 3. Assessing Compound No. 36 on GVHD Target Organs

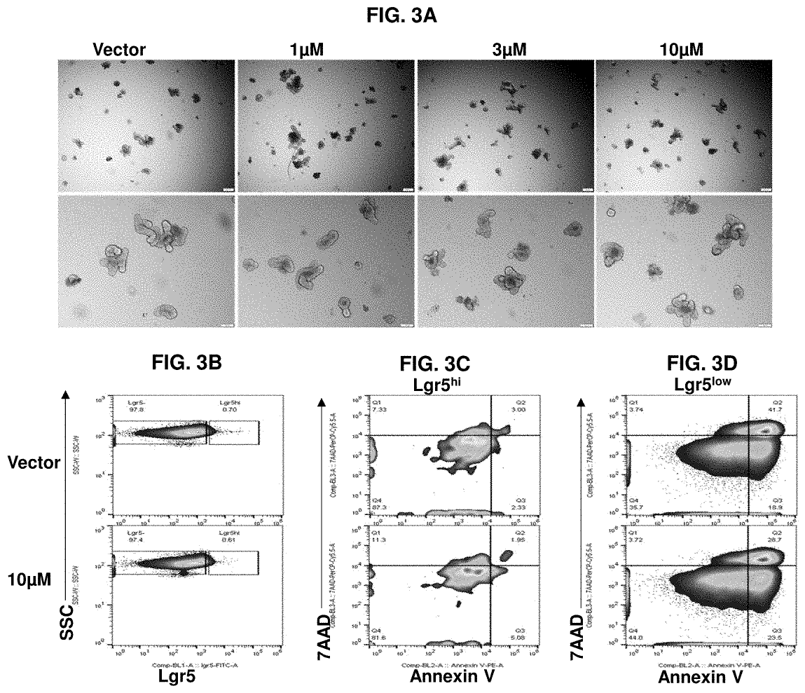

To further determine whether Compound No. 36 has any direct GVHD organ (e.g., GI tract) toxicity or conversely, a regenerative effects, in the context of BMT, various concentrations of Compound No. 36 was cocultured with established small intestinal organoids, in which the intestine crypts were from the GFP-Leucine-rich repeat-containing G protein-coupled receptor 5 (Lgr5) transgenic mouse strain. Intestinal organoids were established from culture of small intestinal crypts in GFP-Lgr5 transgene B6 mice. 3-5 passages of organoids were used to test the effect of Compound No. 36 in vitro. After dissociation of organoids, 100 crypts per well mixed with Matrigel-culture medium were plated in 24-well plate. After the Matrigel polymerized, 500 μL of culture medium with different concentrations of Compound No. 36 or vector were added to each well. Orgonoid growth was assessed daily. Three days after coculture, organoid numbers and budding numbers were counted under the microscope and were photographed (FIG. 3A). No significant changes were observed in series concentrations of Compound No. 36 treatment (FIG. 3A). Organoids were then mechanically disrupted and dissociated into single cells with TrypLE Express. Cells were stained with anti-CD326, annexin V, and 7-AAD to analyze GFP-Lgr5^{hi} cells apoptosis by flow cytometry. Upon analysis of the Lgr5-GFP⁺ stem cells and Annexin V expression by flow cytometry, no differences between vector group and Compound No. 36 treatment group were observed (FIGS. 3B-3D). This data suggests that Compound No. 36 does not have significant toxicity on intestine. 7AAD—7 amino actinomycin D.

### EQUIVALENTS

It is understood that the invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method of treating or preventing GVHD in a subject, comprising administering to the subject a pharmaceutically effective amount of a compound of Formula I: 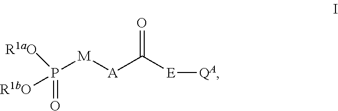
or a pharmaceutically acceptable salt or solvate thereof, wherein:
R^{1a} and R^{1b} are independently selected from the group consisting of hydrogen, C₁-C₄ alkyl, aralkyl, and —CH₂OC(═O)R^{1e};
R^{1e} is selected from the group consisting of C₁-C₆ alkyl, C₃-C₆ cycloalkyl, and C₁-C₆ alkoxy;
M is selected from the group consisting of —O— and —C(R^{2a})(R^{2b})—;
each R^{2a} and R^{2b} are independently selected from the group consisting of hydrogen and fluoro; or
R^{2a} and R^{2b} taken together with the carbon atom to which they are attached form a —C(═O)— group;
A is selected from the group consisting of: 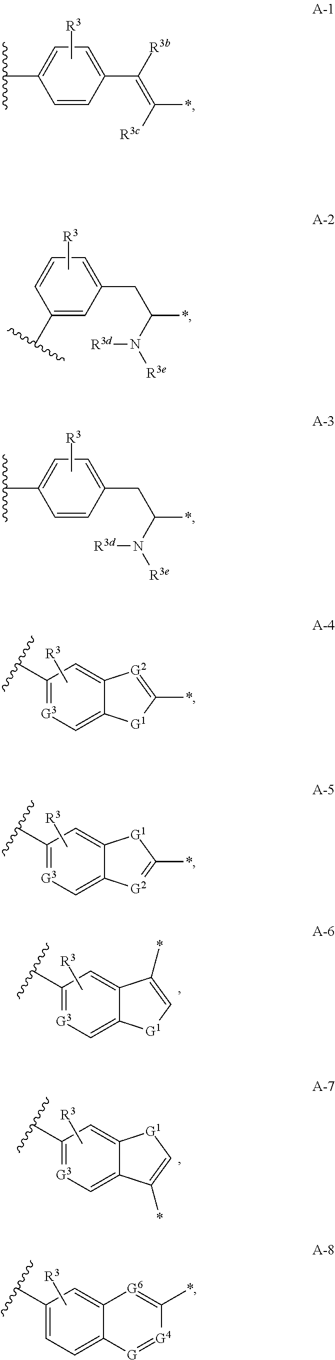 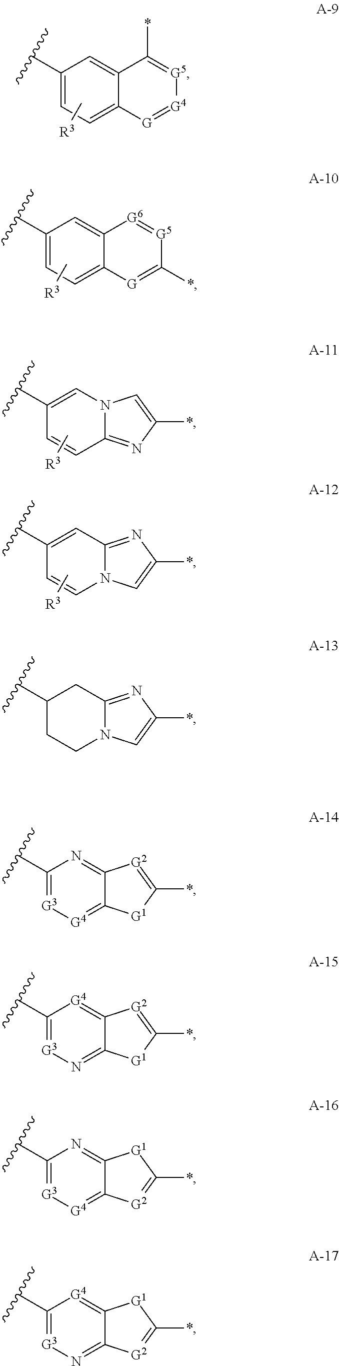 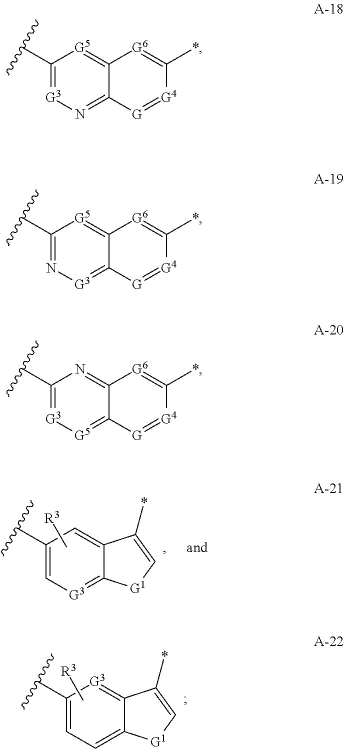
wherein the bond designated with an is attached to —C(═O)-E-Q^{A};
G¹ is selected from the group consisting of —O—, —S—, and —NR¹⁷—;
G² is selected from the group consisting of —N═ and —CR^{18a}—;
G³ is selected from the group consisting of —N═ and —CR^{18b};
G⁴ is selected from the group consisting of —N═ and —CR^{18c}═;
G⁵ is selected from the group consisting of —N═ and —CR^{18d}=;
G⁶ is selected from the group consisting of —N═ and —CR^{18e}=;
G is selected from the group consisting of —N═ and —CR^{18f}—;
R³ is selected from the group consisting of hydrogen, halo, C₁-C₄ alkyl, C₃-C₆ cycloalkyl, —C(═O)R^{3a}, and aralkyl;
R^{3a} is C₁-C₄ alkyl;
R^{3b} and R^{3c} are independently selected from the group consisting of hydrogen and C₁-C₄ alkyl;
R^{3d} is selected from the group consisting of hydrogen, C₁-C₆ alkyl, and —C(═O)R^{3f};
R^{3e} is selected from the group consisting of hydrogen and C₁-C₄ alkyl;
R^{3f} is selected from the group consisting of C₁-C₁₂ alkyl, C₁-C₆ alkoxy, and aralkyloxy;
R^{17a} is selected from the group consisting of hydrogen, C₁-C₄ alkyl, C₃-C₆ cycloalkyl, aralkyl, and —C(═O)R^{17a};
R^{17a} is C₁-C₄ alkyl;
R^{18a}, R^{18b}, R^{18c}, R^{18d}, R^{18e}, and R^{18f} are each independently selected from the group consisting of hydrogen, halo, and C₁-C₄ alkyl,
E is: 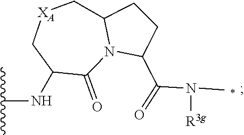
wherein the bond designated with an “*” is attached to Q^{A};
R^{3g} is selected from the group consisting of hydrogen and C₁-C₄ alkyl;
X_{A} is selected from the group consisting of —N(R⁸)CH₂—, —CH₂N(R⁸)—, and —CH₂CH₂—;
R⁸ is selected from the group consisting of hydrogen, C₁-C₆ alkyl, C₁-C₆ haloalkyl, (heterocyclo)alkyl, —C(═O)R⁹, alkylsulfonyl, and -L-B;
R⁹ is selected from the group consisting of C₁-C₆ alkyl, amino, C₁-C₆ alkoxy, aralkyloxy, optionally substituted C₃-C₁₀ cycloalkyl, optionally substituted 4- to 8-membered heterocyclo, optionally substituted aryl, optionally substituted 5- to 10-membered heteroaryl, aralkyl, and (heteroaryl)alkyl;
Q^{A} selected from the group consisting of: 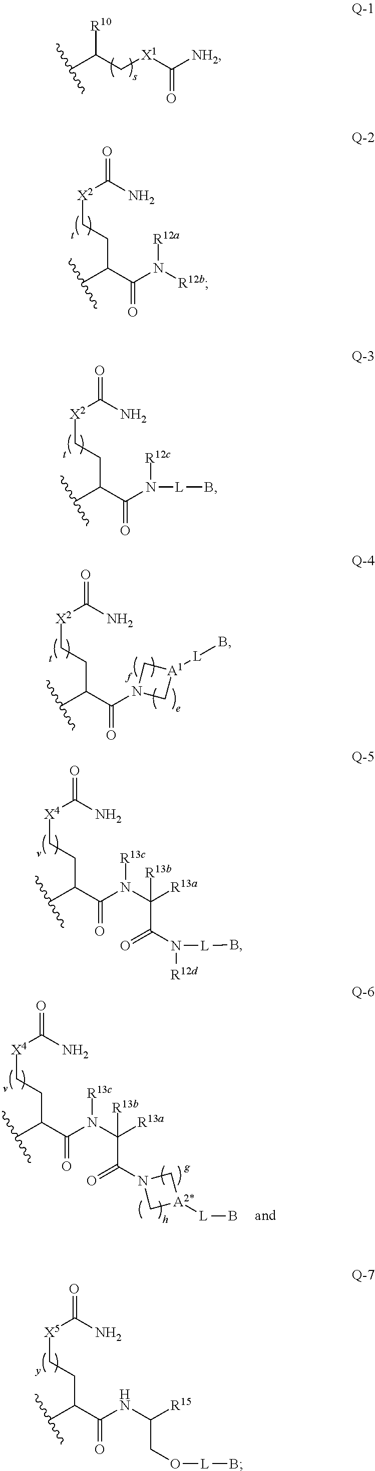
X¹ is selected from the group consisting of —CH₂—, —O—, and —N(R^{11a})—; or
X¹ is absent;
R¹⁰ is selected from the group consisting of hydrogen, C₁-C₆ alkyl, optionally substituted aralkyl, optionally substituted C₃-C₆ cycloalkyl, optionally substituted 4- to 8-membered heterocyclo, and optionally substituted aryl;
R^{11a} is selected from the group consisting of hydrogen and C₁-C₃ alkyl;
s is 1, 2, 3, or 4;
X² is selected from the group consisting of —CH₂—, —O—, and —N(R^{11b})—; or
X² is absent;
t is 0, 1, 2, 3, or 4;
R^{11b} is selected from the group consisting of hydrogen and C₁-C₃ alkyl;
R^{12a} is selected from the group consisting of hydrogen, C₁-C₆ alkyl, optionally substituted C₂-C₆ alkynyl, aralkyl, (heteroaryl)alkyl, optionally substituted C₃-C₆ cycloalkyl, optionally substituted 4- to 8-membered heterocyclo, optionally substituted aryl, (amido)(aryl)alkyl, (amino)(aryl)alkyl, (amino)(heteroaryl)alkyl, and (cycloalkyl)alkyl;
R^{12b} is selected from the group consisting of hydrogen, C₁-C₄ alkyl, optionally substituted aryl, and aralkyl; or
R^{12a} and R^{12b} taken together with the nitrogen atom to which they are attached form a 4- to 8-membered optionally substituted heterocyclo;
R^{12c} is selected from the group consisting of hydrogen, C₁-C₄ alkyl, and aralkyl;
A¹ is selected from the group consisting of —C(R^{14a})— and —N—;
R^{14a} is selected from the group consisting of hydrogen and C₁-C₃ alkyl;
e is 1, 2, or 3;
f is 1, 2, or 3;
X⁴ is selected from the group consisting of —CH₂—, —O—, and —N(R^{11d})—; or
X⁴ is absent;
v is 0, 1, 2, 3, or 4;
R^{11d} is selected from the group consisting of hydrogen and C₁-C₃ alkyl;
R^{12d} is selected from the group consisting of hydrogen, C₁-C₄ alkyl, and aralkyl;
R^{13a} is selected from the group consisting of hydrogen, C₁-C₆ alkyl, optionally substituted C₃-C₁₂ cycloalkyl, optionally substituted aryl, aralkyl, (heteroaryl)alkyl, (cycloalkyl)alkyl, and optionally substituted 5- to 9-membered heteroaryl;
R^{13b} is selected from the group consisting of hydrogen and C₁-C₄ alkyl;
R^{13c} is selected from the group consisting of hydrogen and C₁-C₄ alkyl; or
R^{13a} and R^{13b} taken together form a C₃-C₈ optionally substituted cycloalkyl or C₄-C₉ optionally substituted heterocyclo; or
R^{13b} and R^{13c} taken together form a 4- to 9-membered optionally substituted heterocyclo;
A^{2*} is selected from the group consisting of —C(R^{14b})— and —N—;
R^{14b} is selected from the group consisting of hydrogen and C₁-C₃ alkyl;
g is 1, 2, or 3;
h is 1, 2, or 3;
X⁵ is selected from the group consisting of —CH₂—, —O—, and —N(R^{11e})—; or
X⁵ is absent;
y is 0, 1, 2, 3, or 4;
R^{11e} is selected from the group consisting of hydrogen and C₁-C₃ alkyl;
R¹⁵ is selected from the group consisting of hydrogen, C₁-C₆ alkyl, C₃-C₈ cycloalkyl, optionally substituted aryl, and optionally substituted 5- to 9-membered heteroaryl;
L is -J¹-Y¹-J²-Y²-J³-Z—;
J¹ is selected from the group consisting of alkylenyl, heteroalkylenyl, cycloalkylenyl, heterocyclenyl, phenylenyl, and heteroarylenyl; or
J¹ is absent;
Y¹ is selected from the group consisting of —(CH₂)ₘ—, —C≡C—, —CH═CH—, —N(R^{16a}), —C(═O)—, —S(═O)₂—, —C(═O)O—, —OC(═O)—, —C(═O)N(R^{16b})—, and —N(R^{16b})C(═O)—;
m is 0, 1, 2, or 3;
R^{16a} is selected from the group consisting of hydrogen, C₁-C₄ alkyl, and aralkyl;
R^{16b} is selected from the group consisting of hydrogen and C₁-C₄ alkyl;
J² is selected from the group consisting of alkylenyl, heteroalkylenyl, cycloalkylenyl, heterocyclenyl, phenylenyl, and heteroarylenyl; or
J² is absent;
Y² is selected from the group consisting of —(CH₂)ₙ—, —C≡C—, —CH═CH—, —N(R^{16a})—, —C(═O)—, —S(═O)₂—, —C(═O)O—, —OC(═O)—, —C(═O)N(R^{16b}), and —(R^{16b})C(═O)N—;
n is 0, 1, 2, 3, 4, 5, or 6;
R^{16a} is selected from the group consisting of hydrogen, C₁-C₄ alkyl, and aralkyl;
R^{16b} is selected from the group consisting of hydrogen and C₁-C₄ alkyl;
J³ is selected from the group consisting of alkylenyl, heteroalkylenyl, cycloalkylenyl, heterocyclenyl, phenylenyl, and heteroarylenyl; or
J³ is absent;
Z is selected from the group consisting of —(CH₂)_{d}—, —C≡C—, —CH═CH—, —C(═O)—, —O—, —S—, —N(R^{16c})—, —C(═O)N(R^{16a})—, —N(R^{16a})C(═O)—, —N(R^{16e})C(═O)CH₂O—, and —N(R^{16f})C(═O)CH₂N(R^{16g})—;
d is 0, 1, 2, or 3;
R^{16c}, R^{16d}, R^{16e}, R^{16f}, and R^{16g} are each independently selected from the group consisting of hydrogen, C₁-C₄ alkyl, and aralkyl;
wherein Z is attached to B;
B is selected from the group consisting of: 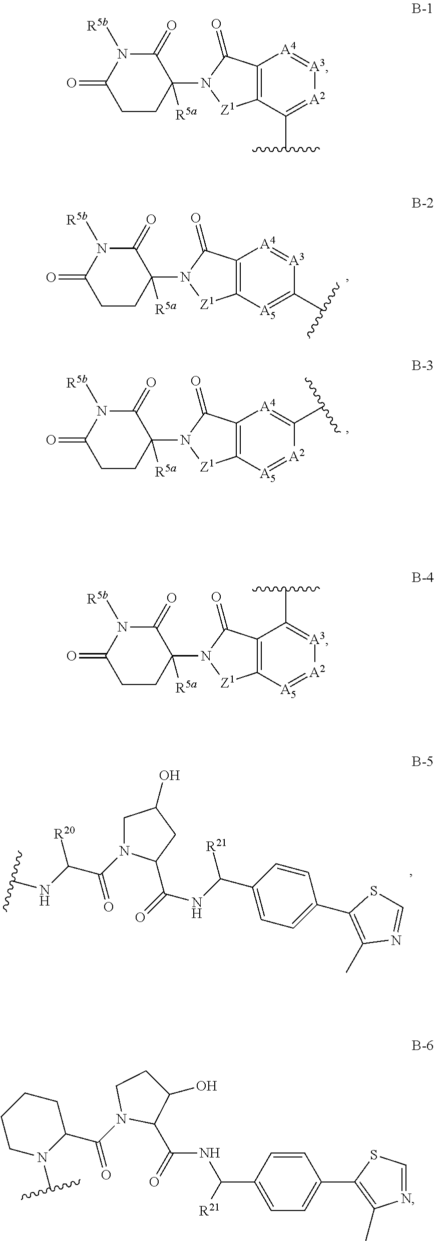 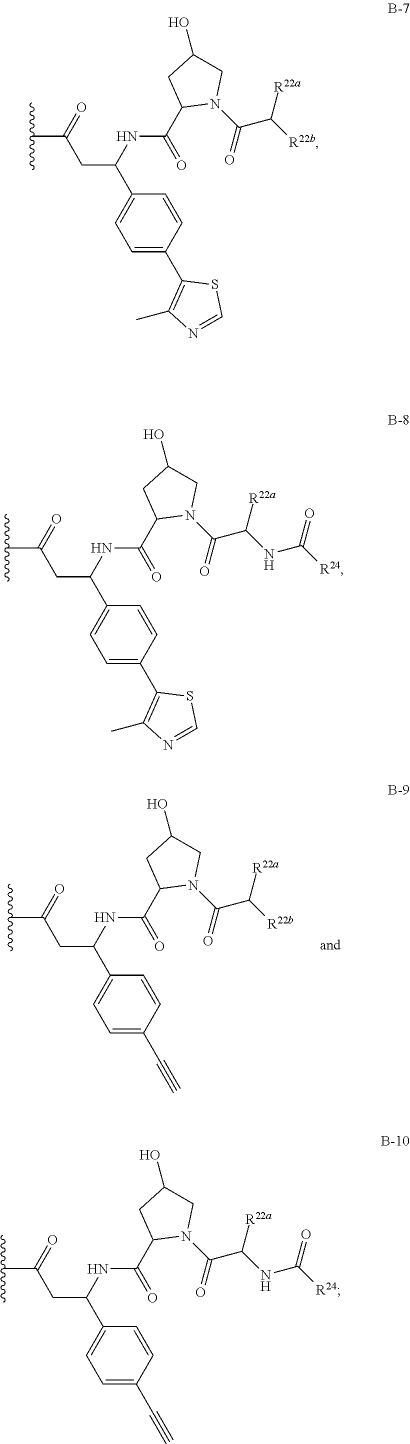
A₅ is selected from the group consisting of —C(R^{19a})═ and —N═;
A² is selected from the group consisting of —C(R^{19b})═ and —N═;
A³ is selected from the group consisting of —C(R^{19c})═ and —N═;
A⁴ is selected from the group consisting of —C(R^{19d})═ and —N═;
Z¹ is selected from the group consisting of —CH₂ and —C(═O)—;
R^{5a} is selected from the group consisting of hydrogen, methyl, and fluoro;
R^{5b} is selected from the group consisting of hydrogen and methyl;
R^{19a}, R^{19b}, R^{19c}, and R^{19d} are each independently selected from the group consisting of hydrogen, halo, and C₁₋₄ alkyl;
R²⁰ is C₁-C₆ alkyl;
R²¹ is selected from the group consisting of hydrogen and C₁-C₄ alkyl;
R^{22a} is selected from the group consisting of C₁-C₆ alkyl and optionally substituted C₃-C₆ cycloalkyl;
R^{22b} is selected from the group consisting of C₁-C₆ alkyl, optionally substituted C₃-C₆ cycloalkyl, optionally substituted aryl, and optionally substituted heteroaryl; and
R²³ is selected from the group consisting of C₁-C₆ alkyl and optionally substituted C₃-C₆ cycloalkyl; and
R²⁴ is selected from the group consisting of C₁-C₆ alkyl, optionally substituted C₃-C₆ cycloalkyl, optionally substituted aryl, and optionally substituted heteroaryl.

2. (canceled)

4. The method of claim 1, wherein the subject is a human.

5. The method of claim 1, wherein the GVHD is acute GVHD, late acute GVHD, chronic GVHD, or late chronic GVHD.

6.-8. (canceled)

9. The method of claim 1, wherein the administration results in reduced STAT3 activation in alloreactive T Cells, preserved graft-versus-leukemia (GVL) effect, or the graft-versus-leukemia (GVL) effect is preserved.

10. (canceled)

11. (canceled)

12. The method of claim 1, wherein a second therapeutic agent is administered.

13. The method of claim 1, wherein:
(1) when X_{A} is —CH₂CH₂—, then Q^{A} is selected from the group consisting of Q-3, Q-4, Q-5, Q-6, and Q-7;
(2) when X_{A} is —N(R⁸)CH₂— or —CH₂N(R⁸)—, and R⁸ is selected from the group consisting of hydrogen, C₁-C₆ alkyl, C₁-C₆ haloalkyl, (heterocyclo)alkyl, alkylsulfonyl, and —C(═O)R⁹, then Q^{A} is selected from the group consisting of Q-3, Q-4, Q-5, Q-6, and Q-7; and/or
(3) when X_{A} is —N(R⁸)CH₂— or —CH₂N(R⁸)—, and R⁸ is -L-B, then Q^{A} is selected from the group consisting of Q-1 and Q-2.

14. The method of claim 1, wherein the compound is of Formula (II) or (III): 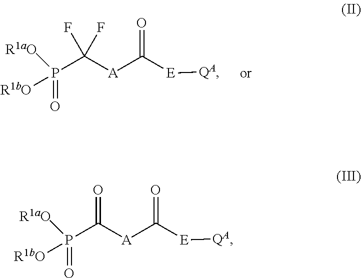
or a pharmaceutically acceptable salt or solvate thereof.

15. (canceled)

16. The method of claim 1, wherein the compound is of Formula (IV), (V), (IV-A), (V-A), (VI), (VII), (VI-A), (VII-A), (VI-B), (VII-B), (VI-C), (VII-C), (VII-D), or (VII-E): 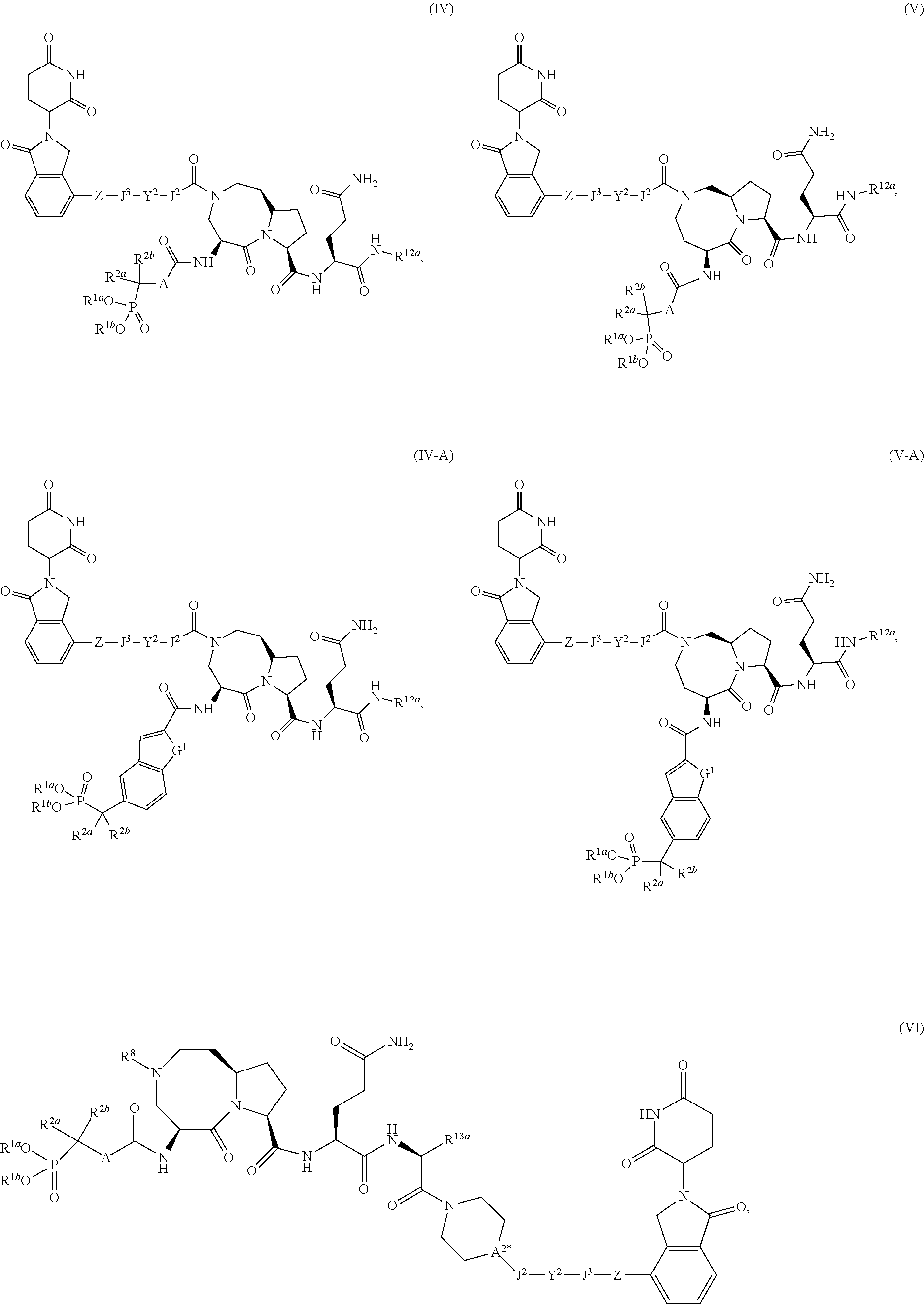 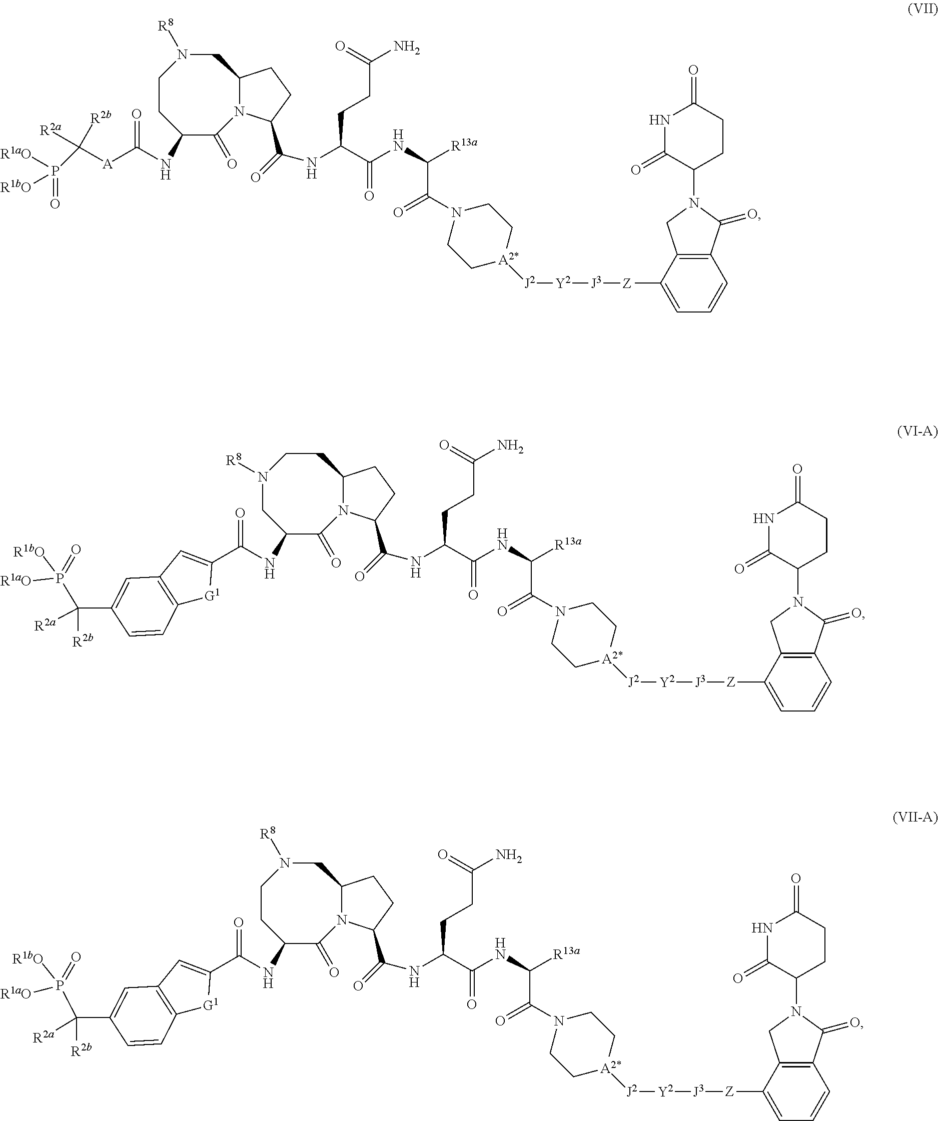 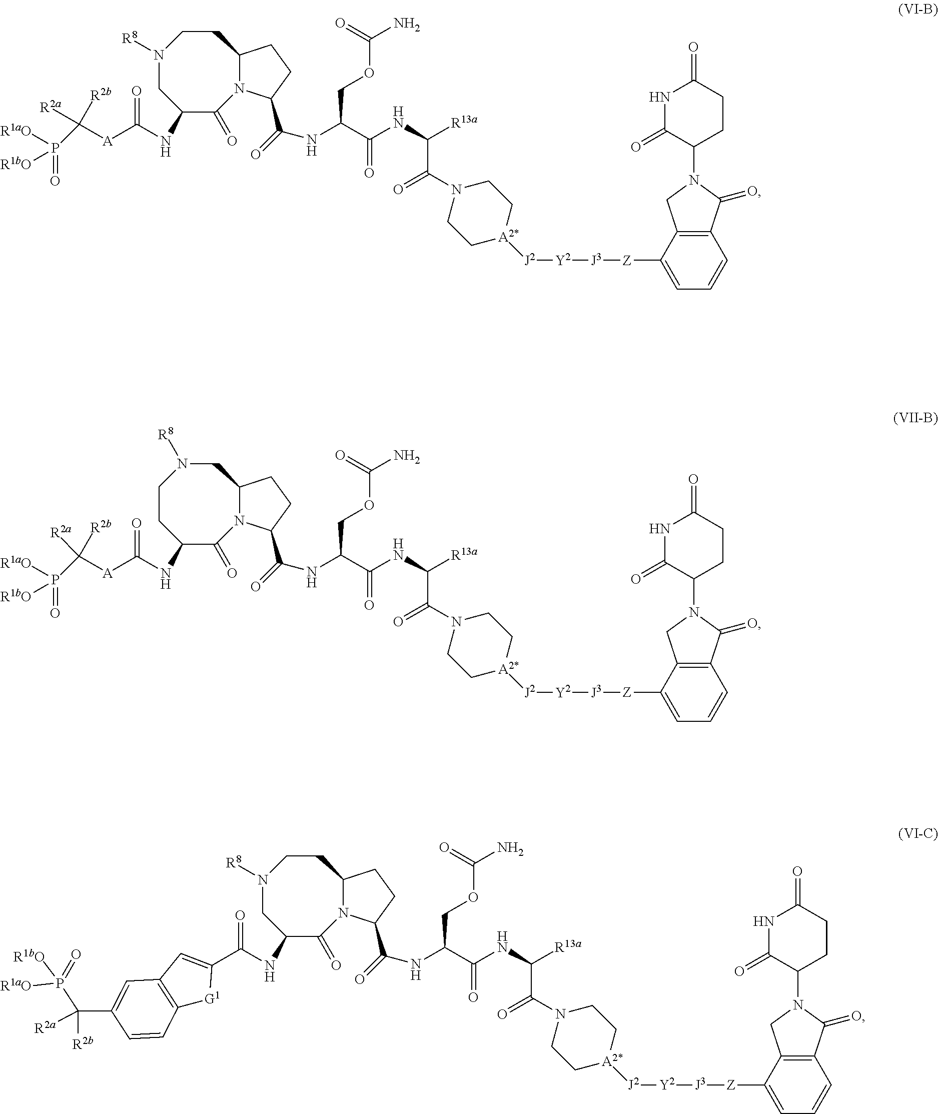 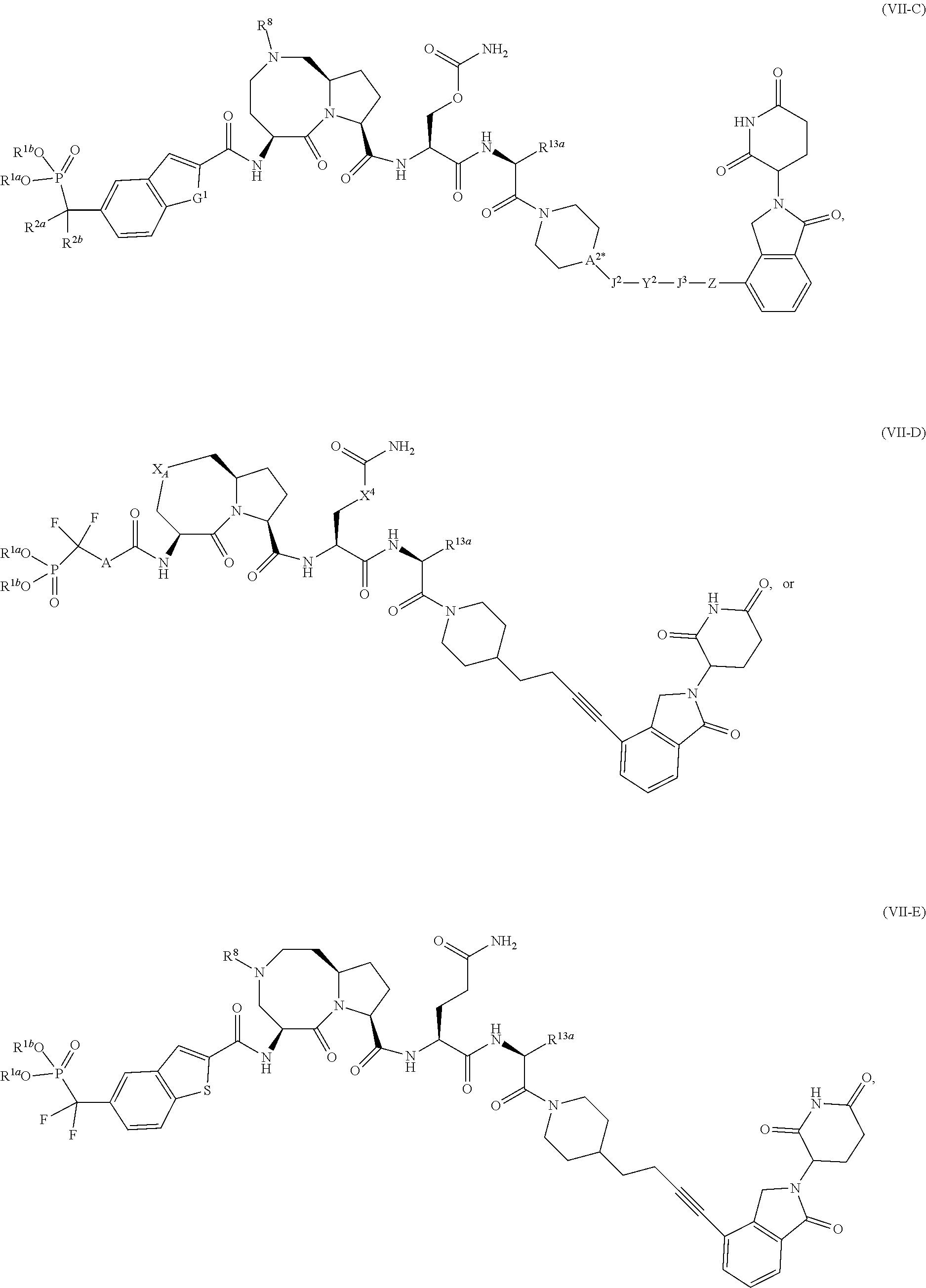
or a pharmaceutically acceptable salt or solvate thereof.

17.-29. (canceled)

30. The method of claim 1, wherein the compound is of Formula (XXII), (XXIII), (XXIV), or (XXVI): 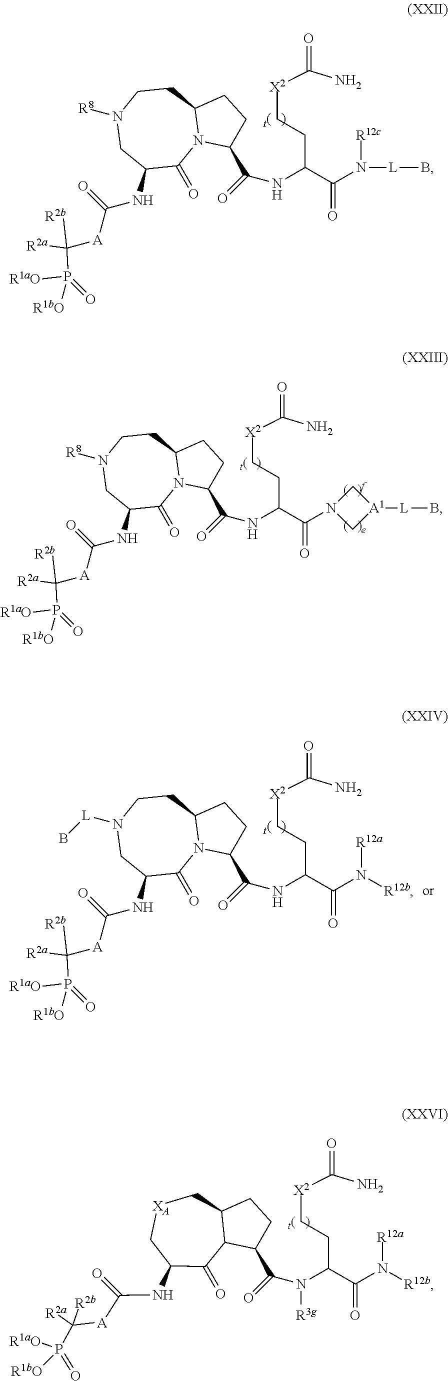
or a pharmaceutically acceptable salt or solvate thereof, wherein:
R^{1a} and R^{1b} are independently selected from the group consisting of hydrogen, C₁-C₆ alkyl, and aralkyl; and
R⁸ is selected from the group consisting of hydrogen, C₁-C₆ alkyl, C₁-C₆ haloalkyl, (heterocyclo)alkyl, alkylsulfonyl, and —C(═O)R⁹.

31.-33. (canceled)

34. The method of claim 1, wherein the compound is selected from the compounds described in Tables 1, 1A and 1B, or pharmaceutically acceptable salts thereof, and solvates thereof.

35.-37. (canceled)

38. The method of claim 1, wherein the compound is selected from group consisting of:
((2-(((5S,8S,10aR)-8-(((S)-5-amino-1-(benzhydrylamino)-1,5-dioxopentan-2-yl)carbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)-1H-indol-5-yl)difluoromethyl)phosphonic acid;
(2-(((5S,8S,10aR)-8-(((S)-5-amino-1-(benzhydrylamino)-1,5-dioxopentan-2-yl)carbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)-1H-indole-5-carbonyl)phosphonic acid;
(2-(((5S,8S,10aR)-8-(((2S)-5-amino-1-(((1S)-2-(4-(4-(2-(2,6-dioxopiperidin-3-yl)-1-oxoisoindolin-4-yl)but-3-yn-1-yl)piperidin-1-yl)-2-oxo-1-phenylethyl)amino)-1,5-dioxopentan-2-yl)carbamoyl)-3-methyl-6-oxodecahydropyrrolo[1,2-a][1,5]diazocin-5-yl)carbamoyl)-1H-indole-5-carbonyl)phosphonic acid;
(2-(((5S,8S,10aR)-8-(((2S)-5-amino-1-(((1S)-1-cyclohexyl-2-(4-(4-(2-(2,6-dioxopiperidin-3-yl)-1-oxoisoindolin-4-yl)but-3-yn-1-yl)piperidin-1-yl)-2-oxoethyl)amino)-1,5-dioxopentan-2-yl)carbamoyl)-3-methyl-6-oxodecahydropyrrolo[1,2-a][1,5]diazocin-5-yl)carbamoyl)-1H-indole-5-carbonyl)phosphonic acid;
((2-(((5S,8S,10aR)-8-(((S)-5-amino-1-(benzhydrylamino)-1,5-dioxopentan-2-yl)carbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)benzo[b]thiophen-5-yl)difluoromethyl)phosphonic acid;
((2-(((5S,8S,10aR)-8-(((2S)-5-amino-1-(((1S)-1-cyclohexyl-2-(4-(4-(2-(2,6-dioxopiperidin-3-yl)-1-oxoisoindolin-4-yl)but-3-yn-1-yl)piperidin-1-yl)-2-oxoethyl)amino)-1,5-dioxopentan-2-yl)carbamoyl)-3-methyl-6-oxodecahydropyrrolo[1,2-a][1,5]diazocin-5-yl)carbamoyl)benzo[b]thiophen-5-yl)difluoromethyl)phosphonic acid;
((2-(((5S,8S,10aR)-8-(((2S)-5-amino-1-(((1S)-2-(4-(4-(2-(2,6-dioxopiperidin-3-yl)-1-oxoisoindolin-4-yl)but-3-yn-1-yl)piperidin-1-yl)-2-oxo-1-phenylethyl)amino)-1,5-dioxopentan-2-yl)carbamoyl)-3-methyl-6-oxodecahydropyrrolo[1,2-a][1,5]diazocin-5-yl)carbamoyl)benzo[b]thiophen-5-yl)difluoromethyl)phosphonic acid
(2-(((5S,8S,10aR)-8-(((S)-5-amino-1-(benzhydrylamino)-1,5-dioxopentan-2-yl)carbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)benzo[b]thiophene-5-carbonyl)phosphonic acid;
(2-(((5S,8S,10aR)-8-(((2S)-5-amino-1-(((1S)-1-cyclohexyl-2-(4-(4-(2-(2,6-dioxopiperidin-3-yl)-1-oxoisoindolin-4-yl)but-3-yn-1-yl)piperidin-1-yl)-2-oxoethyl)amino)-1,5-dioxopentan-2-yl)carbamoyl)-3-methyl-6-oxodecahydropyrrolo[1,2-a][1,5]diazocin-5-yl)carbamoyl)benzo[b]thiophene-5-carbonyl)phosphonic acid; and
(2-(((5S,8S,10aR)-8-(((2S)-5-amino-1-(((1S)-2-(4-(4-(2-(2,6-dioxopiperidin-3-yl)-1-oxoisoindolin-4-yl)but-3-yn-1-yl)piperidin-1-yl)-2-oxo-1-phenylethyl)amino)-1,5-dioxopentan-2-yl)carbamoyl)-3-methyl-6-oxodecahydropyrrolo[1,2-a][1,5]diazocin-5-yl)carbamoyl)benzo[b]thiophene-5-carbonyl)phosphonic acid,
and pharmaceutically acceptable salts and solvates thereof.

39. The method of claim 1, wherein the compound is selected from group consisting of:
((2-(((5S,8S,10aR)-8-(((2S)-5-amino-1-(((2S)-3-(3,4-difluorophenyl)-1-(4-(4-(2-(2,6-dioxopiperidin-3-yl)-1-oxoisoindolin-4-yl)but-3-yn-1-yl)piperidin-1-yl)-1-oxopropan-2-yl)amino)-1,5-dioxopentan-2-yl)carbamoyl)-3-(methoxycarbonyl)-6-oxodecahydropyrrolo[1,2-a][1,5]diazocin-5-yl)carbamoyl)benzo[b]thiophen-5-yl)difluoromethyl)phosphonic acid; and
((2-(((5S,8S,10aR)-8-(((2S)-5-amino-1-(((1S)-1-cyclohexyl-2-(4-(4-(2-(2,6-dioxopiperidin-3-yl)-1-oxoisoindolin-4-yl)but-3-yn-1-yl)piperidin-1-yl)-2-oxoethyl)amino)-1,5-dioxopentan-2-yl)carbamoyl)-3-(methoxycarbonyl)-6-oxodecahydropyrrolo[1,2-a][1,5]diazocin-5-yl)carbamoyl)benzo[b]thiophen-5-yl)difluoromethyl)phosphonic acid, and pharmaceutically acceptable salts and solvates thereof.

40. The method of claim 1, wherein the compound is ((2-(((5S,8S,10aR)-8-(((2S)-5-amino-1-(((2S)-3-(3,4-difluorophenyl)-1-(4-(4-(2-(2,6-dioxopiperidin-3-yl)-1-oxoisoindolin-4-yl)but-3-yn-1-yl)piperidin-1-yl)-1-oxopropan-2-yl)amino)-1,5-dioxopentan-2-yl)carbamoyl)-3-(methoxycarbonyl)-6-oxodecahydropyrrolo[1,2-a][1,5]diazocin-5-yl)carbamoyl)benzo[b]thiophen-5-yl)difluoromethyl)phosphonic acid, or a pharmaceutically acceptable salt thereof.

41. The method of claim 1, wherein the compound is:

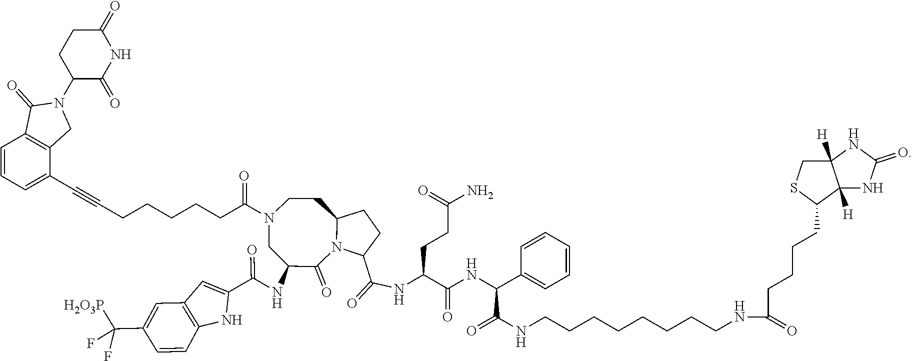

42. The method of claim 1, wherein the compound is:

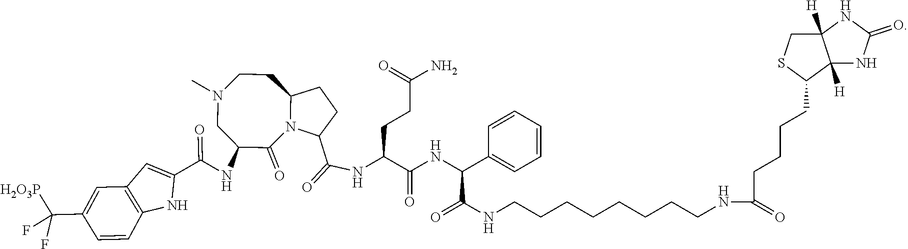

43. The method of claim 1, wherein the compound is:

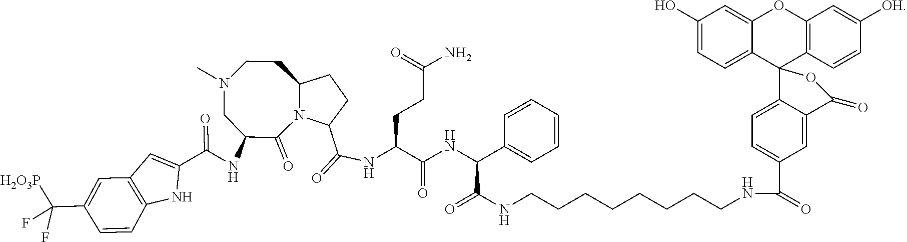

44. The method of claim 1, wherein the compound is of Formula (VIII): 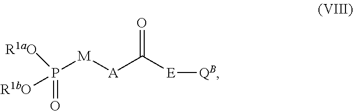
or a pharmaceutically acceptable salt or solvate thereof, wherein:
R^{1a} and R^{1b} are independently selected from the group consisting of hydrogen, C₁-C₆ alkyl, and aralkyl;
R^{2a}, R^{2b}, M, A, and E are as defined in connection with Formula I, except that in A the bond designated with an “*” is attached to —C(═O)-E-Q^{B} and that in E the bond designated with an “*” is attached to Q^{B};
R⁸ is selected from the group consisting of hydrogen, C₁-C₆ alkyl, C₁-C₆ haloalkyl, (heterocyclo)alkyl, alkylsulfonyl, and —C(═O)R⁹; and
Q^{B} is selected from the group consisting of Q-1 and Q-2.

45. The method of claim 1, wherein:
(1) when X_{A} is —CH₂CH₂—, then:
(i) A is selected from the group consisting of A-2, A-5, A-6, A-7, A-8, A-9, A-10, A-11, A-12, A-13, A-14, A-15, A-16, A-17, A-18, A-19, and A-20;
(ii) A is A-4 and G¹ is —S—; or
(iii) R^{2a} and R^{2b} taken together with the carbon atom to which they are attached form a —C(═O)— group;
(2) when X_{A} is —N(R⁸)CH₂—, then:
(i) A is selected from the group consisting of A-1, A-2, A-4, A-5, A-6, A-7, A-8, A-9, A-10, A-11, A-12, A-13, A-14, A-15, A-16, A-17, A-18, A-19, and A-20; or
(ii) R^{2a} and R^{2b} taken together with the carbon atom to which they are attached form a —C(═O)— group; or
(3) when X_{A} is —CH₂N(R⁸)—, then:
(i) A is selected from the group consisting of A-1, A-2, A-4, A-5, A-6, A-7, A-8, A-9, A-10, A-11, A-12, A-13, A-14, A-15, A-16, A-17, A-18, A-19, and A-20; or
(ii) R^{2a} and R^{2b} taken together with the carbon atom to which they are attached form a —C(═O)— group.

46. The method of claim 1, wherein the compound is of Formula (IX), (X), (XI), or (XII): 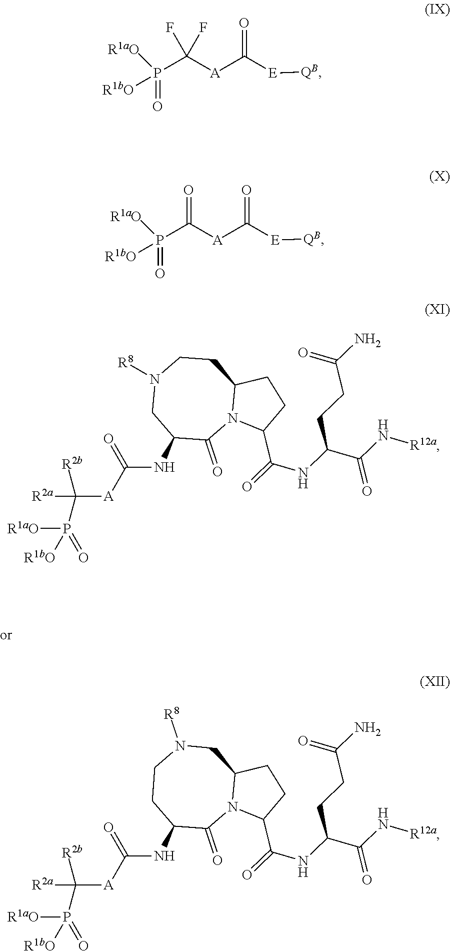
or a pharmaceutically acceptable salt or solvate thereof.

47.-49. (canceled)

50. The method of claim 1, wherein the compound is selected from the compounds described in Table 2, and pharmaceutically acceptable salts and solvates thereof.
